(19) United States
(12) Patent Application Publication
(10) Pub. No.: US 2024/0043489 A1
(43) Pub. Date: Feb. 8, 2024

(54) Cytokine Prodrugs Comprising a Cleavable Linker

(71) Applicant: Trutino Biosciences Inc., San Diego, CA (US)

(72) Inventors: Phillip S. Kim, San Diego, CA (US); Emma Langley, San Diego, CA (US); Hsieng Lu, Thousand Oaks, CA (US); Xinjun Liu, San Diego, CA (US)

(73) Assignee: Trutino Biosciences Inc., San Diego, CA (US)

(21) Appl. No.: 17/792,976

(22) PCT Filed: Jan. 14, 2021
(86) PCT No.: PCT/US2021/013478

§ 371 Date: Jan. 11, 2023

Related U.S. Application Data
(60) Provisional application No. 62/961,537, filed on Jan. 15, 2020.

(51) Int. Cl. C07K 14/55; A61K 47/65; A61K 47/68; C07K 14/715; A61P 35/00; A61K 47/60

(52) U.S. Cl. CPC C07K 14/55; A61K 47/65; A61K 47/6813; C07K 14/7155; A61P 35/00; A61K 47/60

(57) ABSTRACT

This disclosure relates to protease-cleavable cytokine prodrugs. In some embodiments, the prodrugs comprise a targeting sequence. In some embodiments, the prodrugs comprise a pharmacokinetic modulator.

### CROSS-REFERENCE TO RELATED APPLICATIONS

This application claims the benefit of priority of U.S. Provisional Patent Application No. 62/961,537, filed Jan. 15, 2020, which is incorporated herein by reference in its entirety for all purposes.

### INTRODUCTION AND SUMMARY

This disclosure relates to the field of cytokine therapeutics, particularly cytokine prodrugs comprising a cleavable linker.

Cytokines, such as IL-2, are powerful immune growth factors that play a significant role in sustaining an effective immune cell response. IL-2 has been reported to induce complete and durable regressions in cancer patients but immune related adverse effects have reduced its therapeutic potential. In some cases, however, systemic IL-2 administration can activate immune cells throughout the body. Systemic activation can lead to systemic toxicity and indiscriminate activation of immune cells, including immune cells that respond to a variety of epitopes, antigens, and stimuli. The therapeutic potential of IL-2 therapy can be impacted by these severe toxicities.

IL-2 therapies can also suffer from a short serum half-life, sometimes on the order of several minutes. Thus, the high doses of IL-2 that can be necessary to achieve an optimal immune-modulatory effect can contribute to severe toxicities.

As a result, cytokine therapeutics that overcome the hurdles of systemic or untargeted function, severe toxicity, and poor pharmacokinetics, are needed. The present disclosure aims to meet one or more of these needs, provide other benefits, or at least provide the public with a useful choice.

In some aspects, protease-activated pro-cytokines (also referred to as cytokine prodrugs) are provided, which can be administered to a subject in an inactive form. The inactive form can include a cytokine polypeptide sequence, a protease-cleavable polypeptide sequence, and an inhibitory polypeptide sequence capable of blocking an activity of the cytokine polypeptide sequence. Such prodrugs can become activated when the protease-cleavable polypeptide sequence is cleaved by a protease. Cleaving the protease-cleavable polypeptide can allow the inhibitory polypeptide sequence to dissociate from the cytokine polypeptide sequence.

Many tumors and tumor microenvironments exhibit aberrant expression of proteases. The present disclosure provides cytokine prodrugs that are activatable through proteolytic cleavage, such that they become active when they come in contact with proteases in a tumor or tumor microenvironment. In some cases, this can lead to an increase in active cytokines in and around the tumor or tumor microenvironment relative to the rest of a subject's body or healthy tissue. One exemplary advantage that can result is the formation of cytokine gradients. Such a gradient can form when a cytokine prodrug is administered and selectively or preferentially becomes activated in the tumor or tumor microenvironment and subsequently diffuses out of these areas to the rest of the body. These gradients can increase the trafficking of immune cells to the tumor and tumor microenvironment. Immune cells that traffic to the tumor can infiltrate the tumor. Infiltrating immune cells can mount an immune response against the cancer. Infiltrating immune cells can also secrete their own chemokines and cytokines. The cytokines can have either or both of autocrine and paracrine effects within the tumor and tumor microenvironment. In some cases, the immune cells include T cells, such as T effector cells or cytotoxic T cells, or NK cells.

Also described herein are methods of treatment and methods of administrating the cytokine prodrugs described herein. Such administration can be systemic or local. In some embodiments, a cytokine prodrug described herein is administered systemically or locally to treat a cancer.

A further example of local administration is administration of a cytokine prodrug, such as an IL-2 cytokine prodrug, to boost T regulatory cells. In some cases, the local administration of an IL-2 cytokine prodrug is to an area of inflammation. Such a method can be used to treat chronic autoimmune and/or inflammatory diseases.

The following embodiments are encompassed.

Embodiment 1 is a protease-activated pro-cytokine comprising:
a cytokine polypeptide sequence;
a inhibitory polypeptide sequence capable of blocking an activity of the cytokine polypeptide sequence;
a linker between the cytokine polypeptide sequence and the inhibitory polypeptide sequence,
the linker comprising a protease-cleavable polypeptide sequence; and
a targeting sequence, wherein the targeting sequence is configured to bind an extracellular matrix component, an integrin, or a syndecan; or is configured to bind, in a pH-sensitive manner, an extracellular matrix component, IgB (CD79b), an integrin, a cadherin, a heparan sulfate proteoglycan, a syndecan, or a fibronectin; or the targeting sequence comprises the sequence of any one of SEQ ID NOs: 180-662 or a variant having one or two mismatches relative to the sequence of any one of SEQ ID NOs: 180-662.

Embodiment 2 is the protease-activated pro-cytokine of the immediately preceding embodiment, further comprising a pharmacokinetic modulator.

Embodiment 3 is the protease-activated pro-cytokine of the immediately preceding embodiment, wherein the pharmacokinetic modulator comprises an immunoglobulin constant domain.

Embodiment 4 is the protease-activated pro-cytokine of embodiment 2, wherein the pharmacokinetic modulator comprises an immunoglobulin Fc region.

Embodiment 5 is the protease-activated pro-cytokine of the immediately preceding embodiment, wherein the immunoglobulin is a human immunoglobulin.

Embodiment 6 is the protease-activated pro-cytokine of any one of embodiments 4-5, wherein the immunoglobulin is IgG.

Embodiment 7 is the protease-activated pro-cytokine of the immediately preceding embodiment, wherein the IgG is IgG1, IgG2, IgG3, or IgG4.

Embodiment 8 is the protease-activated pro-cytokine of embodiment 2, wherein the pharmacokinetic modulator comprises an albumin.

Embodiment 9 is the protease-activated pro-cytokine of the immediately preceding embodiment, wherein the albumin is a serum albumin.

Embodiment 10 is the protease-activated pro-cytokine of any one of embodiments 8-9, wherein the albumin is a human albumin.

Embodiment 11 is the protease-activated pro-cytokine of embodiment 2, wherein the pharmacokinetic modulator comprises PEG.

Embodiment 12 is the protease-activated pro-cytokine of embodiment 2, wherein the pharmacokinetic modulator comprises XTEN.

Embodiment 13 is the protease-activated pro-cytokine of embodiment 2, wherein the pharmacokinetic modulator comprises CTP.

Embodiment 14 is the protease-activated pro-cytokine of any one of embodiments 2-13, wherein the protease-cleavable polypeptide sequence is between the cytokine polypeptide sequence and the pharmacokinetic modulator.

Embodiment 15 is the protease-activated pro-cytokine of any one of embodiments 2-13, wherein the pharmacokinetic modulator is between the cytokine polypeptide sequence and the protease-cleavable polypeptide sequence.

Embodiment 16 is the protease-activated pro-cytokine of any one of the preceding embodiments, comprising a plurality of protease-cleavable polypeptide sequences.

Embodiment 17 is the protease-activated pro-cytokine of the immediately preceding embodiment, wherein the cytokine polypeptide sequence is flanked by protease cleavable polypeptide sequences.

Embodiment 18 is the protease-activated pro-cytokine of the immediately preceding embodiment, having the structure PM-CL-CY-CL-IN (from N- to C-terminus or from C- to N-terminus), where PM is the pharmacokinetic modulator, each CL independently is a protease-cleavable polypeptide sequence, CY is the cytokine polypeptide sequence, and IN is the inhibitory polypeptide sequence.

Embodiment 19 is the protease-activated pro-cytokine of any one of the preceding embodiments, comprising the targeting sequence, wherein the targeting sequence is between the cytokine polypeptide sequence and the protease-cleavable polypeptide sequence or one of the protease-cleavable polypeptide sequences.

Embodiment 20 is the protease-activated pro-cytokine of any one of the preceding embodiments, wherein the cytokine polypeptide sequence comprises a modification to prevent disulfide bond formation, and optionally otherwise comprises wild-type sequence.

Embodiment 21 is the protease-activated pro-cytokine of any one of the preceding embodiments, wherein the cytokine polypeptide sequence has at least 80, 85, 90, 95, 97, 98, or 99 percent identity to the sequence of a wild-type cytokine polypeptide sequence or to a cytokine polypeptide sequence in Table 1.

Embodiment 22 is the protease-activated pro-cytokine of the immediately preceding embodiment, wherein the cytokine polypeptide sequence is a wild-type cytokine polypeptide sequence.

Embodiment 23 is the protease-activated pro-cytokine of any one of the preceding embodiments, wherein the cytokine polypeptide sequence is a monomeric cytokine, or wherein the cytokine polypeptide sequence is a dimeric cytokine polypeptide sequence comprising monomers that are associated covalently (optionally via a polypeptide linker) or noncovalently.

Embodiment 24 is the protease-activated pro-cytokine of any one of the preceding embodiments, wherein the inhibitory polypeptide sequence comprises a cytokine-binding domain.

Embodiment 25 is the protease-activated pro-cytokine of the immediately preceding embodiment, wherein the cytokine-binding domain is a cytokine-binding domain of a cytokine receptor or a cytokine-binding domain of a fibronectin.

Embodiment 26 is the protease-activated pro-cytokine of embodiment 24, wherein the cytokine-binding domain is an immunoglobulin cytokine-binding domain.

Embodiment 27 is the protease-activated pro-cytokine of the immediately preceding embodiment, wherein the immunoglobulin cytokine-binding domain comprises a light chain variable domain and a heavy chain variable domain that bind the cytokine.

Embodiment 28 is the protease-activated pro-cytokine of any one of embodiments 26-27, wherein the immunoglobulin cytokine-binding domain is an scFv, Fab, or VHH.

Embodiment 29 is the protease-activated pro-cytokine of any one of the preceding embodiments, wherein the protease-cleavable polypeptide sequence is recognized by a metalloprotease, a serine protease, a cysteine protease, an aspartate protease, a threonine protease, a glutamate protease, a gelatinase, an asparagine peptide lyase, a cathepsin, a kallikrein, a plasmin, a collagenase, a hK1, a hK10, a hK15, a stromelysin, a Factor Xa, a chymotrypsin-like protease, a trypsin-like protease, a elastase-like protease, a subtilisin-like protease, an actinidain, a bromelain, a calpain, a caspase, a Mir 1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, an ADAM 10, an ADAM17, an ADAM 12, an urokinase plasminogen activator (uPA), an enterokinase, a prostate-specific target (PSA, hK3), an interleukin-1b converting enzyme, a thrombin, a FAP (FAP-a), a dipeptidyl peptidase, or dipeptidyl peptidase IV (DPPIV/CD26), a type II transmembrane serine protease (TTSP), a neutrophil elastase, a proteinase 3, a mast cell chymase, a mast cell tryptase, or a dipeptidyl peptidase.

Embodiment 30 is the protease-activated pro-cytokine of any one of the preceding embodiments, wherein the protease-cleavable polypeptide sequence comprises the sequence of any one of SEQ ID NOs: 700-741, or a variant having one or two mismatches relative to the sequence of any one of SEQ ID NOs: 700-741.

Embodiment 31 is the protease-activated pro-cytokine of any one of the preceding embodiments, wherein the protease-cleavable polypeptide sequence is recognized by a matrix metalloprotease.

Embodiment 32 is the protease-activated pro-cytokine of any one of the preceding embodiments, wherein the protease-cleavable polypeptide sequence is recognized by MMP-1.

Embodiment 33 is the protease-activated pro-cytokine of any one of the preceding embodiments, wherein the protease-cleavable polypeptide sequence is recognized by MMP-2.

Embodiment 34 is the protease-activated pro-cytokine of any one of the preceding embodiments, wherein the protease-cleavable polypeptide sequence is recognized by MMP-3.

Embodiment 35 is the protease-activated pro-cytokine of any one of the preceding embodiments, wherein the protease-cleavable polypeptide sequence is recognized by MMP-7.

Embodiment 36 is the protease-activated pro-cytokine of any one of the preceding embodiments, wherein the protease-cleavable polypeptide sequence is recognized by MMP-8.

Embodiment 37 is the protease-activated pro-cytokine of any one of the preceding embodiments, wherein the protease-cleavable polypeptide sequence is recognized by MMP-9.

Embodiment 38 is the protease-activated pro-cytokine of any one of the preceding embodiments, wherein the protease-cleavable polypeptide sequence is recognized by MMP-12.

Embodiment 39 is the protease-activated pro-cytokine of any one of the preceding embodiments, wherein the protease-cleavable polypeptide sequence is recognized by MMP-13.

Embodiment 40 is the protease-activated pro-cytokine of any one of the preceding embodiments, wherein the protease-cleavable polypeptide sequence is recognized by MMP-14.

Embodiment 41 is the protease-activated pro-cytokine of any one of the preceding embodiments, wherein the protease-cleavable polypeptide sequence is recognized by more than one MMP.

Embodiment 42 is the protease-activated pro-cytokine of any one of the preceding embodiments, wherein the protease-cleavable polypeptide sequence is recognized by two, three, four, five, six, or seven of MMP-2, MMP-7, MMP-8, MMP-9, MMP-12, MMP-13, and MMP-14.

Embodiment 43 is the protease-activated pro-cytokine of any one of the preceding embodiments, wherein the protease-cleavable polypeptide sequence comprises the sequence of any one of SEQ ID NOs: 80-94 or a variant sequence having one or two mismatches relative to the sequence of any one of SEQ ID NOs: 80-90.

Embodiment 44 is the protease-activated pro-cytokine of the immediately preceding embodiment, wherein the protease-cleavable polypeptide sequence comprises the sequence of SEQ ID NO: 80 or a variant sequence having one or two mismatches relative thereto.

Embodiment 45 is the protease-activated pro-cytokine of any one of embodiments 1-43, wherein the protease-cleavable polypeptide sequence comprises the sequence of SEQ ID NO: 81 or a variant sequence having one or two mismatches relative thereto.

Embodiment 46 is the protease-activated pro-cytokine of any one of embodiments 1-43, wherein the protease-cleavable polypeptide sequence comprises the sequence of SEQ ID NO: 82 or a variant sequence having one or two mismatches relative thereto.

Embodiment 47 is the protease-activated pro-cytokine of any one of embodiments 1-43, wherein the protease-cleavable polypeptide sequence comprises the sequence of SEQ ID NO: 83 or a variant sequence having one or two mismatches relative thereto.

Embodiment 48 is the protease-activated pro-cytokine of any one of embodiments 1-43, wherein the protease-cleavable polypeptide sequence comprises the sequence of SEQ ID NO: 84 or a variant sequence having one or two mismatches relative thereto.

Embodiment 49 is the protease-activated pro-cytokine of any one of embodiments 1-43, wherein the protease-cleavable polypeptide sequence comprises the sequence of SEQ ID NO: 85 or a variant sequence having one or two mismatches relative thereto.

Embodiment 50 is the protease-activated pro-cytokine of any one of embodiments 1-43, wherein the protease-cleavable polypeptide sequence comprises the sequence of SEQ ID NO: 86 or a variant sequence having one or two mismatches relative thereto.

Embodiment 51 is the protease-activated pro-cytokine of any one of embodiments 1-43, wherein the protease-cleavable polypeptide sequence comprises the sequence of SEQ ID NO: 87 or a variant sequence having one or two mismatches relative thereto.

Embodiment 52 is the protease-activated pro-cytokine of any one of embodiments 1-43, wherein the protease-cleavable polypeptide sequence comprises the sequence of SEQ ID NO: 88 or a variant sequence having one or two mismatches relative thereto.

Embodiment 53 is the protease-activated pro-cytokine of any one of embodiments 1-43, wherein the protease-cleavable polypeptide sequence comprises the sequence of SEQ ID NO: 89 or a variant sequence having one or two mismatches relative thereto.

Embodiment 54 is the protease-activated pro-cytokine of any one of embodiments 1-43, wherein the protease-cleavable polypeptide sequence comprises the sequence of SEQ ID NO: 90 or a variant sequence having one or two mismatches relative thereto.

Embodiment 55 is the protease-activated pro-cytokine of any one of embodiments 1-43, wherein the protease-cleavable polypeptide sequence comprises the sequence of SEQ ID NO: 80-89 or 90.

Embodiment 56 is the protease-activated pro-cytokine of any one of embodiments 1-43, wherein the protease-cleavable polypeptide sequence comprises the sequence of SEQ ID NO: 91.

Embodiment 57 is the protease-activated pro-cytokine of any one of embodiments 1-43, wherein the protease-cleavable polypeptide sequence comprises the sequence of SEQ ID NO: 92.

Embodiment 58 is the protease-activated pro-cytokine of any one of embodiments 1-43, wherein the protease-cleavable polypeptide sequence comprises the sequence of SEQ ID NO: 93.

Embodiment 59 is the protease-activated pro-cytokine of any one of embodiments 1-43, wherein the protease-cleavable polypeptide sequence comprises the sequence of SEQ ID NO: 94.

Embodiment 60 is the protease-activated pro-cytokine of any one of the preceding embodiments, wherein the targeting sequence comprises the sequence of any one of SEQ ID NOs: 180-662, or a variant having one or two mismatches relative to the sequence of any one of SEQ ID NOs: 180-662.

Embodiment 61 is the protease-activated pro-cytokine of the immediately preceding embodiment, wherein the targeting sequence comprises the sequence of any one of SEQ ID NOs: 180-662.

Embodiment 62 is the protease-activated pro-cytokine of any one of the preceding embodiments, wherein the targeting sequence binds to denatured collagen.

Embodiment 63 is the protease-activated pro-cytokine of any one of embodiments 1-61, wherein the targeting sequence binds to collagen.

Embodiment 64 is the protease-activated pro-cytokine of any one of embodiments 62-63, wherein the collagen is collagen I.

Embodiment 65 is the protease-activated pro-cytokine of any one of embodiments 62-63, wherein the collagen is collagen II.

Embodiment 66 is the protease-activated pro-cytokine of any one of embodiments 62-63, wherein the collagen is collagen III.

Embodiment 67 is the protease-activated pro-cytokine of any one of embodiments 62-63, wherein the collagen is collagen IV.

Embodiment 68 is the protease-activated pro-cytokine of any one of embodiments 1-61, wherein the targeting sequence binds to integrin.

Embodiment 69 is the protease-activated pro-cytokine of the immediately preceding embodiment, wherein the integrin is one or more of α1β1 integrin, α2β1 integrin, α3β1 integrin, α4β1 integrin, α5β1 integrin, α6β1 integrin, α7β1 integrin, α9β1 integrin, α4β7 integrin, αvβ3 integrin, αvβ5 integrin, αIIbβ3 integrin, αIIIbβ3 integrin, αMβ2 integrin, or αIIbβ3 integrin.

Embodiment 70 is the protease-activated pro-cytokine of any one of embodiments 1-61, wherein the targeting sequence binds to von Willebrand factor.

Embodiment 71 is the protease-activated pro-cytokine of any one of embodiments 1-61, wherein the targeting sequence binds to IgB.

Embodiment 72 is the protease-activated pro-cytokine of any one of embodiments 1-61, wherein the targeting sequence binds to heparin.

Embodiment 73 is the protease-activated pro-cytokine of the immediately preceding embodiment, wherein the targeting sequence binds to heparin and a syndecan, a heparan sulfate proteoglycan, or an integrin, optionally wherein the integrin is one or more of α1β1 integrin, α2β1 integrin, α3β1 integrin, α4β1 integrin, α5β1 integrin, α6β1 integrin, α7β1 integrin, α9β1 integrin, α4β7 integrin, αvβ3 integrin, αvβ5 integrin, αIIbβ3 integrin, αIIIbβ3 integrin, αMβ2 integrin, or αIIbβ3 integrin.

Embodiment 74 is the protease-activated pro-cytokine of any one of embodiments 72-73, wherein the syndecan is one of more of syndecan-1, syndecan-4, and syndecan-2(w).

Embodiment 75 is the protease-activated pro-cytokine of any one of embodiments 1-61, wherein the targeting sequence binds to a heparan sulfate proteoglycan.

Embodiment 76 is the protease-activated pro-cytokine of any one of embodiments 1-61, wherein the targeting sequence binds to a sulfated glycoprotein.

Embodiment 77 is the protease-activated pro-cytokine of any one of embodiments 1-61, wherein the targeting sequence binds to hyaluronic acid.

Embodiment 78 is the protease-activated pro-cytokine of any one of embodiments 1-61, wherein the targeting sequence binds to fibronectin.

Embodiment 79 is the protease-activated pro-cytokine of any one of embodiments 1-61, wherein the targeting sequence binds to cadherin.

Embodiment 80 is the protease-activated pro-cytokine of any one of the preceding embodiments, wherein the targeting sequence is configured to bind its target in a pH-sensitive manner.

Embodiment 81 is the protease-activated pro-cytokine of the immediately preceding embodiment, wherein the targeting sequence has a higher affinity for its target at a pH below normal physiological pH than at normal physiological pH, optionally wherein the pH below normal physiological pH is below 7, or below 6.

Embodiment 82 is the protease-activated pro-cytokine of the immediately preceding embodiment, wherein the targeting sequence has a higher affinity for its target at a pH in the range of 5-7, e.g., 5-5.5, 5.5-6, 6-6.5, or 6.5-7, than at normal physiological pH.

Embodiment 83 is the protease-activated pro-cytokine of any one of the preceding embodiments, wherein the targeting sequence comprises one or more histidines, e.g., 1, 2, 3, 4, 5, 6, 7, 8, 9, or 10 histidines.

Embodiment 84 is the protease-activated pro-cytokine of any one of the preceding embodiments, wherein the targeting sequence comprises the sequence of any one of SEQ ID NOs: 641-662, or a variant having one or two mismatches relative to the sequence of any one of SEQ ID NOs: 641-662.

Embodiment 85 is the protease-activated pro-cytokine of the immediately preceding embodiment, wherein the targeting sequence comprises the sequence of any one of SEQ ID NOs: 641-662.

Embodiment 86 is the protease-activated pro-cytokine of any one of embodiments 80-86, wherein the targeting sequence is configured to bind, in a pH-sensitive manner, an extracellular matrix component, IgB (CD79b), an integrin, a cadherin, a heparan sulfate proteoglycan, a syndecan, or a fibronectin.

Embodiment 87 is the protease-activated pro-cytokine of the immediately preceding embodiment, wherein the extracellular matrix component is hyaluronic acid, heparin, heparan sulfate, or a sulfated glycoprotein.

Embodiment 88 is the protease-activated pro-cytokine of embodiment 86, wherein the targeting sequence is configured to bind a fibronectin in a pH-sensitive manner.

Embodiment 89 is the protease-activated pro-cytokine of any one of the preceding embodiments, wherein the cytokine polypeptide sequence is an interleukin polypeptide sequence.

Embodiment 90 is the protease-activated pro-cytokine of any one of the preceding embodiments, wherein the cytokine polypeptide sequence is capable of binding a receptor comprising CD132.

Embodiment 91 is the protease-activated pro-cytokine of any one of the preceding embodiments, wherein the cytokine polypeptide sequence is capable of binding a receptor comprising CD122.

Embodiment 92 is the protease-activated pro-cytokine of any one of the preceding embodiments, wherein the cytokine polypeptide sequence is capable of binding a receptor comprising CD25.

Embodiment 93 is the protease-activated pro-cytokine of any one of the preceding embodiments, wherein the cytokine polypeptide sequence is an IL-2 polypeptide sequence.

Embodiment 94 is the protease-activated pro-cytokine of the immediately preceding embodiment, wherein the IL-2 polypeptide sequence has at least 80, 85, 90, 95, 97, 98, or 99 percent identity to the sequence of any one of SEQ ID NOs: 1-4.

Embodiment 95 is the protease-activated pro-cytokine of the immediately preceding embodiment, wherein the IL-2 polypeptide sequence comprises the sequence of any one of SEQ ID NOs: 1-4.

Embodiment 96 is the protease-activated pro-cytokine of any one of embodiments 93-95, wherein the IL-2 polypeptide sequence is a human IL-2 polypeptide sequence.

Embodiment 97 is the protease-activated pro-cytokine of the immediately preceding embodiment, wherein the IL-2 polypeptide sequence comprises the sequence of SEQ ID NO: 1.

Embodiment 98 is the protease-activated pro-cytokine of any one of embodiments 93-95, wherein the IL-2 polypeptide sequence comprises the sequence of SEQ ID NO: 2.

Embodiment 99 is the protease-activated pro-cytokine of any one of embodiments 93-98, wherein the inhibitory polypeptide sequence comprises an IL-2 binding domain of an IL-2 receptor (IL-2R).

Embodiment 100 is the protease-activated pro-cytokine of the immediately preceding embodiment, wherein the inhibitory polypeptide sequence comprises an amino acid sequence having at least 80, 85, 90, 95, 97, 98, or 99 percent identity to the sequence of any one of SEQ ID NOs: 10-19.

Embodiment 101 is the protease-activated pro-cytokine of the immediately preceding embodiment, wherein the IL-2R is a human IL-2R.

Embodiment 102 is the protease-activated pro-cytokine of any one of embodiments 93-98, wherein the inhibitory polypeptide sequence comprises an IL-2-binding immunoglobulin domain.

Embodiment 103 is the protease-activated pro-cytokine of any one of embodiments 93-98, wherein the IL-2-binding immunoglobulin domain is a human IL-2-binding immunoglobulin domain.

Embodiment 104 is the protease-activated pro-cytokine of the immediately preceding embodiment, wherein the IL-2-binding immunoglobulin domain comprises a VL region comprising hypervariable regions (HVRs) HVR-1, HVR-2, and HVR-3 having the sequences of SEQ ID NOs: 33, 34, and 35, respectively, and a VH region comprising HVR-1, HVR-2, and HVR-3 having the sequences of SEQ ID NOs: 36, 37, and 38, respectively.

Embodiment 105 is the protease-activated pro-cytokine of any one of embodiments 102-104, wherein the IL-2-binding immunoglobulin domain comprises a VL region comprising an amino acid sequence having at least 80, 85, 90, 95, 97, 98, or 99 percent identity to the sequence of SEQ ID NO: 32 and a VH region comprising an amino acid sequence having at least 80, 85, 90, 95, 97, 98, or 99 percent identity to the sequence of SEQ ID NO: 33.

Embodiment 106 is the protease-activated pro-cytokine of the immediately preceding embodiment, wherein the IL-2-binding immunoglobulin domain comprises a VL region comprising the sequence of SEQ ID NO: 32 and a VH region comprising the sequence of SEQ ID NO: 33.

Embodiment 107 is the protease-activated pro-cytokine of any one of embodiments 102-104, wherein the IL-2-binding immunoglobulin domain is an scFv.

Embodiment 108 is the protease-activated pro-cytokine of the immediately preceding embodiment, wherein the IL-2-binding immunoglobulin domain comprises an amino acid sequence having at least 80, 85, 90, 95, 97, 98, or 99 percent identity to the sequence of SEQ ID NO: 30 or 31.

Embodiment 109 is the protease-activated pro-cytokine of the immediately preceding embodiment, wherein the IL-2-binding immunoglobulin domain comprises the sequence of SEQ ID NO: 30 or 31.

Embodiment 110 is the protease-activated pro-cytokine of embodiment 1, comprising the sequence of any one of SEQ ID NOs: 803-852.

Embodiment 111 is a pharmaceutical composition comprising the protease-activated pro-cytokine of any one of the preceding embodiments.

Embodiment 112 is the protease-activated pro-cytokine or pharmaceutical composition of any one of the preceding embodiments, for use in therapy.

Embodiment 113 is the protease-activated pro-cytokine or pharmaceutical composition of any one of the preceding embodiments, for use in treating a cancer.

Embodiment 114 is a method of treating a cancer, comprising administering the protease-activated pro-cytokine or pharmaceutical composition of any one of the preceding embodiments to a subject in need thereof.

Embodiment 115 is a use of the protease-activated pro-cytokine or pharmaceutical composition of any one of embodiments 1-110 for the manufacture of a medicament for treating cancer.

Embodiment 116 is the method, use, or protease-activated pro-cytokine for use of any one of embodiments 113-115, wherein the cancer is a solid tumor.

Embodiment 117 is the method, use, or protease-activated pro-cytokine for use of the immediately preceding embodiment, wherein the solid tumor is metastatic and/or unresectable.

Embodiment 118 is the method, use, or protease-activated pro-cytokine for use of any one of embodiments 113-117, wherein the cancer is a PD-L1-expressing cancer.

Embodiment 119 is the method, use, or protease-activated pro-cytokine for use of any one of embodiments 113-118, wherein the cancer is a melanoma, a colorectal cancer, a breast cancer, a pancreatic cancer, a lung cancer, a prostate cancer, an ovarian cancer, a cervical cancer, a gastric or gastrointestinal cancer, a lymphoma, a colon or colorectal cancer, an endometrial cancer, a thyroid cancer, or a bladder cancer.

Embodiment 120 is the method, use, or protease-activated pro-cytokine for use of any one of embodiments 113-119, wherein the cancer is a microsatellite instability-high cancer.

Embodiment 121 is the method, use, or protease-activated pro-cytokine for use of any one of embodiments 113-120, wherein the cancer is mismatch repair deficient.

Embodiment 122 is a nucleic acid encoding the protease-activated pro-cytokine of any one of embodiments 1-110.

Embodiment 123 is an expression vector comprising the nucleic acid of embodiment 121.

Embodiment 124 is a host cell comprising the nucleic acid of embodiment 121 or the vector of embodiment 122.

Embodiment 125 is a method of producing a protease-activated pro-cytokine, comprising culturing the host cell of embodiment 124 under conditions wherein the protease-activated pro-cytokine is produced.

Embodiment 126 is the method of the immediately preceding embodiment, further comprising isolating the protease-activated pro-cytokine.

Embodiment 127 is a method of boosting T regulatory cells and/or reducing inflammation or autoimmune activity, comprising administering the protease-activated pro-cytokine of any one of embodiments 1-110 to an area of interest in a subject, e.g., an area of inflammation in the subject.

Embodiment 128 is a method of treating an inflammatory or autoimmune disease or disorder in a subject, comprising administering the protease-activated pro-cytokine of any one of embodiments 1-110 to an area of interest in a subject, e.g., an area of inflammation or autoimmune activity in the subject.

### FIGURE LEGENDS

FIG. 1A shows an illustration of an exemplary cytokine prodrug structure and an SDS-PAGE gel characterizing a purified cytokine prodrug (Construct B). Abbreviations: PM, pharmacokinetic modulator; HMW, high molecular weight.

FIG. 1B shows an illustration of an exemplary cytokine prodrug structure comprising human IL-2 and IL-2Rα sequences and an MMP-cleavable linker, and an SDS-PAGE gel and Western blot characterizing a purified cytokine prodrug (Construct E). Abbreviations: Hu, human; MMP, matrix metalloprotease; other abbreviations are as above.

FIG. 1C shows an illustration of an exemplary cytokine prodrug structure comprising murine IL-2 and IL-2Rα sequences, an MMP-cleavable linker, additional linkers that include a targeting sequence (“RET Linker”), and an SDS-PAGE gel characterizing the indicated purified cytokine prodrugs.

FIG. 1D shows an illustration of an exemplary cytokine prodrug structure comprising human IL-2 and IL-2Rα sequences, an MMP-cleavable linker, additional linkers that include a targeting sequence (“RET Linker”), and an SDS-PAGE gel characterizing the indicated purified cytokine prodrugs.

FIG. 2A illustrates a cleavage reaction of a cytokine prodrug by a protease and shows Western blot evidence of cleavage of Construct A by MMP-9 at time points of 1, 2, and 4 hours and overnight. Each of the Western blots contains +MMP digestion lanes and −MMP mock-digestion lanes. Cleavage product was detectable at 1 hour, and the full-length cytokine prodrug was substantially undetectable at the overnight +MMP time point.

FIG. 2B illustrates a cleavage reaction of a cytokine prodrug comprising a pharmacokinetic modulator by a protease and shows Western blot evidence of cleavage of Construct B by MMP-9 at time points of 1, 4, and 20 hours. Each of the Western blots contains +MMP digestion lanes and −MMP mock-digestion lanes. Cleavage product was detectable at 1 hour, and the full-length cytokine prodrug gave only a faint band at the 20 hour +MMP time point.

FIGS. 2C-E illustrate cleavage reactions of a cytokine prodrug comprising a pharmacokinetic modulator by a protease and shows Western blot evidence of cleavage of Construct E by MMP-9 at time points of 1, 4, and 22 hours (2C); and cleavage of the indicated constructs at 18 hours (2D and 2E). Constructs BBB, CCC, and FFF in FIG. 2E that did not show substantial cleavage had scrambled MMP sites. Each of the Western blots contains +MMP9 digestion lanes and −MMP9 mock-digestion lanes. Cleavage product was detectable at 1 hour, and the full-length cytokine prodrug gave essentially no band at the 22 hour +MMP time point.

FIG. 3A shows results of a CTLL-2 proliferation assay with Construct A or cleavage products thereof. Construct A was cleaved by MMP-9 and the resulting products were incubated with CTLL-2 cells. The data shows that MMP-9 treated Construct A stimulates CTLL-2 cell proliferation in a dose dependent manner and exhibits 10-fold greater activity than untreated Construct A (EC50 comparison). EC50 values are shown in nM.

FIG. 3B shows results of a CTLL-2 proliferation assay with Construct B or cleavage products thereof. Construct B was cleaved by MMP-9 and the resulting products were incubated with CTLL-2 cells. For comparison, mIL2 was also incubated with CTLL-2 cells. The data show that MMP-9 treated Construct B stimulates CTLL-2 cell proliferation in a dose dependent manner. Uncleaved Construct B was minimally stimulatory. EC50 values are shown in nM.

FIG. 3C-FIG. 3J show HEK-Blue™ IL2 assay results. Cells were treated with various concentrations Construct E, uncleaved or cleaved with mMMP9 for 22 hours (FIG. 3C); human IL2 (FIG. 3D); Construct B, uncleaved or cleaved with mMMP9 for 19 hours; Construct J, Construct K, Construct F, Construct L, or Construct I, each uncleaved or cleaved with mMMP9 for 22 hours (FIGS. 3E-J, respectively); and the EC50 was determined based on OD630 as a readout of IL-2 stimulation.

FIG. 3K-FIG. 3L show results of a CTLL-2 proliferation assay with Construct M, Construct N, or cleavage products thereof. Cleavage was by MMP-2 for 2 hr and the resulting products were incubated with CTLL-2 cells. The data show that MMP-2 treated Construct M and Construct N stimulate CTLL-2 cell proliferation in a dose dependent manner. EC50 values are shown in nM.

FIG. 3M shows Coomassie-stained SDS-PAGE results comparing Construct E, Construct M, and Construct N. Construct M and Construct N showed decreased aggregation and greater stability and homogeneity.

FIG. 3N-FIG. 3P show results of a CTLL-2 proliferation assay with Construct 0, Construct P, Construct Q, or cleavage products thereof. Cleavage was by MMP2 for 2 hr and the resulting products were incubated with CTLL-2 cells. The data show that MMP2 treated Construct 0, Construct P, and Construct Q stimulate CTLL-2 cell proliferation in a dose dependent manner. EC50 values are shown in nM.

FIG. 3Q-FIG. 3Y show results of a HEK-Blue™ IL2 assay with the indicated construct or cleavage products thereof. Cleavage was by MMP9 for either 18 hr or 22 hr and the resulting products were incubated with HEK-Blue™ IL2 cells. EC50 was determined based on OD630 as a readout of IL-2 stimulation. The data show that MMP9 treated constructs stimulate IL-2 in a dose dependent manner. EC50 values are shown in nM.

FIG. 4 illustrates a serum stability assay using Construct B and provides results thereof indicating that Construct B was stable when incubated with serum collected from control or tumor-bearing over a time course of 72 hours. Concentrations were measured by quantitative sandwich ELISA using an mIL2 capture antibody and mIL2Rα detection antibody.

FIG. 5 shows a study design, graphical results, and pharmacokinetic (PK) parameters for Construct B in mice. PK parameters were calculated using WinNonlin 7.0 (non-compartmental model).

FIG. 6A shows a study design and results for intratumoral dosing of Construct A in mice injected subcutaneously with MC38 cells at day-7 and then treated with Construct A, vehicle, or human IL-2 on each of days 0-4 and 7-11. Construct A substantially inhibited tumor growth. In contrast, human TL-2 adversely affected tumor control relative to vehicle. Necrosis attributable to tumor growth was observed in the control and human IL-2 groups.

FIG. 6B shows a study design in which mice treated as in FIG. 6A were re-challenged with 2×10⁶ MC38 cells at day 40. Tumor growth was rejected, indicating that the treatment resulted in a durable response including anti-tumor immune memory.

FIG. 7A shows a study design in mice injected subcutaneously with MC38 cells at day-10 where Construct B or vehicle was administered intravenously once per three days (Q3D) during a three week period (eight total administrations). Essentially no systemic toxicity was observed. Construct B-treated mice showed virtually no tumor growth after initiation of treatment, in contrast to vehicle-treated mice where tumor growth continued through day 21. Following day 21, several vehicle-treated mice were euthanized due to tumor volume exceeding 3000 mm³ and accordingly subsequent tumor volume data for vehicle-treated mice is not shown as it would be biased toward mice with smaller tumor volumes relative to the population average through day 21.

FIG. 7B shows body weight data for the same mice as in FIG. 7A. Mouse body weight was substantially constant during treatment with Construct B, consistent with lack of any apparent toxicity.

FIG. 8 shows immunohistochemistry results for tumor-infiltrating immune cells at day 21 for vehicle group tissues and at day 25 for Construct B treated tumors of the study described above for FIG. 7A. Significantly more immune cells of all tested types were observed in Construct B-treated mice compared to vehicle-treated mice. Additionally, the proportion of cells with markers consistent with a effector T cell phenotype was substantially greater than the proportion of CD4+Foxp3+(regulatory T) cells. Statistical analysis was performed using unpaired t test by Prism 5.0 software. P value between groups was calculated, and the differences with p value <0.05 were considered statistically significant. * p<0.05, ** p<0.01, *** p<0.001.

FIG. 9 shows quantification of MMP activity in the indicated tumor-bearing mouse models by fluorescence intensity over time following MMPSense 680™ injection.

FIG. 10A-FIG. 10D show tumor volume over time for mice treated with vehicle or Construct B as indicated in the indicated cancer models.

FIG. 11A-FIG. 11D show tumor volume over time (11A) and levels of the indicated enzymes (11B-D) for mice treated with vehicle or Construct B as indicated in the B16F10 melanoma model.

FIG. 12A-FIG. 12D show tumor volume over time (12A) and levels of the indicated enzymes (12B-D) for mice treated with vehicle or Construct B as indicated in the RM-1 prostate cancer model.

FIG. 13A shows MMP activity, measured as described for FIG. 9, in the indicated groups.

FIG. 13B-FIG. 13C show tumor volume over time for mice treated with vehicle or Construct B as indicated in the indicated cancer models.

FIG. 14A-B show schematic structures of the indicated linkers and binding of MMP linker peptides containing heparin binding motifs to heparin-agarose beads.

FIG. 14C shows cartoons of the structures of the indicated constructs and heparin binding assay results for the indicated constructs. Assays were performed at pH 7.5 unless indicated as performed at pH 6.

FIG. 14D shows schematic structures of the indicated linkers and binding of the indicated peptides to fibronectin at the indicated pH values.

FIG. 14E shows fibronectin binding assay results for the indicated constructs. Assays were performed at pH 7.5 unless indicated as performed at pH 6.

FIG. 14F shows schematic structures of the indicated linkers and binding of MMP linker peptides containing collagen binding motifs to beads associated with collagen IV.

FIG. 14G shows an anti-mIL2 Western blot of input (I), supernatant (S), collagen-bound (C) and control agarose bound (A) fractions from pulldown assays performed with the indicated constructs.

FIG. 15A shows fluorescent images of mice treated with the indicated constructs as described in Example 15.

FIG. 15B shows tumor-associated fluorescence measured in mice treated with the indicated constructs as described in Example 15.

FIG. 15C-H show amounts of the indicated constructs present in tumor lysates prepared as described in Example 16. Here and throughout, mpk means mg/kg.

FIG. 15I-K show amounts of the indicated constructs present in serum samples prepared as described in Example 16.

FIG. 16A-B show tumor volume over time for groups treated with the indicated constructs as described in Example 17.

FIG. 17A-B show IFN-γ levels in tumors following treatment with the indicated constructs as described in Example 18.

FIG. 18A-E show exemplary arrangements of elements in cytokine prodrugs comprising various combinations of a cytokine polypeptide sequence (CYTOKINE), a pharmacokinetic modulator (PK EXT), a protease-cleavable polypeptide sequence in a linker (PRO-LNK), an inhibitory polypeptide sequence (INHIBITOR), and in some cases one or more targeting sequences (RET LNK) or additional linkers (LNK). The targeting sequences are shown as white text on a dark background. In FIGS. 18A and 18C, the pharmacokinetic modulator is on the same side of the protease-cleavable sequence as the inhibitory polypeptide sequence, so that it would not impact the pharmacokinetics of the cytokine polypeptide sequence. In FIGS. 18B and 18D, the pharmacokinetic modulator is on the same side of the protease-cleavable sequence as the cytokine polypeptide sequence, so that it would impact the pharmacokinetics of the cytokine polypeptide sequence. In FIG. 18E, a protease-cleavable sequence is present on each side of the pharmacokinetic modulator. This arrangement can produce intermediate results as the pharmacokinetic modulator would be separated from the cytokine polypeptide sequence more slowly than the inhibitory polypeptide sequence.

### DETAILED DESCRIPTION OF CERTAIN EMBODIMENTS

This specification describes exemplary embodiments and applications of the disclosure. The disclosure, however, is not limited to these exemplary embodiments and applications or to the manner in which the exemplary embodiments and applications operate or are described herein. The term “or” is used in an inclusive sense, i.e., equivalent to “and/or,” unless the context dictates otherwise. It is noted that, as used in this specification and the appended claims, the singular forms “a,” “an,” and “the,” and any singular use of any word, include plural referents unless expressly and unequivocally limited to one referent. As used herein, the terms “comprise,” “include,” and grammatical variants thereof are intended to be non-limiting, such that recitation of items in a list is not to the exclusion of other like items that can be substituted or added to the listed items. Section divisions in the specification are provided for the convenience of the reader only and do not limit any combination of elements discussed. In case of any contradiction or conflict between material incorporated by reference and the expressly described content provided herein, the expressly described content controls.

### Overview

Provided herein are protease-activated pro-cytokines (also referred to herein as cytokine prodrugs) comprising a linker comprising a protease-cleavable linker and a targeting sequence described herein, e.g., a targeting sequence configured to bind an extracellular matrix component, an integrin, or a syndecan; or configured to bind an extracellular matrix component, IgB (CD79b), an integrin, a cadherin, a heparan sulfate proteoglycan, a syndecan, or a fibronectin in a pH-sensitive manner; or a targeting sequence comprising the sequence of any one of SEQ ID NOs: 180-662. The cleavable linker can be between a cytokine polypeptide sequence and an inhibitory polypeptide sequence, such that the ability of the cytokine polypeptide sequence to activate immune cells is reduced or eliminated compared to a free cytokine polypeptide sequence. Proteolysis of the linker can liberate the cytokine so that it can activate immune cells.

In some embodiments, the protease-cleavable linker is cleavable by a protease expressed at higher levels in the tumor microenvironment (TME) than in healthy tissue of the same type. In some embodiments, the protease-cleavable linker is a matrix metalloprotease (MMP)-cleavable linker, such as any of the MMP-cleavable linkers described herein. Without wishing to be bound by any particular theory, increased expression of proteases, including but not necessarily limited to MMPs, in the tumor microenvironment (TME) can provide a mechanism for achieving selective or preferential activation of the cytokine prodrug at or near a tumor site. Certain protease-cleavable linkers described herein are considered particularly suitable for achieving such selective or preferential activation.

In other embodiments, the cytokine prodrug comprises a targeting sequence, e.g., a targeting sequence that binds an extracellular matrix component, an integrin, or a syndecan, or is configured to bind fibronectin in a pH-sensitive manner. The targeting sequence can facilitate accumulation and/or increased residence time of the cytokine prodrug and/or the active cytokine in the ECM. In some embodiments, a targeting sequence is combined with a protease-cleavable linker cleavable by a protease expressed at higher levels in the TME and/or cleavable by an MMP.

In any of the foregoing embodiments, the cytokine prodrug may further comprise a pharmacokinetic modulator, e.g., which extends the half-life of the prodrug and which may optionally also extend the half-life of the active cytokine.

Sequences of exemplary cytokine prodrugs and components thereof are shown in Tables 1 and 2. In Table 1, “X_{Hy}” designates a hydrophobic amino acid residue. In some embodiments, the hydrophobic amino acid residue is any one of glycine (Gly), alanine (Ala), valine (Val), leucine (Leu), isoleucine (Ile), proline (Pro), phenylalanine (Phe), methionine (Met), and tryptophan (Trp). In some embodiments, the hydrophobic amino acid residue is any one of Ala, Leu, Val, Ile, Pro, Phe, Met, and Trp. In some embodiments, the hydrophobic amino acid residue is any one of Leu, Val, Ile, Pro, Phe, Met, and Trp. In some embodiments, the hydrophobic amino acid residue is any one of Ala, Leu, Val, Ile, Phe, Met, and Trp. In some embodiments, the hydrophobic amino acid residue is any one of Leu, Val, Ile, Phe, Met, and Trp. “(Pip)” represents piperidine. “(Hof)” represents homophenylalanine. “(Cit)” represents citrulline. “(Et)” represents ethionine. “C(me)” represents methylcysteine. In certain sequences, underlining is used to indicate mutated positions.

This disclosure further provides uses of these cytokine prodrugs, e.g., for treating cancer. In some embodiments, the cytokine prodrug is selectively or preferentially cleaved in the tumor microenvironment, which may result in beneficial effects, e.g., improved recruitment and/or activation of immune cells in the vicinity of the tumor, and/or reduced systemic exposure to active cytokines.

TABLE 1
Table of Sequences of Cytokine Prodrugs and Components Thereof

| | | | | | |
|---|---|---|---|---|---|
| SEQ | | | | | |
| ID | | | | | |
| NO | Description | Sequence | Species | Function | Notes |

IL-2 sequences

| | | | | | |
|---|---|---|---|---|---|
| 1 | h IL-2 | APTSSSTKKTQLQLEHLLLDLQMILNGINNYKNPKLTRMLTFKFYMPKKATELK | human | cytokine | wild-type |
| | | HLQCLEEELKPLEEVLNLAQSKNFHLRPRDLISNINVIVLELKGSETTFMCEYA | | | |
| | | DETATIVEFLNRWITFCQSIISTLT | | | |
| 2 | h IL-2 | APTSSSTKKTQLQLEHLLLDLQMILNGINNYKNPKLTRMLTFKFYMPKKATELK | human | cytokine | C125 to S |
| | (C125S) | HLQCLEEELKPLEEVLNLAQSKNFHLRPRDLISNINVIVLELKGSETTFMCEYA | | | mutation |
| | | DETATIVEFLNRWITFSQSIISTLT | | | |
| 3 | m IL-2 | APTSSSTSSSTAEAQQQQQQQQQQQQHLEQLLMDLQELLSRMENYRNLKLPRML | mouse | cytokine | wild-type |
| | | TFKFYLPKQATELKDLQCLEDELGPLRHVLDLTQSKSFQLEDAENFISNIRVTV | | | |
| | | VKLKGSDNTFECQFDDESATVVDFLRRWIAFCQSIISTSPQ | | | |
| 4 | m IL-2 | APTSSSTSSSTAEAQQQQQQQQQQQQHLEQLLMDLQELLSRMENYRNLKLPRML | mouse | cytokine | C140 to S |
| | (C140S) | TFKFYLPKQATELKDLQCLEDELGPLRHVLDLTQSKSFQLEDAENFISNIRVTV | | | mutation |
| | | VKLKGSDNTFECQFDDESATVVDFLRRWIAFSQSIISTSPQ | | | |
| 5-9 | Not Used | | | | |

Blockers: IL-2R sequences

| | | | | | |
|---|---|---|---|---|---|
| 10 | h IL-2Ralpha | ELCDDDPPEIPHATFKAMAYKEGTMLNCECKRGFRRIKSGSLYMLCTGNSSHSS | human | blocker | wild-type |
| | | WDNQCQCTSSATRNTTKQVTPQPEEQKERKTTEMQSPMQPVDQASLPGHCREPP | | | amino acids |
| | | PWENEATERIYHFVVGQMVYYQCVQGYRALHRGPAESVCKMTHGKTRWTQPQLI | | | 1-219 |
| | | CTGEMETSQFPGEEKPQASPEGRPESETSCLVTTTDFQIQTEMAATMETSIFTT | | | |
| | | EYQ | | | |
| 11 | h IL-2Ralpha | ELCDDDPPEIPHATFKAMAYKEGTMLNCECKRGFRRIKSGSLYMLCTGNSSHSS | human | blocker | sushi domain |
| | (1-63) | WDNQCQCTS | | | 1 wild-type |
| 12 | h IL-2Ralpha | ELCDDDPPEIPHATFKAMAYKEGTILNCECKRGFRRIKSGSLYMLCTGNSSHSS | human | blocker | M25 to I |
| | (M25I) | WDNQCQCTSSATRNTTKQVTPQPEEQKERKTTEMQSPMQPVDQASLPGHCREPP | | | mutation |
| | | PWENEATERIYHFVVGQMVYYQCVQGYRALHRGPAESVCKMTHGKTRWTQPQLI | | | |
| | | CTGEMETSQFPGEEKPQASPEGRPESETSCLVTTTDFQIQTEMAATMETSIFTT | | | |
| | | EYQ | | | |
| 13 | h IL-2Ralpha | ELCDDDPPEIPHATFKAMAYKEGTMLNCECKRGFRRIKSGSVYMLCTGNSSHSS | human | blocker | L42 to V |
| | (L42V) | WDNQCQCTSSATRNTTKQVTPQPEEQKERKTTEMQSPMQPVDQASLPGHCREPP | | | mutation |
| | | PWENEATERIYHFVVGQMVYYQCVQGYRALHRGPAESVCKMTHGKTRWTQPQLI | | | |
| | | CTGEMETSQFPGEEKPQASPEGRPESETSCLVTTTDFQIQTEMAATMETSIFTT | | | |
| | | EYQ | | | |
| 14 | h IL-2Ralpha | ELCDDDPPEIPHATFKAMAYKEGTILNCECKRGFRRIKSGSVYMLCTGNSSHSS | human | blocker | M25 to I |
| | (M25I; L42V) | WDNQCQCTSSATRNTTKQVTPQPEEQKERKTTEMQSPMQPVDQASLPGHCREPP | | | mutation; L42 |
| | | PWENEATERIYHFVVGQMVYYQCVQGYRALHRGPAESVCKMTHGKTRWTQPQLI | | | to V mutation |
| | | CTGEMETSQFPGEEKPQASPEGRPESETSCLVTTTDFQIQTEMAATMETSIFTT | | | |
| | | EYQ | | | |
| 15 | Human | LNTTILTPNGNEDTTADFFLTTMPTDSLSVSTLPLPEVQCFVFNVEYMNCTWNS | human | blocker | |
| | IL2Rgamma | SSEPQPTNLTLHYWYKNSDNDKVQKCSHYLFSEEITSGCQLQKKEIHLYQTFVV | | | |
| | polypeptide | QLQDPREPRRQATQMLKLQNLVIPWAPENLTLHKLSESQLELNWNNRFLNHCLE | | | |
| | sequence | HLVQYRTDWDHSWTEQSVDYRHKFSLPSVDGQKRYTFRVRSRFNPLCGSAQHWS | | | |
| | | EWSHPIHWGSNTSKENPFLFALEA | | | |
| 16 | Human | AVNGTSQFTCFYNSRANISCVWSQDGALQDTSCQVHAWPDRRRWNQTCELLPVS | human | blocker | |
| | IL2Rbeta | QASWACNLILGAPDSQKLTTVDIVTLRVLCREGVRWRVMAIQDFKPFENLRLMA | | | |
| | polypeptide | PISLQVVHVETHRCNISWEISQASHYFERHLEFEARTLSPGHTWEEAPLLTLKQ | | | |
| | sequence | KQEWICLETLTPDTQYEFQVRVKPLQGEFTTWSPWSQPLAFRTKPAALGKDT | | | |
| 17 | chimeric IL- | ELCLYDPPEVPNATFKALSYKNGTILNCECKRGFRRLKELVYMRCLGNSWSSNC | human/ | blocker | mouse |
| | 2Ralpha | QCTSSATRNTTKQVTPQPEEQKERKTTEMQSPMQPVDQASLPGHCREPPPWENE | mouse | | IL2Ralpha (1- |
| | | ATERIYHFVVGQMVYYQCVQGYRALHRGPAESVCKMTHGKTRWTQPQLICTGEM | | | 58) - hu |
| | | ETSQFPGEEKPQASPEGRPESETSCLVTTTDFQIQTEMAATMETSIFTTEYQ | | | IL2Ralpha |
| | | | | | (64-219) |
| 18 | m IL-2Ralpha | ELCLYDPPEVPNATFKALSYKNGTILNCECKRGFRRLKELVYMRCLGNSWSSNC | mouse | blocker | wild-type |
| | | QCTSNSHDKSRKQVTAQLEHQKEQQTTTDMQKPTQSMHQENLTGHCREPPPWKH | | | amino acids |
| | | EDSKRIYHFVEGQSVHYECIPGYKALQRGPAISICKMKCGKTGWTQPQLTCVDE | | | 1-215 |
| | | REHHRFLASEESQGSRNSSPESETSCPITTTDFPQPTETTAMTETFVLTMEYK | | | |
| 19 | m IL-2Ralpha | ELCLYDPPEVPNATFKALSYKNGTILNCECKRGFRRLKELVYMRCLGNSWSSNC | mouse | blocker | sushi domain |
| | (1-58) | QCTS | | | 1 wild-type |
| 20 | h IL-2Ralpha | ELCLYDPPEIPHATFKAMAYKEGTILNCECKRGFRRIKSGSLYMLCTGNSSH | human | blocker | D4 to L |
| | (1-219) | SSWDNQCQCTSSATRNTTKQVTPQPEEQKERKTTEMQSPMQPVDQASLPGHC | | | mutation; D5 |
| | M25I/D4L/D5Y | REPPPWENEATERIYHFVVGQMVYYQCVQGYRALHRGPAESVCKMTHGKTRW | | | to Y |
| | | TQPQLICTGEMETSQFPGEEKPQASPEGRPESETSCLVTTTDFQ | | | mutation; |
| | | IQTEMAATMETSIFTTEYQ | | | M25 to I |
| | | | | | mutation |
| 21 | h IL-2Ralpha | ELCLYDPPEIPHATFKAMAYKEGTMLNCECKRGFRRIKSGSVYMLCTGNSSH | human | blocker | D4 to L |
| | (1-219) | SSWDNQCQCTSSATRNTTKQVTPQPEEQKERKTTEMQSPMQPVDQASLPGHC | | | mutation; D5 |
| | L42V/D4L/ | REPPPWENEATERIYHFVVGQMVYYQCVQGYRALHRGPAESVCKMTHGKTRW | | | to Y |
| | D5Y | TQPQLICTGEMETSQFPGEEKPQASPEGRPESETSCLVTTTDFQ | | | mutation; |
| | | IQTEMAATMETSIFTTEYQ | | | L42 to V |
| | | | | | mutation |
| 22 | h IL-2Ralpha | ELCLYDPPEIPHATFKAMAYKEGTILNCECKRGFRRIKSGSVYMLCTGNSSH | human | blocker | D4 to L |
| | (1-219) | SSWDNQCQCTSSATRNTTKQVTPQPEEQKERKTTEMQSPMQPVDQASLPGHC | | | mutation; D5 |
| | M25I/L42V/ | REPPPWENEATERIYHFVVGQMVYYQCVQGYRALHRGPAESVCKMTHGKTRW | | | to Y |
| | D4L/D5Y | TQPQLICTGEMETSQFPGEEKPQASPEGRPESETSCLVTTTDFQ | | | mutation; |
| | | IQTEMAATMETSIFTTEYQ | | | M25 to I |
| | | | | | mutation; |
| | | | | | L42 to V |
| | | | | | mutation |
| 23 | h IL-2Ralpha | ELCLYDPPEIPHATFKAMAYKEGTMLNCECKRGFRRIKSGSLYMLCTGNSSH | human | blocker | D4 to L |
| | (1- | SSWDNQCQCTSSATRNTTKQVTPQPEEQKERKTTEMQSPMQPVDQASLPGHC | | | mutation; D5 |
| | 219)D4L/D5Y | REPPPWENEATERIYHFVVGQMVYYQCVQGYRALHRGPAESVCKMTHGKTRW | | | to Y |
| | | TQPQLICTGEMETSQFPGEEKPQASPEGRPESETSCLVTTTDFQ | | | mutation |
| | | IQTEMAATMETSIFTTEYQ | | | |
| 24 | h IL-2Ralpha | ELCDDDPPEIPHATFKAMAYKEGTMLNCECKRGFRRIKELVYMLCTGNSSHS | human | blocker | Wild-type |
| | (1-219) | SWDNQCQCTSSATRNTTKQVTPQPEEQKERKTTEMQSPMQPVDQASLPGHCR | | | residues 39- |
| | SGSL₃₉- | EPPPWENEATERIYHFVVGQMVYYQCVQGYRALHRGPAESVCKMTHGKTRWT | | | 42 replaced |
| | ₄₂ELV | QPQLICTGEMETSQFPGEEKPQASPEGRPESETSCLVTTTDFQ | | | with ELV |
| | | IQTEMAATMETSIFTTEYQ | | | |
| 25 | h IL-2Ralpha | ELCDDDPPEIPHATFKAMAYKEGTMLNCECKRGFRRIKSGSLYMLCTGNSSH | human | blocker | Wild-type |
| | (1-192) | SSWDNQCQCTSSATRNTTKQVTPQPEEQKERKTTEMQSPMQPVDQASLPGHC | | | amino acids |
| | | REPPPWENEATERIYHFVVGQMVYYQCVQGYRALHRGPAESVCKMTHGKTRW | | | 1-192 |
| | | TQPQLICTGEMETSQFPGEEKPQASPEGRPESETSC | | | |
| 26 | h IL-2Ralpha | ELCDDDPPEIPHATFKAMAYKEGTILNCECKRGFRRIKSGSLYMLCTGNSSH | human | blocker | M25 to I |
| | (1-192)M25I | SSWDNQCQCTSSATRNTTKQVTPQPEEQKERKTTEMQSPMQPVDQASLPGHC | | | mutation |
| | | REPPPWENEATERIYHFVVGQMVYYQCVQGYRALHRGPAESVCKMTHGKTRW | | | |
| | | TQPQLICTGEMETSQFPGEEKPQASPEGRPESETSC | | | |
| 27 | h IL-2Ralpha | ELCDDDPPEIPHATFKAMAYKEGTMLNCECKRGFRRIKSGSVYMLCTGNSSH | human | blocker | L42 to V |
| | (1-192)L42V | SSWDNQCQCTSSATRNTTKQVTPQPEEQKERKTTEMQSPMQPVDQASLPGHC | | | mutation |
| | | REPPPWENEATERIYHFVVGQMVYYQCVQGYRALHRGPAESVCKMTHGKTRW | | | |
| | | TQPQLICTGEMETSQFPGEEKPQASPEGRPESETSC | | | |
| 28 | h IL-2Ralpha | ELCLYDPPEIPHATFKAMAYKEGTMLNCECKRGFRRIKSGSLYMLCTGNSSH | human | blocker | D4 to L |
| | (1- | SSWDNQCQCTSSATRNTTKQVTPQPEEQKERKTTEMQSPMQPVDQASLPGHC | | | mutation; D5 |
| | 192)D4L/D5Y | REPPPWENEATERIYHFVVGQMVYYQCVQGYRALHRGPAESVCKMTHGKTRW | | | to Y |
| | | TQPQLICTGEMETSQFPGEEKPQASPEGRPESETSC | | | mutation |
| 29 | h IL-2Ralpha | ELCDDDPPEIPHATFKAMAYKEGTMLNCECKRGFRRIKELVYMLCTGNSSHS | human | blocker | Wild-type |
| | (1-192) | SWDNQCQCTSSATRNTTKQVTPQPEEQKERKTTEMQSPMQPVDQASLPGHCR | | | residues 39- |
| | SGSL₃₉- | EPPPWENEATERIYHFVVGQMVYYQCVQGYRALHRGPAESVCKMTHGKTRWT | | | 42 replaced |
| | ₄₂ELV | QPQLICTGEMETSQFPGEEKPQASPEGRPESETSC | | | with ELV |

IL2 Blockers: anti-IL2 sequences

| | | | | | |
|---|---|---|---|---|---|
| 30 | scFv2 | QSVLTQPPSVSGAPGQRVTISCTGTSSNIGAHYDVHWYQQFPGTAPKRLIYGNN | human | blocker | wild-type |
| | | NRPSGVPARFSGSKSGTSASLAITGLQAEDEADYYCQSYDRSLRGWVFGGGTKL | | | |
| | | TVLGEGKSSGSGSESKASEVQLVESGGGLVQPGRSLRLSCAASGFTFDDYAMHW | | | |
| | | VRQAPGKGLEWVSGISWNSGSIGYADSVKGRFTISRDNSKNTLYLQMNSLRAED | | | |
| | | TAVYYCAKDVNWNYGYYFDYWGQGTLVTVSS | | | |
| 31 | scFv2 (18 mer | QSVLTQPPSVSGAPGQRVTISCTGTSSNIGAHYDVHWYQQFPGTAPKRLIYGNN | human | blocker | 18 mer linker |
| | linker) | NRPSGVPARFSGSKSGTSASLAITGLQAEDEADYYCQSYDRSLRGWVFGGGTKL | | | between VL |
| | | TVLGGSTSGSGKPGSGEGSTKGEVQLVESGGGLVQPGRSLRLSCAASGFTFDDY | | | and VH |
| | | AMHWVRQAPGKGLEWVSGISWNSGSIGYADSVKGRFTISRDNSKNTLYLQMNSL | | | |
| | | RAEDTAVYYCAKDVNWNYGYYFDYWGQGTLVTVSS | | | |
| 32 | VL domain of | QSVLTQPPSVSGAPGQRVTISCTGTSSNIGAHYDVHWYQQFPGTAPKRLIYGNN | human | blocker | wild-type |
| | scFv2 | NRPSGVPARFSGSKSGTSASLAITGLQAEDEADYYCQSYDRSLRGWVFGGGTKL | | | |
| | | TVLG | | | |
| 33 | VH domain of | EVQLVESGGGLVQPGRSLRLSCAASGFTFDDYAMHWVRQAPGKGLEWVSGISWN | human | blocker | wild-type |
| | scFv2 | SGSIGYADSVKGRFTISRDNSKNTLYLQMNSLRAEDTAVYYCAKDVNWNYGYYF | | | |
| | | DYWGQGTLVTVSS | | | |
| 34 | scFv2 VL | TGTSSNIGAHYDVH | | | |
| | HVR1 | | | | |
| 35 | scFv2 VL | GNNNRPS | | | |
| | HVR2 | | | | |
| 36 | scFv2 VL | QSYDRSLRGWV | | | |
| | HVR3 | | | | |
| 37 | scFv2 VH | DDYAMH | | | |
| | HVR1 | | | | |
| 38 | scFv2 VH | GISWNSGSIGYADSVKG | | | |
| | HVR2 | | | | |
| 39 | scFv2 VH | KDVNWNYGYYFDY | | | |
| | HVR3 | | | | |
| 747 | scFv183 | DIVMTQSPDSLAVSLGERATINCKSSQSVLYSNNNKNYLAWYQQKPGQPPKL | human | blocker | linker |
| | | LIYGASTRESWVPDRFSGSGSGTDFTLTISSLQAEDVAVYYCQQWYYYPYTF | | | between VL |
| | | GQGTKVEIKGGGGSGGGGSGGGSGGGGSEVQLLESGGGLVQPGGSLRLSCAA | | | and VH |
| | | SGFTFSSYYMSWVRQAPGKGLEWVSDISGRGGQTNYADSVKGRFTISRDNSK | | | |
| | | NTLYLQMNSLRAEDTAVYYCARGGGSFANWGRGTLVTVSS | | | |
| 748 | VL domain of | DIVMTQSPDSLAVSLGERATINCKSSQSVLYSNNNKNYLAWYQQKPGQPPKL | human | blocker | |
| | scFv183 | LIYGASTRESWVPDRFSGSGSGTDFTLTISSLQAEDVAVYYCQQWYYYPYTF | | | |
| | | GQGTKVEIK | | | |
| 749 | VH domain of | EVQLLESGGGLVQPGGSLRLSCAASGFTFSSYYMSWVRQAPGKGLEWVSDIS | human | blocker | |
| | scFv183 | GRGGQTNYADSVKGRFTISRDNSKNTLYLQMNSLRAEDTAVYYCARGGGSFA | | | |
| | | NWGRGTLVTVSS | | | |
| 750 | scFv183 VL | KSSQSVLYSNNNKNYLA | | | |
| | HVR1 | | | | |
| 751 | scFv183 VL | GASTRES | | | |
| | HVR2 | | | | |
| 752 | scFv183 VL | QQWYYYPYT | | | |
| | HVR3 | | | | |
| 753 | scFv183 VH | SSYYMS | | | |
| | HVR1 | | | | |
| 754 | scFv183 VH | DISGRGGQTNYADSVKG | | | |
| | HVR2 | | | | |
| 755 | scFv183 VH | RGGGSFAN | | | |
| | HVR3 | | | | |

Blockers: IL-2R sequences

| | | | | | |
|---|---|---|---|---|---|
| 40 | h IL-2Ralpha | ELCLYDPPEIPHATFKAMAYKEGTILNCECKRGFRRIKSGSLYMLCTGNSSH | human | blocker | D4 to L |
| | (1- | SSWDNQCQCTSSATRNTTKQVTPQPEEQKERKTTEMQSPMQPVDQASLPGHC | | | mutation; D5 |
| | 192)M25I/ | REPPPWENEATERIYHFVVGQMVYYQCVQGYRALHRGPAESVCKMTHGKTRW | | | to Y |
| | D4L/D5Y | TQPQLICTGEMETSQFPGEEKPQASPEGRPESETSC | | | mutation; |
| | | | | | M25 to I |
| | | | | | mutation |
| 41 | h IL-2Ralpha | ELCDDDPPEIPHATFKAMAYKEGTILNCECKRGFRRIKSGSVYMLCTGNSSH | human | blocker | M25 to I |
| | (1- | SSWDNQCQCTSSATRNTTKQVTPQPEEQKERKTTEMQSPMQPVDQASLPGHC | | | mutation; |
| | 192)M25I/ | REPPPWENEATERIYHFVVGQMVYYQCVQGYRALHRGPAESVCKMTHGKTRW | | | L42 to V |
| | L42V | TQPQLICTGEMETSQFPGEEKPQASPEGRPESETSC | | | mutation |
| 42 | h IL-2Ralpha | ELCLYDPPEIPHATFKAMAYKEGTMLNCECKRGFRRIKSGSVYMLCTGNSSH | human | blocker | D4 to L |
| | (1-192) | SSWDNQCQCTSSATRNTTKQVTPQPEEQKERKTTEMQSPMQPVDQASLPGHC | | | mutation; D5 |
| | D4L/D5Y/ | REPPPWENEATERIYHFVVGQMVYYQCVQGYRALHRGPAESVCKMTHGKTRW | | | to Y |
| | L42V | TQPQLICTGEMETSQFPGEEKPQASPEGRPESETSC | | | mutation; |
| | | | | | L42 to V |
| | | | | | mutation |
| 43 | h IL-2Ralpha | ELCLYDPPEIPHATFKAMAYKEGTILNCECKRGFRRIKSGSVYMLCTGNSSH | human | blocker | D4 to L |
| | (1-192) | SSWDNQCQCTSSATRNTTKQVTPQPEEQKERKTTEMQSPMQPVDQASLPGHC | | | mutation; D5 |
| | M25I/D4L/ | REPPPWENEATERIYHFVVGQMVYYQCVQGYRALHRGPAESVCKMTHGKTRW | | | to Y |
| | D5Y/L42V | TQPQLICTGEMETSQFPGEEKPQASPEGRPESETSC | | | mutation; |
| | | | | | M25 to I |
| | | | | | mutation; |
| | | | | | L42 to V |
| | | | | | mutation |
| 44 | h IL-2Ralpha | ELCDDDPPEIPHATFKAMAYKEGTMLNCECKRGFRRIKSGSLYMLCTGNSSH | human | blocker | Wild-type |
| | (1-178) | SSWDNQCQCTSSATRNTTKQVTPQPEEQKERKTTEMQSPMQPVDQASLPGHC | | | amino acids |
| | | REPPPWENEATERIYHFVVGQMVYYQCVQGYRALHRGPAESVCKMTHGKTRW | | | 1-178 |
| | | TQPQLICTGEMETSQFPGEEKP | | | |
| 45 | h IL-2Ralpha | ELCDDDPPEIPHATFKAMAYKEGTILNCECKRGFRRIKSGSLYMLCTGNSSH | human | blocker | M25 to I |
| | (1-178) M25I | SSWDNQCQCTSSATRNTTKQVTPQPEEQKERKTTEMQSPMQPVDQASLPGHC | | | mutation |
| | | REPPPWENEATERIYHFVVGQMVYYQCVQGYRALHRGPAESVCKMTHGKTRW | | | |
| | | TQPQLICTGEMETSQFPGEEKP | | | |
| 46 | h IL-2Ralpha | ELCDDDPPEIPHATFKAMAYKEGTMLNCECKRGFRRIKSGSVYMLCTGNSSH | human | blocker | L42 to V |
| | (1-178) L42V | SSWDNQCQCTSSATRNTTKQVTPQPEEQKERKTTEMQSPMQPVDQASLPGHC | | | mutation |
| | | REPPPWENEATERIYHFVVGQMVYYQCVQGYRALHRGPAESVCKMTHGKTRW | | | |
| | | TQPQLICTGEMETSQFPGEEKP | | | |
| 47 | h IL-2Ralpha | ELCLYDPPEIPHATFKAMAYKEGTMLNCECKRGFRRIKSGSLYMLCTGNSSH | human | blocker | D4 to L |
| | (1-178) | SSWDNQCQCTSSATRNTTKQVTPQPEEQKERKTTEMQSPMQPVDQASLPGHC | | | mutation; D5 |
| | D4L/D5Y | REPPPWENEATERIYHFVVGQMVYYQCVQGYRALHRGPAESVCKMTHGKTRW | | | to Y |
| | | TQPQLICTGEMETSQFPGEEKP | | | mutation |
| 48 | h IL-2Ralpha | ELCDDDPPEIPHATFKAMAYKEGTMLNCECKRGFRRIKELVYMLCTGNSSHS | human | blocker | Wild-type |
| | (1-178) | SWDNQCQCTSSATRNTTKQVTPQPEEQKERKTTEMQSPMQPVDQASLPGHCR | | | residues 39- |
| | SGSL₃₉- | EPPPWENEATERIYHFVVGQMVYYQCVQGYRALHRGPAESVCKMTHGKTRWT | | | 42 replaced |
| | ₄₂ELV | QPQLICTGEMETSQFPGEEKP | | | with ELV |
| 49 | h IL-2Ralpha | ELCDDDPPEIPHATFKAMAYKEGTILNCECKRGFRRIKSGSVYMLCTGNSSH | human | blocker | M25 to I |
| | (1-178) | SSWDNQCQCTSSATRNTTKQVTPQPEEQKERKTTEMQSPMQPVDQASLPGHC | | | mutation; |
| | M25I/L42V | REPPPWENEATERIYHFVVGQMVYYQCVQGYRALHRGPAESVCKMTHGKTRW | | | L42 to V |
| | | TQPQLICTGEMETSQFPGEEKP | | | mutation |
| 50 | h IL-2Ralpha | ELCLYDPPEIPHATFKAMAYKEGTMLNCECKRGFRRIKSGSVYMLCTGNSSH | human | blocker | D4 to L |
| | (1-178) | SSWDNQCQCTSSATRNTTKQVTPQPEEQKERKTTEMQSPMQPVDQASLPGHC | | | mutation; D5 |
| | D4L/D5Y/ | REPPPWENEATERIYHFVVGQMVYYQCVQGYRALHRGPAESVCKMTHGKTRW | | | to Y |
| | L42V | TQPQLICTGEMETSQFPGEEKP | | | mutation; |
| | | | | | L42 to V |
| | | | | | mutation |
| 51 | h IL-2Ralpha | ELCLYDPPEIPHATFKAMAYKEGTILNCECKRGFRRIKSGSVYMLCTGNSSH | human | blocker | D4 to L |
| | (1-178) | SSWDNQCQCTSSATRNTTKQVTPQPEEQKERKTTEMQSPMQPVDQASLPGHC | | | mutation; D5 |
| | D4L/D5Y/ | REPPPWENEATERIYHFVVGQMVYYQCVQGYRALHRGPAESVCKMTHGKTRW | | | to Y |
| | M25I/ L42V | TQPQLICTGEMETSQFPGEEKP | | | mutation; |
| | | | | | M25 to I |
| | | | | | mutation; |
| | | | | | L42 to V |
| | | | | | mutation |
| 52-69 | Not Used | | | | |

Pharmacokinetic modulators

| | | | | | |
|---|---|---|---|---|---|
| 70 | h IgG1 Fc | DKTHTCPPCPAPELLGGPSVFLFPPKPKDTLMISRTPEVTCVVVDVSHEDPEVK | human | half-life | C-terminal K |
| | | FNWYVDGVEVHNAKTKPREEQYNSTYRVVSVLTVLHQDWLNGKEYKCKVSNKAL | | extension | residue |
| | | PAPIEKTISKAKGQPREPQVYTLPPSRDELTKNQVSLTCLVKGFYPSDIAVEWE | | | deleted |
| | | SNGQPENNYKTTPPVLDSDGSFFLYSKLTVDKSRWQQGNVFSCSVMHEALHNHY | | | |
| | | TQKSLSLSPG | | | |
| 71 | Human IgG1 | DKTHTCPPCPAPELLGGPSVFLFPPKPKDTLMISRTPEVTCVVVDVSHEDPEVK | human | half-life | |
| | K392D | FNWYVDGVEVHNAKTKPREEQYNSTYRVVSVLTVLHQDWLNGKEYKCKVSNKAL | | extension | |
| | K409D Fc | PAPIEKTISKAKGQPREPQVYTLPPSRDELTKNQVSLTCLVKGFYPSDIAVEWE | | | |
| | domain | SNGQPENNYDTTPPVLDSDGSFFLYSDLTVDKSRWQQGNVFSCSVMHEALHNHY | | | |
| | polypeptide | TQKSLSLSPG | | | |
| | sequence | | | | |
| 72 | Human serum | RGVFRRDAHKSEVAHRFKDLGEENFKALVLIA | human | half-life | wild-type |
| | albumin | FAQYLQQCPFEDHVKLVNEVTEFAKTCVADESAENCDKSLHTLFGDKLCT | | extension | |
| | | VATLRETYGEMADCCAKQEPERNECFLQHKDDNPNLPRLVRPEVDVMCTA | | | |
| | | FHDNEETFLKKYLYEIARRHPYFYAPELLFFAKRYKAAFTECCQAADKAACLLP | | | |
| | | KLDELRDEGKASSAKQRLKCASLQKFGERAFKAWAVARLSQRFPKA | | | |
| | | EFAEVSKLVTDLTKVHTECCHGDLLECADDRADLAKYICENQDSISSKLK | | | |
| | | ECCEKPLLEKSHCIAEVENDEMPADLPSLAADFVESKDVCKNYAEAKDVF | | | |
| | | LGMFLYEYARRHPDYSVVLLLRLAKTYETTLEKCCAAADPHECYAKVFDE | | | |
| | | FKPLVEEPQNLIKQNCELFEQLGEYKFQNALLVRYTKKVPQVSTPTLVEV | | | |
| | | SRNLGKVGSKCCKHPEAKRMPCAEDYLSVVLNQLCVLHEKTPVSDRVTKC | | | |
| | | CTESLVNRRPCFSALEVDETYVPKEFNAETFTFHADICTLSEKERQIKKQ | | | |
| | | TALVELVKHKPKATKEQLKAVMDDFAAFVEKCCKADDKETCFAEEGKKLV | | | |
| | | AASQAALGL | | | |
| 73 | m IgG1 Fc | GCKPCICTVPEVSSVFIFPPKPKDVLTITLTPKVTCVVVDISKDDPEVQFSWFV | mouse | half-life | wild-type |
| | | DDVEVHTAQTQPREEQFNSTFRSVSELPIMHQDWLNGKEFKCRVNSAAFPAPIE | | extension | |
| | | KTISKTKGRPKAPQVYTIPPPKEQMAKDKVSLTCMITDFFPEDITVEWQWNGQP | | | |
| | | AENYKNTQPIMDTDGSYFVYSKLNVQKSNWEAGNTFTCSVLHEGLHNHHTEKSL | | | |
| | | SHSPGK | | | |
| 74 | Murine IgG1 | GCKPCICTVPEVSSVFIFPPKPKDVLMITLTPKVTCVVVDISKDDPEVQFSWFV | mouse | half-life | |
| | T252M Fc | DDVEVHTAQTQPREEQFNSTFRSVSELPIMHQDWLNGKEFKCRVNSAAFPAPIE | | extension | |
| | domain | KTISKTKGRPKAPQVYTIPPPKEQMAKDKVSLTCMITDFFPEDITVEWQWNGQP | | | |
| | polypeptide | AENYKNTQPIMDTDGSYFVYSKLNVQKSNWEAGNTFTCSVLHEGLHNHHTEKSL | | | |
| | sequence | SHSPG | | | |
| 75-79 | Not Used | | | | |
| 756 | IgG1 Fc | DKTHTCPPCPAPELLGGPSVFLFPPKPKDTLMISRTPEVTCVVVDVSHEDPE | human | half-life | Knob |
| | (K360E/K409W) | VKFNWYVDGVEVHNAKTKPREEQYNSTYRVVSVLTVLHQDWLNGKEYKCKVS | | extension | mutations |
| | Knob | NKALPAPIEKTISKAKGQPREPQVYTLPPSRDELTENQVSLTCLVKGFYPSD | | | |
| | | IAVEWESNGQPENNYKTTPPVLDSDGSFFLYSWLTVDKSRWQQGNVFSCSVM | | | |
| | | HEALHNHYTQKSLSLSPG | | | |
| 757 | h IgG1 Fc | DKTHTCPPCPAPELLGGPSVFLFPPKPKDTLMISRTPEVTCVVVDVSHEDPE | human | half-life | Hole |
| | (Q347R/D399V/ | VKFNWYVDGVEVHNAKTKPREEQYNSTYRVVSVLTVLHQDWLNGKEYKCKVS | | extension | mutations |
| | F405T) | NKALPAPIEKTISKAKGQPREPRVYTLPPSRDELTKNQVSLTCLVKGFYPSD | | | |
| | Hole | IAVEWESNGQPENNYKTTPPVLVSDGSFTLYSKLTVDKSRWQQGNVFSCSVM | | | |
| | | HEALHNHYTQKSLSLSPG | | | |

MMP cleavable segments

| | | |
|---|---|---|
| 80 | MMP cleavage | GPLGVRG |
| | site | |
| | polypeptide | |
| | sequence | |
| 81 | G112631 | GPLGVRG |
| | polypeptide | |
| | sequence | |
| 82 | G112632 | GPLGLRG |
| | polypeptide | |
| | sequence | |
| 83 | G112633 | GPLGLAR |
| | polypeptide | |
| | sequence | |
| 84 | G112634 | GPAALVGA |
| | polypeptide | |
| | sequence | |
| 85 | G112635 | GPAALIGG |
| | polypeptide | |
| | sequence | |
| 86 | G112636 | GPLNLVGR |
| | polypeptide | |
| | sequence | |
| 87 | G112637 | GPAGLVAD |
| | polypeptide | |
| | sequence | |
| 88 | G112638 | GPANLVAP |
| | polypeptide | |
| | sequence | |
| 89 | G112639 | VPLSLYSG |
| | polypeptide | |
| | sequence | |
| 90 | G112640 | SGESPAYYTA |
| | polypeptide | |
| | sequence | |
| 91 | MMP | PXXX_{Hy} |
| | consensus | |
| | motif | |
| 92 | MMP-2 | (L/I)XXX_{Hy} |
| | consensus | |
| | motif | |
| 93 | MMP-2 | X_{Hy}SXL |
| | consensus | |
| | motif | |
| 94 | MMP-2 | HXXX_{Hy} |
| | consensus | |
| | motif | |
| 95-99 | Not Used | |

IL-2 Fusion polypeptides

| | | |
|---|---|---|
| 100 | Construct A | APTSSSTSSSTAEAQQQQQQQQQQQQHLEQLLMDLQELLSRMENYRNLKLPRML |
| | polypeptide | TFKFYLPKQATELKDLQCLEDELGPLRHVLDLTQSKSFQLEDAENFISNIRVTV |
| | sequence: | VKLKGSDNTFECQFDDESATVVDFLRRWIAFCQSIISTSPQSGGGGSGGGGGPL |
| | mIL2- | GVRGGGGGSGGGGSELCLYDPPEVPNATFKALSYKNGTILNCECKRGFRRLKEL |
| | 2x(SG4) - | VYMRCLGNSWSSNCQCTSNSHDKSRKQVTAQLEHQKEQQTTTDMQKPTQSMHQE |
| | MMPcs1 - | NLTGHCREPPPWKHEDSKRIYHFVEGQSVHYECIPGYKALQRGPAISICKMKCG |
| | 2x(G4S) - | KTGWTQPQLTCVDEREHHRFLASEESQGSRNSSPESETSCPITTTDFPQPTETT |
| | IL2Ralpha - | AMTETFVLTMEYKHHHHHH |
| | 6His | |
| 101 | Construct B | APTSSSTSSSTAEAQQQQQQQQQQQQHLEQLLMDLQELLSRMENYRNLKLPRML |
| | polypeptide | TFKFYLPKQATELKDLQCLEDELGPLRHVLDLTQSKSFQLEDAENFISNIRVTV |
| | sequence: m | VKLKGSDNTFECQFDDESATVVDFLRRWIAFCQSIISTSPQSGGGGSGGGGGPL |
| | IL2-2x(SG4) - | GVRGGGGGSGGGGSELCLYDPPEVPNATFKALSYKNGTILNCECKRGFRRLKEL |
| | MMPcs1 - 2x | VYMRCLGNSWSSNCQCTSNSHDKSRKQVTAQLEHQKEQQTTTDMQKPTQSMHQE |
| | (G4S) - | NLTGHCREPPPWKHEDSKRIYHFVEGQSVHYECIPGYKALQRGPAISICKMKCG |
| | IL2Ralpha - | KTGWTQPQLTCVDEREHHRFLASEESQGSRNSSPESETSCPITTTDFPQPTETT |
| | mIgG1 Fc | AMTETFVLTMEYKGCKPCICTVPEVSSVFIFPPKPKDVLTITLTPKVTCVVVDI |
| | | SKDDPEVQFSWFVDDVEVHTAQTQPREEQFNSTFRSVSELPIMHQDWLNGKEFK |
| | | CRVNSAAFPAPIEKTISKTKGRPKAPQVYTIPPPKEQMAKDKVSLTCMITDFFP |
| | | EDITVEWQWNGQPAENYKNTQPIMDTDGSYFVYSKLNVQKSNWEAGNTFTCSVL |
| | | HEGLHNHHTEKSLSHSPGK |
| 102 | Construct C | APTSSSTSSSTAEAQQQQQQQQQQQQHLEQLLMDLQELLSRMENYRNLKLPRML |
| | polypeptide | TFKFYLPKQATELKDLQCLEDELGPLRHVLDLTQSKSFQLEDAENFISNIRVTV |
| | sequence: | VKLKGSDNTFECQFDDESATVVDFLRRWIAFSQSIISTSPQSGGGGSGGGGGPL |
| | mIL2(C140S) - | GVRGGGGGSGGGGSELCLYDPPEVPNATFKALSYKNGTILNCECKRGFRRLKEL |
| | 2x(SG4) - | VYMRCLGNSWSSNCQCTSNSHDKSRKQVTAQLEHQKEQQTTTDMQKPTQSMHQE |
| | MMPcs1 - | NLTGHCREPPPWKHEDSKRIYHFVEGQSVHYECIPGYKALQRGPAISICKMKCG |
| | 2x(G4S) - | KTGWTQPQLTCVDEREHHRFLASEESQGSRNSSPESETSCPITTTDFPQPTETT |
| | IL2Ralpha - | AMTETFVLTMEYKGCKPCICTVPEVSSVFIFPPKPKDVLMITLTPKVTCVVVDI |
| | mIgG1 | SKDDPEVQFSWFVDDVEVHTAQTQPREEQFNSTFRSVSELPIMHQDWLNGKEFK |
| | Fc(T252M)- | CRVNSAAFPAPIEKTISKTKGRPKAPQVYTIPPPKEQMAKDKVSLTCMITDFFP |
| | 6xHIS | EDITVEWQWNGQPAENYKNTQPIMDTDGSYFVYSKLNVQKSNWEAGNTFTCSVL |
| | | HEGLHNHHTEKSLSHSPGHHHHHH |
| 103 | Construct R | APTSSSTSSSTAEAQQQQQQQQQQQQHLEQLLMDLQELLSRMENYRNLKLPRML |
| | polypeptide | TFKFYLPKQATELKDLQCLEDELGPLRHVLDLTQSKSFQLEDAENFISNIRVTV |
| | sequence: | VKLKGSDNTFECQFDDESATVVDFLRRWIAFSQSIISTSPQSGGGGSGGGGGPL |
| | mIL2(C140S)- | GVRGGGGGSGGGGSELCLYDPPEVPNATFKALSYKNGTILNCECKRGFRRLKEL |
| | 2x(SG4) - | VYMRCLGNSWSSNCQCTSNSHDKSRKQVTAQLEHQKEQQTTTDMQKPTQSMHQE |
| | MMPcs1 - | NLTGHCREPPPWKHEDSKRIYHFVEGQSVHYECIPGYKALQRGPAISICKMKCG |
| | 2x(G4S) - | KTGWTQPQLTCVDEREHHRFLASEESQGSRNSSPESETSCPITTTDFPQPTETT |
| | IL2Ralpha - | AMTETFVLTMEYKDKTHTCPPCPAPELLGGPSVFLFPPKPKDTLMISRTPEVTC |
| | hu IgG1 Fc- | VVVDVSHEDPEVKFNWYVDGVEVHNAKTKPREEQYNSTYRVVSVLTVLHQDWLN |
| | 6xHIS | GKEYKCKVSNKALPAPIEKTISKAKGQPREPQVYTLPPSRDELTKNQVSLTCLV |
| | | KGFYPSDIAVEWESNGQPENNYKTTPPVLDSDGSFFLYSKLTVDKSRWQQGNVF |
| | | SCSVMHEALHNHYTQKSLSLSPGHHHHHH |
| 104 | Construct D | APTSSSTKKTQLQLEHLLLDLQMILNGINNYKNPKLTRMLTFKFYMPKKATELK |
| | polypeptide | HLQCLEEELKPLEEVLNLAQSKNFHLRPRDLISNINVIVLELKGSETTFMCEYA |
| | Sequence: | DETATIVEFLNRWITFSQSIISTLTSGGGGSGGGGGPLGVRGGGGGSGGGGSEL |
| | mIL2(C140S)- | CDDDPPEIPHATFKAMAYKEGTMLNCECKRGFRRIKSGSLYMLCTGNSSHSSWD |
| | 2x(SG4) - | NQCQCTSSATRNTTKQVTPQPEEQKERKTTEMQSPMQPVDQASLPGHCREPPPW |
| | MMPcs1 - | ENEATERIYHFVVGQMVYYQCVQGYRALHRGPAESVCKMTHGKTRWTQPQLICT |
| | 2x(G4S) - | GEMETSQFPGEEKPQASPEGRPESETSCLVTTTDFQIQTEMAATMETSIFTTEY |
| | SIL2Ralpha - | QDKTHTCPPCPAPELLGGPSVFLFPPKPKDTLMISRTPEVTCVVVDVSHEDPEV |
| | mIgG1 | KFNWYVDGVEVHNAKTKPREEQYNSTYRVVSVLTVLHQDWLNGKEYKCKVSNKA |
| | Fc(T252M)- | LPAPIEKTISKAKGQPREPQVYTLPPSRDELTKNQVSLTCLVKGFYPSDIAVEW |
| | 6xHIS | ESNGQPENNYKTTPPVLDSDGSFFLYSKLTVDKSRWQQGNVFSCSVMHEALHNH |
| | | YTQKSLSLSPGHHHHHH |
| 105 | mIgG1 Fc - | GCKPCICTVPEVSSVFIFPPKPKDVLTITLTPKVTCVVVDISKDDPEVQFSWFV |
| | Murine IL2 - | DDVEVHTAQTQPREEQFNSTFRSVSELPIMHQDWLNGKEFKCRVNSAAFPAPIE |
| | 2x(SG4) - | KTISKTKGRPKAPQVYTIPPPKEQMAKDKVSLTCMITDFFPEDITVEWQWNGQP |
| | MMPcs1 - 2x | AENYKNTQPIMDTDGSYFVYSKLNVQKSNWEAGNTFTCSVLHEGLHNHHTEKSL |
| | (G4S) - | SHSPGKAPTSSST`SSSTAEAQQQQQQQQQQQQHLEQLLMDLQELLSRMENYRN |
| | IL2Ralpha | LKLPRMLTFKFYLPKQATELKDLQCLEDELGPLRHVLDLTQSKSFQLEDAENFI |
| | (long kinetic | SNIRVTVVKLKGSDNTFECQFDDESATVVDFLRRWIAFCQSIISTSPQSGGGGS |
| | IL2 post | GGGGGPLGVRGGGGGSGGGGSELCLYDPPEVPNATFKALSYKNGTILNCECKRG |
| | cleavage) | FRRLKELVYMRCLGNSWSSNCQCTSNSHDKSRKQVTAQLEHQKEQQTTTDMQKP |
| | polypeptide | TQSMHQENLTGHCREPPPWKHEDSKRIYHFVEGQSVHYECIPGYKALQRGPAIS |
| | Sequence | ICKMKCGKTGWTQPQLTCVDEREHHRFLASEESQGSRNSSPESETSCPITTTDF |
| | | PQPTETTAMTETFVLTMEYK |
| 106 | Construct E | APTSSSTKKTQLQLEHLLLDLQMILNGINNYKNPKLTRMLTFKFYMPKKATELK |
| | polypeptide | HLQCLEEELKPLEEVLNLAQSKNFHLRPRDLISNINVIVLELKGSETTFMCEYA |
| | sequence: | DETATIVEFLNRWITFSQSIISTLTSGGGGSGGGGGPLGVRGGGGGSGGGGSEL |
| | HuIL2(C125S) - | CDDDPPEIPHATFKAMAYKEGTMLNCECKRGFRRIKSGSLYMLCTGNSSHSSWD |
| | 2x(SG4) - | NQCQCTSSATRNTTKQVTPQPEEQKERKTTEMQSPMQPVDQASLPGHCREPPPW |
| | MMPcs1 - | ENEATERIYHFVVGQMVYYQCVQGYRALHRGPAESVCKMTHGKTRWTQPQLICT |
| | 2x(G4S) - | GEMETSQFPGEEKPQASPEGRPESETSCLVTTTDFQIQTEMAATMETSIFTTEY |
| | IL2Ralpha | QDKTHTCPPCPAPELLGGPSVFLFPPKPKDTLMISRTPEVTCVVVDVSHEDPEV |
| | hu IgG1 Fc - | KFNWYVDGVEVHNAKTKPREEQYNSTYRVVSVLTVLHQDWLNGKEYKCKVSNKA |
| | 6xHIS | LPAPIEKTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMHEALHNH |
| | | YTQKSLSLSPGHHHHHH |
| 107 | Construct S | APTSSSTKKTQLQLEHLLLDLQMILNGINNYKNPKLTRMLTFKFYMPKKATELK |
| | polypeptide | HLQCLEEELKPLEEVLNLAQSKNFHLRPRDLISNINVIVLELKGSETTFMCEYA |
| | sequence: | DETATIVEFLNRWITFCQSIISTLTSGGGGSGGGGGPLGVRGGGGGSGGGGSEL |
| | hIL2- 2x(SG4) - | CDDDPPEIPHATFKAMAYKEGTMLNCECKRGFRRIKSGSLYMLCTGNSSHSSWD |
| | MMPcs1 - | NQCQCTSSATRNTTKQVTPQPEEQKERKTTEMQSPMQPVDQASLPGHCREPPPW |
| | 2x(G4S) - | ENEATERIYHFVVGQMVYYQCVQGYRALHRGPAESVCKMTHGKTRWTQPQLICT |
| | hIL2Ralpha - | GEMETSQFPGEEKPQASPEGRPESETSCLVTTTDFQIQTEMAATMETSIFTTEY |
| | hIgG1Fc_mut | QDKTHTCPPCPAPELLGGPSVFLFPPKPKDTLMISRTPEVTCVVVDVSHEDPEV |
| | 1 (K392D; | KFNWYVDGVEVHNAKTKPREEQYNSTYRVVSVLTVLHQDWLNGKEYKCKVSNKA |
| | K409D) | LPAPIEKTISKAKGQPREPQVYTLPPSRDELTKNQVSLTCLVKGFYPSDIAVEW |
| | | ESNGQPENNYDTTPPVLDSDGSFFLYSDLTVDKSRWQQGNVFSCSVMHEALHNH |
| | | YTQKSLSLSPGHHHHHH |
| 108 | Construct T | DKTHTCPPCPAPELLGGPSVFLFPPKPKDTLMISRTPEVTCVVVDVSHEDPEVK |
| | polypeptide | FNWYVDGVEVHNAKTKPREEQYNSTYRVVSVLTVLHQDWLNGKEYKCKVSNKAL |
| | sequence | PAPIEKTISKAKGQPREPQVYTLPPSRKELTKNQVSLTCLVKGFYPSDIAVEWE |
| | including | SNGQPENNYKTTPPVLKSDGSFFLYSKLTVDKSRWQQGNVFSCSVMHEALHNHY |
| | hlgG1 Fc_mut | TQKSLSLSPGHHHHHH |
| | 2 (D356K; | |
| | D399K) | |
| 109 | hu IgG1 Fc - | DKTHTCPPCPAPELLGGPSVFLFPPKPKDTLMISRTPEVTCVVVDVSHEDPEVK |
| | Hu | FNWYVDGVEVHNAKTKPREEQYNSTYRVVSVLTVLHQDWLNGKEYKCKVSNKAL |
| | IL2(C125S)- | PAPIEKTISKAKGQPREPQVYTLPPSRDELTKNQVSLTCLVKGFYPSDIAVEWE |
| | 2x(SG4) - | SNGQPENNYKTTPPVLDSDGSFFLYSKLTVDKSRWQQGNVFSCSVMHEALHNHY |
| | MMPcs1 - | TQKSLSLSPGAPTSSSTKKTQLQLEHLLLDLQMILNGINNYKNPKLTRMLTFKF |
| | 2x(G4S) - | YMPKKATELKHLQCLEEELKPLEEVLNLAQSKNFHLRPRDLISNINVIVLELKG |
| | IL2Ralpha | SETTFMCEYADETATIVEFLNRWITFSQSIISTLTSGGGGSGGGGGPLGVRGGG |
| | Polypeptide | GGSGGGGSELCDDDPPEIPHATFKAMAYKEGTMLNCECKRGFRRIKSGSLYMLC |
| | Sequence | TGNSSHSSWDNQCQCTSSATRNTTKQVTPQPEEQKERKTTEMQSPMQPVDQASL |
| | | PGHCREPPPWENEATERIYHFVVGQMVYYQCVQGYRALHRGPAESVCKMTHGKT |
| | | RWTQPQLICTGEMETSQFPGEEKPQASPEGRPESETSCLVTTTDFQIQTEMAAT |
| | | METSIFTTEYQ |
| 110 | hIL2- 2x(SG4) - | APTSSSTKKTQLQLEHLLLDLQMILNGINNYKNPKLTRMLTFKFYMPKKATELK |
| | MMPcs1 - | HLQCLEEELKPLEEVLNLAQSKNFHLRPRDLISNINVIVLELKGSETTFMCEYA |
| | 2x(G4S) - | DETATIVEFLNRWITFCQSIISTLTSGGGGSGGGGGPLGVRGGGGGSGGGGSAV |
| | hIL2Rbeta | NGTSQFTCFYNSRANISCVWSQDGALQDTSCQVHAWPDRRRWNQTCELLPVSQA |
| | hIgG1Fc | SWACNLILGAPDSQKLTTVDIVTLRVLCREGVRWRVMAIQDFKPFENLRLMAPI |
| | (Construct U) | SLQVVHVETHRCNISWEISQASHYFERHLEFEARTLSPGHTWEEAPLLTLKQKQ |
| | polypeptide | EWICLETLTPDTQYEFQVRVKPLQGEFTTWSPWSQPLAFRTKPAALGKDTDKTH |
| | Sequence | TCPPCPAPELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNHYTQKS |
| | | LSLSPG |
| 111 | hIL2- 2x(SG4) - | APTSSSTKKTQLQLEHLLLDLQMILNGINNYKNPKLTRMLTFKFYMPKKATELK |
| | MMPcs1 - | HLQCLEEELKPLEEVLNLAQSKNFHLRPRDLISNINVIVLELKGSETTFMCEYA |
| | 2x(G4S) - | DETATIVEFLNRWITFCQSIISTLTSGGGGSGGGGGPLGVRGGGGGSGGGGSLN |
| | hIL2Rgamma - | TTILTPNGNEDTTADFFLTTMPTDSLSVSTLPLPEVQCFVFNVEYMNCTWNSSS |
| | hIgG1Fc | EPQPTNLTLHYWYKNSDNDKVQKCSHYLFSEEITSGCQLQKKEIHLYQTFVVQL |
| | polypeptide | QDPREPRRQATQMLKLQNLVIPWAPENLTLHKLSESQLELNWNNRFLNHCLEHL |
| | sequence | VQYRTDWDHSWTEQSVDYRHKFSLPSVDGQKRYTFRVRSRFNPLCGSAQHWSEW |
| | | SHPIHWGSNTSKENPFLFALEADKTHTCPPCPAPELLGGPSVFLFPPKPKDTLM |
| | | ISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSRDELTK |
| | | NQVSLTCLVKGFYPSDIAVEWESNGQPENNYKTTPPVLDSDGSFFLYSKLTVDK |
| | | SRWQQGNVFSCSVMHEALHNHYTQKSLSLSPG |
| 112- | Not Used | |
| 119 | | |

Other

| | | |
|---|---|---|
| 120 | Gly-Ser rich | SGGGGSGGGG |
| | linker | |
| | polypeptide | |
| | sequence | |
| 121- | Not Used | |
| 129 | | |

Fusion polypeptides (DNA coding sequences)

| | | |
|---|---|---|
| 130 | Construct A | ATGGGTTGGTCCTGCATCATCCTGTTCCTGGTCGCCACCGCCACTGGGGTCCAC |
| | DNA sequence | TCCGCACCTACATCATCATCAACTTCATCCTCCACCGCTGAGGCTCAGCAACAA |
| | (mIL2- | CAGCAACAACAGCAGCAGCAGCAGCAGCATCTGGAGCAGCTGCTGATGGACCTG |
| | 2x(SG4) - | CAGGAGCTGCTGTCCAGAATGGAGAACTACCGCAATCTGAAGCTGCCAAGGATG |
| | MMPcs1 - | CTGACCTTCAAGTTTTATCTGCCCAAGCAGGCCACAGAGCTGAAGGACCTGCAG |
| | 2x(G4S) - | TGCCTGGAGGATGAGCTGGGCCCACTGAGGCACGTGCTGGACCTGACCCAGAGC |
| | IL2Ralpha - | AAGTCTTTCCAGCTGGAGGATGCTGAGAACTTTATCTCCAATATCCGGGTGACC |
| | 6His) | GTGGTGAAGCTGAAGGGCAGCGACAACACATTCGAGTGCCAGTTTGACGATGAG |
| | | TCTGCCACCGTGGTGGATTTCCTGAGGCGGTGGATCGCTTTTTGTCAGAGCATC |
| | | ATCTCCACAAGCCCTCAGTCTGGAGGAGGTGGCAGCGGAGGAGGAGGTGGCCCA |
| | | CTGGGCGTGAGGGGTGGCGGCGGCGGCTCTGGCGGCGGCGGCTCCGAGCTGTGC |
| | | CTGTACGACCCCCCTGAGGTGCCCAATGCCACCTTCAAGGCTCTGTCTTATAAG |
| | | AACGGCACAATCCTGAATTGCGAGTGTAAGAGGGGCTTTAGACGCCTGAAGGAG |
| | | CTGGTGTACATGCGGTGTCTGGGCAACTCCTGGTCCAGCAATTGCCAGTGTACC |
| | | TCTAACTCCCATGACAAGAGCAGAAAGCAGGTGACAGCCCAGCTGGAGCACCAG |
| | | AAGGAGCAGCAGACCACAACCGATATGCAGAAGCCCACCCAGTCTATGCACCAG |
| | | GAGAATCTGACAGGCCATTGCAGAGAGCCACCCCCTTGGAAGCACGAGGATAGC |
| | | AAGCGCATCTATCATTTCGTGGAGGGCCAGTCTGTGCACTACGAGTGTATCCCC |
| | | GGCTATAAGGCCCTGCAGAGAGGCCCTGCTATCTCCATCTGCAAGATGAAGTGT |
| | | GGCAAGACCGGCTGGACACAGCCTCAGCTGACCTGCGTGGACGAGAGGGAGCAC |
| | | CATCGGTTCCTGGCTAGCGAGGAGTCTCAGGGCTCCCGCAACTCTTCCCCTGAG |
| | | AGCGAGACATCTTGTCCAATCACAACCACAGATTTTCCACAGCCCACCGAGACA |
| | | ACCGCTATGACAGAGACCTTCGTGCTGACTATGGAATACAAACACCACCACCAC |
| | | CACCACTAATGA |
| 131 | Construct B | ATGGGTTGGTCCTGCATCATCCTGTTCCTGGTCGCCACCGCCACTGGGGTCCAC |
| | DNA | TCCGCACCTACATCATCATCAACTTCATCCTCCACCGCTGAGGCTCAGCAACAA |
| | sequence: m | CAGCAACAACAGCAGCAGCAGCAGCAGCATCTGGAGCAGCTGCTGATGGACCTG |
| | IL2-2x(SG4) - | CAGGAGCTGCTGTCCAGAATGGAGAACTACCGCAATCTGAAGCTGCCAAGGATG |
| | MMPcs1 - 2x | CTGACCTTCAAGTTTTATCTGCCCAAGCAGGCCACAGAGCTGAAGGACCTGCAG |
| | (G4S) - | TGCCTGGAGGATGAGCTGGGCCCACTGAGGCACGTGCTGGACCTGACCCAGAGC |
| | IL2Ralpha - | AAGTCTTTCCAGCTGGAGGATGCTGAGAACTTTATCTCCAATATCCGGGTGACC |
| | mIgG1 Fc | GTGGTGAAGCTGAAGGGCAGCGACAACACATTCGAGTGCCAGTTTGACGATGAG |
| | | TCTGCCACCGTGGTGGATTTCCTGAGGCGGTGGATCGCTTTTTGTCAGAGCATC |
| | | ATCTCCACAAGCCCTCAGTCTGGAGGAGGTGGCAGCGGAGGAGGAGGTGGCCCA |
| | | CTGGGCGTGAGGGGTGGCGGCGGCGGCTCTGGCGGCGGCGGCTCCGAGCTGTGC |
| | | CTGTACGACCCCCCTGAGGTGCCCAATGCCACCTTCAAGGCTCTGTCTTATAAG |
| | | AACGGCACAATCCTGAATTGCGAGTGTAAGAGGGGCTTTAGACGCCTGAAGGAG |
| | | CTGGTGTACATGCGGTGTCTGGGCAACTCCTGGTCCAGCAATTGCCAGTGTACC |
| | | TCTAACTCCCATGACAAGAGCAGAAAGCAGGTGACAGCCCAGCTGGAGCACCAG |
| | | AAGGAGCAGCAGACCACAACCGATATGCAGAAGCCCACCCAGTCTATGCACCAG |
| | | GAGAATCTGACAGGCCATTGCAGAGAGCCACCCCCTTGGAAGCACGAGGATAGC |
| | | AAGCGCATCTATCATTTCGTGGAGGGCCAGTCTGTGCACTACGAGTGTATCCCC |
| | | GGCTATAAGGCCCTGCAGAGAGGCCCTGCTATCTCCATCTGCAAGATGAAGTGT |
| | | GGCAAGACCGGCTGGACACAGCCTCAGCTGACCTGCGTGGACGAGAGGGAGCAC |
| | | CATCGGTTCCTGGCTAGCGAGGAGTCTCAGGGCTCCCGCAACTCTTCCCCTGAG |
| | | AGCGAGACATCTTGTCCAATCACAACCACAGATTTTCCACAGCCCACCGAGACA |
| | | ACCGCTATGACAGAGACCTTCGTGCTGACTATGGAATACAAAGGATGCAAACCC |
| | | TGTATCTGTACCGTGCCCGAGGTCTCTTCCGTCTTTATTTTCCCCCCCAAGCCT |
| | | AAGGATGTGCTGACTATTACTCTGACCCCCAAGGTGACATGCGTGGTGGTGGAC |
| | | ATCAGCAAGGACGATCCTGAGGTGCAGTTCTCTTGGTTTGTGGACGATGTGGAG |
| | | GTGCACACCGCCCAGACACAGCCAAGGGAGGAGCAGTTCAATAGCACCTTTCGG |
| | | TCCGTGAGCGAGCTGCCCATCATGCATCAGGATTGGCTGAATGGCAAGGAGTTC |
| | | AAGTGCAGAGTGAACTCTGCCGCTTTTCCCGCTCCTATCGAGAAGACCATCTCC |
| | | AAGACAAAGGGCCGCCCAAAGGCTCCACAGGTGTACACCATCCCACCTCCAAAG |
| | | GAGCAGATGGCTAAGGACAAGGTGTCTCTGACCTGTATGATCACAGACTTCTTT |
| | | CCTGAGGACATCACAGTGGAGTGGCAGTGGAACGGCCAGCCTGCCGAGAACTAT |
| | | AAGAATACCCAGCCAATCATGGACACAGATGGCTCTTACTTCGTGTATTCCAAG |
| | | CTGAACGTGCAGAAGTCCAATTGGGAGGCTGGCAACACCTTTACATGTAGCGTG |
| | | CTGCACGAAGGTCTGCATAACCATCATACCGAAAAATCACTGTCACACTCCCCT |
| | | GGAAAATAATGA |
| 132 | Construct C | ATGGGTTGGTCCTGCATCATCCTGTTCCTGGTCGCCACCGCCACTGGGGTCCAC |
| | DNA | TCCGCACCTACATCATCATCAACTTCATCCTCCACCGCTGAGGCTCAGCAACAA |
| | sequence: m | CAGCAACAACAGCAGCAGCAGCAGCAGCATCTGGAGCAGCTGCTGATGGACCTG |
| | IL2(C140S)- | CAGGAGCTGCTGTCCAGAATGGAGAACTACCGCAATCTGAAGCTGCCAAGGATG |
| | 2x(SG4) - | CTGACCTTCAAGTTTTATCTGCCCAAGCAGGCCACAGAGCTGAAGGACCTGCAG |
| | MMPcs1 - | TGCCTGGAGGATGAGCTGGGCCCACTGAGGCACGTGCTGGACCTGACCCAGAGC |
| | 2x(G4S) - | AAGTCTTTCCAGCTGGAGGATGCTGAGAACTTTATCTCCAATATCCGGGTGACC |
| | IL2Ralpha | GTGGTGAAGCTGAAGGGCAGCGACAACACATTCGAGTGCCAGTTTGACGATGAG |
| | mIgG1 | TCTGCCACCGTGGTGGATTTCCTGAGGCGGTGGATCGCTTTTTCCCAGAGCATC |
| | Fc(T252M)- | ATCTCCACAAGCCCTCAGTCTGGAGGAGGTGGCAGCGGAGGAGGAGGTGGCCCA |
| | 6xHIS | CTGGGCGTGAGGGGTGGCGGCGGCGGCTCTGGCGGCGGCGGCTCCGAGCTGTGC |
| | | CTGTACGACCCCCCTGAGGTGCCCAATGCCACCTTCAAGGCTCTGTCTTATAAG |
| | | AACGGCACAATCCTGAATTGCGAGTGTAAGAGGGGCTTTAGACGCCTGAAGGAG |
| | | CTGGTGTACATGCGGTGTCTGGGCAACTCCTGGTCCAGCAATTGCCAGTGTACC |
| | | TCTAACTCCCATGACAAGAGCAGAAAGCAGGTGACAGCCCAGCTGGAGCACCAG |
| | | AAGGAGCAGCAGACCACAACCGATATGCAGAAGCCCACCCAGTCTATGCACCAG |
| | | GAGAATCTGACAGGCCATTGCAGAGAGCCACCCCCTTGGAAGCACGAGGATAGC |
| | | AAGCGCATCTATCATTTCGTGGAGGGCCAGTCTGTGCACTACGAGTGTATCCCC |
| | | GGCTATAAGGCCCTGCAGAGAGGCCCTGCTATCTCCATCTGCAAGATGAAGTGT |
| | | GGCAAGACCGGCTGGACACAGCCTCAGCTGACCTGCGTGGACGAGAGGGAGCAC |
| | | CATCGGTTCCTGGCTAGCGAGGAGTCTCAGGGCTCCCGCAACTCTTCCCCTGAG |
| | | AGCGAGACATCTTGTCCAATCACAACCACAGATTTTCCACAGCCCACCGAGACA |
| | | ACCGCTATGACAGAGACCTTCGTGCTGACTATGGAATACAAAGGATGCAAACCC |
| | | TGTATCTGTACCGTGCCCGAGGTCTCTTCCGTCTTTATTTTCCCCCCCAAGCCT |
| | | AAGGATGTGCTGATGATTACTCTGACCCCCAAGGTGACATGCGTGGTGGTGGAC |
| | | ATCAGCAAGGACGATCCTGAGGTGCAGTTCTCTTGGTTTGTGGACGATGTGGAG |
| | | GTGCACACCGCCCAGACACAGCCAAGGGAGGAGCAGTTCAATAGCACCTTTCGG |
| | | TCCGTGAGCGAGCTGCCCATCATGCATCAGGATTGGCTGAATGGCAAGGAGTTC |
| | | AAGTGCAGAGTGAACTCTGCCGCTTTTCCCGCTCCTATCGAGAAGACCATCTCC |
| | | AAGACAAAGGGCCGCCCAAAGGCTCCACAGGTGTACACCATCCCACCTCCAAAG |
| | | GAGCAGATGGCTAAGGACAAGGTGTCTCTGACCTGTATGATCACAGACTTCTTT |
| | | CCTGAGGACATCACAGTGGAGTGGCAGTGGAACGGCCAGCCTGCCGAGAACTAT |
| | | AAGAATACCCAGCCAATCATGGACACAGATGGCTCTTACTTCGTGTATTCCAAG |
| | | CTGAACGTGCAGAAGTCCAATTGGGAGGCTGGCAACACCTTTACATGTAGCGTG |
| | | CTGCACGAAGGTCTGCATAACCATCATACCGAAAAATCACTGTCACACTCCCCT |
| | | GGACACCACCACCACCACCACTAATGA |
| 133 | Construct R | ATGGGTTGGTCCTGCATCATCCTGTTCCTGGTCGCCACCGCCACTGGGGTCCAC |
| | DNA | TCCGCACCTACATCATCATCAACTTCATCCTCCACCGCTGAGGCTCAGCAACAA |
| | sequence: | CAGCAACAACAGCAGCAGCAGCAGCAGCATCTGGAGCAGCTGCTGATGGACCTG |
| | mIL2(C140S)- | CAGGAGCTGCTGTCCAGAATGGAGAACTACCGCAATCTGAAGCTGCCAAGGATG |
| | 2x(SG4) - | CTGACCTTCAAGTTTTATCTGCCCAAGCAGGCCACAGAGCTGAAGGACCTGCAG |
| | MMPcs1 | TGCCTGGAGGATGAGCTGGGCCCACTGAGGCACGTGCTGGACCTGACCCAGAGC |
| | 2x(G4S) - | AAGTCTTTCCAGCTGGAGGATGCTGAGAACTTTATCTCCAATATCCGGGTGACC |
| | IL2Ralpha | GTGGTGAAGCTGAAGGGCAGCGACAACACATTCGAGTGCCAGTTTGACGATGAG |
| | hu IgG1 Fc- | TCTGCCACCGTGGTGGATTTCCTGAGGCGGTGGATCGCTTTTTCCCAGAGCATC |
| | 6xHIS | ATCTCCACAAGCCCTCAGTCTGGAGGAGGTGGCAGCGGAGGAGGAGGTGGCCCA |
| | | CTGTACGACCCCCCTGAGGTGCCCAATGCCACCTTCAAGGCTCTGTCTTATAAG |
| | | CTGGGCGTGAGGGGTGGCGGCGGCGGCTCTGGCGGGGGGGGCTCCGAGCTGTGC |
| | | AACGGCACAATCCTGAATTGCGAGTGTAAGAGGGGCTTTAGACGCCTGAAGGAG |
| | | CTGGTGTACATGCGGTGTCTGGGCAACTCCTGGTCCAGCAATTGCCAGTGTACC |
| | | TCTAACTCCCATGACAAGAGCAGAAAGCAGGTGACAGCCCAGCTGGAGCACCAG |
| | | AAGGAGCAGCAGACCACAACCGATATGCAGAAGCCCACCCAGTCTATGCACCAG |
| | | GAGAATCTGACAGGCCATTGCAGAGAGCCACCCCCTTGGAAGCACGAGGATAGC |
| | | AAGCGCATCTATCATTTCGTGGAGGGCCAGTCTGTGCACTACGAGTGTATCCCC |
| | | GGCTATAAGGCCCTGCAGAGAGGCCCTGCTATCTCCATCTGCAAGATGAAGTGT |
| | | GGCAAGACCGGCTGGACACAGCCTCAGCTGACCTGCGTGGACGAGAGGGAGCAC |
| | | CATCGGTTCCTGGCTAGCGAGGAGTCTCAGGGCTCCCGCAACTCTTCCCCTGAG |
| | | AGCGAGACATCTTGTCCAATCACAACCACAGATTTTCCACAGCCCACCGAGACA |
| | | ACCGCTATGACAGAGACCTTCGTGCTGACTATGGAATACAAAGATAAGACTCAT |
| | | ACCTGTCCACCCTGTCCTGCTCCTGAACTGCTGGGCGGTCCTTCCGTGTTCCTG |
| | | TTCCCTCCAAAACCTAAAGATACCCTGATGATCTCCAGGACCCCTGAGGTGACA |
| | | TGCGTGGTGGTGGACGTGAGCCACGAGGACCCCGAGGTGAAGTTCAACTGGTAC |
| | | GTGGACGGCGTGGAGGTGCATAATGCCAAGACAAAGCCCAGGGAGGAGCAGTAC |
| | | AACAGCACCTATCGGGTGGTGTCTGTGCTGACAGTGCTGCACCAGGATTGGCTG |
| | | AACGGCAAGGAGTATAAGTGCAAGGTGTCTAATAAGGCCCTGCCTGCTCCAATC |
| | | GAGAAGACCATCTCCAAGGCCAAGGGCCAGCCCAGAGAGCCTCAGGTGTACACA |
| | | CTGCCCCCTAGCCGCGACGAGCTGACCAAGAACCAGGTGTCTCTGACATGTCTG |
| | | GTGAAGGGCTTCTATCCATCTGACATCGCTGTGGAGTGGGAGTCCAATGGCCAG |
| | | CCCGAGAACAATTACAAGACCACACCACCCGTGCTGGACTCTGATGGCTCCTTC |
| | | TTTCTGTATTCCAAGCTGACCGTGGACAAGAGCAGATGGCAGCAGGGCAACGTG |
| | | TTCTCCTGTAGCGTGATGCACGAAGCCCTGCACAACCATTACACTCAGAAAAGC |
| | | CTGTCCCTGTCCCCTGGGCACCACCACCACCACCACTAATGA |
| 134 | Construct D | ATGGGTTGGTCCTGCATCATCCTGTTCCTGGTCGCCACCGCCACTGGGGTCCAC |
| | DNA | TCCGCACCTACATCATCATCAACTTCATCCTCCACCGCTGAGGCTCAGCAACAA |
| | Sequence: | CAGCAACAACAGCAGCAGCAGCAGCAGCATCTGGAGCAGCTGCTGATGGACCTG |
| | mIL2(C140S)- | CAGGAGCTGCTGTCCAGAATGGAGAACTACCGCAATCTGAAGCTGCCAAGGATG |
| | 2x(SG4) - | CTGACCTTCAAGTTTTATCTGCCCAAGCAGGCCACAGAGCTGAAGGACCTGCAG |
| | MMPcs1 - | TGCCTGGAGGATGAGCTGGGCCCACTGAGGCACGTGCTGGACCTGACCCAGAGC |
| | 2x(G4S) - | AAGTCTTTCCAGCTGGAGGATGCTGAGAACTTTATCTCCAATATCCGGGTGACC |
| | SIL2Ralpha | GTGGTGAAGCTGAAGGGCAGCGACAACACATTCGAGTGCCAGTTTGACGATGAG |
| | mIgG1 | TCTGCCACCGTGGTGGATTTCCTGAGGCGGTGGATCGCTTTTTCCCAGAGCATC |
| | Fc(T252M)- | ATCTCCACAAGCCCTCAGTCTGGAGGAGGTGGCAGCGGAGGAGGAGGTGGCCCA |
| | 6xHIS | CTGGGCGTGAGGGGTGGGGGGGGCGGCTCTGGCGGCGGCGGCTCCGAGCTGTGC |
| | | CTGTACGACCCCCCTGAGGTGCCCAATGCCACCTTCAAGGCTCTGTCTTATAAG |
| | | AACGGCACAATCCTGAATTGCGAGTGTAAGAGGGGCTTTAGACGCCTGAAGGAG |
| | | CTGGTGTACATGCGGTGTCTGGGCAACTCCTGGTCCAGCAATTGCCAGTGTACC |
| | | TCTAACTCCCATGACAAGAGCAGAAAGCAGGTGACAGCCCAGCTGGAGCACCAG |
| | | AAGGAGCAGCAGACCACAACCGATATGCAGAAGCCCACCCAGTCTATGCACCAG |
| | | GAGAATCTGACAGGCCATTGCAGAGAGCCACCCCCTTGGAAGCACGAGGATAGC |
| | | AAGCGCATCTATCATTTCGTGGAGGGCCAGTCTGTGCACTACGAGTGTATCCCC |
| | | GGCTATAAGGCCCTGCAGAGAGGCCCTGCTATCTCCATCTGCAAGATGAAGTGT |
| | | GGCAAGACCGGCTGGACACAGCCTCAGCTGACCTGCGTGGACGAGAGGGAGCAC |
| | | CATCGGTTCCTGGCTAGCGAGGAGTCTGGATGCAAACCCTGTATCTGTACCGTG |
| | | CCCGAGGTCTCTTCCGTCTTTATTTTCCCCCCCAAGCCTAAGGATGTGCTGATG |
| | | ATTACTCTGACCCCCAAGGTGACATGCGTGGTGGTGGACATCAGCAAGGACGAT |
| | | CCTGAGGTGCAGTTCTCTTGGTTTGTGGACGATGTGGAGGTGCACACCGCCCAG |
| | | ACACAGCCAAGGGAGGAGCAGTTCAATAGCACCTTTCGGTCCGTGAGCGAGCTG |
| | | CCCATCATGCATCAGGATTGGCTGAATGGCAAGGAGTTCAAGTGCAGAGTGAAC |
| | | TCTGCCGCTTTTCCCGCTCCTATCGAGAAGACCATCTCCAAGACAAAGGGCCGC |
| | | CCAAAGGCTCCACAGGTGTACACCATCCCACCTCCAAAGGAGCAGATGGCTAAG |
| | | GACAAGGTGTCTCTGACCTGTATGATCACAGACTTCTTTCCTGAGGACATCACA |
| | | GTGGAGTGGCAGTGGAACGGCCAGCCTGCCGAGAACTATAAGAATACCCAGCCA |
| | | ATCATGGACACAGATGGCTCTTACTTCGTGTATTCCAAGCTGAACGTGCAGAAG |
| | | TCCAATTGGGAGGCTGGCAACACCTTTACATGTAGCGTGCTGCACGAAGGTCTG |
| | | CATAACCATCATACCGAAAAATCACTGTCACACTCCCCTGGACACCACCACCAC |
| | | CACCACTAATGA |
| 135 | Construct E | ATGGGCTGGTCCTGCATCATTCTGTTTCTGGTGGCTACCGCCACCGGCGTGCAC |
| | DNA | TCTGCTCCTACATCCTCCAGCACCAAGAAAACCCAGCTGCAGTTGGAGCATCTG |
| | sequence: | CTGCTGGACCTGCAGATGATCCTGAACGGCATCAACAACTACAAGAACCCCAAG |
| | HuIL2(C125S) - | CTGACCCGGATGCTGACCTTCAAGTTCTACATGCCCAAGAAGGCCACCGAGCTG |
| | 2x(SG4) - | AAACATCTGCAGTGCCTGGAAGAGGAACTGAAGCCCCTGGAAGAAGTGCTGAAT |
| | MMPcs1 - | CTGGCCCAGTCCAAGAACTTCCACCTGAGGCCTCGGGACCTGATCTCCAACATC |
| | 2x(G4S) - | AACGTGATCGTGCTCGAGCTGAAGGGCTCCGAGACAACCTTCATGTGCGAGTAC |
| | IL2Ralpha | GCCGACGAGACAGCTACCATCGTGGAATTTCTGAACCGGTGGATCACCTTCAGC |
| | hu IgG1 Fc - | CAGTCCATCATCAGCACCCTGACATCTGGCGGCGGAGGATCTGGCGGAGGCGGA |
| | 6xHIS | GGACCTTTGGGAGTTCGCGGCGGTGGTGGTGGCAGCGGAGGTGGTGGATCTGAG |
| | | CTGTGTGACGACGACCCTCCTGAGATCCCTCACGCCACCTTTAAGGCCATGGCT |
| | | TACAAAGAGGGCACCATGCTGAACTGCGAGTGCAAGAGAGGCTTCCGGCGGATC |
| | | AAGTCCGGCAGCCTGTATATGCTGTGCACCGGCAACTCCAGCCACTCCTCTTGG |
| | | GACAACCAGTGCCAGTGCACCAGCTCTGCTACCCGGAACACCACCAAGCAAGTG |
| | | ACCCCTCAGCCTGAGGAACAGAAAGAGCGCAAGACCACCGAGATGCAGAGCCCC |
| | | ATGCAGCCTGTGGATCAGGCTTCTCTGCCTGGCCACTGTAGAGAGCCTCCACCT |
| | | TGGGAGAATGAGGCTACCGAGAGAATCTACCACTTCGTCGTGGGACAGATGGTG |
| | | TACTACCAGTGCGTGCAGGGCTACCGCGCTCTGCATAGAGGACCAGCAGAGTCC |
| | | GTGTGCAAGATGACCCACGGCAAGACCAGATGGACCCAGCCTCAGCTGATCTGC |
| | | ACCGGCGAGATGGAAACCTCTCAGTTCCCCGGCGAGGAAAAGCCTCAGGCCTCT |
| | | CCTGAAGGCAGACCCGAGTCTGAGACATCCTGTCTCGTGACCACCACAGACTTC |
| | | CAGATCCAGACCGAGATGGCCGCTACCATGGAAACCAGCATCTTCACCACCGAG |
| | | TACCAGGACAAGACCCACACCTGTCCTCCATGTCCTGCTCCAGAATTGCTCGGC |
| | | GGACCCTCCGTGTTCCTGTTTCCTCCAAAGCCTAAGGACACCCTGATGATCTCT |
| | | CGGACCCCTGAAGTGACCTGCGTGGTGGTCGATGTGTCTCACGAGGATCCCGAA |
| | | GTGAAGTTCAATTGGTACGTGGACGGCGTGGAAGTGCACAACGCCAAGACCAAG |
| | | CCTAGAGAGGAACAGTACAACTCCACCTACAGAGTGGTGTCCGTGCTGACCGTG |
| | | CTGCACCAGGATTGGCTGAATGGCAAAGAGTACAAGTGCAAGGTGTCCAACAAG |
| | | GCCCTGCCTGCTCCTATCGAAAAGACCATCTCCAAGGCCAAGGGCCAGCCTAGG |
| | | GAACCCCAGGTTTACACCTTGCCTCCATCTCGGGACGAGCTGACCAAGAACCAG |
| | | GTGTCCCTGACCTGTCTGGTCAAGGGCTTCTACCCCTCCGATATCGCCGTGGAA |
| | | TGGGAGTCTAATGGCCAGCCTGAAAACAATTACAAGACAACCCCTCCTGTGCTG |
| | | GACTCCGACGGCTCATTCTTCCTGTACAGCAAGCTGACAGTGGACAAGTCCAGA |
| | | TGGCAGCAGGGCAACGTGTTCTCCTGCTCCGTGATGCATGAGGCCCTGCACAAC |
| | | CACTACACCCAGAAGTCCCTGTCTCTGTCCCCTGGCCACCATCACCATCATCAC |
| | | TGATAA |
| 136 | Construct S | ATGGGTTGGTCCTGCATCATCCTGTTCCTGGTCGCCACCGCCACTGGGGTCCAC |
| | DNA | TCCGCACCTACTTCAAGTTCTACAAAGAAAACACAGCTACAACTGGAGCATTTA |
| | sequence: | CTTCTGGATTTACAGATGATTTTGAATGGAATTAATAATTACAAGAATCCCAAA |
| | hIL2- 2x(SG4) - | CTCACCAGGATGCTCACATTTAAGTTTTACATGCCCAAGAAGGCCACAGAACTG |
| | MMPcs1 - | AAACATCTTCAGTGTCTAGAAGAAGAACTCAAACCTCTGGAGGAAGTGCTAAAT |
| | 2x(G4S) - | TTAGCTCAAAGCAAAAACTTTCACTTAAGACCCAGGGACTTAATCAGCAATATC |
| | hIL2Ralpha - | AACGTAATAGTTCTGGAACTAAAGGGATCTGAAACAACATTCATGTGTGAATAT |
| | hIgG1Fc_mut | GCTGATGAGACAGCAACCATTGTAGAATTTCTGAACAGATGGATTACCTTTTGT |
| | 1 (K392D; | CAAAGCATCATCTCAACACTGACTTCTGGTGGCGGTGGCTCTGGTGGCGGTGGC |
| | K409D) | GGTCCTCTGGGTGTCAGAGGTGGTGGCGGTGGCTCTGGTGGCGGTGGCTCTGAG |
| | | CTCTGTGACGATGACCCGCCAGAGATCCCACACGCCACATTCAAAGCCATGGCC |
| | | TACAAGGAAGGAACCATGTTGAACTGTGAATGCAAGAGAGGTTTCCGCAGAATA |
| | | AAAAGCGGGTCACTCTATATGCTCTGTACAGGAAACTCTAGCCACTCGTCCTGG |
| | | GACAACCAATGTCAATGCACAAGCTCTGCCACTCGGAACACAACGAAACAAGTG |
| | | ACACCTCAACCTGAAGAACAGAAAGAAAGGAAAACCACAGAAATGCAAAGTCCA |
| | | ATGCAGCCAGTGGACCAAGCGAGCCTTCCAGGTCACTGCAGGGAACCTCCACCA |
| | | TGGGAAAATGAAGCCACAGAGAGAATTTATCATTTCGTGGTGGGGCAGATGGTT |
| | | TATTATCAGTGCGTCCAGGGATACAGGGCTCTACACAGAGGTCCTGCTGAGAGC |
| | | GTCTGCAAAATGACCCACGGGAAGACAAGGTGGACCCAGCCCCAGCTCATATGC |
| | | ACAGGTGAAATGGAGACCAGTCAGTTTCCAGGTGAAGAGAAGCCTCAGGCAAGC |
| | | CCCGAAGGCCGTCCTGAGAGTGAGACTTCCTGCCTCGTCACAACAACAGATTTT |
| | | CAAATACAGACAGAAATGGCTGCAACCATGGAGACGTCCATATTTACAACAGAG |
| | | TACCAGGACAAAACTCACACATGCCCACCGTGCCCAGCACCTGAACTCCTGGGG |
| | | GGACCGTCAGTCTTCCTCTTCCCCCCAAAACCCAAGGACACCCTCATGATCTCC |
| | | CGGACCCCTGAGGTCACATGCGTGGTGGTGGACGTGAGCCACGAAGACCCTGAG |
| | | GTCAAGTTCAACTGGTACGTGGACGGCGTGGAGGTGCATAATGCCAAGACAAAG |
| | | CCGCGGGAGGAGCAGTACAACAGCACGTACCGTGTGGTCAGCGTCCTCACCGTC |
| | | CTGCACCAGGACTGGCTGAATGGCAAGGAGTACAAGTGCAAGGTCTCCAACAAA |
| | | GCCCTCCCAGCCCCCATCGAGAAAACCATCTCCAAAGCCAAAGGGCAGCCCCGA |
| | | GAACCACAGGTGTACACCCTGCCCCCATCCCGGGATGAGCTGACCAAGAACCAG |
| | | GTCAGCCTGACCTGCCTGGTCAAAGGCTTCTATCCCAGCGACATCGCCGTGGAG |
| | | TGGGAGAGCAATGGGCAGCCGGAGAACAACTACGACACCACGCCTCCCGTGCTG |
| | | GACTCCGACGGCTCCTTCTTCCTCTATAGCGACCTCACCGTGGACAAGAGCAGG |
| | | TGGCAGCAGGGGAACGTCTTCTCATGCTCCGTGATGCATGAGGCTCTGCACAAC |
| | | CACTACACGCAGAAGAGCCTCTCCCTGTCTCCGGGTCACCACCACCACCACCAC |
| | | TAATGA |
| 137 | Construct T | ATGGGTTGGTCCTGCATCATCCTGTTCCTGGTCGCCACCGCCACTGGGGTCCAC |
| | DNA sequence | TCCGACAAAACTCACACATGCCCACCGTGCCCAGCACCTGAACTCCTGGGGGGA |
| | including | CCGTCAGTCTTCCTCTTCCCCCCAAAACCCAAGGACACCCTCATGATCTCCCGG |
| | hIgG1 Fc_mut | ACCCCTGAGGTCACATGCGTGGTGGTGGACGTGAGCCACGAAGACCCTGAGGTC |
| | 2 (D356K; | AAGTTCAACTGGTACGTGGACGGCGTGGAGGTGCATAATGCCAAGACAAAGCCG |
| | D399K) | CGGGAGGAGCAGTACAACAGCACGTACCGTGTGGTCAGCGTCCTCACCGTCCTG |
| | | CACCAGGACTGGCTGAATGGCAAGGAGTACAAGTGCAAGGTCTCCAACAAAGCC |
| | | CTCCCAGCCCCCATCGAGAAAACCATCTCCAAAGCCAAAGGGCAGCCCCGAGAA |
| | | CCACAGGTGTACACCCTGCCCCCATCCCGGAAAGAGCTGACCAAGAACCAGGTC |
| | | AGCCTGACCTGCCTGGTCAAAGGCTTCTATCCCAGCGACATCGCCGTGGAGTGG |
| | | GAGAGCAATGGGCAGCCGGAGAACAACTACAAGACCACGCCTCCCGTGCTGAAA |
| | | TCCGACGGCTCCTTCTTCCTCTATAGCAAGCTCACCGTGGACAAGAGCAGGTGG |
| | | CAGCAGGGGAACGTCTTCTCATGCTCCGTGATGCATGAGGCTCTGCACAACCAC |
| | | TACACGCAGAAGAGCCTCTCCCTGTCTCCGGGTCACCACCACCACCACCACTAA |
| | | TGA |
| 138 | mIgG1 Fc - | ATGGGTTGGTCCTGCATCATCCTGTTCCTGGTCGCCACCGCCACTGGGGTCCAC |
| | Murine IL2 - | TCCGGATGCAAACCCTGTATCTGTACCGTGCCCGAGGTCTCTTCCGTCTTTATT |
| | 2x(SG4) - | TTCCCCCCCAAGCCTAAGGATGTGCTGACTATTACTCTGACCCCCAAGGTGACA |
| | MMPcs1 - 2x | TGCGTGGTGGTGGACATCAGCAAGGACGATCCTGAGGTGCAGTTCTCTTGGTTT |
| | (G4S) - | GTGGACGATGTGGAGGTGCACACCGCCCAGACACAGCCAAGGGAGGAGCAGTTC |
| | IL2Ralpha | AATAGCACCTTTCGGTCCGTGAGCGAGCTGCCCATCATGCATCAGGATTGGCTG |
| | (long kinetic | AATGGCAAGGAGTTCAAGTGCAGAGTGAACTCTGCCGCTTTTCCCGCTCCTATC |
| | IL2 post | GAGAAGACCATCTCCAAGACAAAGGGCCGCCCAAAGGCTCCACAGGTGTACACC |
| | cleavage) | ATCCCACCTCCAAAGGAGCAGATGGCTAAGGACAAGGTGTCTCTGACCTGTATG |
| | DNA | ATCACAGACTTCTTTCCTGAGGACATCACAGTGGAGTGGCAGTGGAACGGCCAG |
| | Sequence | CCTGCCGAGAACTATAAGAATACCCAGCCAATCATGGACACAGATGGCTCTTAC |
| | | TTCGTGTATTCCAAGCTGAACGTGCAGAAGTCCAATTGGGAGGCTGGCAACACC |
| | | TTTACATGTAGCGTGCTGCACGAAGGTCTGCATAACCATCATACCGAAAAATCA |
| | | CTGTCACACTCCCCTGGAAAAGCACCTACATCATCATCAACTTCATCCTCCACC |
| | | GCTGAGGCTCAGCAACAACAGCAACAACAGCAGCAGCAGCAGCAGCATCTGGAG |
| | | CAGCTGCTGATGGACCTGCAGGAGCTGCTGTCCAGAATGGAGAACTACCGCAAT |
| | | CTGAAGCTGCCAAGGATGCTGACCTTCAAGTTTTATCTGCCCAAGCAGGCCACA |
| | | GAGCTGAAGGACCTGCAGTGCCTGGAGGATGAGCTGGGCCCACTGAGGCACGTG |
| | | CTGGACCTGACCCAGAGCAAGTCTTTCCAGCTGGAGGATGCTGAGAACTTTATC |
| | | TCCAATATCCGGGTGACCGTGGTGAAGCTGAAGGGCAGCGACAACACATTCGAG |
| | | TGCCAGTTTGACGATGAGTCTGCCACCGTGGTGGATTTCCTGAGGCGGTGGATC |
| | | GCTTTTTGTCAGAGCATCATCTCCACAAGCCCTCAGTCTGGAGGAGGTGGCAGC |
| | | GGAGGAGGAGGTGGCCCACTGGGCGTGAGGGGTGGCGGCGGCGGCTCTGGCGGC |
| | | GGCGGCTCCGAGCTGTGCCTGTACGACCCCCCTGAGGTGCCCAATGCCACCTTC |
| | | AAGGCTCTGTCTTATAAGAACGGCACAATCCTGAATTGCGAGTGTAAGAGGGGC |
| | | TTTAGACGCCTGAAGGAGCTGGTGTACATGCGGTGTCTGGGCAACTCCTGGTCC |
| | | AGCAATTGCCAGTGTACCTCTAACTCCCATGACAAGAGCAGAAAGCAGGTGACA |
| | | GCCCAGCTGGAGCACCAGAAGGAGCAGCAGACCACAACCGATATGCAGAAGCCC |
| | | ACCCAGTCTATGCACCAGGAGAATCTGACAGGCCATTGCAGAGAGCCACCCCCT |
| | | TGGAAGCACGAGGATAGCAAGCGCATCTATCATTTCGTGGAGGGCCAGTCTGTG |
| | | CACTACGAGTGTATCCCCGGCTATAAGGCCCTGCAGAGAGGCCCTGCTATCTCC |
| | | ATCTGCAAGATGAAGTGTGGCAAGACCGGCTGGACACAGCCTCAGCTGACCTGC |
| | | GTGGACGAGAGGGAGCACCATCGGTTCCTGGCTAGCGAGGAGTCTCAGGGCTCC |
| | | CGCAACTCTTCCCCTGAGAGCGAGACATCTTGTCCAATCACAACCACAGATTTT |
| | | CCACAGCCCACCGAGACAACCGCTATGACAGAGACCTTCGTGCTGACTATGGAA |
| | | TACAAATAATGA |
| 139 | hIL2- 2x(SG4) - | ATGGGTTGGTCCTGCATCATCCTGTTCCTGGTCGCCACCGCCACTGGGGTCCAC |
| | MMPcs1 - | TCCgcacctacttcaagttctacaaagaaaacacagctacaactggagcattta |
| | 2x(G4S) - | cttctggatttacagatgattttgaatggaattaataattacaagaatcccaaa |
| | hIL2Rbeta - | ctcaccaggatgctcacatttaagttttacatgcccaagaaggccacagaactg |
| | hIgG1Fc | aaacatcttcagtgtctagaagaagaactcaaacctctggaggaagtgctaaat |
| | (Construct U) | ttagctcaaagcaaaaactttcacttaagacccagggacttaatcagcaatatc |
| | DNA | aacgtaatagttctggaactaaagggatctgaaacaacattcatgtgtgaatat |
| | Sequence | gctgatgagacagcaaccattgtagaatttctgaacagatggattaccttttgt |
| | | caaagcatcatctcaacactgactTCTGGTGGCGGTGGCTCTGGTGGCGGTGGC |
| | | GGTCCTCTGGGTGTCAGAGGTGGTGGCGGTGGCTCTGGTGGCGGTGGCTCTGCG |
| | | GTGAATGGCACTTCCCAGTTCACATGCTTCTACAACTCGAGAGCCAACATCTCC |
| | | TGTGTCTGGAGCCAAGATGGGGCTCTGCAGGACACTTCCTGCCAAGTCCATGCC |
| | | TGGCCGGACAGACGGCGGTGGAACCAAACCTGTGAGCTGCTCCCCGTGAGTCAA |
| | | GCATCCTGGGCCTGCAACCTGATCCTCGGAGCCCCAGATTCTCAGAAACTGACC |
| | | ACAGTTGACATCGTCACCCTGAGGGTGCTGTGCCGTGAGGGGGTGCGATGGAGG |
| | | GTGATGGCCATCCAGGACTTCAAGCCCTTTGAGAACCTTCGCCTGATGGCCCCC |
| | | ATCTCCCTCCAAGTTGTCCACGTGGAGACCCACAGATGCAACATAAGCTGGGAA |
| | | ATCTCCCAAGCCTCCCACTACTTTGAAAGACACCTGGAGTTCGAGGCCCGGACG |
| | | CTGTCCCCAGGCCACACCTGGGAGGAGGCCCCCCTGCTGACTCTCAAGCAGAAG |
| | | CAGGAATGGATCTGCCTGGAGACGCTCACCCCAGACACCCAGTATGAGTTTCAG |
| | | GTGCGGGTCAAGCCTCTGCAAGGCGAGTTCACGACCTGGAGCCCCTGGAGCCAG |
| | | CCCCTGGCCTTCAGGACAAAGCCTGCAGCCCTTGGGAAGGACACCGACAAGACC |
| | | CACACCTGTCCTCCATGTCCTGCTCCAGAATTGCTCGGCGGACCCTCCGTGTTC |
| | | CTGTTTCCTCCAAAGCCTAAGGACACCCTGATGATCTCTCGGACCCCTGAAGTG |
| | | ACCTGCGTGGTGGTCGATGTGTCTCACGAGGATCCCGAAGTGAAGTTCAATTGG |
| | | TACGTGGACGGCGTGGAAGTGCACAACGCCAAGACCAAGCCTAGAGAGGAACAG |
| | | TACAACTCCACCTACAGAGTGGTGTCCGTGCTGACCGTGCTGCACCAGGATTGG |
| | | CTGAATGGCAAAGAGTACAAGTGCAAGGTGTCCAACAAGGCCCTGCCTGCTCCT |
| | | ATCGAAAAGACCATCTCCAAGGCCAAGGGCCAGCCTAGGGAACCCCAGGTTTAC |
| | | ACCTTGCCTCCATCTCGGGACGAGCTGACCAAGAACCAGGTGTCCCTGACCTGT |
| | | CTGGTCAAGGGCTTCTACCCCTCCGATATCGCCGTGGAATGGGAGTCTAATGGC |
| | | CAGCCTGAAAACAATTACAAGACAACCCCTCCTGTGCTGGACTCCGACGGCTCA |
| | | TTCTTCCTGTACAGCAAGCTGACAGTGGACAAGTCCAGATGGCAGCAGGGCAAC |
| | | GTGTTCTCCTGCTCCGTGATGCATGAGGCCCTGCACAACCACTACACCCAGAAG |
| | | TCCCTGTCTCTGTCCCCTGGCTAATGA |
| 140 | hIL2- 2x(SG4) - | ATGGGTTGGTCCTGCATCATCCTGTTCCTGGTCGCCACCGCCACTGGGGTCCAC |
| | MMPcs1 - | TCCgcacctacttcaagttctacaaagaaaacacagctacaactggagcattta |
| | 2x(G4S) - | cttctggatttacagatgattttgaatggaattaataattacaagaatcccaaa |
| | hIL2Rgamma - | ctcaccaggatgctcacatttaagttttacatgcccaagaaggccacagaactg |
| | hIgG1Fc | aaacatcttcagtgtctagaagaagaactcaaacctctggaggaagtgctaaat |
| | DNA sequence | ttagctcaaagcaaaaactttcacttaagacccagggacttaatcagcaatatc |
| | | aacgtaatagttctggaactaaagggatctgaaacaacattcatgtgtgaatat |
| | | gctgatgagacagcaaccattgtagaatttctgaacagatggattaccttttgt |
| | | caaagcatcatctcaacactgactTCTGGTGGCGGTGGCTCTGGTGGCGGTGGC |
| | | GGTCCTCTGGGTGTCAGAGGTGGTGGCGGTGGCTCTGGTGGCGGTGGCTCTCTG |
| | | AACACGACAATTCTGACGCCCAATGGGAATGAAGACACCACAGCTGATTTCTTC |
| | | CTGACCACTATGCCCACTGACTCCCTCAGTGTTTCCACTCTGCCCCTCCCAGAG |
| | | GTTCAGTGTTTTGTGTTCAATGTCGAGTACATGAATTGCACTTGGAACAGCAGC |
| | | TCTGAGCCCCAGCCTACCAACCTCACTCTGCATTATTGGTACAAGAACTCGGAT |
| | | AATGATAAAGTCCAGAAGTGCAGCCACTATCTATTCTCTGAAGAAATCACTTCT |
| | | GGCTGTCAGTTGCAAAAAAAGGAGATCCACCTCTACCAAACATTTGTTGTTCAG |
| | | CTCCAGGACCCACGGGAACCCAGGAGACAGGCCACACAGATGCTAAAACTGCAG |
| | | AATCTGGTGATCCCCTGGGCTCCAGAGAACCTAACACTTCACAAACTGAGTGAA |
| | | TCCCAGCTAGAACTGAACTGGAACAACAGATTCTTGAACCACTGTTTGGAGCAC |
| | | TTGGTGCAGTACCGGACTGACTGGGACCACAGCTGGACTGAACAATCAGTGGAT |
| | | TATAGACATAAGTTCTCCTTGCCTAGTGTGGATGGGCAGAAACGCTACACGTTT |
| | | CGTGTTCGGAGCCGCTTTAACCCACTCTGTGGAAGTGCTCAGCATTGGAGTGAA |
| | | TGGAGCCACCCAATCCACTGGGGGAGCAATACTTCAAAAGAGAATCCTTTCCTG |
| | | TTTGCATTGGAAGCCGACAAGACCCACACCTGTCCTCCATGTCCTGCTCCAGAA |
| | | TTGCTCGGCGGACCCTCCGTGTTCCTGTTTCCTCCAAAGCCTAAGGACACCCTG |
| | | ATGATCTCTCGGACCCCTGAAGTGACCTGCGTGGTGGTCGATGTGTCTCACGAG |
| | | GATCCCGAAGTGAAGTTCAATTGGTACGTGGACGGCGTGGAAGTGCACAACGCC |
| | | AAGACCAAGCCTAGAGAGGAACAGTACAACTCCACCTACAGAGTGGTGTCCGTG |
| | | CTGACCGTGCTGCACCAGGATTGGCTGAATGGCAAAGAGTACAAGTGCAAGGTG |
| | | TCCAACAAGGCCCTGCCTGCTCCTATCGAAAAGACCATCTCCAAGGCCAAGGGC |
| | | CAGCCTAGGGAACCCCAGGTTTACACCTTGCCTCCATCTCGGGACGAGCTGACC |
| | | AAGAACCAGGTGTCCCTGACCTGTCTGGTCAAGGGCTTCTACCCCTCCGATATC |
| | | GCCGTGGAATGGGAGTCTAATGGCCAGCCTGAAAACAATTACAAGACAACCCCT |
| | | CCTGTGCTGGACTCCGACGGCTCATTCTTCCTGTACAGCAAGCTGACAGTGGAC |
| | | AAGTCCAGATGGCAGCAGGGCAACGTGTTCTCCTGCTCCGTGATGCATGAGGCC |
| | | CTGCACAACCACTACACCCAGAAGTCCCTGTCTCTGTCCCCTGGCTAATGA |
| 141- | Not Used | |
| 149 | | |

Other DNA sequences

| | | |
|---|---|---|
| 150 | Murine Ig | ATGGGTTGGTCCTGCATCATCCTGTTCCTGGTCGCCACCGCCACTGGGGTCCAC |
| | kappa chain | TCC |
| | leader DNA | |
| | sequence | |
| 151 | Murine IL-2 | GCACCTACATCATCATCAACTTCATCCTCCACCGCTGAGGCTCAGCAACAACAG |
| | DNA sequence | CAACAACAGCAGCAGCAGCAGCAGCATCTGGAGCAGCTGCTGATGGACCTGCAG |
| | | GAGCTGCTGTCCAGAATGGAGAACTACCGCAATCTGAAGCTGCCAAGGATGCTG |
| | | ACCTTCAAGTTTTATCTGCCCAAGCAGGCCACAGAGCTGAAGGACCTGCAGTGC |
| | | CTGGAGGATGAGCTGGGCCCACTGAGGCACGTGCTGGACCTGACCCAGAGCAAG |
| | | TCTTTCCAGCTGGAGGATGCTGAGAACTTTATCTCCAATATCCGGGTGACCGTG |
| | | GTGAAGCTGAAGGGCAGCGACAACACATTCGAGTGCCAGTTTGACGATGAGTCT |
| | | GCCACCGTGGTGGATTTCCTGAGGCGGTGGATCGCTTTTTGTCAGAGCATCATC |
| | | TCCACAAGCCCTCAG |
| 152 | MMP cleavage | GGCCCACTGGGCGTGAGGGGT |
| | site | |
| | GPLGVRG | |
| | DNA sequence | |
| 153 | Gly-Ser rich | TCTGGAGGAGGTGGCAGCGGAGGAGGAGGT |
| | linker DNA | |
| | sequence | |
| 154 | Murine IL- | GAGCTGTGCCTGTACGACCCCCCTGAGGTGCCCAATGCCACCTTCAAGGCTCTG |
| | 2Ralpha DNA | TCTTATAAGAACGGCACAATCCTGAATTGCGAGTGTAAGAGGGGCTTTAGACGC |
| | sequence | CTGAAGGAGCTGGTGTACATGCGGTGTCTGGGCAACTCCTGGTCCAGCAATTGC |
| | | CAGTGTACCTCTAACTCCCATGACAAGAGCAGAAAGCAGGTGACAGCCCAGCTG |
| | | GAGCACCAGAAGGAGCAGCAGACCACAACCGATATGCAGAAGCCCACCCAGTCT |
| | | ATGCACCAGGAGAATCTGACAGGCCATTGCAGAGAGCCACCCCCTTGGAAGCAC |
| | | GAGGATAGCAAGCGCATCTATCATTTCGTGGAGGGCCAGTCTGTGCACTACGAG |
| | | TGTATCCCCGGCTATAAGGCCCTGCAGAGAGGCCCTGCTATCTCCATCTGCAAG |
| | | ATGAAGTGTGGCAAGACCGGCTGGACACAGCCTCAGCTGACCTGCGTGGACGAG |
| | | AGGGAGCACCATCGGTTCCTGGCTAGCGAGGAGTCTCAGGGCTCCCGCAACTCT |
| | | TCCCCTGAGAGCGAGACATCTTGTCCAATCACAACCACAGATTTTCCACAGCCC |
| | | ACCGAGACAACCGCTATGACAGAGACCTTCGTGCTGACTATGGAATACAAA |
| 155 | His tag DNA | CACCACCACCACCACCAC |
| | Sequence | |
| 156 | Stop codons | TAATGA |
| 157 | Murine IL-2 | GCACCTACATCATCATCAACTTCATCCTCCACCGCTGAGGCTCAGCAACAACAG |
| | C140S DNA | CAACAACAGCAGCAGCAGCAGCAGCATCTGGAGCAGCTGCTGATGGACCTGCAG |
| | sequence | GAGCTGCTGTCCAGAATGGAGAACTACCGCAATCTGAAGCTGCCAAGGATGCTG |
| | | ACCTTCAAGTTTTATCTGCCCAAGCAGGCCACAGAGCTGAAGGACCTGCAGTGC |
| | | CTGGAGGATGAGCTGGGCCCACTGAGGCACGTGCTGGACCTGACCCAGAGCAAG |
| | | TCTTTCCAGCTGGAGGATGCTGAGAACTTTATCTCCAATATCCGGGTGACCGTG |
| | | GTGAAGCTGAAGGGCAGCGACAACACATTCGAGTGCCAGTTTGACGATGAGTCT |
| | | GCCACCGTGGTGGATTTCCTGAGGCGGTGGATCGCTTTTTCCCAGAGCATCATC |
| | | TCCACAAGCCCTCA |
| 158 | Murine IgG1 | GGATGCAAACCCTGTATCTGTACCGTGCCCGAGGTCTCTTCCGTCTTTATTTTC |
| | T252M Fc | CCCCCCAAGCCTAAGGATGTGCTGATGATTACTCTGACCCCCAAGGTGACATGC |
| | domain DNA | GTGGTGGTGGACATCAGCAAGGACGATCCTGAGGTGCAGTTCTCTTGGTTTGTG |
| | sequence | GACGATGTGGAGGTGCACACCGCCCAGACACAGCCAAGGGAGGAGCAGTTCAAT |
| | | AGCACCTTTCGGTCCGTGAGCGAGCTGCCCATCATGCATCAGGATTGGCTGAAT |
| | | GGCAAGGAGTTCAAGTGCAGAGTGAACTCTGCCGCTTTTCCCGCTCCTATCGAG |
| | | AAGACCATCTCCAAGACAAAGGGCCGCCCAAAGGCTCCACAGGTGTACACCATC |
| | | CCACCTCCAAAGGAGCAGATGGCTAAGGACAAGGTGTCTCTGACCTGTATGATC |
| | | ACAGACTTCTTTCCTGAGGACATCACAGTGGAGTGGCAGTGGAACGGCCAGCCT |
| | | GCCGAGAACTATAAGAATACCCAGCCAATCATGGACACAGATGGCTCTTACTTC |
| | | GTGTATTCCAAGCTGAACGTGCAGAAGTCCAATTGGGAGGCTGGCAACACCTTT |
| | | ACATGTAGCGTGCTGCACGAAGGTCTGCATAACCATCATACCGAAAAATCACTG |
| | | TCACACTCCCCTGGA |
| 159 | Murine IgG1 | GGATGCAAACCCTGTATCTGTACCGTGCCCGAGGTCTCTTCCGTCTTTATTTTC |
| | Fc domain | CCCCCCAAGCCTAAGGATGTGCTGACTATTACTCTGACCCCCAAGGTGACATGC |
| | DNA sequence | GTGGTGGTGGACATCAGCAAGGACGATCCTGAGGTGCAGTTCTCTTGGTTTGTG |
| | | GACGATGTGGAGGTGCACACCGCCCAGACACAGCCAAGGGAGGAGCAGTTCAAT |
| | | AGCACCTTTCGGTCCGTGAGCGAGCTGCCCATCATGCATCAGGATTGGCTGAAT |
| | | GGCAAGGAGTTCAAGTGCAGAGTGAACTCTGCCGCTTTTCCCGCTCCTATCGAG |
| | | AAGACCATCTCCAAGACAAAGGGCCGCCCAAAGGCTCCACAGGTGTACACCATC |
| | | CCACCTCCAAAGGAGCAGATGGCTAAGGACAAGGTGTCTCTGACCTGTATGATC |
| | | ACAGACTTCTTTCCTGAGGACATCACAGTGGAGTGGCAGTGGAACGGCCAGCCT |
| | | GCCGAGAACTATAAGAATACCCAGCCAATCATGGACACAGATGGCTCTTACTTC |
| | | GTGTATTCCAAGCTGAACGTGCAGAAGTCCAATTGGGAGGCTGGCAACACCTTT |
| | | ACATGTAGCGTGCTGCACGAAGGTCTGCATAACCATCATACCGAAAAATCACTG |
| | | TCACACTCCCCTGGAAAA |
| 160 | Human IL-2 | GCTCCTACATCCTCCAGCACCAAGAAAACCCAGCTGCAGTTGGAGCATCTGCTG |
| | C125S DNA | CTGGACCTGCAGATGATCCTGAACGGCATCAACAACTACAAGAACCCCAAGCTG |
| | sequence | ACCCGGATGCTGACCTTCAAGTTCTACATGCCCAAGAAGGCCACCGAGCTGAAA |
| | | CATCTGCAGTGCCTGGAAGAGGAACTGAAGCCCCTGGAAGAAGTGCTGAATCTG |
| | | GCCCAGTCCAAGAACTTCCACCTGAGGCCTCGGGACCTGATCTCCAACATCAAC |
| | | GTGATCGTGCTCGAGCTGAAGGGCTCCGAGACAACCTTCATGTGCGAGTACGCC |
| | | GACGAGACAGCTACCATCGTGGAATTTCTGAACCGGTGGATCACCTTCAGCCAG |
| | | TCCATCATCAGCACCCTGACA |
| 161 | Human IgG1 | GACAAGACCCACACCTGTCCTCCATGTCCTGCTCCAGAATTGCTCGGCGGACCC |
| | Fc domain | TCCGTGTTCCTGTTTCCTCCAAAGCCTAAGGACACCCTGATGATCTCTCGGACC |
| | DNA sequence | CCTGAAGTGACCTGCGTGGTGGTCGATGTGTCTCACGAGGATCCCGAAGTGAAG |
| | | TTCAATTGGTACGTGGACGGCGTGGAAGTGCACAACGCCAAGACCAAGCCTAGA |
| | | GAGGAACAGTACAACTCCACCTACAGAGTGGTGTCCGTGCTGACCGTGCTGCAC |
| | | CAGGATTGGCTGAATGGCAAAGAGTACAAGTGCAAGGTGTCCAACAAGGCCCTG |
| | | CCTGCTCCTATCGAAAAGACCATCTCCAAGGCCAAGGGCCAGCCTAGGGAACCC |
| | | CAGGTTTACACCTTGCCTCCATCTCGGGACGAGCTGACCAAGAACCAGGTGTCC |
| | | CTGACCTGTCTGGTCAAGGGCTTCTACCCCTCCGATATCGCCGTGGAATGGGAG |
| | | TCTAATGGCCAGCCTGAAAACAATTACAAGACAACCCCTCCTGTGCTGGACTCC |
| | | GACGGCTCATTCTTCCTGTACAGCAAGCTGACAGTGGACAAGTCCAGATGGCAG |
| | | CAGGGCAACGTGTTCTCCTGCTCCGTGATGCATGAGGCCCTGCACAACCACTAC |
| | | ACCCAGAAGTCCCTGTCTCTGTCCCCTGGC |
| 162 | Human IL-2 | GCACCTACTTCAAGTTCTACAAAGAAAACACAGCTACAACTGGAGCATTTACTT |
| | DNA sequence | CTGGATTTACAGATGATTTTGAATGGAATTAATAATTACAAGAATCCCAAACTC |
| | | ACCAGGATGCTCACATTTAAGTTTTACATGCCCAAGAAGGCCACAGAACTGAAA |
| | | CATCTTCAGTGTCTAGAAGAAGAACTCAAACCTCTGGAGGAAGTGCTAAATTTA |
| | | GCTCAAAGCAAAAACTTTCACTTAAGACCCAGGGACTTAATCAGCAATATCAAC |
| | | GTAATAGTTCTGGAACTAAAGGGATCTGAAACAACATTCATGTGTGAATATGCT |
| | | GATGAGACAGCAACCATTGTAGAATTTCTGAACAGATGGATTACCTTTTGTCAA |
| | | AGCATCATCTCAACACTGACT |
| 163 | Human IgG1 | GACAAAACTCACACATGCCCACCGTGCCCAGCACCTGAACTCCTGGGGGGACCG |
| | K392D | TCAGTCTTCCTCTTCCCCCCAAAACCCAAGGACACCCTCATGATCTCCCGGACC |
| | K409D Fc | CCTGAGGTCACATGCGTGGTGGTGGACGTGAGCCACGAAGACCCTGAGGTCAAG |
| | domain DNA | TTCAACTGGTACGTGGACGGCGTGGAGGTGCATAATGCCAAGACAAAGCCGCGG |
| | sequence | GAGGAGCAGTACAACAGCACGTACCGTGTGGTCAGCGTCCTCACCGTCCTGCAC |
| | | CAGGACTGGCTGAATGGCAAGGAGTACAAGTGCAAGGTCTCCAACAAAGCCCTC |
| | | CCAGCCCCCATCGAGAAAACCATCTCCAAAGCCAAAGGGCAGCCCCGAGAACCA |
| | | CAGGTGTACACCCTGCCCCCATCCCGGGATGAGCTGACCAAGAACCAGGTCAGC |
| | | CTGACCTGCCTGGTCAAAGGCTTCTATCCCAGCGACATCGCCGTGGAGTGGGAG |
| | | AGCAATGGGCAGCCGGAGAACAACTACGACACCACGCCTCCCGTGCTGGACTCC |
| | | GACGGCTCCTTCTTCCTCTATAGCGACCTCACCGTGGACAAGAGCAGGTGGCAG |
| | | CAGGGGAACGTCTTCTCATGCTCCGTGATGCATGAGGCTCTGCACAACCACTAC |
| | | ACGCAGAAGAGCCTCTCCCTGTCTCCGGGT |
| 164 | Human IL- | GAGCTCTGTGACGATGACCCGCCAGAGATCCCACACGCCACATTCAAAGCCATG |
| | 2Raplha DNA | GCCTACAAGGAAGGAACCATGTTGAACTGTGAATGCAAGAGAGGTTTCCGCAGA |
| | Sequence | ATAAAAAGCGGGTCACTCTATATGCTCTGTACAGGAAACTCTAGCCACTCGTCC |
| | | TGGGACAACCAATGTCAATGCACAAGCTCTGCCACTCGGAACACAACGAAACAA |
| | | GTGACACCTCAACCTGAAGAACAGAAAGAAAGGAAAACCACAGAAATGCAAAGT |
| | | CCAATGCAGCCAGTGGACCAAGCGAGCCTTCCAGGTCACTGCAGGGAACCTCCA |
| | | CCATGGGAAAATGAAGCCACAGAGAGAATTTATCATTTCGTGGTGGGGCAGATG |
| | | GTTTATTATCAGTGCGTCCAGGGATACAGGGCTCTACACAGAGGTCCTGCTGAG |
| | | AGCGTCTGCAAAATGACCCACGGGAAGACAAGGTGGACCCAGCCCCAGCTCATA |
| | | TGCACAGGTGAAATGGAGACCAGTCAGTTTCCAGGTGAAGAGAAGCCTCAGGCA |
| | | AGCCCCGAAGGCCGTCCTGAGAGTGAGACTTCCTGCCTCGTCACAACAACAGAT |
| | | TTTCAAATACAGACAGAAATGGCTGCAACCATGGAGACGTCCATATTTACAACA |
| | | GAGTACCAG |
| 165 | Gly-Ser Linker | TCTGGTGGCGGTGGCTCTGGTGGCGGTGGC |
| | DNA sequence | |
| 166 | Human MMP | GGTCCTCTGGGTGTCAGAGGT |
| | Cleavage Site | |
| | DNA sequence | |
| 167- | Not Used | |
| 179 | | |
| 180- | See Table 2 | |
| 700 | | |

Additional Protease-cleavable sequences

| SEQ | | |
|---|---|---|
| ID | | |
| NO | Cleavable by | Sequence |
| 701 | MMP7 | KRALGLPG |
| 702 | MMP7 | (DE)₈RPLALWRS(DR)₈ |
| 703 | MMP9 | PR(S/T)(L/I)(S/T) |
| 704 | MMP9 | LEATA |
| 705 | MMP11 | GGAANLVRGG |
| 706 | MMP14 | SGRIGFLRTA |
| 707 | MMP | PLGLAG |
| 708 | MMP | PLGLAX |
| 709 | MMP | PLGC(me)AG |
| 710 | MMP | ESPAYYTA |
| 711 | MMP | RLQLKL |
| 712 | MMP | RLQLKAC |
| 713 | MMP, MMP9, | EP(Cit)G(Hof)YL |
| | MMP14 | |
| 714 | Urokinase | SGRSA |
| | plasminogen | |
| | activator | |
| | (uPA) | |
| 715 | Urokinase | DAFK |
| | plasminogen | |
| | activator | |
| | (uPA) | |
| 716 | Urokinase | GGGRR |
| | plasminogen | |
| | activator | |
| | (uPA) | |
| 717 | Lysomal | GFLG |
| | Enzyme | |
| 718 | Lysomal | ALAL |
| | Enzyme | |
| 719 | Lysomal | FK |
| | Enzyme | |
| 720 | Cathepsin B | NLL |
| 721 | Cathepsin D | PIC(Et)FF |
| 722 | Cathepsin K | GGPRGLPG |
| 723 | Prostate | HSSKLQ |
| | Specific | |
| | Antigen | |
| 724 | Prostate | HSSKLQL |
| | Specific | |
| | Antigen | |
| 725 | Prostate | HSSKLQEDA |
| | Specific | |
| | Antigen | |
| 726 | Herpes | LVLASSSFGY |
| | Simplex Virus | |
| | Protease | |
| 727 | HIV Protease | GVSQNYPIVG |
| 728 | CMV Protease | GVVQASCRLA |
| 729 | Thrombin | F(Pip)RS |
| 730 | Thrombin | DPRSFL |
| 731 | Thrombin | PPRSFL |
| 732 | Caspase-3 | DEVD |
| 733 | Caspase-3 | DEVDP |
| 734 | Caspase-3 | KGSGDVEG |
| 735 | Interleukin 1β | GWEHDG |
| | converting | |
| | enzyme | |
| 736 | Enterokinase | EDDDDKA |
| 737 | FAP | KQEQNPGST |
| 738 | Kallikrein 2 | GKAFRR |
| 739 | Plasmin | DAFK |
| 740 | Plasmin | DVLK |
| 741 | Plasmin | DAFK |
| 742 | TOP | ALLLALL |
| 743- | Not Used | |
| 799 | | |

Additional fusion polypeptides

| SEQ | | | | | |
|---|---|---|---|---|---|
| ID | | | | | |
| NO | Description | Sequence | Species | Function | Notes |
| 800 | TBM01 | MVSKGEELFTGVVPILVELDGDVNGHKFSVSGEGEGDATYGKLTLKFICT | | fusion | tool |
| | | TGKLPVPWPTLVTTLTYGVQCFSRYPDHMKQHDFFKSAMPEGYVQERTIF | | protein | |
| | | FKDDGNYKTRAEVKFEGDTLVNRIELKGIDFKEDGNILGHKLEYNYNSHN | | | |
| | | VYIMADKQKNGIKVNFKIRHNIEDGSVQLADHYQQNTPIGDGPVLLPDNH | | | |
| | | YLSTQSKLSKDPNEKRDHMVLLEFVTAAGITLGMDELYKGGGGSASKAQK | | | |
| | | AQAKQWKQAQKAQKAQAKQAKQAKQWSGGGGSGGGGGPLGVRGGGGGSGG | | | |
| | | GGSMVSKGEELIKENMHMKLYMEGTVNNHHFKCTSEGEGKPYEGTQTMRI | | | |
| | | KVVEGGPLPFAFDILATSFMYGSRTFINHTQGIPDFFKQSFPEGFTWERV | | | |
| | | TTYEDGGVLTATQDTSLQDGCLIYNVKIRGVNFPSNGPVMQKKTLGWEAN | | | |
| | | TEMLYPADGGLEGRSDMALKLVGGGHLICNFKTTYRSKKPAKNLKMPGVY | | | |
| | | YVDHRLERIKEADKETYVEQHEVAVARYCDLPSKLGHKLNGSGGGGGCKP | | | |
| | | CICTVPEVSSVFIFPPKPKDVLTITLTPKVTCVVVDISKDDPEVQFSWFV | | | |
| | | DDVEVHTAQTQPREEQFNSTFRSVSELPIMHQDWLNGKEFKCRVNSAAFP | | | |
| | | APIEKTISKTKGRPKAPQVYTIPPPKEQMAKDKVSLTCMITDFFPEDITV | | | |
| | | EWQWNGQPAENYKNTQPIMDTDGSYFVYSKLNVQKSNWEAGNTFTCSVLH | | | |
| | | EGLHNHHTEKSLSHSPGK | | | |
| 801 | TBM02 | MVSKGEELFTGVVPILVELDGDVNGHKFSVSGEGEGDATYGKLTLKFICT | | fusion | tool |
| | | TGKLPVPWPTLVTTLTYGVQCFSRYPDHMKQHDFFKSAMPEGYVQERTIF | | protein | |
| | | FKDDGNYKTRAEVKFEGDTLVNRIELKGIDFKEDGNILGHKLEYNYNSHN | | | |
| | | VYIMADKQKNGIKVNFKIRHNIEDGSVQLADHYQQNTPIGDGPVLLPDNH | | | |
| | | YLSTQSKLSKDPNEKRDHMVLLEFVTAAGITLGMDELYKSGGGGSGGGGG | | | |
| | | PLGVRGGGGGSGGGGSMVSKGEELIKENMHMKLYMEGTVNNHHFKCTSEG | | | |
| | | EGKPYEGTQTMRIKVVEGGPLPFAFDILATSFMYGSRTFINHTQGIPDFF | | | |
| | | KQSFPEGFTWERVTTYEDGGVLTATQDTSLQDGCLIYNVKIRGVNFPSNG | | | |
| | | PVMQKKTLGWEANTEMLYPADGGLEGRSDMALKLVGGGHLICNFKTTYRS | | | |
| | | KKPAKNLKMPGVYYVDHRLERIKEADKETYVEQHEVAVARYCDLPSKLGH | | | |
| | | KLNGSGGGGGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNG | | | |
| | | KEFKCRVNSAAFPAPIEKTISKTKGRPKAPQVYTIPPPKEQMAKDKVSLT | | | |
| | | CMITDFFPEDITVEWQWNGQPAENYKNTQPIMDTDGSYFVYSKLNVQKSN | | | |
| | | WEAGNTFTCSVLHEGLHNHHTEKSLSHSPGK | | | |
| 802 | TBM05 | MVSKGEELFTGVVPILVELDGDVNGHKFSVSGEGEGDATYGKLTLKFICT | | fusion | tool |
| | | TGKLPVPWPTLVTTLTYGVQCFSRYPDHMKQHDFFKSAMPEGYVQERTIF | | protein | |
| | | FKDDGNYKTRAEVKFEGDTLVNRIELKGIDFKEDGNILGHKLEYNYNSHN | | | |
| | | VYIMADKQKNGIKVNFKIRHNIEDGSVQLADHYQQNTPIGDGPVLLPDNH | | | |
| | | YLSTQSKLSKDPNEKRDHMVLLEFVTAAGITLGMDELYKSGGGGSGGGGG | | | |
| | | PLGVRGGGGGSGGGGSMVSKGEELIKENMHMKLYMEGTVNNHHFKCTSEG | | | |
| | | EGKPYEGTQTMRIKVVEGGPLPFAFDILATSFMYGSRTFINHTQGIPDFF | | | |
| | | KQSFPEGFTWERVTTYEDGGVLTATQDTSLQDGCLIYNVKIRGVNFPSNG | | | |
| | | PVMQKKTLGWEANTEMLYPADGGLEGRSDMALKLVGGGHLICNFKTTYRS | | | |
| | | KKPAKNLKMPGVYYVDHRLERIKEADKETYVEQHEVAVARYCDLPSKLGH | | | |
| | | KLNGSGGGGGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNG | | | |
| | | KEFKCRVNSAAFPAPIEKTISKTKGRPKAPQVYTIPPPKEQMAKDKVSLT | | | |
| | | CMITDFFPEDITVEWQWNGQPAENYKNTQPIMDTDGSYFVYSKLNVQKSN | | | |
| | | WEAGNTFTCSVLHEGLHNHHTEKSLSHSPGKGGGGSASKAQKAQAKQWKQ | | | |
| | | AQKAQKAQAKQAKQAKQW | | | |
| 803 | Construct F | APTSSSTKKTQLQLEHLLLDLQMILNGINNY | | fusion | |
| | | KNPKLTRMLTFKFYMPKKATELKHLQCLEEELKPLEEVLNLAQSKNFHLR | | protein | |
| | | PRDLISNINVIVLELKGSETTFMCEYADETATIVEFLNRWITFSQSIIST | | | |
| | | LTSGGGGSGGGGGPLGVRGGGGGSGGGGSELCLYDPPEVPNATFKALSYK | | | |
| | | NGTILNCECKRGFRRLKELVYMRCLGNSWSSNCQCTSSATRNTTKQVTPQ | | | |
| | | PEEQKERKTTEMQSPMQPVDQASLPGHCREPPPWENEATERIYHFVVGQM | | | |
| | | VYYQCVQGYRALHRGPAESVCKMTHGKTRWTQPQLICTGEMETSQFPGEE | | | |
| | | KPQASPEGRPESETSCLVTTTDFQIQTEMAATMETSIFTTEYQDKTHTCP | | | |
| | | PCPAPELLGGPSVFLFPPKPKDTLMISRTPEVTCVVVDVSHEDPEVKFNW | | | |
| | | YVDGVEVHNAKTKPREEQYNSTYRVVSVLTVLHQDWLNGKEYKCKVSNKA | | | |
| | | LPAPIEKTISKAKGQPREPQVYTLPPSRDELTKNQVSLTCLVKGFYPSDI | | | |
| | | AVEWESNGQPENNYKTTPPVLDSDGSFFLYSKLTVDKSRWQQGNVFSCSV | | | |
| | | MHEALHNHYTQKSLSLSPG | | | |
| 804 | Construct G | APTSSSTKKTQLQLEHLLLDLQMILNGINNY | | fusion | |
| | | KNPKLTRMLTFKFYMPKKATELKHLQCLEEELKPLEEVLNLAQSKNFHLR | | protein | |
| | | PRDLISNINVIVLELKGSETTFMCEYADETATIVEFLNRWITFSQSIIST | | | |
| | | LTSGGGGSGGGGGPLGVRGGGGGSGGGGSELCDDDPPEIPHATFKAMAYK | | | |
| | | EGTMLNCECKRGFRRIKSGSLYMLCTGNSSHSSWDNQCQCTSSATRNTTK | | | |
| | | QVTPQPEEQKERKTTEMQSPMQPVDQASLPGHCREPPPWENEATERIYHF | | | |
| | | VVGQMVYYQCVQGYRALHRGPAESVCKMTHGKTRWTQPQLICTGEMETSQ | | | |
| | | FPGEEKPQASPEGRPESETSCLVTTTDFQIQTEMAATMETSIFTTEYQGS | | | |
| | | GGGGDKTHTCPPCPAPELLGGPSVFLFPPKPKDTLMISRTPEVTCVVVDV | | | |
| | | SHEDPEVKFNWYVDGVEVHNAKTKPREEQYNSTYRVVSVLTVLHQDWLNG | | | |
| | | KEYKCKVSNKALPAPIEKTISKAKGQPREPQVYTLPPSRDELTKNQVSLT | | | |
| | | CLVKGFYPSDIAVEWESNGQPENNYKTTPPVLDSDGSFFLYSKLTVDKSR | | | |
| | | WQQGNVFSCSVMHEALHNHYTQKSLSLSPG | | | |
| 805 | Construct H | APTSSSTKKTQLQLEHLLLDLQMILNGINNYKNPKLTRMLTFKFYMPKKA | | fusion | |
| | | TELKHLQCLEEELKPLEEVLNLAQSKNFHLRPRDLISNINVIVLELKGSE | | protein | |
| | | TTFMCEYADETATIVEFLNRWITFSQSIISTLTSGGGGSGGGGGPLGVRG | | | |
| | | GGGGSGGGGSELCDDDPPEIPHATFKAMAYKEGTMLNCECKRGFRRIKSG | | | |
| | | SLYMLCTGNSSHSSWDNQCQCTSSATRNTTKQVTPQPEEQKERKTTEMQS | | | |
| | | PMQPVDQASLPGHCREPPPWENEATERIYHFVVGQMVYYQCVQGYRALHR | | | |
| | | GPAESVCKMTHGKTRWTQPQLICTGEMETSQFPGEEKPGSGGGGDKTHTC | | | |
| | | PPCPAPELLGGPSVFLFPPKPKDTLMISRTPEVTCVVVDVSHEDPEVKFN | | | |
| | | WYVDGVEVHNAKTKPREEQYNSTYRVVSVLTVLHQDWLNGKEYKCKVSNK | | | |
| | | ALPAPIEKTISKAKGQPREPQVYTLPPSRDELTKNQVSLTCLVKGFYPSD | | | |
| | | IAVEWESNGQPENNYKTTPPVLDSDGSFFLYSKLTVDKSRWQQGNVFSCS | | | |
| | | VMHEALHNHYTQKSLSLSPG | | | |
| 806 | Construct V | APTSSSTKKTQLQLEHLLLDLQMILNGINNYKNPKLTRMLTFKFYMPKKA | | fusion | |
| | | TELKHLQCLEEELKPLEEVLNLAQSKNFHLRPRDLISNINVIVLELKGSE | | protein | |
| | | TTFMCEYADETATIVEFLNRWITFSQSIISTLTSGGGGSGGGGGPLGVRG | | | |
| | | GGGGSGGGGSGGGGSGGGGSELCDDDPPEIPHATFKAMAYKEGTMLNCEC | | | |
| | | KRGFRRIKSGSLYMLCTGNSSHSSWDNQCQCTSSATRNTTKQVTPQPEEQ | | | |
| | | KERKTTEMQSPMQPVDQASLPGHCREPPPWENEATERIYHFVVGQMVYYQ | | | |
| | | CVQGYRALHRGPAESVCKMTHGKTRWTQPQLICTGEMETSQFPGEEKPQA | | | |
| | | SPEGRPESETSCLVTTTDFQIQTEMAATMETSIFTTEYQGSGGGGDKTHT | | | |
| | | CPPCPAPELLGGPSVFLFPPKPKDTLMISRTPEVTCVVVDVSHEDPEVKF | | | |
| | | NWYVDGVEVHNAKTKPREEQYNSTYRVVSVLTVLHQDWLNGKEYKCKVSN | | | |
| | | KALPAPIEKTISKAKGQPREPQVYTLPPSRDELTKNQVSLTCLVKGFYPS | | | |
| | | DIAVEWESNGQPENNYKTTPPVLDSDGSFFLYSKLTVDKSRWQQGNVFSC | | | |
| | | SVMHEALHNHYTQKSLSLSPG | | | |
| 807 | Construct W | APTSSSTKKTQLQLEHLLLDLQMILNGINNYKNPKLTRMLTFKFYMPKKA | | fusion | |
| | | TELKHLQCLEEELKPLEEVLNLAQSKNFHLRPRDLISNINVIVLELKGSE | | protein | |
| | | TTFMCEYADETATIVEFLNRWITFSQSIISTLTSGGGGSGGGGGPLGVRG | | | |
| | | GGGGSGGGGSGGGGSGGGGSGGGGSGGGGSELCDDDPPEIPHATFKAMAY | | | |
| | | KEGTMLNCECKRGFRRIKSGSLYMLCTGNSSHSSWDNQCQCTSSATRNTT | | | |
| | | KQVTPQPEEQKERKTTEMQSPMQPVDQASLPGHCREPPPWENEATERIYH | | | |
| | | FVVGQMVYYQCVQGYRALHRGPAESVCKMTHGKTRWTQPQLICTGEMETS | | | |
| | | QFPGEEKPQASPEGRPESETSCLVTTTDFQIQTEMAATMETSIFTTEYQG | | | |
| | | SGGGGDKTHTCPPCPAPELLGGPSVFLFPPKPKDTLMISRTPEVTCVVVD | | | |
| | | VSHEDPEVKFNWYVDGVEVHNAKTKPREEQYNSTYRVVSVLTVLHQDWLN | | | |
| | | GKEYKCKVSNKALPAPIEKTISKAKGQPREPQVYTLPPSRDELTKNQVSL | | | |
| | | TCLVKGFYPSDIAVEWESNGQPENNYKTTPPVLDSDGSFFLYSKLTVDKS | | | |
| | | RWQQGNVFSCSVMHEALHNHYTQKSLSLSPG | | | |
| 808 | Construct X | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFSQSIISTSPQSGGGGSGGGGGPA | | | |
| | | ALIGGGGGSGGGGSELCLYDPPEVPNATFKALSYKNGTILNCECKRGFRRLKEL | | | |
| | | VYMRCLGNSWSSNCQCTSNSHDKSRKQVTAQLEHQKEQQTTTDMQKPTQSMHQE | | | |
| | | NLTGHCREPPPWKHEDSKRIYHFVEGQSVHYECIPGYKALQRGPAISICKMKCG | | | |
| | | KTGWTQPQLTCVDEREHHRFLASEESQGSRNSSPESETSCPITTTDFPQPTETT | | | |
| | | AMTETFVLTMEYKGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNGKEFK | | | |
| | | CRVNSAAFPAPIEKTISKTKGRPKAPQVYTIPPPKEQMAKDKVSLTCMITDFFP | | | |
| | | EDITVEWQWNGQPAENYKNTQPIMDTDGSYFVYSKLNVQKSNWEAGNTFTCSVL | | | |
| | | HEGLHNHHTEKSLSHSPGK | | | |
| 809 | Construct Y | APTSSSTSSSTAEAQQQQQQQQQQQQHLEQLLMDLQELLSRMENYRNLKL | | fusion | |
| | | PRMLTFKFYLPKQATELKDLQCLEDELGPLRHVLDLTQSKSFQLEDAENF | | protein | |
| | | ISNIRVTVVKLKGSDNTFECQFDDESATVVDFLRRWIAFSQSIISTSPQV | | | |
| | | RIQRKKEKMKETGPLGVRGGGGGSGGGGSELCLYDPPEVPNATFKALSYK | | | |
| | | NGTILNCECKRGFRRLKELVYMRCLGNSWSSNCQCTSNSHDKSRKQVTAQ | | | |
| | | LEHQKEQQTTTDMQKPTQSMHQENLTGHCREPPPWKHEDSKRIYHFVEGQ | | | |
| | | SVHYECIPGYKALQRGPAISICKMKCGKTGWTQPQLTCVDEREHHRFLAS | | | |
| | | EESQGSRNSSPESETSCPITTTDFPQPTETTAMTETFVLTMEYKIEGRMD | | | |
| | | GCKPCICTVPEVSSVFIFPPKPKDVLTITLTPKVTCVVVDISKDDPEVQF | | | |
| | | SWFVDDVEVHTAQTQPREEQFNSTFRSVSELPIMHQDWLNGKEFKCRVNS | | | |
| | | AAFPAPIEKTISKTKGRPKAPQVYTIPPPKEQMAKDKVSLTCMITDFFPE | | | |
| | | DITVEWQWNGQPAENYKNTQPIMDTDGSYFVYSKLNVQKSNWEAGNTFTC | | | |
| | | SVLHEGLHNHHTEKSLSHSPGK | | | |
| 810 | Construct Z | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSGGGGSGGGGGPL | | | |
| | | GLARGGGGSGGGGSELCLYDPPEVPNATFKALSYKNGTILNCECKRGFRRLKEL | | | |
| | | VYMRCLGNSWSSNCQCTSNSHDKSRKQVTAQLEHQKEQQTTTDMQKPTQSMHQE | | | |
| | | NLTGHCREPPPWKHEDSKRIYHFVEGQSVHYECIPGYKALQRGPAISICKMKCG | | | |
| | | KTGWTQPQLTCVDEREHHRFLASEESQGSRNSSPESETSCPITTTDFPQPTETT | | | |
| | | AMTETFVLTMEYKGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNGKEFK | | | |
| | | CRVNSAAFPAPIEKTISKTKGRPKAPQVYTIPPPKEQMAKDKVSLTCMITDFFP | | | |
| | | EDITVEWQWNGQPAENYKNTQPIMDTDGSYFVYSKLNVQKSNWEAGNTFTCSVL | | | |
| | | HEGLHNHHTEKSLSHSPGK | | | |
| 811 | Construct AA | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSGGFHRRIKAGPL | | | |
| | | GVRGGGGGSGGGGSELCLYDPPEVPNATFKALSYKNGTILNCECKRGFRRLKEL | | | |
| | | VYMRCLGNSWSSNCQCTSNSHDKSRKQVTAQLEHQKEQQTTTDMQKPTQSMHQE | | | |
| | | NLTGHCREPPPWKHEDSKRIYHFVEGQSVHYECIPGYKALQRGPAISICKMKCG | | | |
| | | KTGWTQPQLTCVDEREHHRFLASEESQGSRNSSPESETSCPITTTDFPQPTETT | | | |
| | | AMTETFVLTMEYKGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNGKEFK | | | |
| | | CRVNSAAFPAPIEKTISKTKGRPKAPQVYTIPPPKEQMAKDKVSLTCMITDFFP | | | |
| | | EDITVEWQWNGQPAENYKNTQPIMDTDGSYFVYSKLNVQKSNWEAGNTFTCSVL | | | |
| | | HEGLHNHHTEKSLSHSPGK | | | |
| 812 | Construct BB | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSGGFHRRIKAGVR | | | |
| | | LGPGGGGGSGGGGSELCLYDPPEVPNATFKALSYKNGTILNCECKRGFRRLKEL | | | |
| | | VYMRCLGNSWSSNCQCTSNSHDKSRKQVTAQLEHQKEQQTTTDMQKPTQSMHQE | | | |
| | | NLTGHCREPPPWKHEDSKRIYHFVEGQSVHYECIPGYKALQRGPAISICKMKCG | | | |
| | | KTGWTQPQLTCVDEREHHRFLASEESQGSRNSSPESETSCPITTTDFPQPTETT | | | |
| | | AMTETFVLTMEYKGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNGKEFK | | | |
| | | CRVNSAAFPAPIEKTISKTKGRPKAPQVYTIPPPKEQMAKDKVSLTCMITDFFP | | | |
| | | EDITVEWQWNGQPAENYKNTQPIMDTDGSYFVYSKLNVQKSNWEAGNTFTCSVL | | | |
| | | HEGLHNHHTEKSLSHSPGK | | | |
| 813 | Construct CC | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQGHHPHGHHPHGPL | | | |
| | | GVRGGGGGSGGGGSELCLYDPPEVPNATFKALSYKNGTILNCECKRGFRRLKEL | | | |
| | | VYMRCLGNSWSSNCQCTSNSHDKSRKQVTAQLEHQKEQQTTTDMQKPTQSMHQE | | | |
| | | NLTGHCREPPPWKHEDSKRIYHFVEGQSVHYECIPGYKALQRGPAISICKMKCG | | | |
| | | KTGWTQPQLTCVDEREHHRFLASEESQGSRNSSPESETSCPITTTDFPQPTETT | | | |
| | | AMTETFVLTMEYKGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNGKEFK | | | |
| | | CRVNSAAFPAPIEKTISKTKGRPKAPQVYTIPPPKEQMAKDKVSLTCMITDFFP | | | |
| | | EDITVEWQWNGQPAENYKNTQPIMDTDGSYFVYSKLNVQKSNWEAGNTFTCSVL | | | |
| | | HEGLHNHHTEKSLSHSPGK | | | |
| 814 | Construct DD | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQGHHPHGHHPHGVR | | | |
| | | LGPGGGGGSGGGGSELCLYDPPEVPNATFKALSYKNGTILNCECKRGFRRLKEL | | | |
| | | VYMRCLGNSWSSNCQCTSNSHDKSRKQVTAQLEHQKEQQTTTDMQKPTQSMHQE | | | |
| | | NLTGHCREPPPWKHEDSKRIYHFVEGQSVHYECIPGYKALQRGPAISICKMKCG | | | |
| | | KTGWTQPQLTCVDEREHHRFLASEESQGSRNSSPESETSCPITTTDFPQPTETT | | | |
| | | AMTETFVLTMEYKGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNGKEFK | | | |
| | | CRVNSAAFPAPIEKTISKTKGRPKAPQVYTIPPPKEQMAKDKVSLTCMITDFFP | | | |
| | | EDITVEWQWNGQPAENYKNTQPIMDTDGSYFVYSKLNVQKSNWEAGNTFTCSVL | | | |
| | | HEGLHNHHTEKSLSHSPGK | | | |
| 815 | Construct EE | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSGGGGWSHWGPLG | | | |
| | | VRGGGGGSGGGGSELCLYDPPEVPNATFKALSYKNGTILNCECKRGFRRLKELV | | | |
| | | YMRCLGNSWSSNCQCTSNSHDKSRKQVTAQLEHQKEQQTTTDMQKPTQSMHQEN | | | |
| | | LTGHCREPPPWKHEDSKRIYHFVEGQSVHYECIPGYKALQRGPAISICKMKCGK | | | |
| | | TGWTQPQLTCVDEREHHRFLASEESQGSRNSSPESETSCPITTTDFPQPTETTA | | | |
| | | MTETFVLTMEYKGCKPCICTVPEVSSVFIFPPKPKDVLTITLTPKVTCVVVDIS | | | |
| | | KDDPEVQFSWFVDDVEVHTAQTQPREEQFNSTFRSVSELPIMHQDWLNGKEFKC | | | |
| | | RVNSAAFPAPIEKTISKTKGRPKAPQVYTIPPPKEQMAKDKVSLTCMITDFFPE | | | |
| | | DITVEWQWNGQPAENYKNTQPIMDTDGSYFVYSKLNVQKSNWEAGNTFTCSVLH | | | |
| | | EGLHNHHTEKSLSHSPGK | | | |
| 816 | Construct FF | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSGGGGWSHWGVRL | | | |
| | | GPGGGGGSGGGGSELCLYDPPEVPNATFKALSYKNGTILNCECKRGFRRLKELV | | | |
| | | YMRCLGNSWSSNCQCTSNSHDKSRKQVTAQLEHQKEQQTTTDMQKPTQSMHQEN | | | |
| | | LTGHCREPPPWKHEDSKRIYHFVEGQSVHYECIPGYKALQRGPAISICKMKCGK | | | |
| | | TGWTQPQLTCVDEREHHRFLASEESQGSRNSSPESETSCPITTTDFPQPTETTA | | | |
| | | MTETFVLTMEYKGCKPCICTVPEVSSVFIFPPKPKDVLTITLTPKVTCVVVDIS | | | |
| | | KDDPEVQFSWFVDDVEVHTAQTQPREEQFNSTFRSVSELPIMHQDWLNGKEFKC | | | |
| | | RVNSAAFPAPIEKTISKTKGRPKAPQVYTIPPPKEQMAKDKVSLTCMITDFFPE | | | |
| | | DITVEWQWNGQPAENYKNTQPIMDTDGSYFVYSKLNVQKSNWEAGNTFTCSVLH | | | |
| | | EGLHNHHTEKSLSHSPGK | | | |
| 817 | Construct GG | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSGGKLWVLPKGPL | | | |
| | | GVRGGGGGSGGGGSELCLYDPPEVPNATFKALSYKNGTILNCECKRGFRRLKEL | | | |
| | | VYMRCLGNSWSSNCQCTSNSHDKSRKQVTAQLEHQKEQQTTTDMQKPTQSMHQE | | | |
| | | NLTGHCREPPPWKHEDSKRIYHFVEGQSVHYECIPGYKALQRGPAISICKMKCG | | | |
| | | KTGWTQPQLTCVDEREHHRFLASEESQGSRNSSPESETSCPITTTDFPQPTETT | | | |
| | | AMTETFVLTMEYKGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNGKEFK | | | |
| | | CRVNSAAFPAPIEKTISKTKGRPKAPQVYTIPPPKEQMAKDKVSLTCMITDFFP | | | |
| | | EDITVEWQWNGQPAENYKNTQPIMDTDGSYFVYSKLNVQKSNWEAGNTFTCSVL | | | |
| | | HEGLHNHHTEKSLSHSPGK | | | |
| 818 | Construct HH | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSGGKLWVLPKGVR | | | |
| | | LGPGGGGGSGGGGSELCLYDPPEVPNATFKALSYKNGTILNCECKRGFRRLKEL | | | |
| | | VYMRCLGNSWSSNCQCTSNSHDKSRKQVTAQLEHQKEQQTTTDMQKPTQSMHQE | | | |
| | | NLTGHCREPPPWKHEDSKRIYHFVEGQSVHYECIPGYKALQRGPAISICKMKCG | | | |
| | | KTGWTQPQLTCVDEREHHRFLASEESQGSRNSSPESETSCPITTTDFPQPTETT | | | |
| | | AMTETFVLTMEYKGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNGKEFK | | | |
| | | CRVNSAAFPAPIEKTISKTKGRPKAPQVYTIPPPKEQMAKDKVSLTCMITDFFP | | | |
| | | EDITVEWQWNGQPAENYKNTQPIMDTDGSYFVYSKLNVQKSNWEAGNTFTCSVL | | | |
| | | HEGLHNHHTEKSLSHSPGK | | | |
| 819 | Construct II | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSLHERHLNNNGPL | | | |
| | | GVRGGGGGSGGGGSELCLYDPPEVPNATFKALSYKNGTILNCECKRGFRRLKEL | | | |
| | | VYMRCLGNSWSSNCQCTSNSHDKSRKQVTAQLEHQKEQQTTTDMQKPTQSMHQE | | | |
| | | NLTGHCREPPPWKHEDSKRIYHFVEGQSVHYECIPGYKALQRGPAISICKMKCG | | | |
| | | KTGWTQPQLTCVDEREHHRFLASEESQGSRNSSPESETSCPITTTDFPQPTETT | | | |
| | | AMTETFVLTMEYKGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNGKEFK | | | |
| | | CRVNSAAFPAPIEKTISKTKGRPKAPQVYTIPPPKEQMAKDKVSLTCMITDFFP | | | |
| | | EDITVEWQWNGQPAENYKNTQPIMDTDGSYFVYSKLNVQKSNWEAGNTFTCSVL | | | |
| | | HEGLHNHHTEKSLSHSPGK | | | |
| 820 | Construct JJ | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSLHERHLNNNGVR | | | |
| | | LGPGGGGGSGGGGSELCLYDPPEVPNATFKALSYKNGTILNCECKRGFRRLKEL | | | |
| | | VYMRCLGNSWSSNCQCTSNSHDKSRKQVTAQLEHQKEQQTTTDMQKPTQSMHQE | | | |
| | | NLTGHCREPPPWKHEDSKRIYHFVEGQSVHYECIPGYKALQRGPAISICKMKCG | | | |
| | | KTGWTQPQLTCVDEREHHRFLASEESQGSRNSSPESETSCPITTTDFPQPTETT | | | |
| | | AMTETFVLTMEYKGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNGKEFK | | | |
| | | CRVNSAAFPAPIEKTISKTKGRPKAPQVYTIPPPKEQMAKDKVSLTCMITDFFP | | | |
| | | EDITVEWQWNGQPAENYKNTQPIMDTDGSYFVYSKLNVQKSNWEAGNTFTCSVL | | | |
| | | HEGLHNHHTEKSLSHSPGK | | | |
| 821 | Construct KK | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQVRIQRKKEKMKET | | | |
| | | GVRLGPGGGGGSGGGGSELCLYDPPEVPNATFKALSYKNGTILNCECKRGFRRL | | | |
| | | KELVYMRCLGNSWSSNCQCTSNSHDKSRKQVTAQLEHQKEQQTTTDMQKPTQSM | | | |
| | | HQENLTGHCREPPPWKHEDSKRIYHFVEGQSVHYECIPGYKALQRGPAISICKM | | | |
| | | KCGKTGWTQPQLTCVDEREHHRFLASEESQGSRNSSPESETSCPITTTDFPQPT | | | |
| | | ETTAMTETFVLTMEYKGCKPCICTVPEVSSVFIFPPKPKDVLTITLTPKVTCVV | | | |
| | | VDISKDDPEVQFSWFVDDVEVHTAQTQPREEQFNSTFRSVSELPIMHQDWLNGK | | | |
| | | EFKCRVNSAAFPAPIEKTISKTKGRPKAPQVYTIPPPKEQMAKDKVSLTCMITD | | | |
| | | FFPEDITVEWQWNGQPAENYKNTQPIMDTDGSYFVYSKLNVQKSNWEAGNTFTC | | | |
| | | SVLHEGLHNHHTEKSLSHSPGK | | | |
| 822 | Construct LL | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSGGGGSGGGGGPL | | | |
| | | GVRGFHRRIKAGGSELCLYDPPEVPNATFKALSYKNGTILNCECKRGFRRLKEL | | | |
| | | VYMRCLGNSWSSNCQCTSNSHDKSRKQVTAQLEHQKEQQTTTDMQKPTQSMHQE | | | |
| | | NLTGHCREPPPWKHEDSKRIYHFVEGQSVHYECIPGYKALQRGPAISICKMKCG | | | |
| | | KTGWTQPQLTCVDEREHHRFLASEESQGSRNSSPESETSCPITTTDFPQPTETT | | | |
| | | AMTETFVLTMEYKGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNGKEFK | | | |
| | | CRVNSAAFPAPIEKTISKTKGRPKAPQVYTIPPPKEQMAKDKVSLTCMITDFFP | | | |
| | | EDITVEWQWNGQPAENYKNTQPIMDTDGSYFVYSKLNVQKSNWEAGNTFTCSVL | | | |
| | | HEGLHNHHTEKSLSHSPGK | | | |
| 823 | Construct MM | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSGGGGSGGGGGVR | | | |
| | | LGPGFHRRIKAGGSELCLYDPPEVPNATFKALSYKNGTILNCECKRGFRRLKEL | | | |
| | | VYMRCLGNSWSSNCQCTSNSHDKSRKQVTAQLEHQKEQQTTTDMQKPTQSMHQE | | | |
| | | NLTGHCREPPPWKHEDSKRIYHFVEGQSVHYECIPGYKALQRGPAISICKMKCG | | | |
| | | KTGWTQPQLTCVDEREHHRFLASEESQGSRNSSPESETSCPITTTDFPQPTETT | | | |
| | | AMTETFVLTMEYKGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNGKEFK | | | |
| | | CRVNSAAFPAPIEKTISKTKGRPKAPQVYTIPPPKEQMAKDKVSLTCMITDFFP | | | |
| | | EDITVEWQWNGQPAENYKNTQPIMDTDGSYFVYSKLNVQKSNWEAGNTFTCSVL | | | |
| | | HEGLHNHHTEKSLSHSPGK | | | |
| 824 | Construct NN | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSGGGGSGGGGGPL | | | |
| | | GVRGGHHPHGHHPHELCLYDPPEVPNATFKALSYKNGTILNCECKRGFRRLKEL | | | |
| | | VYMRCLGNSWSSNCQCTSNSHDKSRKQVTAQLEHQKEQQTTTDMQKPTQSMHQE | | | |
| | | NLTGHCREPPPWKHEDSKRIYHFVEGQSVHYECIPGYKALQRGPAISICKMKCG | | | |
| | | KTGWTQPQLTCVDEREHHRFLASEESQGSRNSSPESETSCPITTTDFPQPTETT | | | |
| | | AMTETFVLTMEYKGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNGKEFK | | | |
| | | CRVNSAAFPAPIEKTISKTKGRPKAPQVYTIPPPKEQMAKDKVSLTCMITDFFP | | | |
| | | EDITVEWQWNGQPAENYKNTQPIMDTDGSYFVYSKLNVQKSNWEAGNTFTCSVL | | | |
| | | HEGLHNHHTEKSLSHSPGK | | | |
| 825 | Construct 00 | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSGGGGSGGGGGVR | | | |
| | | LGPGGHHPHGHHPHELCLYDPPEVPNATFKALSYKNGTILNCECKRGFRRLKEL | | | |
| | | VYMRCLGNSWSSNCQCTSNSHDKSRKQVTAQLEHQKEQQTTTDMQKPTQSMHQE | | | |
| | | NLTGHCREPPPWKHEDSKRIYHFVEGQSVHYECIPGYKALQRGPAISICKMKCG | | | |
| | | KTGWTQPQLTCVDEREHHRFLASEESQGSRNSSPESETSCPITTTDFPQPTETT | | | |
| | | AMTETFVLTMEYKGCKPCICTVPEVSSVFIFPPKPKDVLTITLTPKVTCVVVDI | | | |
| | | SKDDPEVQFSWFVDDVEVHTAQTQPREEQFNSTFRSVSELPIMHQDWLNGKEFK | | | |
| | | CRVNSAAFPAPIEKTISKTKGRPKAPQVYTIPPPKEQMAKDKVSLTCMITDFFP | | | |
| | | EDITVEWQWNGQPAENYKNTQPIMDTDGSYFVYSKLNVQKSNWEAGNTFTCSVL | | | |
| | | HEGLHNHHTEKSLSHSPGK | | | |
| 826 | Construct PP | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSGGGGSGGGGGPL | | | |
| | | GVRGGGWSHWGGSELCLYDPPEVPNATFKALSYKNGTILNCECKRGFRRLKELV | | | |
| | | YMRCLGNSWSSNCQCTSNSHDKSRKQVTAQLEHQKEQQTTTDMQKPTQSMHQEN | | | |
| | | LTGHCREPPPWKHEDSKRIYHFVEGQSVHYECIPGYKALQRGPAISICKMKCGK | | | |
| | | TGWTQPQLTCVDEREHHRFLASEESQGSRNSSPESETSCPITTTDFPQPTETTA | | | |
| | | MTETFVLTMEYKGCKPCICTVPEVSSVFIFPPKPKDVLTITLTPKVTCVVVDIS | | | |
| | | KDDPEVQFSWFVDDVEVHTAQTQPREEQFNSTFRSVSELPIMHQDWLNGKEFKC | | | |
| | | RVNSAAFPAPIEKTISKTKGRPKAPQVYTIPPPKEQMAKDKVSLTCMITDFFPE | | | |
| | | DITVEWQWNGQPAENYKNTQPIMDTDGSYFVYSKLNVQKSNWEAGNTFTCSVLH | | | |
| | | EGLHNHHTEKSLSHSPGK | | | |
| 827 | Construct QQ | APTSSSTSSSTAEAQQQQQQQQQQQQHLEQLLMDLQELLSRMENYRNLKLPRML | | fusion | |
| | | TFKFYLPKQATELKDLQCLEDELGPLRHVLDLTQSKSFQLEDAENFISNIRVTV | | protein | |
| | | VKLKGSDNTFECQFDDESATVVDFLRRWIAFCQSIISTSPQSGGGGSGGGGGVR | | | |
| | | LGPGGGWSHWGGSELCLYDPPEVPNATFKALSYKNGTILNCECKRGFRRLKELV | | | |
| | | YMRCLGNSWSSNCQCTSNSHDKSRKQVTAQLEHQKEQQTTTDMQKPTQSMHQEN | | | |
| | | LTGHCREPPPWKHEDSKRIYHFVEGQSVHYECIPGYKALQRGPAISICKMKCGK | | | |
| | | TGWTQPQLTCVDEREHHRFLASEESQGSRNSSPESETSCPITTTDFPQPTETTA | | | |
| | | MTETFVLTMEYKGCKPCICTVPEVSSVFIFPPKPKDVLTITLTPKVTCVVVDIS | | | |
| | | KDDPEVQFSWFVDDVEVHTAQTQPREEQFNSTFRSVSELPIMHQDWLNGKEFKC | | | |
| | | RVNSAAFPAPIEKTISKTKGRPKAPQVYTIPPPKEQMAKDKVSLTCMITDFFPE | | | |
| | | DITVEWQWNGQPAENYKNTQPIMDTDGSYFVYSKLNVQKSNWEAGNTFTCSVLH | | | |
| | | EGLHNHHTEKSLSHSPGK | | | |
| 828 | Construct RR | MGWSCIILFLVATATGVHSAPTSSSTSSSTAEAQQQQQQQQQQQQHLEQLLMDL | | fusion | |
| | | QELLSRMENYRNLKLPRMLTFKFYLPKQATELKDLQCLEDELGPLRHVLDLTQS | | protein | |
| | | KSFQLEDAENFISNIRVTVVKLKGSDNTFECQFDDESATVVDFLRRWIAFCQSI | | | |
| | | ISTSPQSGGGGSGGGGGPLGVRGKLWVLPKGGSELCLYDPPEVPNATFKALSYK | | | |
| | | NGTILNCECKRGFRRLKELVYMRCLGNSWSSNCQCTSNSHDKSRKQVTAQLEHQ | | | |
| | | KEQQTTTDMQKPTQSMHQENLTGHCREPPPWKHEDSKRIYHFVEGQSVHYECIP | | | |
| | | GYKALQRGPAISICKMKCGKTGWTQPQLTCVDEREHHRFLASEESQGSRNSSPE | | | |
| | | SETSCPITTTDFPQPTETTAMTETFVLTMEYKGCKPCICTVPEVSSVFIFPPKP | | | |
| | | KDVLTITLTPKVTCVVVDISKDDPEVQFSWFVDDVEVHTAQTQPREEQFNSTFR | | | |
| | | SVSELPIMHQDWLNGKEFKCRVNSAAFPAPIEKTISKTKGRPKAPQVYTIPPPK | | | |
| | | EQMAKDKVSLTCMITDFFPEDITVEWQWNGQPAENYKNTQPIMDTDGSYFVYSK | | | |
| | | LNVQKSNWEAGNTFTCSVLHEGLHNHHTEKSLSHSPGK | | | |
| 829 | Construct SS | MGWSCIILFLVATATGVHSAPTSSSTSSSTAEAQQQQQQQQQQQQHLEQLLMDL | | fusion | |
| | | QELLSRMENYRNLKLPRMLTFKFYLPKQATELKDLQCLEDELGPLRHVLDLTQS | | protein | |
| | | KSFQLEDAENFISNIRVTVVKLKGSDNTFECQFDDESATVVDFLRRWIAFCQSI | | | |
| | | ISTSPQSGGGGSGGGGGVRLGPGKLWVLPKGGSELCLYDPPEVPNATFKALSYK | | | |
| | | NGTILNCECKRGFRRLKELVYMRCLGNSWSSNCQCTSNSHDKSRKQVTAQLEHQ | | | |
| | | KEQQTTTDMQKPTQSMHQENLTGHCREPPPWKHEDSKRIYHFVEGQSVHYECIP | | | |
| | | GYKALQRGPAISICKMKCGKTGWTQPQLTCVDEREHHRFLASEESQGSRNSSPE | | | |
| | | SETSCPITTTDFPQPTETTAMTETFVLTMEYKGCKPCICTVPEVSSVFIFPPKP | | | |
| | | KDVLTITLTPKVTCVVVDISKDDPEVQFSWFVDDVEVHTAQTQPREEQFNSTFR | | | |
| | | SVSELPIMHQDWLNGKEFKCRVNSAAFPAPIEKTISKTKGRPKAPQVYTIPPPK | | | |
| | | EQMAKDKVSLTCMITDFFPEDITVEWQWNGQPAENYKNTQPIMDTDGSYFVYSK | | | |
| | | LNVQKSNWEAGNTFTCSVLHEGLHNHHTEKSLSHSPGK | | | |
| 830 | Construct TT | MGWSCIILFLVATATGVHSAPTSSSTSSSTAEAQQQQQQQQQQQQHLEQLLMDL | | fusion | |
| | | QELLSRMENYRNLKLPRMLTFKFYLPKQATELKDLQCLEDELGPLRHVLDLTQS | | protein | |
| | | KSFQLEDAENFISNIRVTVVKLKGSDNTFECQFDDESATVVDFLRRWIAFCQSI | | | |
| | | ISTSPQSGGGGSGGGGGPLGVRGLHERHLNNNGELCLYDPPEVPNATFKALSYK | | | |
| | | NGTILNCECKRGFRRLKELVYMRCLGNSWSSNCQCTSNSHDKSRKQVTAQLEHQ | | | |
| | | KEQQTTTDMQKPTQSMHQENLTGHCREPPPWKHEDSKRIYHFVEGQSVHYECIP | | | |
| | | GYKALQRGPAISICKMKCGKTGWTQPQLTCVDEREHHRFLASEESQGSRNSSPE | | | |
| | | SETSCPITTTDFPQPTETTAMTETFVLTMEYKGCKPCICTVPEVSSVFIFPPKP | | | |
| | | KDVLTITLTPKVTCVVVDISKDDPEVQFSWFVDDVEVHTAQTQPREEQFNSTFR | | | |
| | | SVSELPIMHQDWLNGKEFKCRVNSAAFPAPIEKTISKTKGRPKAPQVYTIPPPK | | | |
| | | EQMAKDKVSLTCMITDFFPEDITVEWQWNGQPAENYKNTQPIMDTDGSYFVYSK | | | |
| | | LNVQKSNWEAGNTFTCSVLHEGLHNHHTEKSLSHSPGK | | | |
| 831 | Construct UU | MGWSCIILFLVATATGVHSAPTSSSTSSSTAEAQQQQQQQQQQQQHLEQLLMDL | | fusion | |
| | | QELLSRMENYRNLKLPRMLTFKFYLPKQATELKDLQCLEDELGPLRHVLDLTQS | | protein | |
| | | KSFQLEDAENFISNIRVTVVKLKGSDNTFECQFDDESATVVDFLRRWIAFCQSI | | | |
| | | ISTSPQSGGGGSGGGGGVRLGPGLHERHLNNNGELCLYDPPEVPNATFKALSYK | | | |
| | | NGTILNCECKRGFRRLKELVYMRCLGNSWSSNCQCTSNSHDKSRKQVTAQLEHQ | | | |
| | | KEQQTTTDMQKPTQSMHQENLTGHCREPPPWKHEDSKRIYHFVEGQSVHYECIP | | | |
| | | GYKALQRGPAISICKMKCGKTGWTQPQLTCVDEREHHRFLASEESQGSRNSSPE | | | |
| | | SETSCPITTTDFPQPTETTAMTETFVLTMEYKGCKPCICTVPEVSSVFIFPPKP | | | |
| | | KDVLTITLTPKVTCVVVDISKDDPEVQFSWFVDDVEVHTAQTQPREEQFNSTFR | | | |
| | | SVSELPIMHQDWLNGKEFKCRVNSAAFPAPIEKTISKTKGRPKAPQVYTIPPPK | | | |
| | | EQMAKDKVSLTCMITDFFPEDITVEWQWNGQPAENYKNTQPIMDTDGSYFVYSK | | | |
| | | LNVQKSNWEAGNTFTCSVLHEGLHNHHTEKSLSHSPGK | | | |
| 832 | Construct VV | MGWSCIILFLVATATGVHSAPTSSSTSSSTAEAQQQQQQQQQQQQHLEQLLMDL | | fusion | |
| | | QELLSRMENYRNLKLPRMLTFKFYLPKQATELKDLQCLEDELGPLRHVLDLTQS | | protein | |
| | | KSFQLEDAENFISNIRVTVVKLKGSDNTFECQFDDESATVVDFLRRWIAFCQSI | | | |
| | | ISTSPQSGGGGGHHPHGPLGVRGGGGGSGGGGSELCLYDPPEVPNATFKALSYK | | | |
| | | NGTILNCECKRGFRRLKELVYMRCLGNSWSSNCQCTSNSHDKSRKQVTAQLEHQ | | | |
| | | KEQQTTTDMQKPTQSMHQENLTGHCREPPPWKHEDSKRIYHFVEGQSVHYECIP | | | |
| | | GYKALQRGPAISICKMKCGKTGWTQPQLTCVDEREHHRFLASEESQGSRNSSPE | | | |
| | | SETSCPITTTDFPQPTETTAMTETFVLTMEYKGCKPCICTVPEVSSVFIFPPKP | | | |
| | | KDVLTITLTPKVTCVVVDISKDDPEVQFSWFVDDVEVHTAQTQPREEQFNSTFR | | | |
| | | SVSELPIMHQDWLNGKEFKCRVNSAAFPAPIEKTISKTKGRPKAPQVYTIPPPK | | | |
| | | EQMAKDKVSLTCMITDFFPEDITVEWQWNGQPAENYKNTQPIMDTDGSYFVYSK | | | |
| | | LNVQKSNWEAGNTFTCSVLHEGLHNHHTEKSLSHSPGK | | | |
| 833 | Construct WW | MGWSCIILFLVATATGVHSAPTSSSTSSSTAEAQQQQQQQQQQQQHLEQLLMDL | | fusion | |
| | | QELLSRMENYRNLKLPRMLTFKFYLPKQATELKDLQCLEDELGPLRHVLDLTQS | | protein | |
| | | KSFQLEDAENFISNIRVTVVKLKGSDNTFECQFDDESATVVDFLRRWIAFCQSI | | | |
| | | ISTSPQGHHPHSGGGGGPLGVRGGGGGSGGGGSELCLYDPPEVPNATFKALSYK | | | |
| | | NGTILNCECKRGFRRLKELVYMRCLGNSWSSNCQCTSNSHDKSRKQVTAQLEHQ | | | |
| | | KEQQTTTDMQKPTQSMHQENLTGHCREPPPWKHEDSKRIYHFVEGQSVHYECIP | | | |
| | | GYKALQRGPAISICKMKCGKTGWTQPQLTCVDEREHHRFLASEESQGSRNSSPE | | | |
| | | SETSCPITTTDFPQPTETTAMTETFVLTMEYKGCKPCICTVPEVSSVFIFPPKP | | | |
| | | KDVLTITLTPKVTCVVVDISKDDPEVQFSWFVDDVEVHTAQTQPREEQFNSTFR | | | |
| | | SVSELPIMHQDWLNGKEFKCRVNSAAFPAPIEKTISKTKGRPKAPQVYTIPPPK | | | |
| | | EQMAKDKVSLTCMITDFFPEDITVEWQWNGQPAENYKNTQPIMDTDGSYFVYSK | | | |
| | | LNVQKSNWEAGNTFTCSVLHEGLHNHHTEKSLSHSPGK | | | |
| 834 | Construct XX | MGWSCIILFLVATATGVHSAPTSSSTSSSTAEAQQQQQQQQQQQQHLEQLLMDL | | fusion | |
| | | QELLSRMENYRNLKLPRMLTFKFYLPKQATELKDLQCLEDELGPLRHVLDLTQS | | protein | |
| | | KSFQLEDAENFISNIRVTVVKLKGSDNTFECQFDDESATVVDFLRRWIAFCQSI | | | |
| | | ISTSPQSGGGGSGGGGGPLGVRGGHHPHGGGGSELCLYDPPEVPNATFKALSYK | | | |
| | | NGTILNCECKRGFRRLKELVYMRCLGNSWSSNCQCTSNSHDKSRKQVTAQLEHQ | | | |
| | | KEQQTTTDMQKPTQSMHQENLTGHCREPPPWKHEDSKRIYHFVEGQSVHYECIP | | | |
| | | GYKALQRGPAISICKMKCGKTGWTQPQLTCVDEREHHRFLASEESQGSRNSSPE | | | |
| | | SETSCPITTTDFPQPTETTAMTETFVLTMEYKGCKPCICTVPEVSSVFIFPPKP | | | |
| | | KDVLTITLTPKVTCVVVDISKDDPEVQFSWFVDDVEVHTAQTQPREEQFNSTFR | | | |
| | | SVSELPIMHQDWLNGKEFKCRVNSAAFPAPIEKTISKTKGRPKAPQVYTIPPPK | | | |
| | | EQMAKDKVSLTCMITDFFPEDITVEWQWNGQPAENYKNTQPIMDTDGSYFVYSK | | | |
| | | LNVQKSNWEAGNTFTCSVLHEGLHNHHTEKSLSHSPGK | | | |
| 835 | Construct YY | MGWSCIILFLVATATGVHSAPTSSSTSSSTAEAQQQQQQQQQQQQHLEQLLMDL | | fusion | |
| | | QELLSRMENYRNLKLPRMLTFKFYLPKQATELKDLQCLEDELGPLRHVLDLTQS | | protein | |
| | | KSFQLEDAENFISNIRVTVVKLKGSDNTFECQFDDESATVVDFLRRWIAFCQSI | | | |
| | | ISTSPQSGGGGSGGGGGPLGVRGGGGGSGGGGSELCLYDPPEVPNATFKALSYK | | | |
| | | NGTILNCECKRGFRRLKELVYMRCLGNSWSSNCQCTSNSHDKSRKQVTAQLEHQ | | | |
| | | KEQQTTTDMQKPTQSMHQENLTGHCREPPPWKHEDSKRIYHFVEGQSVHYECIP | | | |
| | | GYKALQRGPAISICKMKCGKTGWTQPQLTCVDEREHHRFLASEESQGSRNSSPE | | | |
| | | SETSCPITTTDFPQPTETTAMTETFVLTMEYKGCKPCICTVPEVSSVFIFPPKP | | | |
| | | KDVLTITLTPKVTCVVVDISKDDPEVQFSWFVDDVEVHTAQTQPREEQFNSTFR | | | |
| | | SVSELPIMHQDWLNGKEFKCRVNSAAFPAPIEKTISKTKGRPKAPQVYTIPPPK | | | |
| | | EQMAKDKVSLTCMITDFFPEDITVEWQWNGQPAENYKNTQPIMDTDGSYFVYSK | | | |
| | | LNVQKSNWEAGNTFTCSVLHEGLHNHHTEKSLSHSPGKGHHPHGHHPH | | | |
| 836 | Construct ZZ | MGWSCIILFLVATATGVHSAPTSSSTSSSTAEAQQQQQQQQQQQQHLEQLLMDL | | fusion | |
| | | QELLSRMENYRNLKLPRMLTFKFYLPKQATELKDLQCLEDELGPLRHVLDLTQS | | protein | |
| | | KSFQLEDAENFISNIRVTVVKLKGSDNTFECQFDDESATVVDFLRRWIAFCQSI | | | |
| | | ISTSPQSGGGGSGGGGGPLGVRGGGGGSGGGGSELCLYDPPEVPNATFKALSYK | | | |
| | | NGTILNCECKRGFRRLKELVYMRCLGNSWSSNCQCTSNSHDKSRKQVTAQLEHQ | | | |
| | | KEQQTTTDMQKPTQSMHQENLTGHCREPPPWKHEDSKRIYHFVEGQSVHYECIP | | | |
| | | GYKALQRGPAISICKMKCGKTGWTQPQLTCVDEREHHRFLASEESQGSRNSSPE | | | |
| | | SETSCPITTTDFPQPTETTAMTETFVLTMEYKGCKPCICTVPEVSSVFIFPPKP | | | |
| | | KDVLTITLTPKVTCVVVDISKDDPEVQFSWFVDDVEVHTAQTQPREEQFNSTFR | | | |
| | | SVSELPIMHQDWLNGKEFKCRVNSAAFPAPIEKTISKTKGRPKAPQVYTIPPPK | | | |
| | | EQMAKDKVSLTCMITDFFPEDITVEWQWNGQPAENYKNTQPIMDTDGSYFVYSK | | | |
| | | LNVQKSNWEAGNTFTCSVLHEGLHNHHTEKSLSHSPGKGHHPH | | | |
| 837 | Construct I | APTSSSTKKTQLQLEHLLLDLQMILNGINNYKNPKLTRMLTFKFYMPKKATELK | | fusion | |
| | | HLQCLEEELKPLEEVLNLAQSKNFHLRPRDLISNINVIVLELKGSETTFMCEYA | | protein | |
| | | DETATIVEFLNRWITFSQSIISTLTSGGGGSGGGGGPLGVRGGGGGSGGGGSEL | | | |
| | | CDDDPPEIPHATFKAMAYKEGTILNCECKRGFRRIKSGSLYMLCTGNSSHSSWD | | | |
| | | NQCQCTSSATRNTTKQVTPQPEEQKERKTTEMQSPMQPVDQASLPGHCREPPPW | | | |
| | | ENEATERIYHFVVGQMVYYQCVQGYRALHRGPAESVCKMTHGKTRWTQPQLICT | | | |
| | | GEMETSQFPGEEKPQASPEGRPESETSCLVTTTDFQIQTEMAATMETSIFTTEY | | | |
| | | QDKTHTCPPCPAPELLGGPSVFLFPPKPKDTLMISRTPEVTCVVVDVSHEDPEV | | | |
| | | KFNWYVDGVEVHNAKTKPREEQYNSTYRVVSVLTVLHQDWLNGKEYKCKVSNKA | | | |
| | | LPAPIEKTISKAKGQPREPQVYTLPPSRDELTKNQVSLTCLVKGFYPSDIAVEW | | | |
| | | ESNGQPENNYKTTPPVLDSDGSFFLYSKLTVDKSRWQQGNVFSCSVMHEALHNH | | | |
| | | YTQKSLSLSPGHHHHHH | | | |
| 838 | Construct J | APTSSSTKKTQLQLEHLLLDLQMILNGINNYKNPKLTRMLTFKFYMPKKATELK | | fusion | |
| | | HLQCLEEELKPLEEVLNLAQSKNFHLRPRDLISNINVIVLELKGSETTFMCEYA | | protein | |
| | | DETATIVEFLNRWITFSQSIISTLTSGGGGSGGGGGPLGVRGGGGGSGGGGSEL | | | |
| | | CDDDPPEIPHATFKAMAYKEGTMLNCECKRGFRRIKSGSVYMLCTGNSSHSSWD | | | |
| | | NQCQCTSSATRNTTKQVTPQPEEQKERKTTEMQSPMQPVDQASLPGHCREPPPW | | | |
| | | ENEATERIYHFVVGQMVYYQCVQGYRALHRGPAESVCKMTHGKTRWTQPQLICT | | | |
| | | GEMETSQFPGEEKPQASPEGRPESETSCLVTTTDFQIQTEMAATMETSIFTTEY | | | |
| | | QDKTHTCPPCPAPELLGGPSVFLFPPKPKDTLMISRTPEVTCVVVDVSHEDPEV | | | |
| | | KFNWYVDGVEVHNAKTKPREEQYNSTYRVVSVLTVLHQDWLNGKEYKCKVSNKA | | | |
| | | LPAPIEKTISKAKGQPREPQVYTLPPSRDELTKNQVSLTCLVKGFYPSDIAVEW | | | |
| | | ESNGQPENNYKTTPPVLDSDGSFFLYSKLTVDKSRWQQGNVFSCSVMHEALHNH | | | |
| | | YTQKSLSLSPGHHHHHH | | | |
| 839 | Construct K | APTSSSTKKTQLQLEHLLLDLQMILNGINNYKNPKLTRMLTFKFYMPKKATELK | | fusion | |
| | | HLQCLEEELKPLEEVLNLAQSKNFHLRPRDLISNINVIVLELKGSETTFMCEYA | | protein | |
| | | DETATIVEFLNRWITFSQSIISTLTSGGGGSGGGGGPLGVRGGGGGSGGGGSEL | | | |
| | | CLYDPPEIPHATFKAMAYKEGTMLNCECKRGFRRIKSGSLYMLCTGNSSHSSWD | | | |
| | | NQCQCTSSATRNTTKQVTPQPEEQKERKTTEMQSPMQPVDQASLPGHCREPPPW | | | |
| | | ENEATERIYHFVVGQMVYYQCVQGYRALHRGPAESVCKMTHGKTRWTQPQLICT | | | |
| | | GEMETSQFPGEEKPQASPEGRPESETSCLVTTTDFQIQTEMAATMETSIFTTEY | | | |
| | | QDKTHTCPPCPAPELLGGPSVFLFPPKPKDTLMISRTPEVTCVVVDVSHEDPEV | | | |
| | | KFNWYVDGVEVHNAKTKPREEQYNSTYRVVSVLTVLHQDWLNGKEYKCKVSNKA | | | |
| | | LPAPIEKTISKAKGQPREPQVYTLPPSRDELTKNQVSLTCLVKGFYPSDIAVEW | | | |
| | | ESNGQPENNYKTTPPVLDSDGSFFLYSKLTVDKSRWQQGNVFSCSVMHEALHNH | | | |
| | | YTQKSLSLSPGHHHHHH | | | |
| 840 | Construct L | APTSSSTKKTQLQLEHLLLDLQMILNGINNYKNPKLTRMLTFKFYMPKKATELK | | fusion | |
| | | HLQCLEEELKPLEEVLNLAQSKNFHLRPRDLISNINVIVLELKGSETTFMCEYA | | protein | |
| | | DETATIVEFLNRWITFSQSIISTLTSGGGGSGGGGGPLGVRGGGGGSGGGGSEL | | | |
| | | CDDDPPEIPHATFKAMAYKEGTMLNCECKRGFRRIKELVYMLCTGNSSHSSWDN | | | |
| | | QCQCTSSATRNTTKQVTPQPEEQKERKTTEMQSPMQPVDQASLPGHCREPPPWE | | | |
| | | NEATERIYHFVVGQMVYYQCVQGYRALHRGPAESVCKMTHGKTRWTQPQLICTG | | | |
| | | EMETSQFPGEEKPQASPEGRPESETSCLVTTTDFQIQTEMAATMETSIFTTEYQ | | | |
| | | DKTHTCPPCPAPELLGGPSVFLFPPKPKDTLMISRTPEVTCVVVDVSHEDPEVK | | | |
| | | FNWYVDGVEVHNAKTKPREEQYNSTYRVVSVLTVLHQDWLNGKEYKCKVSNKAL | | | |
| | | PAPIEKTISKAKGQPREPQVYTLPPSRDELTKNQVSLTCLVKGFYPSDIAVEWE | | | |
| | | SNGQPENNYKTTPPVLDSDGSFFLYSKLTVDKSRWQQGNVFSCSVMHEALHNHY | | | |
| | | TQKSLSLSPGHHHHHH | | | |
| 841 | Construct M | APTSSSTKKTQLQLEHLLLDLQMILNGINNYKNPKLTRMLTFKFYMPKKATELK | | fusion | |
| | | HLQCLEEELKPLEEVLNLAQSKNFHLRPRDLISNINVIVLELKGSETTFMCEYA | | protein | |
| | | DETATIVEFLNRWITFSQSIISTLTSGGGGSGGGGGPLGVRGGGGGSGGGGSEL | | | |
| | | CDDDPPEIPHATFKAMAYKEGTMLNCECKRGFRRIKSGSLYMLCTGNSSHSSWD | | | |
| | | NQCQCTSSATRNTTKQVTPQPEEQKERKTTEMQSPMQPVDQASLPGHCREPPPW | | | |
| | | ENEATERIYHFVVGQMVYYQCVQGYRALHRGPAESVCKMTHGKTRWTQPQLICT | | | |
| | | GEMETSQFPGEEKPQASPEGRPESETSCDKTHTCPPCPAPELLGGPSVFLFPPK | | | |
| | | PKDTLMISRTPEVTCVVVDVSHEDPEVKFNWYVDGVEVHNAKTKPREEQYNSTY | | | |
| | | RVVSVLTVLHQDWLNGKEYKCKVSNKALPAPIEKTISKAKGQPREPQVYTLPPS | | | |
| | | RDELTKNQVSLTCLVKGFYPSDIAVEWESNGQPENNYKTTPPVLDSDGSFFLYS | | | |
| | | KLTVDKSRWQQGNVFSCSVMHEALHNHYTQKSLSLSPG | | | |
| 842 | Construct N | APTSSSTKKTQLQLEHLLLDLQMILNGINNYKNPKLTRMLTFKFYMPKKATELK | | fusion | |
| | | HLQCLEEELKPLEEVLNLAQSKNFHLRPRDLISNINVIVLELKGSETTFMCEYA | | protein | |
| | | DETATIVEFLNRWITFSQSIISTLTSGGGGSGGGGGPLGVRGGGGGSGGGGSEL | | | |
| | | CDDDPPEIPHATFKAMAYKEGTMLNCECKRGFRRIKSGSLYMLCTGNSSHSSWD | | | |
| | | NQCQCTSSATRNTTKQVTPQPEEQKERKTTEMQSPMQPVDQASLPGHCREPPPW | | | |
| | | ENEATERIYHFVVGQMVYYQCVQGYRALHRGPAESVCKMTHGKTRWTQPQLICT | | | |
| | | GEMETSQFPGEEKPQASPEGRPESETSCGSGGGGDKTHTCPPCPAPELLGGPSV | | | |
| | | FLFPPKPKDTLMISRTPEVTCVVVDVSHEDPEVKFNWYVDGVEVHNAKTKPREE | | | |
| | | QYNSTYRVVSVLTVLHQDWLNGKEYKCKVSNKALPAPIEKTISKAKGQPREPQV | | | |
| | | YTLPPSRDELTKNQVSLTCLVKGFYPSDIAVEWESNGQPENNYKTTPPVLDSDG | | | |
| | | SFFLYSKLTVDKSRWQQGNVFSCSVMHEALHNHYTQKSLSLSPG | | | |
| 843 | Construct O | APTSSSTKKTQLQLEHLLLDLQMILNGINNYKNPKLTRMLTFKFYMPKKATELK | | fusion | |
| | | HLQCLEEELKPLEEVLNLAQSKNFHLRPRDLISNINVIVLELKGSETTFMCEYA | | protein | |
| | | DETATIVEFLNRWITFSQSIISTLTSGGGGSGGGGGPLGVRGGGGGSGGGGSEL | | | |
| | | CDDDPPEIPHATFKAMAYKEGTILNCECKRGFRRIKSGSLYMLCTGNSSHSSWD | | | |
| | | NQCQCTSSATRNTTKQVTPQPEEQKERKTTEMQSPMQPVDQASLPGHCREPPPW | | | |
| | | ENEATERIYHFVVGQMVYYQCVQGYRALHRGPAESVCKMTHGKTRWTQPQLICT | | | |
| | | GEMETSQFPGEEKPQASPEGRPESETSCGSGGGGDKTHTCPPCPAPELLGGPSV | | | |
| | | FLFPPKPKDTLMISRTPEVTCVVVDVSHEDPEVKFNWYVDGVEVHNAKTKPREE | | | |
| | | QYNSTYRVVSVLTVLHQDWLNGKEYKCKVSNKALPAPIEKTISKAKGQPREPQV | | | |
| | | YTLPPSRDELTKNQVSLTCLVKGFYPSDIAVEWESNGQPENNYKTTPPVLDSDG | | | |
| | | SFFLYSKLTVDKSRWQQGNVFSCSVMHEALHNHYTQKSLSLSPG | | | |
| 844 | Construct P | APTSSSTKKTQLQLEHLLLDLQMILNGINNYKNPKLTRMLTFKFYMPKKATELK | | fusion | |
| | | HLQCLEEELKPLEEVLNLAQSKNFHLRPRDLISNINVIVLELKGSETTFMCEYA | | protein | |
| | | DETATIVEFLNRWITFSQSIISTLTSGGGGSGGGGGPLGVRGGGGGSGGGGSEL | | | |
| | | CDDDPPEIPHATFKAMAYKEGTMLNCECKRGFRRIKSGSVYMLCTGNSSHSSWD | | | |
| | | NQCQCTSSATRNTTKQVTPQPEEQKERKTTEMQSPMQPVDQASLPGHCREPPPW | | | |
| | | ENEATERIYHFVVGQMVYYQCVQGYRALHRGPAESVCKMTHGKTRWTQPQLICT | | | |
| | | GEMETSQFPGEEKPQASPEGRPESETSCGSGGGGDKTHTCPPCPAPELLGGPSV | | | |
| | | FLFPPKPKDTLMISRTPEVTCVVVDVSHEDPEVKFNWYVDGVEVHNAKTKPREE | | | |
| | | QYNSTYRVVSVLTVLHQDWLNGKEYKCKVSNKALPAPIEKTISKAKGQPREPQV | | | |
| | | YTLPPSRDELTKNQVSLTCLVKGFYPSDIAVEWESNGQPENNYKTTPPVLDSDG | | | |
| | | SFFLYSKLTVDKSRWQQGNVFSCSVMHEALHNHYTQKSLSLSPG | | | |
| 845 | Construct Q | APTSSSTKKTQLQLEHLLLDLQMILNGINNYKNPKLTRMLTFKFYMPKKATELK | | fusion | |
| | | HLQCLEEELKPLEEVLNLAQSKNFHLRPRDLISNINVIVLELKGSETTFMCEYA | | protein | |
| | | DETATIVEFLNRWITFSQSIISTLTSGGGGSGGGGGPLGVRGGGGGSGGGGSEL | | | |
| | | CLYDPPEIPHATFKAMAYKEGTMLNCECKRGFRRIKSGSLYMLCTGNSSHSSWD | | | |
| | | NQCQCTSSATRNTTKQVTPQPEEQKERKTTEMQSPMQPVDQASLPGHCREPPPW | | | |
| | | ENEATERIYHFVVGQMVYYQCVQGYRALHRGPAESVCKMTHGKTRWTQPQLICT | | | |
| | | GEMETSQFPGEEKPQASPEGRPESETSCGSGGGGDKTHTCPPCPAPELLGGPSV | | | |
| | | FLFPPKPKDTLMISRTPEVTCVVVDVSHEDPEVKFNWYVDGVEVHNAKTKPREE | | | |
| | | QYNSTYRVVSVLTVLHQDWLNGKEYKCKVSNKALPAPIEKTISKAKGQPREPQV | | | |
| | | YTLPPSRDELTKNQVSLTCLVKGFYPSDIAVEWESNGQPENNYKTTPPVLDSDG | | | |
| | | SFFLYSKLTVDKSRWQQGNVFSCSVMHEALHNHYTQKSLSLSPG | | | |
| 846 | Construct | APTSSSTKKTQLQLEHLLLDLQMILNGINNY | | fusion | |
| | AAA | KNPKLTRMLTFKFYMPKKATELKHLQCLEEELKPLEEVLNLAQSKNFHLR | | protein | |
| | | PRDLISNINVIVLELKGSETTFMCEYADETATIVEFLNRWITFSQSIIST | | | |
| | | LTSGGGGSGGGGGPLGVRGGGGGSGGGGSELCDDDPPEIPHATFKAMAYK | | | |
| | | EGTILNCECKRGFRRIKSGSLYMLCTGNSSHSSWDNQCQCTSSATRNTTK | | | |
| | | QVTPQPEEQKERKTTEMQSPMQPVDQASLPGHCREPPPWENEATERIYHF | | | |
| | | VVGQMVYYQCVQGYRALHRGPAESVCKMTHGKTRWTQPQLICTGEMETSQ | | | |
| | | FPGEEKPQASPEGRPESETSCLVTTTDFQIQTEMAATMETSIFTTEYQGS | | | |
| | | GGGGDKTHTCPPCPAPEAAGGPSVFLFPPKPKDTLMISRTPEVTCVVVDV | | | |
| | | SHEDPEVKFNWYVDGVEVHNAKTKPREEQYNSTYRVVSVLTVLHQDWLNG | | | |
| | | KEYKCKVSNKALPAPIEKTISKAKGQPREPQVYTLPPSRDELTKNQVSLT | | | |
| | | CLVKGFYPSDIAVEWESNGQPENNYKTTPPVLDSDGSFFLYSKLTVDKSR | | | |
| | | WQQGNVFSCSVMHEALHNHYTQKSLSLSPG | | | |
| 847 | Construct | APTSSSTKKTQLQLEHLLLDLQMILNGINNY | | fusion | |
| | BBB | KNPKLTRMLTFKFYMPKKATELKHLQCLEEELKPLEEVLNLAQSKNFHLR | | protein | |
| | | PRDLISNINVIVLELKGSETTFMCEYADETATIVEFLNRWITFSQSIIST | | | |
| | | LTSGGGGSGGGGGVRLGPGGGGGSGGGGSELCDDDPPEIPHATFKAMAYK | | | |
| | | EGTILNCECKRGFRRIKSGSLYMLCTGNSSHSSWDNQCQCTSSATRNTTK | | | |
| | | QVTPQPEEQKERKTTEMQSPMQPVDQASLPGHCREPPPWENEATERIYHF | | | |
| | | VVGQMVYYQCVQGYRALHRGPAESVCKMTHGKTRWTQPQLICTGEMETSQ | | | |
| | | FPGEEKPQASPEGRPESETSCLVTTTDFQIQTEMAATMETSIFTTEYQGS | | | |
| | | GGGGDKTHTCPPCPAPEAAGGPSVFLFPPKPKDTLMISRTPEVTCVVVDV | | | |
| | | SHEDPEVKFNWYVDGVEVHNAKTKPREEQYNSTYRVVSVLTVLHQDWLNG | | | |
| | | KEYKCKVSNKALPAPIEKTISKAKGQPREPQVYTLPPSRDELTKNQVSLT | | | |
| | | CLVKGFYPSDIAVEWESNGQPENNYKTTPPVLDSDGSFFLYSKLTVDKSR | | | |
| | | WQQGNVFSCSVMHEALHNHYTQKSLSLSPG | | | |
| 848 | Construct | APTSSSTKKTQLQLEHLLLDLQMILNGINNY | | fusion | |
| | CCC | KNPKLTRMLTFKFYMPKKATELKHLQCLEEELKPLEEVLNLAQSKNFHLR | | protein | |
| | | PRDLISNINVIVLELKGSETTFMCEYADETATIVEFLNRWITFSQSIIST | | | |
| | | LTGHHPHGHHPHGVRLGPGGGGGSGGGGSELCDDDPPEIPHATFKAMAYK | | | |
| | | EGTILNCECKRGFRRIKSGSLYMLCTGNSSHSSWDNQCQCTSSATRNTTK | | | |
| | | QVTPQPEEQKERKTTEMQSPMQPVDQASLPGHCREPPPWENEATERIYHF | | | |
| | | VVGQMVYYQCVQGYRALHRGPAESVCKMTHGKTRWTQPQLICTGEMETSQ | | | |
| | | FPGEEKPQASPEGRPESETSCLVTTTDFQIQTEMAATMETSIFTTEYQGS | | | |
| | | GGGGDKTHTCPPCPAPEAAGGPSVFLFPPKPKDTLMISRTPEVTCVVVDV | | | |
| | | SHEDPEVKFNWYVDGVEVHNAKTKPREEQYNSTYRVVSVLTVLHQDWLNG | | | |
| | | KEYKCKVSNKALPAPIEKTISKAKGQPREPQVYTLPPSRDELTKNQVSLT | | | |
| | | CLVKGFYPSDIAVEWESNGQPENNYKTTPPVLDSDGSFFLYSKLTVDKSR | | | |
| | | WQQGNVFSCSVMHEALHNHYTQKSLSLSPG | | | |
| 849 | Construct | APTSSSTKKTQLQLEHLLLDLQMILNGINNY | | fusion | |
| | DDD | KNPKLTRMLTFKFYMPKKATELKHLQCLEEELKPLEEVLNLAQSKNFHLR | | protein | |
| | | PRDLISNINVIVLELKGSETTFMCEYADETATIVEFLNRWITFSQSIIST | | | |
| | | LTGHHPHGHHPHGPLGVRGGGGGSGGGGSELCDDDPPEIPHATFKAMAYK | | | |
| | | EGTILNCECKRGFRRIKSGSLYMLCTGNSSHSSWDNQCQCTSSATRNTTK | | | |
| | | QVTPQPEEQKERKTTEMQSPMQPVDQASLPGHCREPPPWENEATERIYHF | | | |
| | | VVGQMVYYQCVQGYRALHRGPAESVCKMTHGKTRWTQPQLICTGEMETSQ | | | |
| | | FPGEEKPQASPEGRPESETSCLVTTTDFQIQTEMAATMETSIFTTEYQGS | | | |
| | | GGGGDKTHTCPPCPAPEAAGGPSVFLFPPKPKDTLMISRTPEVTCVVVDV | | | |
| | | SHEDPEVKFNWYVDGVEVHNAKTKPREEQYNSTYRVVSVLTVLHQDWLNG | | | |
| | | KEYKCKVSNKALPAPIEKTISKAKGQPREPQVYTLPPSRDELTKNQVSLT | | | |
| | | CLVKGFYPSDIAVEWESNGQPENNYKTTPPVLDSDGSFFLYSKLTVDKSR | | | |
| | | WQQGNVFSCSVMHEALHNHYTQKSLSLSPG | | | |
| 850 | Construct EEE | APTSSSTKKTQLQLEHLLLDLQMILNGINNY | | fusion | |
| | | KNPKLTRMLTFKFYMPKKATELKHLQCLEEELKPLEEVLNLAQSKNFHLR | | protein | |
| | | PRDLISNINVIVLELKGSETTFMCEYADETATIVEFLNRWITFSQSIIST | | | |
| | | LTVRIQRKKEKMKETGPLGVRGGGGGSGGGGSELCDDDPPEIPHATFKAMAYK | | | |
| | | EGTILNCECKRGFRRIKSGSLYMLCTGNSSHSSWDNQCQCTSSATRNTTK | | | |
| | | QVTPQPEEQKERKTTEMQSPMQPVDQASLPGHCREPPPWENEATERIYHF | | | |
| | | VVGQMVYYQCVQGYRALHRGPAESVCKMTHGKTRWTQPQLICTGEMETSQ | | | |
| | | FPGEEKPQASPEGRPESETSCLVTTTDFQIQTEMAATMETSIFTTEYQGS | | | |
| | | GGGGDKTHTCPPCPAPEAAGGPSVFLFPPKPKDTLMISRTPEVTCVVVDV | | | |
| | | SHEDPEVKFNWYVDGVEVHNAKTKPREEQYNSTYRVVSVLTVLHQDWLNG | | | |
| | | KEYKCKVSNKALPAPIEKTISKAKGQPREPQVYTLPPSRDELTKNQVSLT | | | |
| | | CLVKGFYPSDIAVEWESNGQPENNYKTTPPVLDSDGSFFLYSKLTVDKSR | | | |
| | | WQQGNVFSCSVMHEALHNHYTQKSLSLSPG | | | |
| 851 | Construct FFF | APTSSSTKKTQLQLEHLLLDLQMILNGINNY | | fusion | |
| | | KNPKLTRMLTFKFYMPKKATELKHLQCLEEELKPLEEVLNLAQSKNFHLR | | protein | |
| | | PRDLISNINVIVLELKGSETTFMCEYADETATIVEFLNRWITFSQSIIST | | | |
| | | LTVRIQRKKEKMKETGPLGVRGGGGGSGGGGSELCDDDPPEIPHATFKAMAYK | | | |
| | | EGTILNCECKRGFRRIKSGSLYMLCTGNSSHSSWDNQCQCTSSATRNTTK | | | |
| | | QVTPQPEEQKERKTTEMQSPMQPVDQASLPGHCREPPPWENEATERIYHF | | | |
| | | VVGQMVYYQCVQGYRALHRGPAESVCKMTHGKTRWTQPQLICTGEMETSQ | | | |
| | | FPGEEKPQASPEGRPESETSCLVTTTDFQIQTEMAATMETSIFTTEYQGS | | | |
| | | GGGGDKTHTCPPCPAPEAAGGPSVFLFPPKPKDTLMISRTPEVTCVVVDV | | | |
| | | SHEDPEVKFNWYVDGVEVHNAKTKPREEQYNSTYRVVSVLTVLHQDWLNG | | | |
| | | KEYKCKVSNKALPAPIEKTISKAKGQPREPQVYTLPPSRDELTKNQVSLT | | | |
| | | CLVKGFYPSDIAVEWESNGQPENNYKTTPPVLDSDGSFFLYSKLTVDKSR | | | |
| | | WQQGNVFSCSVMHEALHNHYTQKSLSLSPG | | | |
| 852 | Construct | APTSSSTSSSTAEAQQQQQQQQQQQQHLEQLLMDLQELLSRMENYRNLKL | | fusion | |
| | GGG | PRMLTFKFYLPKQATELKDLQCLEDELGPLRHVLDLTQSKSFQLEDAENF | | protein | |
| | | ISNIRVTVVKLKGSDNTFECQFDDESATVVDFLRRWIAFSQSIISTSPQS | | | |
| | | GGGGSGGGGGVRLGPGGGGGSGGGGSELCLYDPPEVPNATFKALSYKNGT | | | |
| | | ILNCECKRGFRRLKELVYMRCLGNSWSSNCQCTSNSHDKSRKQVTAQLEH | | | |
| | | QKEQQTTTDMQKPTQSMHQENLTGHCREPPPWKHEDSKRIYHFVEGQSVH | | | |
| | | YECIPGYKALQRGPAISICKMKCGKTGWTQPQLTCVDEREHHRFLASEES | | | |
| | | QGSRNSSPESETSCPITTTDFPQPTETTAMTETFVLTMEYKGCKPCICTV | | | |
| | | PEVSSVFIFPPKPKDVLTITLTPKVTCVVVDISKDDPEVQFSWFVDDVEV | | | |
| | | HTAQTQPREEQFNSTFRSVSELPIMHQDWLNGKEFKCRVNSAAFPAPIEK | | | |
| | | TISKTKGRPKAPQVYTIPPPKEQMAKDKVSLTCMITDFFPEDITVEWQWN | | | |
| | | GQPAENYKNTQPIMDTDGSYFVYSKLNVQKSNWEAGNTFTCSVLHEGLHN | | | |
| | | HHTEKSLSHSPGK | | | |

TABLE 2
Table of Targeting Sequences

| SEQ ID | | | | |
|---|---|---|---|---|
| NO | Sequence | Binds to | Note 1 | Note 2 |
| 180 | (TLTYTWS)n | denatured collagen IV | binding to MMP degraded collagen | |
| 181 | (CREKA)n | denatured collagen IV | binding to MMP degraded | inhibit tumor |
| | | | collagen | vasculature formation |
| 182 | (GXY)n | denatured Collagen | Gly = Glycine/X = | This peptide binds to |
| | | | Proline or modified | collagen |
| | | | Proline/Y = Proline | preteolytically |
| | | | or modified Proline | digested by MMP |
| 183 | GHCVTDSGVVYSVGMQ | denatured Collagen | from Fibronectin Domain | |
| | WLKTQGNKQMLCTCLG | | 1-6 | |
| | NGVSCQET | | | |
| 184 | EICTTNEGVMYRIGDQW | denatured Collagen | from Fibronectin Domain | |
| | DKQHDMGHMMRCTCV | | 1-7 | |
| | GNGRGEWTCIAY | | | |
| 185 | DQCIVDDITYNVNDTFH | denatured Collagen | from Fibronectin Domain | |
| | KRHEEGHMLNCTCFGQ | | 1-8 | |
| | GRGRWKCDPV | | | |
| 186 | DQCQDSETGTFYQIGDS | denatured Collagen | from Fibronectin Domain | |
| | WEKYVHGVRYQCYCYG | | 1-9 | |
| | RGIGEWHCQPL | | | |
| 187 | SNGEPCVLPFTYNGRTF | denatured Collagen | from Fibronectin Domain | |
| | YSCTTEGRQDGHLWCST | | 2-1 | |
| | TSNYEQDQKYSFCTD | | | |
| 188 | SNGALCHFPFLYNNHNY | denatured Collagen | from Fibronectin Domain | |
| | TDCTSEGRRDNMKWCG | | 2-2 | |
| | TTQNYDADQKFGFCPM | | | |
| 189 | RRANAALKAGELYKSIL | Collagen type I | Kd 0.86 uM//860 nM | Differential binding |
| | YGC | | | affinity to Collagen |
| 190 | RRANAALKAGELYKCIL | Collagen type I | Kd: 10 nM (tight | Differential binding |
| | YGC | | binding) | affinity to Collagen |
| 191 | MIVIELGTNPLKSSGIEN | Collagen type I | Kd 0.394 uM//394 nM | Differential binding |
| | GAFQGMKK | | | affinity to Collagen |
| 192 | LRELHLNNN | Collagen type I | Kd 0.17 uM//170 nM | Differential binding |
| | | | | affinity to Collagen |
| 193 | WREPSFCALS | Collagen type I | Kd 100 uM//100,000 nM | Differential binding |
| | | | | affinity to Collagen |
| 194 | TKKTLRT | Collagen type I | Kd ≤ 100 uM | Differential binding |
| | | | | affinity to Collagen |
| 195 | CPKESCNLFVLKD | Collagen type I | Kd 0.681 uM//681 nM | Differential binding |
| | | | | affinity to Collagen |
| 196 | WREPSFCALS | Collagen type I | Kd: 100 uM//100,000 nM | Differential binding |
| | | | | affinity to Collagen |
| 197 | HVWMQAPGGGK | Collagen type I | Kd 61 uM//61,000 nM | H-V-F/W-Q/M-Q-P/A-P/K |
| | | | | motif |
| 198 | HVWMQAPGGGC | Collagen type I | | |
| 199 | WYRGRL | Collagen type II | | |
| 200 | KLWVLPK | Collagen type IV | | |
| 201 | RRANAALKAGELYKSIL | Collagen | | |
| | Y | | | |
| 202 | GELYKSILY | Collagen | | |
| 203 | RRANAALKAGELYKCIL | Collagen | | |
| | Y | | | |
| 204 | GELYKCILY | Collagen | | |
| 205 | RLDGNEIKR | Collagen | | |
| 206 | AHEEISTTNEGVM | Collagen | | |
| 207 | NGVFKYRPRYFLYKHAY | Collagen | | |
| | FYPPLKRFPVQ | | | |
| 208 | CQDSETRTFY | Collagen | | |
| 209 | TKKTLRT | Collagen | | |
| 210 | GLRSKSKKFRRPDIQYPD | Collagen | | |
| | ATDEDITSHM | | | |
| 211 | SQNPVQP | Collagen | | |
| 212 | SYIRIADTNIT | Collagen | | |
| 213 | KELNLVYT | Collagen | | |
| 214 | GSIT | Collagen | | |
| 215 | GSITTIDVPWNV | Collagen | | |
| 216 | GQLYKSILY | Collagen | | |
| 217 | RRANAALKAGQLYKSIL | Collagen | | |
| | Y | | | |
| 218 | WREPSFCALS | Collagen | | |
| 219 | WHCTTKFPHHYCLY | Collagen | | |
| 220 | AHKCPWHLYTTHYCFT | Collagen | | |
| 221 | PAHKCPWHLYTHYCFT | Collagen | | |
| 222 | GROGER | Collagen | O is 4-hydroxyproline (see, Raynal, N., et | |
| | | | al., J. Biol. Chem., 2006, 281(7), 3821- | |
| | | | 3831) | |
| 223 | GMOGER | Collagen | O is 4-hydroxyproline (see, Raynal, N., et | |
| | | | al., J. Biol. Chem., 2006, 281(7), 3821- | |
| | | | 3831) | |
| 224 | GLOGEN | Collagen | O is 4-hydroxyproline (see, Raynal, N., et | |
| | | | al., J. Biol. Chem., 2006, 281(7), 3821- | |
| | | | 3831) | |
| 225 | GLOGER | Collagen | O is 4-hydroxyproline (see, Raynal, N., et | |
| | | | al., J. Biol. Chem., 2006, 281(7), 3821- | |
| | | | 3831) | |
| 226 | GLKGEN | Collagen | O is 4-hydroxyproline (see, Raynal, N., et | |
| | | | al., J. Biol. Chem., 2006, 281(7), 3821- | |
| | | | 3831) | |
| 227 | GFOGERGVEGPOGPA | Collagen | O is 4-hydroxyproline (see, Raynal, N., et | |
| | | | al., J. Biol. Chem., 2006, 281(7), 3821- | |
| | | | 3831) | |
| 228 | WREPSFCALS | Collagen | | Takagi, J., et al, |
| | | | | Biochemistry, 1992, |
| | | | | 31, 8530-8534 |
| 229 | WYRGRL | Collagen | | Rothenfluh D.A., et |
| | | | | al, Nat Mater. 2008, |
| | | | | 7(3), 748-54 |
| 230 | WTCSGDEYTWHC | Collagen | | |
| 231 | WTCVGDHKTWKC | Collagen | | |
| 232 | QWHCTTRFPHHYCLYG | Collagen | | U.S. 2007/0293656) |
| 233 | STWTWNGSAWTWNEG | Collagen | | |
| | GK | | | |
| 234 | STWTWNGTNWTRNDGG | Collagen | | WO/2014/059530 |
| | K | | | |
| 235 | CVWLWEQC | Collagen | | |
| 236 | CMTSPWRC | Collagen | | Vanhoorelbeke, K., et |
| | | | | al, J. Biol. Chem., |
| | | | | 2003, 278, 37815- |
| | | | | 37821 |
| 237 | CPGRVMHGLHLGDDEG | Collagen | | Muzzard, J., et al, |
| | PC | | | PLoS one. 4(e5585) |
| | | | | I- 10) |
| 238 | KLWLLPK | Collagen | | Chan, J. M., et al, |
| | | | | Proc Natl Acad Sci |
| | | | | U.S.A., 2010, 107, |
| | | | | 2213-2218) |
| 239 | CQDSETRTFY | Collagen | | U.S. 2013/0243700 |
| 240 | LSELRLHEN | Collagen | | Fredrico, S., Angew. |
| | | | | Chem. Int. Ed. 2015, |
| | | | | 37, 10980-10984 |
| 241 | LTELHLDNN | Collagen | | Fredrico, S., Angew. |
| | | | | Chem. Int. Ed. 2015, |
| | | | | 37, 10980-10985 |
| 242 | LSELRLHNN | Collagen | | Fredrico, S., Angew. |
| | | | | Chem. Int. Ed. 2015, |
| | | | | 37, 10980-10986 |
| 243 | LSELRLHAN | Collagen | | Fredrico, S., Angew. |
| | | | | Chem. Int. Ed. 2015, |
| | | | | 37, 10980-10987 |
| 244 | LRELHLNNN | Collagen | | Fredrico, S., Angew. |
| | | | | Chem. Int. Ed. 2015, |
| | | | | 37, 10980-10988 |
| 245 | RVMHGLHLGDDE | Collagen | | |
| 246 | RVMHGLHLGNNQ | Collagen | | |
| 747 | RVMHGLHLGNNQ | Collagen | | |
| 748 | GQLYKSILYGSG-4K2K | Collagen | (a 4-branch peptide) which can be conjugated | |
| | | | to a fusion polypeptide | |
| 749 | GSGQLYKSILY | Collagen | | |
| 250 | GSGGQLYKSILY | Collagen | | |
| 251 | KQLNLVYT | Collagen | | |
| 252 | CVWLWQQC | Collagen | | |
| 253 | WREPSFSALS | Collagen | | |
| 254 | GHRPLDKKREEAPSLRP | Collagen | | |
| | APPPISGGGYR | | | |
| 255 | GHRPLNKKRQQ | Collagen | | |
| | APSLRPAPPPISGGGYR | | | |
| 256 | GELYKSILYGSG | Collagen | | |
| 257 | GQLYKSILYGSG | Collagen | | |
| 258 | RYPISRPRKRGSG | Collagen | | |
| 259 | GELYKSILYGC | Collagen | | |
| 260 | RLDGNEIKRGC | Collagen | | |
| 261 | AHEEISTTNEGVMGC | Collagen | | |
| 262 | GCGGELYKSILY | Collagen | | |
| 263 | NGVFKYRPRYFLYKHAY | Collagen | | |
| | FYPPLKRFPVQGC | | | |
| 264 | CQDSETRTFYGC | Collagen | | |
| 265 | TKKTLRTGC | Collagen | | |
| 266 | GLRSKSKKFRRPDIQYPD | Collagen | | |
| | ATDEDITSHMGC | | | |
| 267 | SQNPVQPGC | Collagen | | |
| 268 | SYIRIADTNITGC | Collagen | | |
| 269 | KELNLVYTGC | Collagen | | |
| 270 | GSITTIDVPWNVGC | Collagen | | |
| 271 | GCGGELYKSILYGC | Collagen | | |
| 272 | RRANAALKAGELYKSIL | Collagen | | |
| | YGSG | | | |
| 273 | cyclic CVWLWENC | Collagen | cyclic peptides can be conjugated to a fusion | |
| | | | polypeptide | |
| 274 | cyclic CVWLWEQC | Collagen | cyclic peptides can be | Depraetere H., et al, |
| | | | conjugated to a fusion | Blood. 1998, 92, 4207- |
| | | | polypeptide | 421 1; and Duncan R., |
| | | | | Nat Rev Drug Discov, |
| | | | | 2003, 2(5), 347-360 |
| 275 | D-amino acid | Collagen | D-amino acid-containing peptides can be conjugated | |
| | EDDGLHLGHMVR | | to ODC | |
| 276 | D-amino acid | Collagen | D-amino acid-containing peptides can be conjugated | |
| | QNNGLHLGHMVR | | to ODC | |
| 277 | PPTDLRFTNIGPDTMRVT | integrin | from Fibronectin Domain III-9 | |
| | WAPPPSIDLTNFLVRYSP | | | |
| | VKNEEDVAELSISPSDNA | | | |
| | VVLTNLLPGTEYVVSVS | | | |
| | SVYEQHESTPLRGRQKT | | | |
| | GLDSP | | | |
| 278 | TGIDFSDITANSFTVHWI | integrin | from Fibronectin Domain III-10 | |
| | APRATITGYRIRHHPEHF | | | |
| | SGRPREDRVPHSRNSITL | | | |
| | TNLTPGTEYVVSIVALN | | | |
| | GREESPLLIGQQSTVSD | | | |
| 279 | PGCYDNGKHYQINQQW | integrin | from Fibronectin Domain | |
| | ERTYLGNALVCTCYGGS | | 1-1 | |
| | RGFNCESK | | | |
| 280 | ETCFDKYTGNTYRVGDT | integrin | from Fibronectin Domain | |
| | YERPKDSMIWDCTCIGA | | 1-2 | |
| | GRGRISCTIA | | | |
| 281 | NRCHEGGQSYKIGDTWR | integrin | from Fibronectin Domain | |
| | RPHETGGYMLECVCLGN | | 1-3 | |
| | GKGEWTCKPI | | | |
| 282 | EKCFDHAAGTSYVVGET | integrin | from Fibronectin Domain | |
| | WEKPYQGWMMVDCTC | | 1-4 | |
| | LGEGSGRITCTSR | | | |
| 283 | NRCNDQDTRTSYRIGDT | integrin | from Fibronectin Domain | |
| | WSKKDNRGNLLQCICTG | | 1-5 | |
| | NGRGEWKCERH | | | |
| 284 | GHCVTDSGVVYSVGMQ | denatured Collagen/ | from Fibronectin Domain | duplicated in collagen |
| | WLKTQGNKQMLCTCLG | integrin | 1-6 | |
| | NGVSCQET | | | |
| 285 | EICTTNEGVMYRIGDQW | denatured Collagen/ | from Fibronectin Domain | duplicated in collagen |
| | DKQHDMGHMMRCTCV | integrin | 1-7 | |
| | GNGRGEWTCIAY | | | |
| 286 | DQCIVDDITYNVNDTFH | denatured Collagen/ | from Fibronectin Domain | duplicated in collagen |
| | KRHEEGHMLNCTCFGQ | integrin | 1-8 | |
| | GRGRWKCDPV | | | |
| 287 | DQCQDSETGTFYQIGDS | denatured Collagen/ | from Fibronectin Domain | duplicated in collagen |
| | WEKYVHGVRYQCYCYG | integrin | 1-9 | |
| | RGIGEWHCQPL | | | |
| 288 | APTDLKFTQVTPTSLSAQ | integrin | from Fibronectin Domain | |
| | WTPPNVQLTGYRVRVTP | | III-14 | |
| | KEKTGPMKEINLAPDSSS | | | |
| | VVVSGLMVATKYEVSV | | | |
| | YALKDTLTSRPAQGVVT | | | |
| | TLENVSPP | | | |
| 289 | APTNLQFVNETDSTVLV | integrin | from Fibronectin Domain | |
| | RWTPPRAQITGYRLTVG | | III-5 | |
| | LTRRGQPRQYNVGPSVS | | | |
| | KYPLRNLQPASEYTVSL | | | |
| | VAIKGNQESPKATGVFT | | | |
| | TLQPG | | | |
| 290 | KGHRGF | integrin | Derived from Collagen I | |
| 291 | GFPGER | integrin | Derived from Collagen I | |
| 292 | GTPGPQGIAGQRDVV | integrin | Derived from Collagen alpha1(I) | |
| 293 | EKGPD | integrin | Derived from Collagen II | |
| 294 | EKGPDP | integrin | Derived from Collagen II | |
| 295 | EKGPDPL | integrin | Derived from Collagen II | |
| 296 | TAGSCLRKFSTM | integrin | Derived from Collagen IV | |
| 297 | TAIPSCPEGTVPLYS | integrin | Derived from Collagen alpha3(IV)-NC1 | |
| 298 | TDIPPCPHGWISLWK | integrin | Derived from Collagen IV | |
| 299 | PHSRN | integrin | Derived from Fibronectin | |
| 300 | RGD | integrin | Derived from Fibronectin | |
| 301 | GRGDSP | integrin | Derived from Fibronectin | |
| 302 | YRVRVTPKEKTGPMKE | integrin | Derived from Fibronectin | |
| 303 | SPPRRARVT | integrin | Derived from Fibronectin | |
| 304 | WQPPRARI | integrin | Derived from Fibronectin | |
| 305 | KNNQKSEPLIGRKKT | integrin | Derived from Fibronectin | |
| 306 | EILDVPST | integrin | Derived from Fibronectin | |
| 307 | REDV | integrin | Derived from Fibronectin | |
| 308 | RQVFQVAYIIIKA | integrin | Derived from Laminin Alpha-1 chain | |
| 309 | SINNTAVMQRLT | integrin | Derived from Laminin Alpha-1 chain | |
| 310 | IKVAV | integrin | Derived from Laminin Alpha-1 chain | |
| 311 | NRWHSIYITRFG | integrin | Derived from Laminin Alpha-1 chain | |
| 312 | TWYKIAFQRNRK | integrin | Derived from Laminin Alpha-1 chain | |
| 313 | RKRLQVQLSIRT | integrin | Derived from Laminin Alpha-1 chain | |
| 314 | KNRLTIELEVRT | integrin | Derived from Laminin Alpha-2 chain | |
| 315 | SYWYRIEASRTG | integrin | Derived from Laminin Alpha-2 chain | |
| 316 | DFGTVQLRNGFPFFSYD | integrin | Derived from Laminin Alpha-2 chain | |
| | LG | | | |
| 317 | GQLFHVAYILIKF | integrin | Derived from Laminin Alpha-3 chain | |
| 318 | KNSFMALYLSKG | integrin | Derived from Laminin Alpha-3 chain | |
| 319 | TLFLAHGRLVFM | integrin | Derived from Laminin Alpha-4 chain | |
| 320 | GQVFHVAYVLIKF | integrin | Derived from Laminin Alpha-5 chain | |
| 321 | GIIFFL | integrin | Derived from Laminin Alpha-5 chain | |
| 322 | LALFLSNGHFVA | integrin | Derived from Laminin Alpha-5 chain | |
| 323 | RYVVLPR | integrin | Derived from Laminin Beta-1 chain | |
| 324 | PDSGR | integrin | Derived from Laminin Beta-1 chain | |
| 325 | YIGSR | integrin | Derived from Laminin Beta-1 chain | |
| 326 | KAFDITYVRLKF | integrin | Derived from Laminin Gamma-1 chain | |
| 327 | RNIAEIIKDI | integrin | Derived from Laminin Gamma-1 chain | |
| 328 | FRHRNRKGY | integrin | Derived from Vitronectin | |
| 329 | KKQRFRHRNRKGYRSQ | integrin | Derived from Vitronectin | |
| 330 | FHRRIKA | integrin | Derived from Sialoprotein | |
| 331 | KRSR | integrin | Derived from Sialoprotein | |
| 332 | GLPGER | α1β1, α2β1 | Derived from Collagen α1(I) 7S | |
| 333 | GFPGER | α1β1, α2β1 | Derived from Collagen alpha1(I) | |
| 334 | GLSGER | α2β1 | Derived from Collagen alpha1(I) | |
| 335 | DGEA | α2β1 | Derived from Collagen alpha1(I) | |
| 336 | GPAGKDGEAGAQG | α2β1 | Derived from Collagen alpha1(I) | |
| 337 | GPKGAAGEPGKP | α1β1, α3β1 | Derived from Collagen alpha1(I) | |
| 338 | GAPGPKGARGSA | α1β1, α3β1 | Derived from Collagen alpha1(I) | |
| 339 | GPQGIAGQRGVVGLP | α1β1 | Derived from Collagen alpha1(I) | |
| 340 | PKGQKGEKG | Poly(I) | Derived from Collagen alpha1(I) | |
| 341 | GASGER | α2β1 | Derived from Collagen alpha1(I) | |
| 342 | GQRGER | α2β1 | Derived from Collagen alpha1(I) | |
| 343 | GMPGER | integrin | Derived from Collagen alpha1(I) | |
| 344 | RGQPGVMGF | VWF | Derived from Collagen alpha1(III) | |
| 345 | GKDGES | α2β1 | Derived from Collagen alpha1(III) | |
| 346 | GLKGEN | α2β1 | Derived from Collagen alpha1(III) | |
| 347 | GLPGEN | α2β1 | Derived from Collagen alpha1(III) | |
| 348 | GLPGEA | α2β1 | Derived from Collagen alpha1(III) | |
| 349 | GPPGDQGPPGIP | α1β1 | Derived from Collagen alpha1(IV) | |
| 350 | GAKGRAGFPGLP | α1β1 | Derived from Collagen alpha1(IV) | |
| 351 | MFKKPTPSTLKAGELR | integrin | Derived from Collagen alpha1(IV) | |
| 352 | GFPGSRGDTGPP | integrin | Derived from Collagen alpha1(IV) | |
| 353 | GVKGDKGNPGWPGAP | integrin | Derived from Collagen alpha1(IV) | |
| 354 | FYFDLR | α1β1, α2β1 | Derived from Collagen alpha1(IV) | |
| 355 | MFKKPTPSTLKAGELR | integrin | Derived from Collagen alpha1(IV) | |
| 356 | GFPGSRGDTGPP | integrin | Derived from Collagen alpha1(IV) | |
| 357 | GVKGDKGNPGWPGAP | integrin | Derived from Collagen alpha1(IV) | |
| 358 | FYFDLR | α1β1, α2β1 | Derived from Collagen alpha1(IV) | |
| 359 | RGQPGVPGVPGMKGD | integrin | Derived from Collagen alpha2(IV) | |
| 360 | TDIPPCPHGWISLWK | integrin | Derived from Collagen alpha3(IV)-NC1 | |
| 361 | MNYYSNS | integrin | Derived from Collagen alpha3(IV)-NC1 | |
| 362 | CNYYSNSYSFWLASLNP | integrin | Derived from Collagen alpha3(IV)-NC1 | |
| | ER | | | |
| 363 | ISRCQVCMKKRH | integrin | Derived from Collagen alpha3(IV)-NC1 | |
| 364 | TLGSCLQRFTTM | integrin | Derived from Collagen alpha3(IV)-NC1 | |
| 365 | GRRGKT | integrin | Derived from Collagen alpha3(IV)-NC1 | |
| 366 | RGQPGRKGL | integrin | Derived from Collagen alpha3(IV)-NC1 | |
| 367 | MFRKPIPSTVKA | integrin | Derived from Collagen alpha3(IV)-NC1 | |
| 368 | IISRCQVCMKMRP | integrin | Derived from Collagen alpha3(IV)-NC1 | |
| 369 | LAGSCLPVFSTL | integrin | Derived from Collagen alpha4(IV)-NC1 | |
| 370 | TAGSCLRRFSTM | integrin | Derived from Collagen alpha5(IV)-NC1 | |
| 371 | NKRAHG | integrin | Derived from Collagen alpha5(IV)-NC2 | |
| 372 | WTPPRAQITGYRLTVGL | α5β1 | Derived from Fibronectin III-5 | |
| | TRR | | | |
| 373 | KLDAPT | α4β1, α4β7 | Derived from Fibronectin III-5 | |
| 374 | PHSRN | α5β1 | Derived from Fibronectin III-9 | |
| 375 | RGD | α5β1, αvβ3 | Derived from Fibronectin III-10 | |
| 376 | RGDS | αIIbβ3 | Derived from Fibronectin III-10 | |
| 377 | GRGDSP | α5β1 | Derived from Fibronectin III-10 | |
| 378 | EDGIHEL | α4β1, α9β1 | Derived from Fibronectin EDA | |
| 379 | PRARITGYIIKYEKPGSPP | integrin | Derived from Fibronectin III-14 | |
| | REVVPRPRPGV | | | |
| 380 | IDAPS | α4β1 | Derived from Fibronectin IIICS-1 | |
| 381 | VVIDASTAIDAPSNL | α4β1 | Derived from Fibronectin IIICS-1 | |
| 382 | LDVPS | α4β1 | Derived from Fibronectin IIICS-1 | |
| 383 | REDV | α4β1 | Derived from Fibronectin IIICS-5 | |
| 384 | PHSRN-RGDSP | α5β1 | Derived from Fibronectin III-10 | |
| 385 | PLDREAIAKY | integrin | Derived from E-Cadherin EC1 | |
| 386 | HAVDI | integrin | Derived from E-Cadherin EC1, groove | |
| 387 | LFSHAVSSNG | integrin | Derived from E-Cadherin EC1, groove | |
| 388 | ADTPPV | integrin | Derived from E-Cadherin EC1, bulge | |
| 389 | QGADTPPVGV | integrin | Derived from E-Cadherin EC1, bulge | |
| 390 | PLDREAIAKY | integrin | Derived from E-Cadherin EC1 | |
| 391 | DQNDN | integrin | Derived from E-Cadherin EC1 | |
| 392 | HAVDI | integrin | Derived from E-Cadherin EC1 | |
| 393 | LRAHAVDING | integrin | Derived from E-Cadherin EC1 | |
| 394 | LRAHAVDVNG | integrin | Derived from E-Cadherin EC1 | |
| 395 | VITVKDINDN | integrin | Derived from E-Cadherin EC2 | |
| 396 | GLDRESYPYY | integrin | Derived from E-Cadherin EC2 | |
| 397 | MKVSATDADD | integrin | Derived from E-Cadherin EC2 | |
| 398 | QDPELPDKNM | integrin | Derived from E-Cadherin EC2, bulge | |
| 399 | LVVQAADLQG | integrin | Derived from E-Cadherin EC2, groove | |
| 400 | NDDGGQFVVT | integrin | Derived from E-Cadherin EC3, bulge | |
| 401 | LVVQAADLQG | integrin | Derived from E-Cadherin EC2, groove | |
| 402 | TYRIWRDTAN | integrin | Derived from E-Cadherin EC4, bulge | |
| 403 | YILHVAVTNY | integrin | Derived from E-Cadherin EC3, groove | |
| 404 | YTALIIATDN | integrin | Derived from E-Cadherin EC4, groove | |
| 405 | QDPELPDKNM | integrin | Derived from E-Cadherin EC2, bulge | |
| 406 | RGDV | αvβ3, αvβ5 | Somatomedin B | |
| 407 | PQVTRGDVFTMP | αvβ3, αvβ5 | Somatomedin B | |
| 408 | LNRQELFPFG | integrin | Nidogen G2 | |
| 409 | SIGFRGDGQTC | integrin | Nidogen G2 | |
| 410 | TWSKVGGHLRPGIVQSG | IgB | Perlecan IV | |
| 411 | VAEIDGIEL | α9β1 | Tenascin-C | |
| 412 | VFDNFVLK | α7β1 | Tenascin-C | |
| 413 | VGVAPG | integrin | Elastin | |
| 414 | PGVGV | integrin | Elastin | |
| 415 | TTSWSQCSKS | α6β1 | CCN-1 | |
| 416 | SVVYGLR | α9β1 | Osteopontin | |
| 417 | DGRGDSVAYG | αvβ3 | Osteopontin | |
| 418 | LALERKDHSG | α6β1 | Thrombospondin | |
| 419 | RGDF | αIIIbβ3 | Fibrinogen | |
| 420 | KRLDGSV | αMβ2 | Fibrinogen | |
| 421 | HHLGGAKQAGDV | αIIbβ3 | Fibrinogen | |
| 422 | YSMKKTTMKIIPFNRLTI | αIIbβ3 | Fibrinogen | |
| | G | | | |
| 423 | GVYYQGGTYSKAS | αMβ2 | Fibrinogen | |
| 424 | LWVTVRSQQRGLF | α5β1 | Laminin α1 LN (A3) | |
| 425 | GTNNWWQSPSIQN | α4β1, α4β7 | Laminin α1 LN (A10) | |
| 426 | WVTVTLDLRQVFQ | α5β1 | Laminin α1 LN (A12) | |
| 427 | RQVFQVAYIIIKA | α1β1, α2β1 | Laminin α1 LN (A13) | |
| 428 | LTRYKITPRRGPPT | α5β1 | Laminin α1 LN (A18) | |
| 429 | LLEFTSARYIRL | integrin | Laminin Laminin α1 LN (A24) | |
| 430 | YIRLRLQRIRTL | integrin | Laminin α1 LN (A25) | |
| 431 | RRYYYSIKDISV | integrin | Laminin α1 V? (A29) | |
| 432 | GGFLKYTVSYDI | integrin | Laminin α1 L4a (A55) | |
| 433 | RDQLMTVLANVT | integrin | Laminin α1 L4a (A64) | |
| 434 | VLIKGGRARKHV | α5β1 | Laminin α1 L4a (A112) | |
| 435 | NLLLLLVKANLK | integrin | Laminin α1 L1 (A167) | |
| 436 | HRDELLLWARKI | integrin | Laminin α1 L1 (A174) | |
| 437 | KRRARDLVHRAE | integrin | Laminin α1 L1 (A177) | |
| 438 | SQFQESVDNITK | integrin | Laminin α1 L1 (A191) | |
| 439 | PGGMREKGRKAR | integrin | Laminin α1 L1 (A194) | |
| 440 | MEMQANLLLDRL | integrin | Laminin α1 L1 (A203) | |
| 441 | LSEIKLLISRAR | integrin | Laminin α1 L1 (A206) | |
| 442 | IKVAV | αvβ3 | Laminin α1 L1 (A208) | |
| 443 | AASIKVAVSADR | αvβ3 | Laminin α1 L1 (A208) | |
| 444 | NRWHSIYITRFG | α6β1 | Laminin α1 LG1 (AG10) | |
| 445 | SSFHFDGSGYAM | integrin | Laminin α1 LG2 (AG22) | |
| 446 | IAFQRN | α6β1 | Laminin α1 LG2 (AG32) | |
| 447 | TWYKIAFQRNRK | α6β1 | Laminin α1 LG2 (AG32) | |
| 448 | SLVRNRRVITIQ | integrin | Laminin α1 LG2 (AG56) | |
| 449 | DYATLQLQEGRLHFMFD | α2β1 | Laminin EF-1 | |
| | LG | | | |
| 450 | KKGSYNNIVVHV | integrin | Laminin α2 LG (A2G2) | |
| 451 | ADNLLFYLGSAK | integrin | Laminin α2 LG (A2G4) | |
| 452 | GSAKFIDFLAIE | integrin | Laminin α2 LG (A2G5) | |
| 453 | KVSFLWWVGSGV | integrin | Laminin α2 LG (A2G7) | |
| 454 | SYWYRIEASRTG | integrin | Laminin α2 LG (A2G10) | |
| 455 | ISTVMFKFRTFS | integrin | Laminin α2 LG (A2G25) | |
| 456 | KQANISIVDIDSN | integrin | Laminin α2 LG (A2G34) | |
| 457 | FSTRNESGIILL | integrin | Laminin α2 LG (A2G48) | |
| 458 | RRQTTQAYYAIF | integrin | Laminin α2 LG (A2G51) | |
| 459 | YAIFLNKGRLEV | integrin | Laminin α2 LG (A2G52) | |
| 460 | KNRLTIELEVRT | integrin | Laminin α2 LG (A2G76) | |
| 461 | GLLFYMARINHA | integrin | Laminin α2 LG (A2G78) | |
| 462 | VQLRNGFPYFSY | integrin | Laminin α2 LG (A2G80) | |
| 463 | HKIKIVRVKQEG | integrin | Laminin α2 LG (A2G84) | |
| 464 | DFGTVQLRNGFPFFSYD | integrin | Laminin EF-2 | |
| | LG | | | |
| 465 | YFDGTGFAKAVG | integrin | Laminin α2 LG (A2G94) | |
| 466 | NGQWHKVTAKKI | integrin | Laminin α2 LG (A2G103) | |
| 467 | AKKIKNRLELVV | integrin | Laminin α2 LG (A2G104) | |
| 468 | GFPGGLNQFGLTTN | integrin | Laminin α2 LG (A2G109) | |
| 469 | IRSLKLTKGTGKP | integrin | Laminin α2 LG (A2G111) | |
| 470 | AKALELRGVQPVS | integrin | Laminin α2 LG (A2G113) | |
| 471 | GOLFHVAYILIKF | integrin | Laminin α3 (A3-10) | |
| 472 | SQRIYQFAKLNYT | integrin | Laminin α3 LG (MA3G13) | |
| 473 | NVLSLYNFKTTF | integrin | Laminin α3 LG (MA3G22) | |
| 474 | NAPFPKLSWTIQ | integrin | Laminin α3 LG (MA3G27) | |
| 475 | WTIQTTVDRGLL | integrin | Laminin α3 LG (MA3G28) | |
| 476 | DTINNGRDHMILI | integrin | Laminin α3 LG (MA3G34) | |
| 477 | MILISIGKSQKRM | integrin | Laminin α3 LG (MA3G35) | |
| 478 | PPFLMLLKGSTR | integrin | Laminin α3 LG (A3GXX) | |
| 479 | NQRLASFSNAQQS | integrin | Laminin α3 LG (MA3G57) | |
| 480 | ISNVFVQRMSQSPEVLD | integrin | Laminin α3 LG (MA3G59) | |
| 481 | KARSFNVNOLLQD | integrin | Laminin α3 LG (MA3G63) | |
| 482 | KNSFMALYLSKG | integrin | Laminin α3 LG A3G75 | |
| 483 | KNSFMALYLSKGRLVFA | integrin | Laminin α3 LG A3G756 | |
| | LG | | | |
| 484 | RDSFVALYLSEGHVIFAL | integrin | Laminin EF-3 | |
| | G | | | |
| 485 | KPRLQFSLDIQT | integrin | Laminin α3 LG MA3G70 | |
| 486 | DGQWHSVTVSIK | integrin | Laminin α3 LG MA3G97 | |
| 487 | FVLYLGSKNAKK | integrin | Laminin α4 LG (A4G4) | |
| 488 | LAIKNDNLVYVY | integrin | Laminin α4 LG (A4G6) | |
| 489 | AYFSIVKIERVG | integrin | Laminin α4 LG (A4G10) | |
| 490 | DVISLYNFKHIY | integrin | Laminin α4 LG (A4G20) | |
| 491 | FFDGSSYAVVRD | integrin | Laminin α4 LG (A4G24) | |
| 492 | LHVFYDFGFSNG | integrin | Laminin α4 LG (A4G31) | |
| 493 | LKKAQINDAKYREISIIY | integrin | | |
| | HN | | | |
| 494 | RAYFNGQSFIAS | integrin | Laminin α4 LG (A4G47) | |
| 495 | SRLRGKNPTKGK | integrin | Laminin α4 LG (A4G59) | |
| 496 | LHKKGKNSSKPK | integrin | Laminin α4 LG (A4G69) | |
| 497 | RLKTRSSHGMIF | integrin | | |
| 498 | GEKSQFSIRLKT | integrin | Laminin α4 LG (A4G78) | |
| 499 | TLFLAHGRLVFM | integrin | Laminin α4 LG (A4G82) | |
| 500 | LVFMFNVGHKKL | integrin | Laminin α4 LG (A4G83) | |
| 501 | TLFLAHGRLVFMFNVGH | integrin | Laminin α4 LG (A4G823) | |
| | KKL | | | |
| 502 | DFMTLFLAHGRLVFMFN | integrin | Laminin EF-4 | |
| | VG | | | |
| 503 | HKKLKIRSQEKY | integrin | Laminin α4 LG (A4G84) | |
| 504 | GAAWKIKGPIYL | integrin | Laminin α4 LG (A4G90) | |
| 505 | VIRDSNVVQLDV | integrin | Laminin α4 LG (A4G107) | |
| 506 | EVNVTLDLGQVFH | α5β1 | Laminin Laminin α5 LN (S1) | |
| 507 | GQVFHVAYVLIKF | α4β1, α4β7 | Laminin Laminin α5 LN (S2) | |
| 508 | RDFTKATNIRLRFLR | α5β1 | Laminin Laminin α5 LN (S6) | |
| 509 | NIRLRFLRTNTL | α5β1 | Laminin Laminin α5 LN (S7) | |
| 510 | GKNTGDHFVLYM | α5β1 | Laminin α5 LG1 (A5G3) | |
| 511 | VVSLYNFEQTFML | integrin | Laminin α5 LG1 (A5G19) | |
| 512 | RFDQELRLVSYN | integrin | Laminin α5 LG2 (A5G26) | |
| 513 | ASKAIQVFLLGG | integrin | Laminin α5 LG2 (A5G33) | |
| 514 | TVFSVDQDNMLE | integrin | Laminin α5 LG2 (A5G36) | |
| 515 | RLRGPQRVFDLH | α5β1 | Laminin α5 LG3 (A5G63) | |
| 516 | SRATAQKVSRRS | integrin | Laminin α5 LG3 (A5G66) | |
| 517 | GSLSSHLEFVGI | integrin | Laminin α5 LG4 (A5G71) | |
| 518 | RNRLHLSMLVRP | integrin | Laminin α5 LG4 (A5G73) | |
| 519 | APMSGRSPSLVLK | integrin | Laminin α5 LG4 (A5G76) | |
| 520 | LALFLSNGHEVA | integrin | Laminin α5 LG4 (A5G77) | |
| 521 | PGRWHKVSVRWE | integrin | Laminin α5 LG4 (A5G81) | |
| 522 | VRWGMQQIQLVV | integrin | Laminin α5 LG4 (A5G82) | |
| 523 | KMPYVSLELEMR | integrin | Laminin α5 LG5 (A5G94) | |
| 524 | VLLQANDGAGEF | integrin | Laminin α5 LG5 (A5G99) | |
| 525 | DGRWHRVAVIMG | integrin | Laminin α5 LG5 (A5G101) | |
| 526 | APVNVTASVQIQ | integrin | Laminin α5 LG5 (A5G109) | |
| 527 | KQGKALTQRHAK | integrin | Laminin α5 LG5 (A5G112) | |
| 528 | AFGVLALWGTRV | integrin | Laminin Laminin VI (B-7) | |
| 529 | IENVVTTFAPNR | integrin | Laminin Laminin VI (B-15) | |
| 530 | LEAEFHFTHLIM | integrin | Laminin Laminin VI (B-19) | |
| 531 | HLIMTFKTFRPA | integrin | Laminin Laminin VI (B-20) | |
| 532 | KTWGVYRYFAYD | integrin | Laminin Laminin VI (B-23) | |
| 533 | TNLRIKFVKLHT | integrin | Laminin Laminin VI (B-31) | |
| 534 | REKYYYAVYDMV | integrin | Laminin Laminin VI (B-34) | |
| 535 | KRLVTGQR | integrin | Laminin Laminin V (B-54) | |
| 536 | KDISEKVAVYST | integrin | I (B-187) | |
| 537 | PDSGR | integrin | Laminin III (B-96) | |
| 538 | YIGSR | α1β1, α3β1 | Laminin III (B-98) | |
| 539 | DPGYIGSR | α1β1, α3β1 | Laminin III (B-98) | |
| 540 | FALWDAIIGEL | integrin | Laminin III (B-116) | |
| 541 | AAEPLKNIGILF | integrin | Laminin II (B-123) | |
| 542 | DSITKYFQMSLE | integrin | Laminin II (B-133) | |
| 543 | VILQQSAADIAR | integrin | Laminin I (B-160) | |
| 544 | SPYTFIDSLVLMPY | integrin | Laminin Laminin IV (B-77) | |
| 545 | KDISEKVAVYST | integrin | Laminin I (B-187) | |
| 546 | LGTIPG | integrin | | |
| 547 | LWPLLAVLAAVA | integrin | Laminin VI (C-3) | |
| 548 | KAFDITYVRLKF | αvβ3, α5β1 | Laminin VI (C-16) | |
| 549 | AFSTLEGRPSAY | integrin | Laminin VI (C-25) | |
| 550 | TDIRVTLNRLNTF | integrin | Laminin VI (C-28) | |
| 551 | NEPKVLKSYYYAI | integrin | Laminin VI (C-30) | |
| 552 | YYAISDFAVGGR | integrin | Laminin VI (C-31) | |
| 553 | LPFFNDRPWRRAT | integrin | Laminin VI (C-35) | |
| 554 | FDPELYRSTGHGGH | integrin | Laminin V (C-38) | |
| 555 | TNAVGYSVYDIS | integrin | Laminin V (C-50) | |
| 556 | APVKFLGNQVLSY | integrin | Laminin IV (C-57) | |
| 557 | SFSFRVDRRDTR | integrin | Laminin IV (C-59) | |
| 558 | SETTVKYIFRLHE | integrin | Laminin IV (C-64) | |
| 559 | FQKLLNNLTSIK | integrin | Laminin IV (C-67) | |
| 560 | TSIKIRGTYSER | integrin | Laminin IV (C-68) | |
| 561 | DPETGV | integrin | Laminin III (C75) | |
| 562 | TSAEAYNLLLRT | integrin | Laminin II (C-118) | |
| 563 | KEAEREVTDLLR | integrin | Laminin II (C102) | |
| 564 | SLLSQLNNLLDQ | integrin | Laminin II (C-155) | |
| 565 | RNIAEIIKDI | integrin | Laminin | |
| 566 | RDIAEIIKDI | integrin | Laminin | |
| 567 | GAPGER | integrin | Derived from Collagen alpha1 (I) | |
| 568 | FNKHTEIIEEDTNKDKPS | Fibronectin | (FAB D3: 1-37)-highest | Differential binding |
| | YQFGGHNSVDFEEDTLP | | affinity | affinity to Collagen |
| | KV | | | |
| 569 | PSYQFGGHNSVDFEEDT | Fibronectin | (FAB D3: 16-36)-high | Differential binding |
| | LPK | | affinity | affinity to Collagen |
| 570 | SYQFGGHNSVDFEEDT | Fibronectin | (FAB D3: 17-33)-medium | Differential binding |
| | | | affinity | affinity to Collagen |
| 571 | QFGGHNSVDFEEDTLPK | Fibronectin | (FAB D3: 20-36)-medium | Differential binding |
| | | | affinity | affinity to Collagen |
| 572 | FGGHNSVDFEEDTLPK | Fibronectin | (FAB D3: 21-36)-low | Differential binding |
| | | | affinity | affinity to Collagen |
| 573 | NAPQPSHISKYILRWRPK | Fibronectin | Fibronectin Type III(1) | |
| | NSVGRWKEATIPGHLNS | | | |
| | YTIKGLKPGVVYEGQLIS | | | |
| | IQQYGHQEVTRFDFTTTS | | | |
| | TSTPVTSNTVTGETTPFS | | | |
| | PLVATSESVTEITASSFV | | | |
| | VS | | | |
| 574 | NAPQPSHISKYILRWRPK | Fibronectin | Fibronectin Type III(1) fragment | |
| | NSVGRWKEATIPG | | | |
| 575 | EATIPGHLNSYTIKGLKP | Fibronectin | Fibronectin Type III(1) fragment | |
| | GVVYEGQLISIQQ | | | |
| 576 | LISIQQYGHQEVTRFDFT | Fibronectin | Fibronectin Type III(1) fragment | |
| | TTSTSTPVTSNTV | | | |
| 577 | VTSNTVTGETTPFSPLVA | Fibronectin | Fibronectin Type III(1) fragment | |
| | TSESVTEITASSFVVS | | | |
| 578 | RWSHDNGVNYKIGEKW | Fibronectin | Fibronectin Type III(1) fragment (synthetic) | |
| | DRQGENGQMMSSTSLG | | | |
| | NGKGEFKSDPHE | | | |
| 579 | ATSYDDGKTYHVGEQW | Fibronectin | Fibronectin Type III(1) fragment (synthetic) | |
| | QKEYLGAISSSTSFGGQR | | | |
| | GWRSDNSR | | | |
| 580 | DKPSYQFGGHNSVDFEE | Fibronectin | | |
| | DT | | | |
| 581 | DKPSYQFGGHNSVDFEE | Fibronectin | | |
| | DTL | | | |
| 582 | DKPSYQFGGHNSVDFEE | Fibronectin | | |
| | DTLP | | | |
| 583 | DKPSYQFGGHNSVDFEE | Fibronectin | | |
| | DTLPK | | | |
| 584 | KPSYQFGGHNSVDFEED | Fibronectin | | |
| | T | | | |
| 585 | KPSYQFGGHNSVDFEED | Fibronectin | | |
| | TL | | | |
| 586 | KPSYQFGGHNSVDFEED | Fibronectin | | |
| | TLP | | | |
| 587 | KPSYQFGGHNSVDFEED | Fibronectin | | |
| | TLPK | | | |
| 588 | PSYQFGGHNSVDFEEDT | Fibronectin | | |
| 589 | PSYQFGGHNSVDFEEDT | Fibronectin | | |
| | L | | | |
| 590 | PSYQFGGHNSVDFEEDT | Fibronectin | | |
| | LP | | | |
| 591 | PSYQFGGHNSVDFEEDT | Fibronectin | | |
| | LPK | | | |
| 592 | PPFLMLLKGSTRFNKTK | Heparin/syndecans | Derived from Heparin | Differential binding |
| | TFR | | Binding Domans of Laminin | affinity to |
| | | | | Heparin/syndecans |
| 593 | RLVFALGTDGKKLRIKS | Heparin/syndecans | Derived from Heparin | Differential binding |
| | KEKCNDGK | | Binding Domans of Laminin | affinity to |
| | | | | Heparin/syndecans |
| 594 | PLFLLHKKGKNLSKPKA | Heparin/syndecans | Derived from Heparin | Differential binding |
| | SQNKKGGKSK | | Binding Domans of Laminin | affinity to |
| | | | | Heparin/syndecans |
| 595 | TLFLAHGRLVYMFNVG | Heparin/syndecans | Derived from Heparin | Differential binding |
| | HKKLKIR | | Binding Domans of Laminin | affinity to |
| | | | | Heparin/syndecans |
| 596 | TPGLGPRGLQATARKAS | Heparin/syndecans | Derived from Heparin | Differential binding |
| | RRSRQPARHPACML | | Binding Domans of Laminin | affinity to |
| | | | | Heparin/syndecans |
| 597 | RQRSRPGRWHKVSVRW | Heparin/syndecans | Derived from Heparin | Differential binding |
| | EKNR | | Binding Domans of Laminin | affinity to |
| | | | | Heparin/syndecans |
| 598 | LAGSCLARFSTM | α2β1, Heparin | Derived from Collagen alpha1(IV) HepII | |
| 599 | KGHRGF | Heparin | Derived from Collagen alpha1(I) | |
| 600 | GDRGIKGHRGFSG | Heparin | Derived from Collagen alpha1(I) | |
| 601 | GDLGRPGRKGRPGPP | Heparin | Derived from Collagen alpha1(I) | |
| 602 | GHRGPTGRPGKRGKQG | Heparin | Derived from Collagen alpha1(I) | |
| | QKGDS | | | |
| 603 | KGIRGH | Heparin | Derived from Collagen alpha2(I) | |
| 604 | GEFYFDLRLKGDK | α2β1, Heparin | Derived from Collagen alpha1(IV) HepIII | |
| 605 | KYILRWRPKNS | Heparin | Derived from Fibronectin III-1 | |
| 606 | YRVRVTPKEKTGPMKE | Heparin | Derived from Fibronectin III-13 (FN-C/H-III) | |
| 607 | SPPRRARVT | α5β1, Heparin | Derived from Fibronectin III-13 (FN-C/H-IV) | |
| 608 | ATETTITIS | Heparin | Derived from Fibronectin III-13 | |
| 609 | VSPPRRARVTDATETTIT | α5β1, Heparin | Derived from Fibronectin III-13 | |
| | ISWRTKTETITGFG | | | |
| 610 | KPDVRSYTITG | α4β1, Heparin | Derived from Fibronectin III-13 | |
| 611 | ANGQTPIQRYIK | α4β1, Heparin | Derived from Fibronectin III-13 | |
| 612 | YEKPGSPPREVVPRPRPG | Heparin | Derived from Fibronectin III-14 (FN-C/H-I) | |
| | V | | | |
| 613 | KNNQKSEPLIGRKKT | Heparin | Derived from Fibronectin III-14 (FN-C/H-II) | |
| 614 | EILDVPST | integrin | Derived from Fibronectin IIICS-1 | |
| 615 | TAGSCLRKFSTM | α2β1, Heparin | Derived from Collagen alphal (IV) HepI | |
| 616 | FRHRNRKGY | Heparin | HPV | |
| 617 | KKQRFRHRNRKGYRSQ | Heparin | HPV | |
| 618 | KRSR | Heparin | Bone sialoprotein | |
| 619 | FHRRIKA | Heparin, HSP | Bone sialoprotein | |
| 620 | SINNTAVMQRLT | Heparin | Laminin Laminin α1 L4a (A51) | |
| 621 | ANVTHLLIRANY | Heparin | Laminin α1 L4a (A65) | |
| 622 | AGTFALRGDNPQG | integrin | Laminin α1 L4a (A99) | |
| 623 | RLVSYSGVLFFLK | Heparin | Laminin α5 LG2 (A5G27) | |
| 624 | GIIFFL | Heparin | Laminin α5 LG2 (A5G) | |
| 625 | VLVRVERATVES | Heparin | Laminin α5 LG2 (A5G35) | |
| 626 | RIQNLLKITNLRIKFVK | Heparin | Laminin Laminin VI (B-30) | |
| 627 | GPGVVVVERQYI | Heparin | Laminin IV (B-62) | |
| 628 | RYVVLPR | Heparin | Laminin IV (B-73) | |
| 629 | LSNIDYILIKAS | SDC-4 | Laminin α1 L4a (A119) | |
| 630 | LQQSRIANISME | SDC-4 | Laminin α1 L4a (A121) | |
| 631 | LQVQLSIR | SDC-1, -4 | Laminin αl LG4 (AG73) | |
| 632 | RKRLQVQLSIRT | SDC-1, -4 | Laminin α1 LG4 (AG73) | |
| 633 | GLIYYVAHQNQM | SDC-1, -4 | Laminin α1 LG4 (AG75) | |
| 634 | FDLHQNMGSVN | SDC-4 | Laminin α5 LG3 (A5G64) | |
| 635 | QQNLGSVNVSTG | SDC-4 | Laminin α5 LG3 (A5G65) | |
| 636 | WQPPRARI | SDC-4 | Derived from Fibronectin III-14 (FN-C/H-V) | |
| 637 | WQPPRARITGYIIKYEKP | SDC-4 | Derived from Fibronectin III-14 (FN-C/H-V) | |
| | G | | | |
| 638 | KNSFMALYLSKGR | syndecan 2(w) | Derived from Heparin | Differential binding |
| | | | Binding Domans of Laminin | affinity to |
| | | | | Heparin/syndecans |

| | | | |
|---|---|---|---|
| 639 | NGRKIRMRCRAIDGD | Heparan sulfate | binds to HSGP with high affinity (DTx protein) |
| | | proteoglycans | |
| 640 | DVIRDKTKTKIESLK | Heparan sulfate | binds to HSGP with low affinity (DTx protein) |
| | | proteoglycans | |

pH-sensitive targeting sequences

| 641 | GVYHREARSGKYKLTY | hyaluronic acid | pH dependent (Link_TGS6) | binds better at lower |
|---|---|---|---|---|
| | AEAKAVCEFEGGHLATY | | | pH |
| | KGLEAARKIGFHVCAAG | | | |
| | WMAKGRVGYPIVKPGPP | | | |
| | NCGFGKTGIIDYGIRLNR | | | |
| | SERWDAYCYNPHA | | | |
| 642 | KHAHLKKQVSDHIAVY | Heparin | binds to heparin at low pH (high affinity) | |
| 643 | TTEPSEEHNHHK | Heparin | binds to heparin at low pH (low affinity) | |
| 644 | KHAHL | Heparin | binds to heparin at low pH (lower affinity) | |
| 645 | TTEPSEEHNHHK | Heparin | binds to heparin at low pH (lower affinity) | |
| 646 | TTEPSEEHNHHKHHDK | Heparin | binds to heparin at low pH (lower affinity) | |
| 647 | HKGQHR | Heparin | binds to heparin at low pH (lower affinity) | |
| 648 | KVEHRVKKRPPTWRHN | Heparin | binds to heparin at low pH | |
| | VRAKYT | | | |
| 649 | GGKVEHRVKKRPPTWR | Heparin | binds to heparin at low pH | |
| | HNVRAKYT | | | |
| 650 | KKRPPTWRHNV | Heparin | binds to heparin at low pH | |
| 651 | GTWSEW | heparin | derived from thrombospondin | |
| 652 | GFWSEW | heparin | derived from thrombospondin | |
| 653 | GGWSHW | Fibronectin | derived from | binds better at |
| | | | thrombospondin | lower pH |
| | | | (highest affinity) | |
| 654 | KRFKQDGGWSHWSPWS | Fibronectin | derived from thrombospondin (low affinity) | |
| | S | | | |
| 655 | KRFKQDGGWSHWSP | Fibronectin | derived from thrombospondin (medium affinity) | |
| 656 | GGWSHWSPWSS | Fibronectin | derived from thrombospondin (medium affinity) | |
| 657 | WSXWS | Sulfated Glycoprotein | derived from thrombospondin (X = any amino acids) | |
| 658 | WSHW | Sulfated Glycoprotein | derived from thrombospondin | |
| 659 | Xaa Xaa Pro His Glu | heparin/heparan sulfate | Xaa = any amino acid | |
| 660 | (H/P)(H/P)PHG | heparin/heparan sulfate | tandem repeat-pH dependent HRGP (Histidine Rich | |
| | | | Glyco Protein) | |
| 661 | HPHKHHSHEQHPHGHHP | heparin/heparan sulfate | Histidine Rich Glycoprotein (Histidine Rich | |
| | HAHHPHEHDTHRQHPH | | Domain) | |
| | GHHPHGHHPHGHHPHG | | | |
| | HHPHGHHPHCHDFQDY | | | |
| | GPCDPPPHNQGHCCHGH | | | |
| | GPPPGHLRRRGPGKGPR | | | |
| | PFHCRQIGSVYRLPPLRK | | | |
| | GEVLPLPEANFPSFPLPH | | | |
| | HKHPLKPDNQPFP | | | |
| 662 | DLHPHKHHSHEQHPHGH | heparin/heparan sulfate | Histidine Rich Glycoprotein (Histidine Rich | |
| | HPHAHHPHEHDTHRQHP | | Domain) | |
| | H | | | |
| 663-679 | Not Used | | | |

Control sequence

| 680 | QFGGHNSVDFEEDT | Fibronectin | non-binding control |
|---|---|---|---|
| 681-700 | Not Used | | |

### Definitions

As used herein, a “cytokine polypeptide sequence” refers to a polypeptide sequence (which may be part of a larger sequence, e.g., a fusion polypeptide) with significant sequence identity to a wild-type cytokine and which can bind and activate a cytokine receptor when separated from an inhibitory polypeptide sequence. In some embodiments, a cytokine polypeptide sequence has at least 80, 85, 90, 95, 97, 98, or 99 percent identity to the sequence of a wild-type cytokine, e.g., a wild-type human cytokine. In some embodiments, a cytokine polypeptide sequence has no more than one, two, three, four, five, six, seven, eight, nine, or ten amino acid differences from a wild-type cytokine, e.g., a wild-type human cytokine. Cytokines include but are not limited to chemokines. Exemplary cytokine polypeptide sequences are provided in Table 1. This definition applies to IL-2 polypeptide sequences with substitution of “IL-2” for “cytokine.”

As used herein, an “inhibitory polypeptide sequence” is a sequence in a cytokine prodrug that inhibits the activity of the cytokine polypeptide sequence in the prodrug. The inhibitory polypeptide sequence binds the cytokine polypeptide sequence, and such binding is reduced or eliminated by action of an appropriate protease on the protease-cleavable polypeptide sequence. Exemplary inhibitory polypeptide sequences are provided in Table 1.

As used herein, a “protease-cleavable polypeptide sequence” is a sequence that is a substrate for cleavage by a protease. The protease-cleavable polypeptide sequence is located in a cytokine prodrug such that its cleavage reduces or eliminates binding of the inhibitory polypeptide sequence to the cytokine polypeptide sequence.

As used herein, a protease-cleavable polypeptide sequence “is recognized by” a given protease or class thereof if exposing a polypeptide comprising the protease-cleavable polypeptide sequence to the protease under conditions permissive for cleavage by the protease results in a significantly greater amount of cleavage than is seen for a control polypeptide having an unrelated sequence, and/or if the protease-cleavable polypeptide sequence corresponds to a known recognition sequence for the protease (e.g., as described elsewhere herein for various exemplary proteases).

As used herein, a “pharmacokinetic modulator” is a moiety that extends the in vivo half-life of a cytokine prodrug. The pharmacokinetic modulator may be a fused domain in a cytokine prodrug or may be a chemical entity attached post-translationally. The attachment may be, but is not necessarily, covalent. Exemplary pharmacokinetic modulator polypeptide sequences are provided in Table 1. Exemplary non-polypeptide pharmacokinetic modulators are described elsewhere herein.

As used herein, a “targeting sequence” is a sequence that results in a greater fraction of a cytokine prodrug localizing to an area of interest, e.g., a tumor microenvironment. The targeting sequence may bind an extracellular matrix component or other entity found in the area of interest, e.g., an integrin or syndecan. Exemplary targeting sequences are provided in Table 2.

As used herein, an “extracellular matrix component” refers to an extracellular protein or polysaccharide found in vivo. Integral and peripheral membrane proteins on a cell, including fibronectins, cadherins, integrins, and syndecans, are not considered extracellular matrix components.

As used herein, an “immunoglobulin constant domain” refers to a domain that occurs in or has significant sequence identity to a domain of a constant region of an immunoglobulin, such as an IgG. Exemplary constant domains are C_{H}2 and C_{H}3 domains. Unless indicated otherwise, a polypeptide or prodrug comprising an immunoglobulin constant domain may comprise more than one immunoglobulin constant domain. In some embodiments, an immunoglobulin constant domain has at least 80, 85, 90, 95, 97, 98, or 99 percent identity to the sequence of a wild-type immunoglobulin constant domain, e.g., a wild-type human immunoglobulin constant domain. In some embodiments, an immunoglobulin constant domain has no more than one, two, three, four, five, six, seven, eight, nine, or ten amino acid differences from a wild-type immunoglobulin constant domain, e.g., a wild-type human immunoglobulin constant domain. In some embodiments, immunoglobulin constant domain has an identical sequence to a wild-type immunoglobulin constant domain, e.g., a wild-type human immunoglobulin constant domain. Exemplary immunoglobulin constant domains are contained within sequences provided in Table 1. This definition applies to C_{H}2 and C_{H}3 domains, respectively, with substitution of “C_{H}2” or “C_{H}3” for “immunoglobulin constant,” with the qualification that a C_{H}2 domain sequence does not have greater percent identity to a non-C_{H}2 immunoglobulin constant domain wild-type sequence than to a C_{H}2 domain wild-type sequence, and a C_{H}3 domain sequence does not have greater percent identity to a non-C_{H}3 immunoglobulin constant domain wild-type sequence than to a C_{H}3 domain wild-type sequence. These definitions also include domains having minor truncations relative to wild-type sequences, to the extent that the truncation does not abrogate substantially normal folding of the domain.

As used herein, a “immunoglobulin Fc region” refers to a region of an immunoglobulin heavy chain comprising a C_{H}2 and a C_{H}3 domain, as defined above. The Fc region does not include a variable domain or a C_{H}1 domain.

As used herein, a given component is “between” a first component and a second component if the first component is on one side of the given component and the second component is on the other component, e.g., in the primary sequence of a polypeptide. This term does not require immediate adjacency. Thus, in the structure 1-2-3-4, 2 is between 1 and 4, and is also between 1 and 3.

As used herein, a “domain” may refer, depending on the context, to a structural domain of a polypeptide or to a functional assembly of at least one domain (but possibly a plurality of structural domains). For example, a C_{H}2 domain refers to a part of a sequence that qualifies as such. An immunoglobulin cytokine-binding domain may comprise VH and VL structural domains.

As used herein, “denatured collagen” encompasses gelatin and cleavage products resulting from action of an MMP on collagen, and more generally refers to a form of collagen or fragments thereof that does not exist in the native structure of full-length collagen.

As used herein, “configured to bind . . . in a pH-sensitive manner” means that a polypeptide sequence (e.g., a targeting sequence) shows differential binding affinity for its binding partner depending on pH. For example, the polypeptide sequence may have a higher affinity at a relatively acidic pH than at normal physiological pH (about 7.4). The higher affinity may occur at a pH below 7, e.g., in the range of pH 5.5-7, 6-7, or 5.5-6.5, or below pH 6.

As used herein, a “cytokine-binding domain of a cytokine receptor” refers to an extracellular portion of a cytokine receptor, or a fragment or truncation thereof that can bind a cytokine polypeptide sequence. In some embodiments, the sequence of a cytokine binding domain of a cytokine receptor has at least 80, 85, 90, 95, 97, 98, or 99 percent identity to the sequence of a cytokine binding domain of wild-type cytokine receptor, e.g., a cytokine binding domain of a wild-type human cytokine receptor. Exemplary sequences of a cytokine binding domain of a cytokine receptor are provided in Table 1. This definition applies to IL-2-binding domains of an IL-2 receptor with substitution of “IL-2” for “cytokine.”

As used herein, a “cytokine-binding immunoglobulin domain” refers to one or more immunoglobulin variable domains (e.g., a VH and a VL domain) that can bind a cytokine polypeptide sequence. Exemplary sequences of a cytokine-binding immunoglobulin domain are provided in Table 1. This definition applies to IL-2-binding immunoglobulin domains with substitution of “IL-2” for “cytokine.”

As used herein, “substantially” and other grammatical forms thereof mean sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “plurality” can be 2, 3, 4, 5, 6, 7, 8, 9, 10, or more.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence QLYV comprises a sequence with 100% identity to the sequence QLY because an alignment would give 100% identity in that there are matches to all three positions of the second sequence. Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

As used herein, a “subject” refers to any member of the animal kingdom. In some embodiments, “subject” refers to humans. In some embodiments, “subject” refers to non-human animals. In some embodiments, “subject” refers to primates. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically-engineered animal, and/or a clone. In certain embodiments of the present invention the subject is an adult, an adolescent or an infant. In some embodiments, the terms “individual” or “patient” are used and are intended to be interchangeable with “subject”.

### Cytokine Polypeptide Sequence

The cytokine polypeptide sequence may be a wild-type cytokine polypeptide sequence or a sequence with one or more differences from the wild-type cytokine polypeptide sequence. In some embodiments, the cytokine polypeptide sequence is a human cytokine polypeptide sequence (which may be wild-type or may have one or more differences). In some embodiments, the cytokine comprises a modification to prevent disulfide bond formation, and optionally otherwise comprises wild-type sequence. In some embodiments, the cytokine polypeptide sequence has at least 80, 85, 90, 95, 97, 98, or 99 percent identity to the sequence of a wild-type cytokine polypeptide sequence or to a cytokine polypeptide sequence in Table 1. In some embodiments, the cytokine is a dimeric cytokine, e.g., a heterodimeric cytokine. In some embodiments, the cytokine is a homodimeric cytokine. The monomers may be linked as a fusion protein, e.g., with a linker, or by a covalent bond (e.g., disulfide bond), or by a noncovalent interaction. In some embodiments, the cytokine polypeptide sequence is an interleukin polypeptide sequence. In some embodiments, the cytokine polypeptide sequence is capable of binding a receptor comprising CD132. In some embodiments, the cytokine polypeptide sequence is capable of binding a receptor comprising CD122. In some embodiments, the cytokine polypeptide sequence is capable of binding a receptor comprising CD25.

### IL-2

In some embodiments, the cytokine polypeptide sequence is an IL-2 polypeptide sequence. The IL-2 polypeptide sequence may be a wild-type IL-2 polypeptide sequence or a sequence with one or more differences from the wild-type IL-2 polypeptide sequence. In some embodiments, the IL-2 polypeptide sequence is a human IL-2 polypeptide sequence (which may be wild-type or may have one or more differences). In some embodiments, the IL-2 comprises a modification to prevent disulfide bond formation (e.g., the sequence of aldesleukin (marketed as Proleukin®), and optionally otherwise comprises wild-type sequence. In some embodiments, the IL-2 polypeptide sequence has at least 80, 85, 90, 95, 97, 98, or 99 percent identity to the sequence of a wild-type IL-2 polypeptide sequence or to a IL-2 polypeptide sequence in Table 1.

### Inhibitory Polypeptide Sequence

Various types of inhibitory polypeptide sequences may be used in a cytokine prodrug according to the disclosure. In some embodiments, the inhibitory polypeptide sequence comprises a cytokine-binding domain.

The cytokine-binding domain may be the cytokine-binding domain of a cytokine receptor. The cytokine-binding domain of a cytokine receptor may be provided as an extracellular portion of the cytokine receptor or a portion thereof sufficient to bind the cytokine polypeptide sequence of the cytokine prodrug. In some embodiments, the cytokine-binding domain of a cytokine receptor has at least 80, 85, 90, 95, 97, 98, or 99 percent identity to the sequence of a wild-type cytokine-binding domain of a cytokine receptor, e.g., a wild-type cytokine-binding domain of a human cytokine receptor.

The cytokine-binding domain may be a fibronectin cytokine-binding domain. In some embodiments, the fibronectin cytokine-binding domain has at least 80, 85, 90, 95, 97, 98, or 99 percent identity to the sequence of a wild-type fibronectin cytokine-binding domain of a cytokine receptor, e.g., a wild-type human fibronectin cytokine-binding domain.

The cytokine-binding domain may be an immunoglobulin cytokine-binding domain. The immunoglobulin cytokine-binding domain may be an Fv, scFv, Fab, VHH, or other immunoglobulin sequence having antigen-binding activity for the cytokine polypeptide sequence. A VHH antibody (or nanobody) is an antigen binding fragment of a heavy chain only antibody.

Additional examples of inhibitory polypeptide sequences that may be provided to inhibit the cytokine polypeptide sequence of the cytokine prodrug are anticalins, affilins, affibody molecules, affimers, affitins, alphabodies, avimers, DARPins, fynomers, kunitz domain peptides, monobodies, and binding domains based on other engineered scaffolds such as SpA, GroEL, lipocallin and CTLA4 scaffolds.

IL-2 Inhibitory Polypeptide Sequence

In cytokine prodrugs comprising an IL-2 polypeptide sequence, the inhibitory polypeptide sequence may be an IL-2 inhibitory polypeptide sequence of any of the types described above. In some embodiments, the IL-2 inhibitory polypeptide sequence is an immunoglobulin IL-2 inhibitory polypeptide sequence. In some embodiments, the IL-2 inhibitory polypeptide sequence comprises an anti-IL-2 antibody or a functional fragment thereof. In some embodiments, the immunoglobulin IL-2 inhibitory polypeptide sequence comprises a set of six anti-IL2 hypervariable regions (HVRs) set forth in Table 1 (e.g., SEQ ID NOs: 34-39 or 750-755). In some embodiments, the IL-2 inhibitory polypeptide sequence comprises a set of anti-IL2 VH and VL sequences having at least 80, 85, 90, 95, 97, 98, or 99 percent identity to the sequence of a set of anti-IL2 VH and VL sequences set forth in Table 1, either as individual sequences or as part of an scFv. In some embodiments, the IL-2 inhibitory polypeptide sequence comprises a set of anti-IL2 VH and VL sequences having the sequence of a set of anti-IL2 VH and VL sequences set forth in Table 1, either as individual sequences or as part of an scFv. Exemplary IL-2 inhibitory polypeptide sequences include SEQ ID NOS: 10-31, 40-51, and 747, and a combination of SEQ ID NOs 32 and 33 or a combination of SEQ ID NOs 748 and 749.

### Protease-Cleavable Sequence

The protease-cleavable sequence may be selected from sequences cleavable by various types of proteases, e.g., a metalloprotease, a serine protease, a cysteine protease, an aspartate protease, a threonine protease, a glutamate protease, a gelatinase, an asparagine peptide lyase, a cathepsin, a kallikrein, a plasmin, a collagenase, a hK1, a hK10, a hK15, a stromelysin, a Factor Xa, a chymotrypsin-like protease, a trypsin-like protease, a elastase-like protease, a subtilisin-like protease, an actinidain, a bromelain, a calpain, a caspase, a Mir 1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, an ADAM 10, an ADAM17, an ADAM 12, an urokinase plasminogen activator (uPA), an enterokinase, a prostate-specific target (PSA, hK3), an interleukin-1b converting enzyme, a thrombin, a FAP (FAP-a), a dipeptidyl peptidase, or dipeptidyl peptidase IV (DPPIV/CD26), a type II transmembrane serine protease (TTSP), a neutrophil elastase, a proteinase 3, a mast cell chymase, a mast cell tryptase, or a dipeptidyl peptidase. In some embodiments, the protease-cleavable sequence comprises the sequence of any one of those in Table 1 (e.g., SEQ ID NOs: 80-90 or 700-741), or a variant having one or two mismatches relative to the sequence of any one of those in Table 1 (e.g., SEQ ID NOs: 80-90 or 700-741). Proteases generally do not require an exact copy of the recognition sequence, and as such, the exemplary sequences may be varied at a portion of their amino acid positions. In some embodiments, the protease-cleavable sequence comprises a sequence that matches an MMP consensus sequence, such as any one of SEQ ID NOs: 91-94. One skilled in the art will be familiar with additional sequences recognized by these types of proteases.

Matrix Metalloprotease-Cleavable Sequence

In some embodiments, the protease-cleavable sequence is a matrix metalloprotease (MMP)-cleavable sequence. Exemplary MMP-cleavable sequences are provided in Table 1. In some embodiments, the MMP-cleavable sequence is cleavable by a plurality of MMPs and/or one or more of MMP-1, MMP-2, MMP-3, MMP-7, MMP-8, MMP-9, MMP-12, MMP-13, and/or MMP-14. Table 1, e.g., SEQ ID NOs: 80-90, provides exemplary MMP-cleavable sequences.

### Targeting Sequence

In some embodiments, the targeting sequence facilitates localization, accumulation, and/or retention of the cytokine prodrug and/or the cytokine polypeptide sequence (e.g., after proteolysis of the protease-cleavable sequence) in an area of interest, e.g., a tumor microenvironment (TME). The targeting sequence may be a sequence that binds an extracellular matrix component. Exemplary extracellular matrix components are a collagen or denatured collagen (in either case, the collagen may be collagen I, II, III, or IV), poly(I), von Willebrand factor, IgB (CD79b), heparin, a sulfated glycoprotein, or hyaluronic acid.

In other embodiments, the targeting sequence binds a target other than an extracellular matrix component. In some embodiments, the targeting sequence binds IgB (CD79b), a fibronectin, an integrin, a cadherin, a heparan sulfate proteoglycan, or a syndecan. In some embodiments, the targeting sequence binds at least one integrin, such as one or more of all integrin, α2β1 integrin, α3β1 integrin, α4β1 integrin, α5β1 integrin, α6β1 integrin, α7β1 integrin, α9β1 integrin, α4β7 integrin, αvβ3 integrin, αvβ5 integrin, αIIbβ3 integrin, αIIIbβ3 integrin, αMβ2 integrin, or αIIbβ3 integrin. In some embodiments, the targeting sequence binds at least one syndecan, such as one of more of syndecan-1, syndecan-4, and syndecan-2(w). Cytokine prodrugs comprising such targeting sequences may also comprise an MMP-cleavable linker as set forth elsewhere herein, such as an MMP-cleavable linker comprising any one of SEQ ID NOs: 80-90, or a variant having one or two mismatches relative to the sequence of any one of SEQ ID NOs: 80-90.

In some embodiments, the targeting sequence comprises a sequence set forth in Table 2 (e.g., any one of SEQ ID NOs: 180-640), or a variant having one or two mismatches relative to such a sequence.

pH-Sensitive Targeting Sequences

In some embodiments, the targeting sequence is configured to bind its target in a pH-sensitive manner. In some embodiments, the targeting sequence has a higher affinity for its target at a relatively acidic pH than at normal physiological pH (about 7.4). The higher affinity may occur at a pH below 7, e.g., in the range of pH 5.5-7, 6-7, or 5.5-6.5, or below pH 6. The presence of histidines in the targeting sequence can confer pH-sensitive binding. Without wishing to be bound by any particular theory, histidines are considered more likely to be protonated at lower pH and can render binding a negatively-charged target more energetically favorable. Accordingly, in some embodiments, a targeting sequence comprises one or more histidines, e.g., 1, 2, 3, 4, 5, 6, 7, 8, 9, or 10 histidines. Including a pH-sensitive targeting sequence can enhance discrimination between tumor versus normal tissue by the cytokine prodrug, such that the cytokine prodrug is more preferentially retained in the tumor microenvironment compared to normal extracellular matrix. Thus, a pH-sensitive targeting element can further facilitate tumor specific delivery of the cytokine prodrug and thereby further reduce or eliminate toxicity that may result from cytokine activity in normal extracellular matrix.

Binding a target in a pH-sensitive manner can be useful where it is desired to localize or retain a cytokine prodrug or the cytokine polypeptide sequence thereof in an area with a pH different from normal physiological pH. For example, the tumor microenvironment may be more acidic than the blood and/or healthy tissue. As such, binding to a target in a pH-sensitive manner may improve the retention of the cytokine prodrug or the cytokine polypeptide sequence thereof in the area of interest, which can facilitate lower doses than would otherwise be needed and/or reduce systemic exposure and/or adverse effects.

In some embodiments, the targeting sequence is configured to bind any target described herein in a pH-sensitive manner. In particular embodiments, the target is an extracellular matrix component such as a hyaluronic acid, heparin, heparan sulfate, or a sulfated glycoprotein. In another particular embodiment, the target is a fibronectin.

Exemplary targeting sequences for conferring target binding in a pH-sensitive manner are provided in Table 2 (e.g., SEQ ID NOs: 641-662). In some embodiments, the targeting sequence comprises the sequence of any one of SEQ ID NOs: 641-662, or a variant having one or two mismatches relative to the sequence of any one of SEQ ID NOs: 641-662.

### Pharmacokinetic Modulators

In some embodiments, the cytokine prodrug comprises a pharmacokinetic modulator. The pharmacokinetic modulator may be covalently or noncovalently associated with the cytokine prodrug. The pharmacokinetic modulator can extend the half-life of the cytokine prodrug and optionally the cytokine polypeptide sequence, e.g., so that fewer doses are necessary and less of the prodrug needs to be administered over time to achieve a desired result. Various forms of pharmacokinetic modulator are known in the art and may be used in cytokine prodrugs of this disclosure. In some embodiments, the pharmacokinetic modulator comprises a polypeptide (see examples below). In some embodiments, the pharmacokinetic modulator comprises a non-polypeptide moiety (e.g., polyethylene glycol, a polysaccharide, or hyaluronic acid). A non-polypeptide moiety can be associated with the prodrug using known approaches, e.g., conjugation to the prodrug; for example, a reactive amino acid residue can be used or added to the prodrug to facilitate conjugation.

In some embodiments, the pharmacokinetic modulator alters the size, shape, and/or charge of the prodrug, e.g., in a manner that reduces clearance. For example, a pharmacokinetic modulator with a negative charge may inhibit renal clearance. In some embodiments, the pharmacokinetic modulator increases the hydrodynamic volume of the prodrug. In some embodiments, the pharmacokinetic modulator reduces renal clearance, e.g., by increasing the hydrodynamic volume of the prodrug.

In some embodiments, the cytokine prodrug comprising the pharmacokinetic modulator (e.g., any of the pharmacokinetic modulators described herein) has a molecular weight of at least 70 kDa, e.g., at least 75 or 80 kDa.

For further discussion of various approaches for providing a pharmacokinetic modulator, see, e.g., Strohl, BioDrugs 29:215-19 (2015) and Podust et al., J. Controlled Release 240:52-66 (2016).

Polypeptide Pharmacokinetic Modulators

In some embodiments, the pharmacokinetic modulator comprises a polypeptide, e.g., an immunoglobulin sequence (see exemplary embodiments below), an albumin, a CTP (a negatively-charged carboxy-terminal peptide of the chorionic gonadotropin 3-chain that undergoes sialylation in vivo and in appropriate host cells), an inert polypeptide (e.g., an unstructured polypeptide such as an XTEN, a polypeptide comprising the residues Ala, Glu, Gly, Pro, Ser, and Thr), a transferrin, a homo-amino-acid polypeptide, or an elastin-like polypeptide.

Exemplary polypeptide sequences suitable for use as a pharmacokinetic modulator are provided in Table 1 (e.g., any one of SEQ ID NOs: 70-74). In some embodiments, the pharmacokinetic modulator has at least 80, 85, 90, 95, 97, 98, or 99 percent identity to the sequence of a pharmacokinetic modulator in Table 1 (e.g., any one of SEQ ID NOs: 70-74).

In any embodiment where the pharmacokinetic modulator comprises a polypeptide sequence from an organism, the polypeptide sequence may be a human polypeptide sequence.

Immunoglobulin Pharmacokinetic Modulators

In some embodiments, the pharmacokinetic modulator comprises an immunoglobulin sequence, e.g., one or more immunoglobulin constant domains. In some embodiments, the pharmacokinetic modulator comprises an Fc region. The immunoglobulin sequence (e.g., one or more immunoglobulin constant domains or Fc region) may be a human immunoglobulin sequence. The immunoglobulin sequence (e.g., one or more immunoglobulin constant domains or Fc region) may have has at least 80, 85, 90, 95, 97, 98, or 99 percent identity to the sequence of a wild-type immunoglobulin sequence (e.g., one or more immunoglobulin constant domains or Fc region), such as a wild-type human immunoglobulin sequence. In any of such embodiments, the immunoglobulin sequence may be an IgG sequence (e.g., IgG1, IgG2, IgG3, or IgG4). Exemplary immunoglobulin pharmacokinetic modulator sequences include SEQ ID NOS: 70-74 and the combination of SEQ ID NOs 756 and 757.

### Arrangement of Components

The recitation of components of a cytokine prodrug herein does not imply any particular order beyond what is explicitly stated (for example, it may be explicitly stated that a protease-cleavable sequence is between the cytokine polypeptide sequence and the inhibitory polypeptide sequence). The components of the cytokine prodrug may be arranged in various ways to provide properties suitable for a particular use. The components of the cytokine prodrug may be all in one polypeptide chain or they may be in a plurality of polypeptide chains bridged by covalent bonds, such as disulfide bonds. For example, where a pharmacokinetic modulator comprises an Fc, one or more components may be bound to one chain while one or more other components may be bound to the other chain. The Fc may be a heterodimeric Fe, such as a knob-into-hole Fc (in which one chain of the Fc comprises knob mutations and the other chain of the Fc comprises hole mutations). For an exemplary general discussion of knob and hole mutations, see, e.g., Xu et al., mAbs 7:1, 231-242 (2015). Exemplary knob mutations (e.g., for a human IgG1 Fc) are K360E/K409W. Exemplary hole mutations (e.g., for a human IgG1 Fc) are Q347R/D399V/F405T. See SEQ ID NOs: 756 and 757.

For example, a pharmacokinetic modulator can be present on the same side of the protease-cleavable sequence as the cytokine polypeptide sequence, meaning that cleavage of the protease-cleavable sequence does not separate the pharmacokinetic modulator from the cytokine polypeptide sequence. Examples of such structures include CY-PM-CL-IN, IN-CL-CY-PM, and any other permutation (or variation in which additional elements are included between, before, or after the listed components) in which CL is not between CY and PM, where CY is the cytokine polypeptide sequence, PM is the pharmacokinetic modulator, CL is the protease-cleavable sequence, and IN is the inhibitory polypeptide sequence. In such embodiments, the pharmacokentic modulator will modulate the pharmacokinetics of both the prodrug and the active cytokine polypeptide sequence. In some embodiments, the pharmacokinetic modulator is an Fc, in which case the components preceding and following PM in the exemplary structures above may be bound to the same or different chains of the Fc, as discussed above.

In some embodiments, a pharmacokinetic modulator is present on the same side of the protease-cleavable sequence as the inhibitory polypeptide sequence, meaning that cleavage of the protease-cleavable sequence does separate the pharmacokinetic modulator from the cytokine polypeptide sequence. Such embodiments can be useful to provide a longer half-life for the prodrug than for the active form.

In some embodiments, a targeting sequence can be present on the same side of the protease-cleavable sequence as the cytokine polypeptide sequence, meaning that cleavage of the protease-cleavable sequence does not separate the targeting sequence from the cytokine polypeptide sequence. Such embodiments can be useful to facilitate localizing or retaining both the prodrug and the active form in an area of interest, e.g., a tumor microenvironment. Where a pharmacokinetic modulator is used, it can be on the same side of the protease-cleavable linker as the targeting sequence (e.g., to facilitate lower and/or less frequent dosing) or on the other side (e.g., to avoid long-duration immune stimulation), depending on the desired effects.

In some embodiments, a targeting sequence is present on the same side of the protease-cleavable sequence as the inhibitory polypeptide sequence, meaning that cleavage of the protease-cleavable sequence does separate the targeting sequence from the cytokine polypeptide sequence. Such embodiments can be useful to provide a gradient of cytokine emanating from an area of interest, or to provide such a gradient more rapidly than would occur if the targeting sequence were on the same side of the protease-cleavable sequence. Where a pharmacokinetic modulator is used, it can be on the same side of the protease-cleavable linker as the targeting sequence (e.g., to minimize systemic exposure to the active form of the cytokine and/or avoid long-duration immune stimulation) or on the other side (e.g., to facilitate lower and/or less frequent dosing), depending on the desired effects.

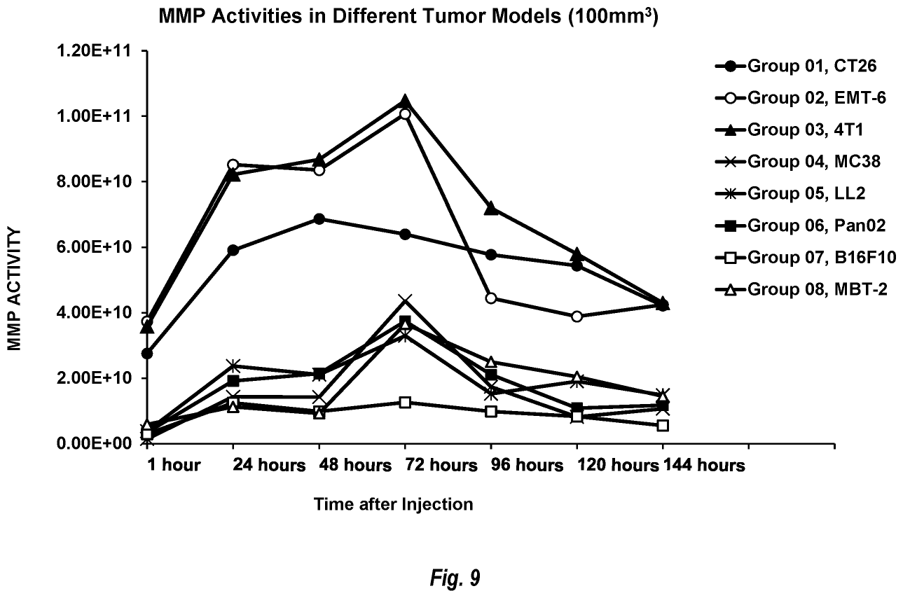

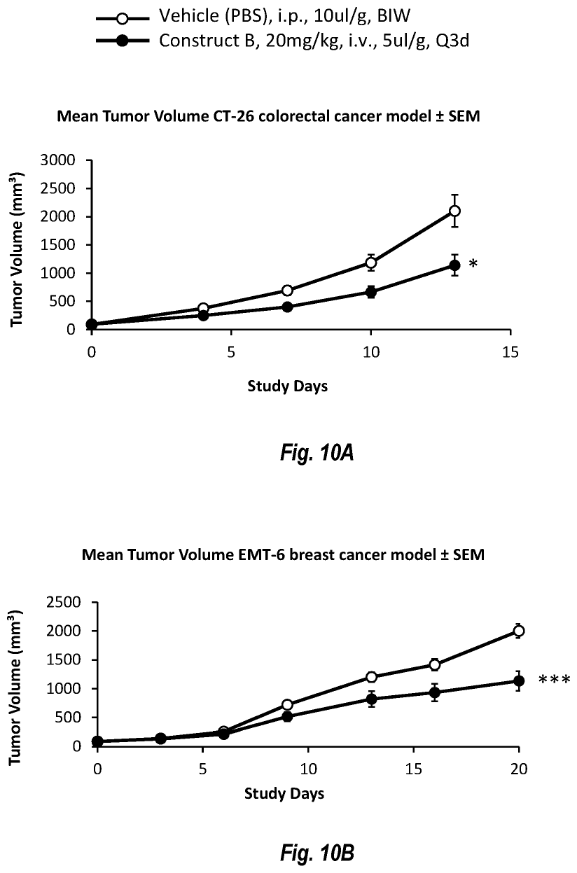

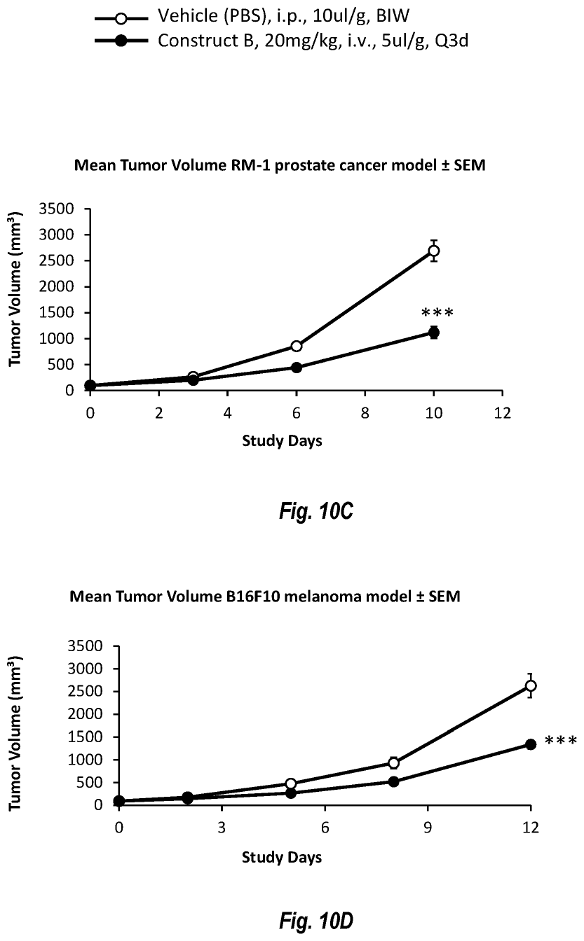

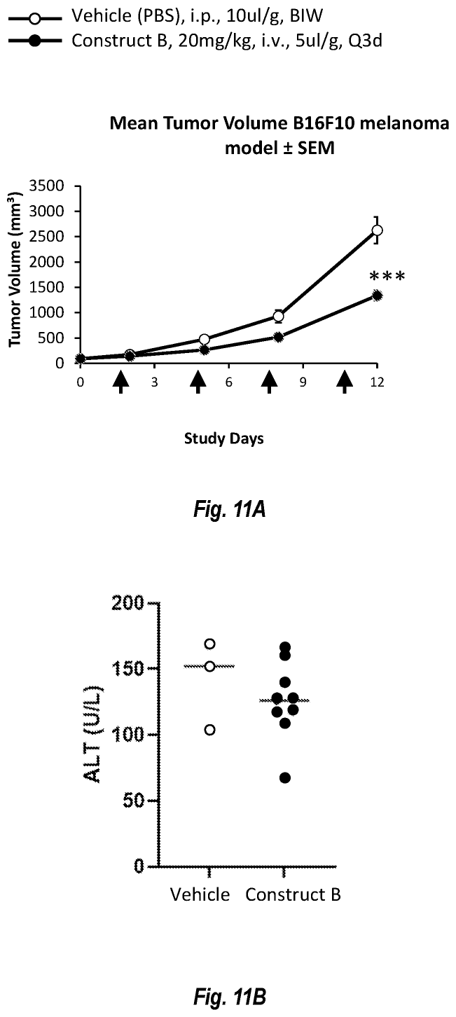

A number of exemplary arrangements are illustrated in FIGS. 9 and 10A-E. In some embodiments, the cytokine prodrug comprises components arranged according to any of the examples in FIGS. 9 and 10A-E, ordered from N- to C-terminus or from C- to N-terminus, optionally with additional components inserted between any of the illustrated components.

### Exemplary Prodrugs

IL-2

The following table shows exemplary combinations of components according to certain embodiments of the disclosed cytokine prodrugs. The numbers indicate SEQ ID NOs for a given component. CY is the cytokine polypeptide sequence, CL is the protease-cleavable sequence, and IN is the inhibitory polypeptide sequence, and, where present, PM is the pharmacokinetic modulator. Where a range is given, any one of the listed SEQ ID NOs may be selected. Where two SEQ ID NOs are recited conjunctively (using “and”), both SEQ ID NOs are present and can function together (they may or may not be fused to each other, optionally with an intervening linker, or bridged, e.g., by a covalent bond). For example, SEQ ID NOs 32 and 33 are VL and VH domains that can function together to form a cytokine-binding immunoglobulin domain, as are SEQ ID NOs 748 and 749. SEQ ID NOs 256 and 257 are Fc polypeptide chains for forming a heterodimeric knob-into-hole Fc that can serve as a pharmacokinetic modulator. The components may be arranged in any manner consistent with the disclosure, e.g., as indicated elsewhere herein. In some embodiments, a cytokine prodrug comprises a combination of sequences as set forth in Table 3A.

TABLE 3A
Exemplary IL-2 prodrugs

| CY | CL | IN | PM |
|---|---|---|---|
| 1-2 | 80-90 or | 10-16, 30, 31, 40-51, 747, | |
| | 201-242 | (32 and 33), or (748 and 749) | |
| 1-2 | 80-90 or | 10-16, 30, 31, 40-51, 747, | 70-74 or |
| | 201-242 | (32 and 33), or (748 and 749) | (756 and 757) |
| 1 | 80-90 or | 10 | |
| | 201-242 | | |
| 1 | 80-90 or | 11 | |
| | 201-242 | | |
| 1 | 80-90 or | 12 | |
| | 201-242 | | |
| 1 | 80-90 or | 13 | |
| | 201-242 | | |
| 1 | 80-90 or | 14 | |
| | 201-242 | | |
| 1 | 80-90 or | 15 | |
| | 201-242 | | |
| 1 | 80-90 or | 16 | |
| | 201-242 | | |
| 1 | 80-90 or | 20 | |
| | 201-242 | | |
| 1 | 80-90 or | 21 | |
| | 201-242 | | |
| 1 | 80-90 or | 22 | |
| | 201-242 | | |
| 1 | 80-90 or | 23 | |
| | 201-242 | | |
| 1 | 80-90 or | 24 | |
| | 201-242 | | |
| 1 | 80-90 or | 25 | |
| | 201-242 | | |
| 1 | 80-90 or | 26 | |
| | 201-242 | | |
| 1 | 80-90 or | 27 | |
| | 201-242 | | |
| 1 | 80-90 or | 28 | |
| | 201-242 | | |
| 1 | 80-90 or | 29 | |
| | 201-242 | | |
| 1 | 80-90 or | 30 | |
| | 201-242 | | |
| 1 | 80-90 or | 31 | |
| | 201-242 | | |
| 1 | 80-90 or | 32 and 33 | |
| | 201-242 | | |
| 1 | 80-90 or | 40 | |
| | 201-242 | | |
| 1 | 80-90 or | 41 | |
| | 201-242 | | |
| 1 | 80-90 or | 42 | |
| | 201-242 | | |
| 1 | 80-90 or | 43 | |
| | 201-242 | | |
| 1 | 80-90 or | 44 | |
| | 201-242 | | |
| 1 | 80-90 or | 45 | |
| | 201-242 | | |
| 1 | 80-90 or | 46 | |
| | 201-242 | | |
| 1 | 80-90 or | 47 | |
| | 201-242 | | |
| 1 | 80-90 or | 48 | |
| | 201-242 | | |
| 1 | 80-90 or | 49 | |
| | 201-242 | | |
| 1 | 80-90 or | 50 | |
| | 201-242 | | |
| 1 | 80-90 or | 51 | |
| | 201-242 | | |
| 1 | 80-90 or | 747 | |
| | 201-242 | | |
| 1 | 80-90 or | 748 and 749 | |
| | 201-242 | | |
| 1 | 80-90 or | 10 | 70 |
| | 201-242 | | |
| 1 | 80-90 or | 10 | 71 |
| | 201-242 | | |
| 1 | 80-90 or | 10 | 72 |
| | 201-242 | | |
| 1 | 80-90 or | 10 | 73 |
| | 201-242 | | |
| 1 | 80-90 or | 10 | 74 |
| | 201-242 | | |
| 1 | 80-90 or | 10 | 756 and 757 |
| | 201-242 | | |
| 1 | 80-90 or | 11 | 70 |
| | 201-242 | | |
| 1 | 80-90 or | 11 | 71 |
| | 201-242 | | |
| 1 | 80-90 or | 11 | 72 |
| | 201-242 | | |
| 1 | 80-90 or | 11 | 73 |
| | 201-242 | | |
| 1 | 80-90 or | 11 | 74 |
| | 201-242 | | |
| 1 | 80-90 or | 11 | 756 and 757 |
| | 201-242 | | |
| 1 | 80-90 or | 12 | 70 |
| | 201-242 | | |
| 1 | 80-90 or | 12 | 71 |
| | 201-242 | | |
| 1 | 80-90 or | 12 | 72 |
| | 201-242 | | |
| 1 | 80-90 or | 12 | 73 |
| | 201-242 | | |
| 1 | 80-90 or | 12 | 74 |
| | 201-242 | | |
| 1 | 80-90 or | 12 | 756 and 757 |
| | 201-242 | | |
| 1 | 80-90 or | 13 | 70 |
| | 201-242 | | |
| 1 | 80-90 or | 13 | 71 |
| | 201-242 | | |
| 1 | 80-90 or | 13 | 72 |
| | 201-242 | | |
| 1 | 80-90 or | 13 | 73 |
| | 201-242 | | |
| 1 | 80-90 or | 13 | 74 |
| | 201-242 | | |
| 1 | 80-90 or | 13 | 756 and 757 |
| | 201-242 | | |
| 1 | 80-90 or | 14 | 70 |
| | 201-242 | | |
| 1 | 80-90 or | 14 | 71 |
| | 201-242 | | |
| 1 | 80-90 or | 14 | 72 |
| | 201-242 | | |
| 1 | 80-90 or | 14 | 73 |
| | 201-242 | | |
| 1 | 80-90 or | 14 | 74 |
| | 201-242 | | |
| 1 | 80-90 or | 14 | 756 and 757 |
| | 201-242 | | |
| 1 | 80-90 or | 15 | 70 |
| | 201-242 | | |
| 1 | 80-90 or | 15 | 71 |
| | 201-242 | | |
| 1 | 80-90 or | 15 | 72 |
| | 201-242 | | |
| 1 | 80-90 or | 15 | 73 |
| | 201-242 | | |
| 1 | 80-90 or | 15 | 74 |
| | 201-242 | | |
| 1 | 80-90 or | 15 | 756 and 757 |
| | 201-242 | | |
| 1 | 80-90 or | 16 | 70 |
| | 201-242 | | |
| 1 | 80-90 or | 16 | 71 |
| | 201-242 | | |
| 1 | 80-90 or | 16 | 72 |
| | 201-242 | | |
| 1 | 80-90 or | 16 | 73 |
| | 201-242 | | |
| 1 | 80-90 or | 16 | 74 |
| | 201-242 | | |
| 1 | 80-90 or | 16 | 756 and 757 |
| | 201-242 | | |
| 1 | 80-90 or | 20 | 70 |
| | 201-242 | | |
| 1 | 80-90 or | 20 | 71 |
| | 201-242 | | |
| 1 | 80-90 or | 20 | 72 |
| | 201-242 | | |
| 1 | 80-90 or | 20 | 73 |
| | 201-242 | | |
| 1 | 80-90 or | 20 | 74 |
| | 201-242 | | |
| 1 | 80-90 or | 20 | 756 and 757 |
| | 201-242 | | |
| 1 | 80-90 or | 21 | 70 |
| | 201-242 | | |
| 1 | 80-90 or | 21 | 71 |
| | 201-242 | | |
| 1 | 80-90 or | 21 | 72 |
| | 201-242 | | |
| 1 | 80-90 or | 21 | 73 |
| | 201-242 | | |
| 1 | 80-90 or | 21 | 74 |
| | 201-242 | | |
| 1 | 80-90 or | 21 | 756 and 757 |
| | 201-242 | | |
| 1 | 80-90 or | 22 | 70 |
| | 201-242 | | |
| 1 | 80-90 or | 22 | 71 |
| | 201-242 | | |
| 1 | 80-90 or | 22 | 72 |
| | 201-242 | | |
| 1 | 80-90 or | 22 | 73 |
| | 201-242 | | |
| 1 | 80-90 or | 22 | 74 |
| | 201-242 | | |
| 1 | 80-90 or | 22 | 756 and 757 |
| | 201-242 | | |
| 1 | 80-90 or | 23 | 70 |
| | 201-242 | | |
| 1 | 80-90 or | 23 | 71 |
| | 201-242 | | |
| 1 | 80-90 or | 23 | 72 |
| | 201-242 | | |
| 1 | 80-90 or | 23 | 73 |
| | 201-242 | | |
| 1 | 80-90 or | 23 | 74 |
| | 201-242 | | |
| 1 | 80-90 or | 23 | 756 and 757 |
| | 201-242 | | |
| 1 | 80-90 or | 24 | 70 |
| | 201-242 | | |
| 1 | 80-90 or | 24 | 71 |
| | 201-242 | | |
| 1 | 80-90 or | 24 | 72 |
| | 201-242 | | |
| 1 | 80-90 or | 24 | 73 |
| | 201-242 | | |
| 1 | 80-90 or | 24 | 74 |
| | 201-242 | | |
| 1 | 80-90 or | 24 | 756 and 757 |
| | 201-242 | | |
| 1 | 80-90 or | 25 | 70 |
| | 201-242 | | |
| 1 | 80-90 or | 25 | 71 |
| | 201-242 | | |
| 1 | 80-90 or | 25 | 72 |
| | 201-242 | | |
| 1 | 80-90 or | 25 | 73 |
| | 201-242 | | |
| 1 | 80-90 or | 25 | 74 |
| | 201-242 | | |
| 1 | 80-90 or | 25 | 756 and 757 |
| | 201-242 | | |
| 1 | 80-90 or | 26 | 70 |
| | 201-242 | | |
| 1 | 80-90 or | 26 | 71 |
| | 201-242 | | |
| 1 | 80-90 or | 26 | 72 |
| | 201-242 | | |
| 1 | 80-90 or | 26 | 73 |
| | 201-242 | | |
| 1 | 80-90 or | 26 | 74 |
| | 201-242 | | |
| 1 | 80-90 or | 26 | 756 and 757 |
| | 201-242 | | |
| 1 | 80-90 or | 27 | 70 |
| | 201-242 | | |
| 1 | 80-90 or | 27 | 71 |
| | 201-242 | | |
| 1 | 80-90 or | 27 | 72 |
| | 201-242 | | |
| 1 | 80-90 or | 27 | 73 |
| | 201-242 | | |
| 1 | 80-90 or | 27 | 74 |
| | 201-242 | | |
| 1 | 80-90 or | 27 | 756 and 757 |
| | 201-242 | | |
| 1 | 80-90 or | 28 | 70 |
| | 201-242 | | |
| 1 | 80-90 or | 28 | 71 |
| | 201-242 | | |
| 1 | 80-90 or | 28 | 72 |
| | 201-242 | | |
| 1 | 80-90 or | 28 | 73 |
| | 201-242 | | |
| 1 | 80-90 or | 28 | 74 |
| | 201-242 | | |
| 1 | 80-90 or | 28 | 756 and 757 |
| | 201-242 | | |
| 1 | 80-90 or | 29 | 70 |
| | 201-242 | | |
| 1 | 80-90 or | 29 | 71 |
| | 201-242 | | |
| 1 | 80-90 or | 29 | 72 |
| | 201-242 | | |
| 1 | 80-90 or | 29 | 73 |
| | 201-242 | | |
| 1 | 80-90 or | 29 | 74 |
| | 201-242 | | |
| 1 | 80-90 or | 29 | 756 and 757 |
| | 201-242 | | |
| 1 | 80-90 or | 30 | 70 |
| | 201-242 | | |
| 1 | 80-90 or | 30 | 71 |
| | 201-242 | | |
| 1 | 80-90 or | 30 | 72 |
| | 201-242 | | |
| 1 | 80-90 or | 30 | 73 |
| | 201-242 | | |
| 1 | 80-90 or | 30 | 74 |
| | 201-242 | | |
| 1 | 80-90 or | 30 | 756 and 757 |
| | 201-242 | | |
| 1 | 80-90 or | 31 | 70 |
| | 201-242 | | |
| 1 | 80-90 or | 31 | 71 |
| | 201-242 | | |
| 1 | 80-90 or | 31 | 72 |
| | 201-242 | | |
| 1 | 80-90 or | 31 | 73 |
| | 201-242 | | |
| 1 | 80-90 or | 31 | 74 |
| | 201-242 | | |
| 1 | 80-90 or | 31 | 756 and 757 |
| | 201-242 | | |
| 1 | 80-90 or | 32 and 33 | 70 |
| | 201-242 | | |
| 1 | 80-90 or | 32 and 33 | 71 |
| | 201-242 | | |
| 1 | 80-90 or | 32 and 33 | 72 |
| | 201-242 | | |
| 1 | 80-90 or | 32 and 33 | 73 |
| | 201-242 | | |
| 1 | 80-90 or | 32 and 33 | 74 |
| | 201-242 | | |
| 1 | 80-90 or | 32 and 33 | 756 and 757 |
| | 201-242 | | |
| 1 | 80-90 or | 40 | 70 |
| | 201-242 | | |
| 1 | 80-90 or | 40 | 71 |
| | 201-242 | | |
| 1 | 80-90 or | 40 | 72 |
| | 201-242 | | |
| 1 | 80-90 or | 40 | 73 |
| | 201-242 | | |
| 1 | 80-90 or | 40 | 74 |
| | 201-242 | | |
| 1 | 80-90 or | 40 | 756 and 757 |
| | 201-242 | | |
| 1 | 80-90 or | 41 | 70 |
| | 201-242 | | |
| 1 | 80-90 or | 41 | 71 |
| | 201-242 | | |
| 1 | 80-90 or | 41 | 72 |
| | 201-242 | | |
| 1 | 80-90 or | 41 | 73 |
| | 201-242 | | |
| 1 | 80-90 or | 41 | 74 |
| | 201-242 | | |
| 1 | 80-90 or | 41 | 756 and 757 |
| | 201-242 | | |
| 1 | 80-90 or | 42 | 70 |
| | 201-242 | | |
| 1 | 80-90 or | 42 | 71 |
| | 201-242 | | |
| 1 | 80-90 or | 42 | 72 |
| | 201-242 | | |
| 1 | 80-90 or | 42 | 73 |
| | 201-242 | | |
| 1 | 80-90 or | 42 | 74 |
| | 201-242 | | |
| 1 | 80-90 or | 42 | 756 and 757 |
| | 201-242 | | |
| 1 | 80-90 or | 43 | 70 |
| | 201-242 | | |
| 1 | 80-90 or | 43 | 71 |
| | 201-242 | | |
| 1 | 80-90 or | 43 | 72 |
| | 201-242 | | |
| 1 | 80-90 or | 43 | 73 |
| | 201-242 | | |
| 1 | 80-90 or | 43 | 74 |
| | 201-242 | | |
| 1 | 80-90 or | 43 | 756 and 757 |
| | 201-242 | | |
| 1 | 80-90 or | 44 | 70 |
| | 201-242 | | |
| 1 | 80-90 or | 44 | 71 |
| | 201-242 | | |
| 1 | 80-90 or | 44 | 72 |
| | 201-242 | | |
| 1 | 80-90 or | 44 | 73 |
| | 201-242 | | |
| 1 | 80-90 or | 44 | 74 |
| | 201-242 | | |
| 1 | 80-90 or | 44 | 756 and 757 |
| | 201-242 | | |
| 1 | 80-90 or | 45 | 70 |
| | 201-242 | | |
| 1 | 80-90 or | 45 | 71 |
| | 201-242 | | |
| 1 | 80-90 or | 45 | 72 |
| | 201-242 | | |
| 1 | 80-90 or | 45 | 73 |
| | 201-242 | | |
| 1 | 80-90 or | 45 | 74 |
| | 201-242 | | |
| 1 | 80-90 or | 45 | 756 and 757 |
| | 201-242 | | |
| 1 | 80-90 or | 46 | 70 |
| | 201-242 | | |
| 1 | 80-90 or | 46 | 71 |
| | 201-242 | | |
| 1 | 80-90 or | 46 | 72 |
| | 201-242 | | |
| 1 | 80-90 or | 46 | 73 |
| | 201-242 | | |
| 1 | 80-90 or | 46 | 74 |
| | 201-242 | | |
| 1 | 80-90 or | 46 | 756 and 757 |
| | 201-242 | | |
| 1 | 80-90 or | 47 | 70 |
| | 201-242 | | |
| 1 | 80-90 or | 47 | 71 |
| | 201-242 | | |
| 1 | 80-90 or | 47 | 72 |
| | 201-242 | | |
| 1 | 80-90 or | 47 | 73 |
| | 201-242 | | |
| 1 | 80-90 or | 47 | 74 |
| | 201-242 | | |
| 1 | 80-90 or | 47 | 756 and 757 |
| | 201-242 | | |
| 1 | 80-90 or | 48 | 70 |
| | 201-242 | | |
| 1 | 80-90 or | 48 | 71 |
| | 201-242 | | |
| 1 | 80-90 or | 48 | 72 |
| | 201-242 | | |
| 1 | 80-90 or | 48 | 73 |
| | 201-242 | | |
| 1 | 80-90 or | 48 | 74 |
| | 201-242 | | |
| 1 | 80-90 or | 48 | 756 and 757 |
| | 201-242 | | |
| 1 | 80-90 or | 49 | 70 |
| | 201-242 | | |
| 1 | 80-90 or | 49 | 71 |
| | 201-242 | | |
| 1 | 80-90 or | 49 | 72 |
| | 201-242 | | |
| 1 | 80-90 or | 49 | 73 |
| | 201-242 | | |
| 1 | 80-90 or | 49 | 74 |
| | 201-242 | | |
| 1 | 80-90 or | 49 | 756 and 757 |
| | 201-242 | | |
| 1 | 80-90 or | 50 | 70 |
| | 201-242 | | |
| 1 | 80-90 or | 50 | 71 |
| | 201-242 | | |
| 1 | 80-90 or | 50 | 72 |
| | 201-242 | | |
| 1 | 80-90 or | 50 | 73 |
| | 201-242 | | |
| 1 | 80-90 or | 50 | 74 |
| | 201-242 | | |
| 1 | 80-90 or | 50 | 756 and 757 |
| | 201-242 | | |
| 1 | 80-90 or | 51 | 70 |
| | 201-242 | | |
| 1 | 80-90 or | 51 | 71 |
| | 201-242 | | |
| 1 | 80-90 or | 51 | 72 |
| | 201-242 | | |
| 1 | 80-90 or | 51 | 73 |
| | 201-242 | | |
| 1 | 80-90 or | 51 | 74 |
| | 201-242 | | |
| 1 | 80-90 or | 51 | 756 and 757 |
| | 201-242 | | |
| 1 | 80-90 or | 747 | 70 |
| | 201-242 | | |
| 1 | 80-90 or | 747 | 71 |
| | 201-242 | | |
| 1 | 80-90 or | 747 | 72 |
| | 201-242 | | |
| 1 | 80-90 or | 747 | 73 |
| | 201-242 | | |
| 1 | 80-90 or | 747 | 74 |
| | 201-242 | | |
| 1 | 80-90 or | 747 | 756 and 757 |
| | 201-242 | | |
| 1 | 80-90 or | 748 and 749 | 70 |
| | 201-242 | | |
| 1 | 80-90 or | 748 and 749 | 71 |
| | 201-242 | | |
| 1 | 80-90 or | 748 and 749 | 72 |
| | 201-242 | | |
| 1 | 80-90 or | 748 and 749 | 73 |
| | 201-242 | | |
| 1 | 80-90 or | 748 and 749 | 74 |
| | 201-242 | | |
| 1 | 80-90 or | 748 and 749 | 756 and 757 |
| | 201-242 | | |
| 2 | 80-90 or | 10 | |
| | 201-242 | | |
| 2 | 80-90 or | 11 | |
| | 201-242 | | |
| 2 | 80-90 or | 12 | |
| | 201-242 | | |
| 2 | 80-90 or | 13 | |
| | 201-242 | | |
| 2 | 80-90 or | 14 | |
| | 201-242 | | |
| 2 | 80-90 or | 15 | |
| | 201-242 | | |
| 2 | 80-90 or | 16 | |
| | 201-242 | | |
| 2 | 80-90 or | 20 | |
| | 201-242 | | |
| 2 | 80-90 or | 21 | |
| | 201-242 | | |
| 2 | 80-90 or | 22 | |
| | 201-242 | | |
| 2 | 80-90 or | 23 | |
| | 201-242 | | |
| 2 | 80-90 or | 24 | |
| | 201-242 | | |
| 2 | 80-90 or | 25 | |
| | 201-242 | | |
| 2 | 80-90 or | 26 | |
| | 201-242 | | |
| 2 | 80-90 or | 27 | |
| | 201-242 | | |
| 2 | 80-90 or | 28 | |
| | 201-242 | | |
| 2 | 80-90 or | 29 | |
| | 201-242 | | |
| 2 | 80-90 or | 30 | |
| | 201-242 | | |
| 2 | 80-90 or | 31 | |
| | 201-242 | | |
| 2 | 80-90 or | 32 and 33 | |
| | 201-242 | | |
| 2 | 80-90 or | 40 | |
| | 201-242 | | |
| 2 | 80-90 or | 41 | |
| | 201-242 | | |
| 2 | 80-90 or | 42 | |
| | 201-242 | | |
| 2 | 80-90 or | 43 | |
| | 201-242 | | |
| 2 | 80-90 or | 44 | |
| | 201-242 | | |
| 2 | 80-90 or | 45 | |
| | 201-242 | | |
| 2 | 80-90 or | 46 | |
| | 201-242 | | |
| 2 | 80-90 or | 47 | |
| | 201-242 | | |
| 2 | 80-90 or | 48 | |
| | 201-242 | | |
| 2 | 80-90 or | 49 | |
| | 201-242 | | |
| 2 | 80-90 or | 50 | |
| | 201-242 | | |
| 2 | 80-90 or | 51 | |
| | 201-242 | | |
| 2 | 80-90 or | 747 | |
| | 201-242 | | |
| 2 | 80-90 or | 748 and 749 | |
| | 201-242 | | |
| 2 | 80-90 or | 10 | 70 |
| | 201-242 | | |
| 2 | 80-90 or | 10 | 71 |
| | 201-242 | | |
| 2 | 80-90 or | 10 | 72 |
| | 201-242 | | |
| 2 | 80-90 or | 10 | 73 |
| | 201-242 | | |
| 2 | 80-90 or | 10 | 74 |
| | 201-242 | | |
| 2 | 80-90 or | 10 | 756 and 757 |
| | 201-242 | | |
| 2 | 80-90 or | 11 | 70 |
| | 201-242 | | |
| 2 | 80-90 or | 11 | 71 |
| | 201-242 | | |
| 2 | 80-90 or | 11 | 72 |
| | 201-242 | | |
| 2 | 80-90 or | 11 | 73 |
| | 201-242 | | |
| 2 | 80-90 or | 11 | 74 |
| | 201-242 | | |
| 2 | 80-90 or | 11 | 756 and 757 |
| | 201-242 | | |
| 2 | 80-90 or | 12 | 70 |
| | 201-242 | | |
| 2 | 80-90 or | 12 | 71 |
| | 201-242 | | |
| 2 | 80-90 or | 12 | 72 |
| | 201-242 | | |
| 2 | 80-90 or | 12 | 73 |
| | 201-242 | | |
| 2 | 80-90 or | 12 | 74 |
| | 201-242 | | |
| 2 | 80-90 or | 12 | 756 and 757 |
| | 201-242 | | |
| 2 | 80-90 or | 13 | 70 |
| | 201-242 | | |
| 2 | 80-90 or | 13 | 71 |
| | 201-242 | | |
| 2 | 80-90 or | 13 | 72 |
| | 201-242 | | |
| 2 | 80-90 or | 13 | 73 |
| | 201-242 | | |
| 2 | 80-90 or | 13 | 74 |
| | 201-242 | | |
| 2 | 80-90 or | 13 | 756 and 757 |
| | 201-242 | | |
| 2 | 80-90 or | 14 | 70 |
| | 201-242 | | |
| 2 | 80-90 or | 14 | 71 |
| | 201-242 | | |
| 2 | 80-90 or | 14 | 72 |
| | 201-242 | | |
| 2 | 80-90 or | 14 | 73 |
| | 201-242 | | |
| 2 | 80-90 or | 14 | 74 |
| | 201-242 | | |
| 2 | 80-90 or | 14 | 756 and 757 |
| | 201-242 | | |
| 2 | 80-90 or | 15 | 70 |
| | 201-242 | | |
| 2 | 80-90 or | 15 | 71 |
| | 201-242 | | |
| 2 | 80-90 or | 15 | 72 |
| | 201-242 | | |
| 2 | 80-90 or | 15 | 73 |
| | 201-242 | | |
| 2 | 80-90 or | 15 | 74 |
| | 201-242 | | |
| 2 | 80-90 or | 15 | 756 and 757 |
| | 201-242 | | |
| 2 | 80-90 or | 16 | 70 |
| | 201-242 | | |
| 2 | 80-90 or | 16 | 71 |
| | 201-242 | | |
| 2 | 80-90 or | 16 | 72 |
| | 201-242 | | |
| 2 | 80-90 or | 16 | 73 |
| | 201-242 | | |
| 2 | 80-90 or | 16 | 74 |
| | 201-242 | | |
| 2 | 80-90 or | 16 | 756 and 757 |
| | 201-242 | | |
| 2 | 80-90 or | 20 | 70 |
| | 201-242 | | |
| 2 | 80-90 or | 20 | 71 |
| | 201-242 | | |
| 2 | 80-90 or | 20 | 72 |
| | 201-242 | | |
| 2 | 80-90 or | 20 | 73 |
| | 201-242 | | |
| 2 | 80-90 or | 20 | 74 |
| | 201-242 | | |
| 2 | 80-90 or | 20 | 756 and 757 |
| | 201-242 | | |
| 2 | 80-90 or | 21 | 70 |
| | 201-242 | | |
| 2 | 80-90 or | 21 | 71 |
| | 201-242 | | |
| 2 | 80-90 or | 21 | 72 |
| | 201-242 | | |
| 2 | 80-90 or | 21 | 73 |
| | 201-242 | | |
| 2 | 80-90 or | 21 | 74 |
| | 201-242 | | |
| 2 | 80-90 or | 21 | 756 and 757 |
| | 201-242 | | |
| 2 | 80-90 or | 22 | 70 |
| | 201-242 | | |
| 2 | 80-90 or | 22 | 71 |
| | 201-242 | | |
| 2 | 80-90 or | 22 | 72 |
| | 201-242 | | |
| 2 | 80-90 or | 22 | 73 |
| | 201-242 | | |
| 2 | 80-90 or | 22 | 74 |
| | 201-242 | | |
| 2 | 80-90 or | 22 | 756 and 757 |
| | 201-242 | | |
| 2 | 80-90 or | 23 | 70 |
| | 201-242 | | |
| 2 | 80-90 or | 23 | 71 |
| | 201-242 | | |
| 2 | 80-90 or | 23 | 72 |
| | 201-242 | | |
| 2 | 80-90 or | 23 | 73 |
| | 201-242 | | |
| 2 | 80-90 or | 23 | 74 |
| | 201-242 | | |
| 2 | 80-90 or | 23 | 756 and 757 |
| | 201-242 | | |
| 2 | 80-90 or | 24 | 70 |
| | 201-242 | | |
| 2 | 80-90 or | 24 | 71 |
| | 201-242 | | |
| 2 | 80-90 or | 24 | 72 |
| | 201-242 | | |
| 2 | 80-90 or | 24 | 73 |
| | 201-242 | | |
| 2 | 80-90 or | 24 | 74 |
| | 201-242 | | |
| 2 | 80-90 or | 24 | 756 and 757 |
| | 201-242 | | |
| 2 | 80-90 or | 25 | 70 |
| | 201-242 | | |
| 2 | 80-90 or | 25 | 71 |
| | 201-242 | | |
| 2 | 80-90 or | 25 | 72 |
| | 201-242 | | |
| 2 | 80-90 or | 25 | 73 |
| | 201-242 | | |
| 2 | 80-90 or | 25 | 74 |
| | 201-242 | | |
| 2 | 80-90 or | 25 | 756 and 757 |
| | 201-242 | | |
| 2 | 80-90 or | 26 | 70 |
| | 201-242 | | |
| 2 | 80-90 or | 26 | 71 |
| | 201-242 | | |
| 2 | 80-90 or | 26 | 72 |
| | 201-242 | | |
| 2 | 80-90 or | 26 | 73 |
| | 201-242 | | |
| 2 | 80-90 or | 26 | 74 |
| | 201-242 | | |
| 2 | 80-90 or | 26 | 756 and 757 |
| | 201-242 | | |
| 2 | 80-90 or | 27 | 70 |
| | 201-242 | | |
| 2 | 80-90 or | 27 | 71 |
| | 201-242 | | |
| 2 | 80-90 or | 27 | 72 |
| | 201-242 | | |
| 2 | 80-90 or | 27 | 73 |
| | 201-242 | | |
| 2 | 80-90 or | 27 | 74 |
| | 201-242 | | |
| 2 | 80-90 or | 27 | 756 and 757 |
| | 201-242 | | |
| 2 | 80-90 or | 28 | 70 |
| | 201-242 | | |
| 2 | 80-90 or | 28 | 71 |
| | 201-242 | | |
| 2 | 80-90 or | 28 | 72 |
| | 201-242 | | |
| 2 | 80-90 or | 28 | 73 |
| | 201-242 | | |
| 2 | 80-90 or | 28 | 74 |
| | 201-242 | | |
| 2 | 80-90 or | 28 | 756 and 757 |
| | 201-242 | | |
| 2 | 80-90 or | 29 | 70 |
| | 201-242 | | |
| 2 | 80-90 or | 29 | 71 |
| | 201-242 | | |
| 2 | 80-90 or | 29 | 72 |
| | 201-242 | | |
| 2 | 80-90 or | 29 | 73 |
| | 201-242 | | |
| 2 | 80-90 or | 29 | 74 |
| | 201-242 | | |
| 2 | 80-90 or | 29 | 756 and 757 |
| | 201-242 | | |
| 2 | 80-90 or | 30 | 70 |
| | 201-242 | | |
| 2 | 80-90 or | 30 | 71 |
| | 201-242 | | |
| 2 | 80-90 or | 30 | 72 |
| | 201-242 | | |
| 2 | 80-90 or | 30 | 73 |
| | 201-242 | | |
| 2 | 80-90 or | 30 | 74 |
| | 201-242 | | |
| 2 | 80-90 or | 30 | 756 and 757 |
| | 201-242 | | |
| 2 | 80-90 or | 31 | 70 |
| | 201-242 | | |
| 2 | 80-90 or | 31 | 71 |
| | 201-242 | | |
| 2 | 80-90 or | 31 | 72 |
| | 201-242 | | |
| 2 | 80-90 or | 31 | 73 |
| | 201-242 | | |
| 2 | 80-90 or | 31 | 74 |
| | 201-242 | | |
| 2 | 80-90 or | 31 | 756 and 757 |
| | 201-242 | | |
| 2 | 80-90 or | 32 and 33 | 70 |
| | 201-242 | | |
| 2 | 80-90 or | 32 and 33 | 71 |
| | 201-242 | | |
| 2 | 80-90 or | 32 and 33 | 72 |
| | 201-242 | | |
| 2 | 80-90 or | 32 and 33 | 73 |
| | 201-242 | | |
| 2 | 80-90 or | 32 and 33 | 74 |
| | 201-242 | | |
| 2 | 80-90 or | 32 and 33 | 756 and 757 |
| | 201-242 | | |
| 2 | 80-90 or | 40 | 70 |
| | 201-242 | | |
| 2 | 80-90 or | 40 | 71 |
| | 201-242 | | |
| 2 | 80-90 or | 40 | 72 |
| | 201-242 | | |
| 2 | 80-90 or | 40 | 73 |
| | 201-242 | | |
| 2 | 80-90 or | 40 | 74 |
| | 201-242 | | |
| 2 | 80-90 or | 40 | 756 and 757 |
| | 201-242 | | |
| 2 | 80-90 or | 41 | 70 |
| | 201-242 | | |
| 2 | 80-90 or | 41 | 71 |
| | 201-242 | | |
| 2 | 80-90 or | 41 | 72 |
| | 201-242 | | |
| 2 | 80-90 or | 41 | 73 |
| | 201-242 | | |
| 2 | 80-90 or | 41 | 74 |
| | 201-242 | | |
| 2 | 80-90 or | 41 | 756 and 757 |
| | 201-242 | | |
| 2 | 80-90 or | 42 | 70 |
| | 201-242 | | |
| 2 | 80-90 or | 42 | 71 |
| | 201-242 | | |
| 2 | 80-90 or | 42 | 72 |
| | 201-242 | | |
| 2 | 80-90 or | 42 | 73 |
| | 201-242 | | |
| 2 | 80-90 or | 42 | 74 |
| | 201-242 | | |
| 2 | 80-90 or | 42 | 756 and 757 |
| | 201-242 | | |
| 2 | 80-90 or | 43 | 70 |
| | 201-242 | | |
| 2 | 80-90 or | 43 | 71 |
| | 201-242 | | |
| 2 | 80-90 or | 43 | 72 |
| | 201-242 | | |
| 2 | 80-90 or | 43 | 73 |
| | 201-242 | | |
| 2 | 80-90 or | 43 | 74 |
| | 201-242 | | |
| 2 | 80-90 or | 43 | 756 and 757 |
| | 201-242 | | |
| 2 | 80-90 or | 44 | 70 |
| | 201-242 | | |
| 2 | 80-90 or | 44 | 71 |
| | 201-242 | | |
| 2 | 80-90 or | 44 | 72 |
| | 201-242 | | |
| 2 | 80-90 or | 44 | 73 |
| | 201-242 | | |
| 2 | 80-90 or | 44 | 74 |
| | 201-242 | | |
| 2 | 80-90 or | 44 | 756 and 757 |
| | 201-242 | | |
| 2 | 80-90 or | 45 | 70 |
| | 201-242 | | |
| 2 | 80-90 or | 45 | 71 |
| | 201-242 | | |
| 2 | 80-90 or | 45 | 72 |
| | 201-242 | | |
| 2 | 80-90 or | 45 | 73 |
| | 201-242 | | |
| 2 | 80-90 or | 45 | 74 |
| | 201-242 | | |
| 2 | 80-90 or | 45 | 756 and 757 |
| | 201-242 | | |
| 2 | 80-90 or | 46 | 70 |
| | 201-242 | | |
| 2 | 80-90 or | 46 | 71 |
| | 201-242 | | |
| 2 | 80-90 or | 46 | 72 |
| | 201-242 | | |
| 2 | 80-90 or | 46 | 73 |
| | 201-242 | | |
| 2 | 80-90 or | 46 | 74 |
| | 201-242 | | |
| 2 | 80-90 or | 46 | 756 and 757 |
| | 201-242 | | |
| 2 | 80-90 or | 47 | 70 |
| | 201-242 | | |
| 2 | 80-90 or | 47 | 71 |
| | 201-242 | | |
| 2 | 80-90 or | 47 | 72 |
| | 201-242 | | |
| 2 | 80-90 or | 47 | 73 |
| | 201-242 | | |
| 2 | 80-90 or | 47 | 74 |
| | 201-242 | | |
| 2 | 80-90 or | 47 | 756 and 757 |
| | 201-242 | | |
| 2 | 80-90 or | 48 | 70 |
| | 201-242 | | |
| 2 | 80-90 or | 48 | 71 |
| | 201-242 | | |
| 2 | 80-90 or | 48 | 72 |
| | 201-242 | | |
| 2 | 80-90 or | 48 | 73 |
| | 201-242 | | |
| 2 | 80-90 or | 48 | 74 |
| | 201-242 | | |
| 2 | 80-90 or | 48 | 756 and 757 |
| | 201-242 | | |
| 2 | 80-90 or | 49 | 70 |
| | 201-242 | | |
| 2 | 80-90 or | 49 | 71 |
| | 201-242 | | |
| 2 | 80-90 or | 49 | 72 |
| | 201-242 | | |
| 2 | 80-90 or | 49 | 73 |
| | 201-242 | | |
| 2 | 80-90 or | 49 | 74 |
| | 201-242 | | |
| 2 | 80-90 or | 49 | 756 and 757 |
| | 201-242 | | |
| 2 | 80-90 or | 50 | 70 |
| | 201-242 | | |
| 2 | 80-90 or | 50 | 71 |
| | 201-242 | | |
| 2 | 80-90 or | 50 | 72 |
| | 201-242 | | |
| 2 | 80-90 or | 50 | 73 |
| | 201-242 | | |
| 2 | 80-90 or | 50 | 74 |
| | 201-242 | | |
| 2 | 80-90 or | 50 | 756 and 757 |
| | 201-242 | | |
| 2 | 80-90 or | 51 | 70 |
| | 201-242 | | |
| 2 | 80-90 or | 51 | 71 |
| | 201-242 | | |
| 2 | 80-90 or | 51 | 72 |
| | 201-242 | | |
| 2 | 80-90 or | 51 | 73 |
| | 201-242 | | |
| 2 | 80-90 or | 51 | 74 |
| | 201-242 | | |
| 2 | 80-90 or | 51 | 756 and 757 |
| | 201-242 | | |
| 2 | 80-90 or | 747 | 70 |
| | 201-242 | | |
| 2 | 80-90 or | 747 | 71 |
| | 201-242 | | |
| 2 | 80-90 or | 747 | 72 |
| | 201-242 | | |
| 2 | 80-90 or | 747 | 73 |
| | 201-242 | | |
| 2 | 80-90 or | 747 | 74 |
| | 201-242 | | |
| 2 | 80-90 or | 747 | 756 and 757 |
| | 201-242 | | |
| 2 | 80-90 or | 748 and 749 | 70 |
| | 201-242 | | |
| 2 | 80-90 or | 748 and 749 | 71 |
| | 201-242 | | |
| 2 | 80-90 or | 748 and 749 | 72 |
| | 201-242 | | |
| 2 | 80-90 or | 748 and 749 | 73 |
| | 201-242 | | |
| 2 | 80-90 or | 748 and 749 | 74 |
| | 201-242 | | |
| 2 | 80-90 or | 748 and 749 | 756 and 757 |
| | 201-242 | | |

Additionally, any cytokine prodrug described herein, in Table 3A or elsewhere, may further comprise a targeting sequence, such as any of the targeting sequences described herein. In some embodiments, the targeting sequence is any one of SEQ ID NOs: 180-662.

Additionally, any one of the cytokine prodrugs described in Table 3A may comprise a consensus sequence according to any one of SEQ ID NOs: 91-94 in place of the listed protease-cleavable sequences.

Also encompassed by this disclosure are cytokine prodrugs comprising a sequence with at least 80, 85, 90, 95, 97, 98, or 99 percent identity to the sequence of any one of the cytokine prodrugs described above.

In some embodiments, the cytokine prodrug comprises a sequence with at least 80, 85, 90, 95, 97, 98, or 99 percent identity to the sequence of any one of SEQ ID NOs: 100-111. In some embodiments, the cytokine prodrug comprises the sequence of any one of SEQ ID NOs: 100-111. In some embodiments, the cytokine prodrug comprises the sequence of any one of SEQ ID NOs: 803-852.

Combinations of a Protease-Cleavable Sequence and a Targeting Sequence

Any compatible embodiment of a cytokine prodrug described herein, in Table 3A or elsewhere, may comprise a combination of a protease-cleavable sequence and a targeting sequence set forth in Table 4. Where a range is given, any one of the listed SEQ ID NOs may be selected. The components may be arranged in any manner consistent with the disclosure, e.g., as indicated elsewhere herein (e.g., FIGS. 9 and 10A-E and the section regarding Arrangement of components).

TABLE 4
Exemplary combinations of protease-cleavable
sequence and targeting sequence

| Protease-cleavable sequence | Targeting sequence |
|---|---|
| 80 | 180-662 |
| 81 | 180-662 |
| 82 | 180-662 |
| 83 | 180-662 |
| 84 | 180-662 |
| 85 | 180-662 |
| 86 | 180-662 |
| 87 | 180-662 |
| 88 | 180-662 |
| 89 | 180-662 |
| 90 | 180-662 |
| 91 | 180-662 |
| 92 | 180-662 |
| 93 | 180-662 |
| 94 | 180-662 |
| 700 | 180-662 |
| 701 | 180-662 |
| 702 | 180-662 |
| 703 | 180-662 |
| 704 | 180-662 |
| 705 | 180-662 |
| 706 | 180-662 |
| 707 | 180-662 |
| 708 | 180-662 |
| 709 | 180-662 |
| 710 | 180-662 |
| 711 | 180-662 |
| 712 | 180-662 |
| 713 | 180-662 |
| 714 | 180-662 |
| 715 | 180-662 |
| 716 | 180-662 |
| 717 | 180-662 |
| 718 | 180-662 |
| 719 | 180-662 |
| 720 | 180-662 |
| 721 | 180-662 |
| 722 | 180-662 |
| 723 | 180-662 |
| 724 | 180-662 |
| 725 | 180-662 |
| 726 | 180-662 |
| 727 | 180-662 |
| 728 | 180-662 |
| 729 | 180-662 |
| 730 | 180-662 |
| 731 | 180-662 |
| 732 | 180-662 |
| 733 | 180-662 |
| 734 | 180-662 |
| 735 | 180-662 |
| 736 | 180-662 |
| 737 | 180-662 |
| 738 | 180-662 |
| 739 | 180-662 |
| 740 | 180-662 |
| 741 | 180-662 |

Also encompassed by this disclosure are cytokine prodrugs comprising a sequence with at least 80, 85, 90, 95, 97, 98, or 99 percent identity to the sequence of any one of the cytokine prodrugs described above.

### Pharmaceutical Formulations

Pharmaceutical formulations of a cytokine prodrug as described herein may be prepared by mixing such cytokine prodrug having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

The formulations to be used for in vivo administration are generally sterile. Sterility may be readily accomplished, e.g., by filtration through sterile filtration membranes.

### Uses

In some embodiments, any one or more of the cytokine prodrugs, compositions, or pharmaceutical formulations described herein is for use in preparing a medicament for treating or preventing a disease or disorder in a subject. In some embodiments, any one or more of the cytokine prodrugs, compositions, or pharmaceutical formulations described herein is for use in a method of creating a cytokine gradient in a subject, comprising administering the protease-activated pro-cytokine or pharmaceutical composition to a subject, wherein the subject comprises a site having an abnormally high level of a protease that cleaves the protease-cleavable polypeptide sequence, optionally wherein the site comprises a cancer. In some embodiments, the abnormally high level is higher than the level of the protease in a healthy tissue of the same type as the site with the abnormally high level (e.g., in the subject being treated or in a healthy subject). In some embodiments, the abnormally high level is higher than the average level of the protease in soft tissue.

In some embodiments, a method of treating or preventing a disease or disorder in subject is provided, comprising administering to a subject any of the cytokine prodrugs or pharmaceutical compositions described herein. In some embodiments, the disease or disorder is a cancer, e.g., a solid tumor. In some embodiments, the cancer is a melanoma, a colorectal cancer, a breast cancer, a pancreatic cancer, a lung cancer, a prostate cancer, an ovarian cancer, a cervical cancer, a gastric or gastrointestinal cancer, a lymphoma, a colon or colorectal cancer, an endometrial cancer, a thyroid cancer, or a bladder cancer. The cancer (e.g., any of the foregoing cancers) may have one or more of the following features: being PD-L1-positive; being metastatic; being unresectable; being mismatch repair defective (MMRd); and/or being microsatellite-instability high (MSI-H).

In some embodiments, a method of boosting T regulatory cells and/or reducing inflammation or autoimmune activity is provided comprising administering a cytokine prodrug to an area of interest, e.g., an area of inflammation. The cytokine prodrug for use in such methods may comprise an IL-2 polypeptide sequence. In some embodiments, a method of treating an autoimmune and/or inflammatory disease is provided, comprising administering a cytokine prodrug to an area of interest, e.g., an area of inflammation or autoimmune activity. The cytokine prodrug for use in such methods may comprise an IL-2 polypeptide sequence. These methods take advantage of the ability of certain cytokines at relatively low levels to stimulate T regulatory cells, which can exert anti-inflammatory effects and reduce or suppress autoimmune activity.

The cytokine prodrugs in any of the foregoing methods and uses may be delivered to a subject using any suitable route of administration. In some embodiments, the cytokine prodrug is delivered parenterally. In some embodiments, the cytokine prodrug is delivered intravenously.

A cytokine prodrug provided herein can be used either alone or in combination with other agents in a therapy. For instance, a cytokine prodrug provided herein may be co-administered with at least one additional therapeutic agent.

Such combination therapies noted above encompass combined administration (where two or more therapeutic agents are included in the same or separate formulations), and separate administration, in which case, administration of the cytokine prodrug provided herein can occur prior to, simultaneously, and/or following, administration of the additional therapeutic agent and/or adjuvant.

Cytokine prodrugs would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The cytokine prodrug need not be, but is optionally formulated with one or more agents currently used to prevent or treat the disorder in question. The effective amount of such other agents depends on the amount of cytokine prodrug present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an cytokine prodrug (when used alone or in combination with one or more other additional therapeutic agents) will depend on the type of disease to be treated, the type of cytokine prodrug, the severity and course of the disease, whether the cytokine prodrug is administered for preventive or therapeutic purposes, previous therapy, the patient's clinical history and response to the antibody or immunoconjugate, and the discretion of the attending physician. The cytokine prodrug is suitably administered to the patient at one time or over a series of treatments.

### Nucleic Acids, Host Cells, and Production Methods

Cytokine prodrugs or precursors thereof may be produced using recombinant methods and compositions. In some embodiments, isolated nucleic acid encoding a cytokine prodrug described herein is provided. Such nucleic acid may encode an amino acid sequence comprising the cytokine polypeptide sequence, the linker, and the inhibitory polypeptide sequence, and any other polypeptide components of the cytokine prodrug that may be present. Exemplary nucleic acid sequences are provided in Table 1. In a further embodiment, one or more vectors (e.g., expression vectors) comprising such nucleic acid are provided. In a further embodiment, a host cell comprising such nucleic acid is provided. In some such embodiments, a host cell comprises (e.g., has been transformed with) a vector comprising a nucleic acid that encodes a cytokine prodrug according to the disclosure. In some embodiments, the host cell is eukaryotic, e.g. a Chinese Hamster Ovary (CHO) cell or lymphoid cell (e.g., Y0, NS0, Sp20 cell). In some embodiments, a method of making a cytokine prodrug disclosed herein is provided, wherein the method comprises culturing a host cell comprising a nucleic acid encoding the cytokine prodrug, as provided above, under conditions suitable for expression of the cytokine prodrug, and optionally recovering the antibody from the host cell (or host cell culture medium).

For recombinant production of a cytokine prodrug, nucleic acid encoding the cytokine prodrug, e.g., as described above, is prepared and/or isolated (e.g., following construction using synthetic and/or molecular cloning techniques) and inserted into one or more vectors for further cloning and/or expression in a host cell. Such nucleic acid may be readily prepared and/or isolated using known techniques.

Suitable host cells for cloning or expression of cytokine prodrug-encoding vectors include prokaryotic or eukaryotic cells described herein. For example, a cytokine prodrug may be produced in bacteria, in particular when glycosylation is not needed. For expression of polypeptides in bacteria, see, e.g., U.S. Pat. Nos. 5,648,237, 5,789,199, and 5,840,523. After expression, the cytokine prodrug may be isolated from the bacterial cell paste in a soluble fraction and can be further purified.

In addition to prokaryotes, eukaryotic microbes such as filamentous fungi or yeast are suitable cloning or expression hosts for cytokine prodrug-encoding vectors, including fungi and yeast strains whose glycosylation pathways have been “humanized,” resulting in the production of polypeptides with a partially or fully human glycosylation pattern. See Gerngross, Nat. Biotech. 22:1409-1414 (2004), and Li et al., Nat. Biotech. 24:210-215 (2006).

Suitable host cells for the expression of cytokine prodrugs are also derived from multicellular organisms (plants, invertebrates, and vertebrates). Examples of invertebrate cells include insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. “About” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

### EXAMPLES

The following examples are provided to illustrate certain disclosed embodiments and are not to be construed as limiting the scope of this disclosure in any way.

### Example 1: Construction of Mammalian Expression Vectors Encoding Fusion Proteins

Coding sequences for all protein domains including linker sequences were synthesized as an entire gene (Genscript, NJ). All synthetic genes were designed to contain a coding sequence for an N-terminal signal peptide (to facilitate protein secretion), a 5′ Kozak sequence, and unique restriction sites at the 5′ and 3′ ends. These genes were then directionally cloned into the mammalian expression vector pcDNA3.1 (Invitrogen, Carlsbad, CA). Examples of fusion protein constructs are listed in table 5A. Site directed mutagenesis was performed using standard molecular biology techniques and appropriate kit (GeneArt, Regensburg).

TABLE 5A
Exemplary cytokine prodrug constructs

| | SEQ ID | |
|---|---|---|
| Name | NO | Features |
| Construct | 100 | mIL2-2x(SG4)-MMPcs1-2x(G4S)-IL2Ralpha-6His |
| A | | |
| Construct | 101 | m IL2-2x(SG4)-MMPcs1-2x(G4S)-IL2Ralpha-mIgG1 Fc |
| B | | |
| Construct | 102 | mIL2(C140S)-2x(SG4)-MMPcs1-2x(G4S)-IL2Ralpha- |
| C | | mIgG1 Fc(T252M)-6xHIS |
| Construct | 104 | mIL2(C140S)-2x(SG4)-MMPcs1-2x(G4S)-soluble |
| D | | IL2Ralpha-mIgG1 Fc(T252M)-6xHIS |
| Construct | 106 | Hu IL2(C125S)-2x(SG4)-MMPcs1-2x(G4S)-IL2Ralpha-hu |
| E | | IgG1 Fc-6xHIS |
| Construct | 803 | h IL2(C125S)-2x(SG4)-MMPcs1-2x(G4S)-chimeric IL2Ra(sushi |
| F | | mouse)-hIgG1 Fc |
| Construct | 804 | hIL2(C125S)-2x(SG4)-MMPcs1-2x(G4S)-hIL2Ra(1-219)- |
| G | | GSGGGG-hu IgG1 Fc |
| Construct | 805 | hIL2(C125S)-2x(SG4)-MMPcs1-2x(G4S)-hIL2Ra(1-178)- |
| H | | GSGGGG-hu IgG1 Fc |
| Construct | 806 | hIL2(C125S)-2x(SG4)-MMPcs1-4x(G4S)-hIL2Ra(1-219)- |
| V | | GSGGGG-hu IgG1 Fc |
| Construct | 807 | hIL2(C125S)-2x(SG4)-MMPcs1-6x(G4S)-hIL2Ra(1-219)- |
| W | | GSGGGG-hu IgG1 Fc |
| Construct | 837 | h IL2(C125S)-2x(SG4)-MMPcs1-2x(G4S)-hIL2Ra(1-219; M25I)- |
| I | | hIgG1 Fc-6xHis |
| Construct | 838 | h IL2(C125S)-2x(SG4)-MMPcs1-2x(G4S)-hIL2Ra(1-219; L42V)- |
| J | | hIgG1 Fc-6xHis |
| Construct | 808 | m IL2(C140S)-2x(SG4)-MMPik-2x(G4S)-mIL2Ralpha(1- |
| X | | 215)-mu IgG1 Fc |
| Construct | 809 | m IL2(C140S)-VRIQRKKEKMKET-MMPcs1-2x(G4S)-mIL2Ra |
| Y | | (1-215)-mu IgG1 Fc |
| Construct | 810 | m IL2-2x(SG4)-MMPlk-2x(G4S)-mIL2Ralpha(1-215)-mu |
| Z | | IgG1 Fc |
| Construct | 811 | m IL2-SGG-FHRRIKA-MMPcs1-2x(G4S)-mIL2Ra(1-215)-mu |
| AA | | IgG1 Fc |
| Construct | 812 | m IL2-SGG-FHRRIKA-MMPscr-2x(G4S)-mIL2Ra(1-215)-mu |
| BB | | IgG1 Fc |
| Construct | 813 | m IL2-2x(GHHPH)-MMPcs1-2x(G4S)-mIL2Ra(1-215)-mu |
| CC | | IgG1 Fc |
| Construct | 814 | m IL2-2x(GHHPH)-MMPscr-2x(G4S)-mIL2Ra(1-215)-mu |
| DD | | IgG1 Fc |
| Construct | 815 | m IL2-SGG-GGWSHW-MMPcs1-2x(G4S)-mIL2Ra(1-215)- |
| EE | | mu IgG1 Fc |
| Construct | 816 | m IL2-SGG-GGWSHW-MMPscr-2x(G4S)-mIL2Ra(1-215)-mu |
| FF | | IgG1 Fc |
| Construct | 817 | m IL2-SGG-KLWVLPK-MMPcs1-2x(G4S)-mIL2Ra(1-215)- |
| GG | | mu IgG1 Fc |
| Construct | 818 | m IL2-SGG-KLWVLPK-MMPscr-2x(G4S)-mIL2Ra(1-215)- |
| HH | | mu IgG1 Fc |
| Construct | 819 | m IL2-LHERHLNNN-MMPcs1-2x(G4S)-mIL2Ra(1-215)-mu |
| II | | IgG1 Fc |
| Construct | 820 | m IL2-LHERHLNNN-MMPscr-2x(G4S)-mIL2Ra(1-215)-mu |
| JJ | | IgG1 Fc |
| Construct | 821 | m IL2-VRIQRKKEKMKET-MMPscr-2x(G4S)-mIL2Ra(1- |
| KK | | 215)-mu IgG1 Fc |
| Construct | 822 | m IL2-2x(SG4)-MMPcs1-FHRRIKAGGS-mIL2Ralpha(1- |
| LL | | 215)-mu IgG1 Fc |
| Construct | 823 | m IL2-2x(SG4)-MMPscr-FHRRIKAGGS-mIL2Ralpha(1- |
| MM | | 215)-mu IgG1 Fc |
| Construct | 824 | m IL2-2x(SG4)-MMPcs1-2x(GHHPH)-mIL2Ra(1-215)-mu |
| NN | | IgG1 Fc |
| Construct | 825 | m IL2-2x(SG4)-MMPscr-2x(GHHPH)-mIL2Ra(1-215)-mu IgG1 |
| OO | | Fc |
| Construct | 826 | m IL2-2x(SG4)-MMPcs1-GGWSHWGGS-mIL2Ralpha(1- |
| PP | | 215)-mu IgG1 Fc |
| Construct | 827 | m IL2-2x(SG4)-MMPscr-GGWSHWGGS-mIL2Ralpha(1- |
| QQ | | 215)-mu IgG1 Fc |
| Construct | 828 | m IL2-2x(SG4)-MMPcs1-KLWVLPKGGS-mIL2Ralpha(1- |
| RR | | 215)-mu IgG1 Fc |
| Construct | 829 | m IL2-2x(SG4)-MMPscr-KLWVLPKGGS-mIL2Ralpha(1- |
| SS | | 215)-mu IgG1 Fc |
| Construct | 830 | m IL2-2x(SG4)-MMPcs1-LHERHLNNNG-mIL2Ralpha(1- |
| TT | | 215)-mu IgG1 Fc |
| Construct | 831 | m IL2-2x(SG4)-MMPscr-LHERHLNNNG-mIL2Ralpha(1- |
| UU | | 215)-mu IgG1 Fc |
| Construct | 832 | m IL2-SGGGGGHHPH-MMPcs1-2x(G4S)-mIL2Ra-mu IgG1 |
| VV | | Fc |
| Construct | 833 | m IL2-GHHPHSGGGG-MMPcs1-2x(G4S)-mIL2Ra-mu IgG1 |
| WW | | Fc |
| Construct | 834 | m IL2-2x(SG4)-MMPcs1-GHHPHGGGGS-mIL2Ra-mu IgG1 Fc |
| XX | | |
| Construct | 835 | m IL2-2x(SG4)-MMPcs1-2x(G4S)-mIL2Ra-mu IgG1 Fc- |
| YY | | 2x(GHHPH) |
| Construct | 836 | m IL2-2x(SG4)-MMPcs1-2x(G4S)-mIL2Ra-mu IgG1 Fc- |
| ZZ | | (GHHPH) |
| Construct | 840 | h IL2(C125S)-2x(SG4)-MMPcs1-2x(G4S)-hIL2Ra(1-219; |
| L | | SGSL39-42ELV)-hIgG1 Fc-6xHis |
| Construct | 839 | h IL2(C125S)-2x(SG4)-MMPcs1-2x(G4S)-hIL2Ra(1-219; DD4- |
| K | | 5LY)-hIgG1 Fc-6xHis |
| Construct | 841 | hIL2(C125S)-2x(SG4)-MMPcs1-2x(G4S)-hIL2Ra(1-192)-hu |
| M | | IgG1 Fc-6xHis |
| Construct | 842 | hIL2(C125S)-2x(SG4)-MMPcs1-2x(G4S)-hIL2Ra(1-192)- |
| N | | GSGGGG-hu IgG1 Fc-6xHis |
| Construct | 843 | hIL2(C125S)-2x(SG4)-MMPcs1-2x(G4S)-hIL2Ra(1-192/M25I)- |
| O | | GSGGGG-hu IgG1 Fc-6xHis |
| Construct | 844 | hIL2(C125S)-2x(SG4)-MMPcs1-2x(G4S)-hIL2Ra(1-192/L42V)- |
| P | | GSGGGG-hu IgG1 Fc-6xHis |
| Construct | 845 | hIL2(C125S)-2x(SG4)-MMPcs1-2x(G4S)-hIL2Ra(1-192/D4L, |
| Q | | D5Y)-GSGGGG-hu IgG1 Fc-6xHis |
| Construct | 846 | h IL2(C125S)-2x(SG4)-MMPcs1-2x(G4S)-hIL2Ra(M25I)- |
| AAA | | GSGGGG-hu IgG1 Fc (LALA) |
| Construct | 847 | h IL2(C125S)-2x(SG4)-MMPscr-2x(G4S)-hIL2Ra(M25I)- |
| BBB | | GSGGGG-hu IgG1 Fc (LALA) |
| Construct | 848 | h IL2(C125S)-2x(GHHPH)-MMPscr-2x(G4S)-hIL2Ra(M25I)- |
| CCC | | GSGGGG-hu IgG1 Fc (LALA) |
| Construct | 849 | h IL2(C125S)-2x(GHHPH)-MMPcs1-2x(G4S)-hIL2Ra(M25I)- |
| DDD | | GSGGGG-hu IgG1 Fc (LALA) |
| Construct | 850 | h IL2(C125S)-VRIQRKKEKMKET-MMPcs1-2x(G4S)- |
| EEE | | hIL2Ra(M25I)-GSGGGG-hu IgG1 Fc(LALA) |
| Construct | 851 | h IL2(C125S)-VRIQRKKEKMKET-MMPscr-2x(G4S)- |
| FFF | | hIL2Ra(M25I)-GSGGGG-hu IgG1 Fc(LALA) |
| Construct | 852 | m IL2(C140S)-2x(SG4)-MMPscr-2x(G4S)-mIL2Ralpha(1- |
| GGG | | 215)-mIgG1 Fc |

### Example 2: Expression and Purification of Fusion Proteins

Transient Expression of Fusion Proteins

Different mammalian cell expression systems were used to produce fusion proteins (ExpiCHO-S™, Expi293F™ and Freestyle CHO-S™, Life Technologies). Briefly, expression constructs were transiently transfected into cells following manufacturer's protocol and using reagents provided in respective expression kits. Fusion proteins were then expressed and secreted into the cell culture supernatant. Samples were collected from the production cultures every day and cell density and viability were assessed. Protein expression titers and product integrity in cell culture supernatants were analyzed by SDS-PAGE to determine the optimal harvesting time. Cell culture supernatants were generally harvested between 4 and 12 days at culture viabilities of typically >75%. On day of harvest, cell culture supernatants were cleared by centrifugation and vacuum filtration before further use.

Purification of Fusion Proteins

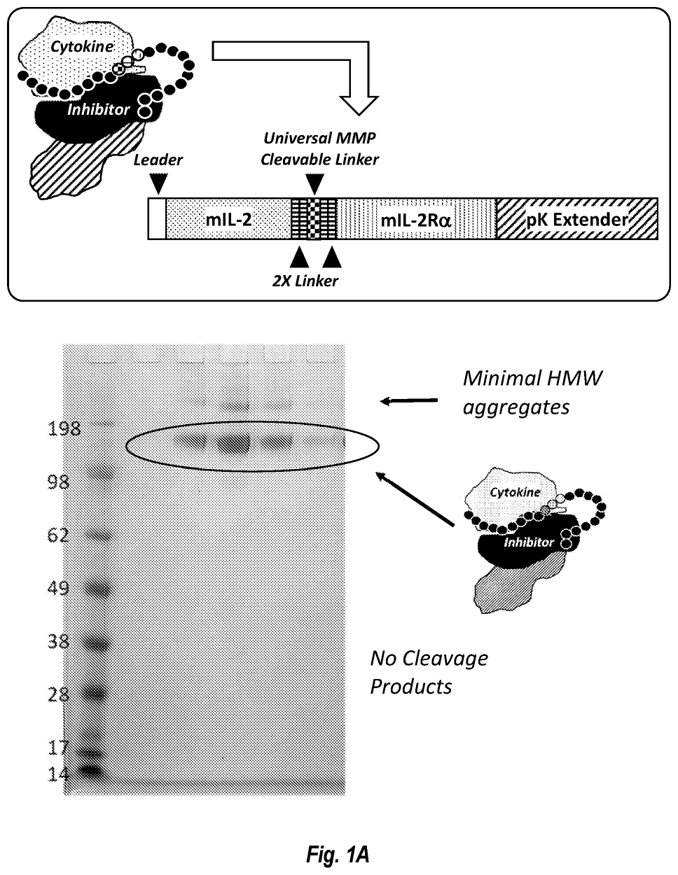

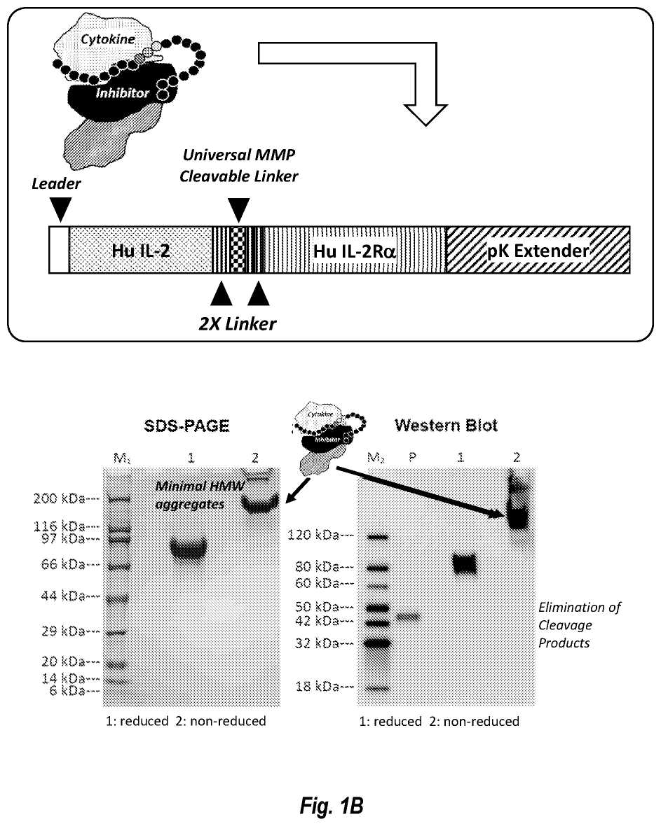

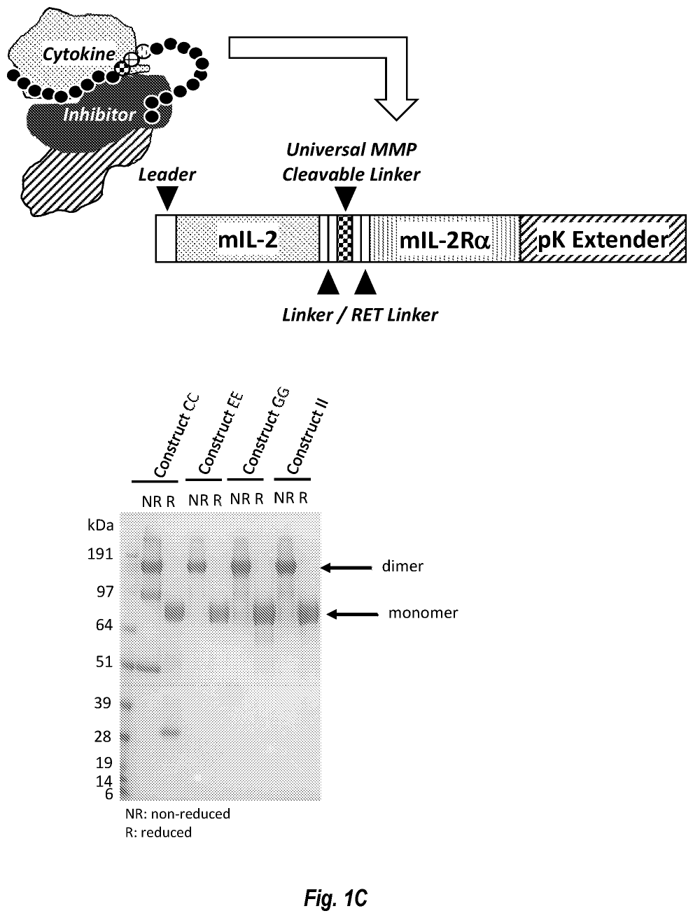

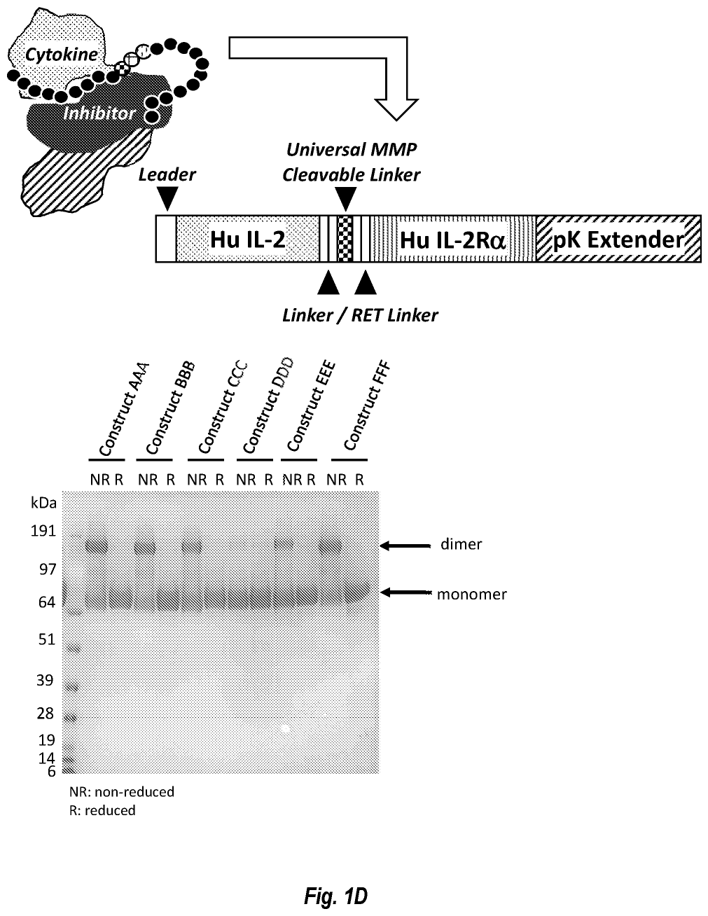

Fusion proteins were purified from cell culture supernatants in either a one-step or two-step procedure. Briefly, Fc domain containing proteins were purified by Protein A affinity chromatography (HiTrap MabSelect SuRe, GE Healthcare). His-tagged proteins were first purified on a Nickel-agarose column (Ni-NTA Agarose, Qiagen), followed by anion ion exchange chromatography (HiTrap Capto Q ImpRes, Sigma). All purified samples were buffer-exchanged and concentrated by ultrafiltration to a typical concentration of >1 mg/mL. Purity and homogeneity (typically >90%) of final samples were assessed by SDS PAGE under reducing and non-reducing conditions, followed by immunoblotting using an anti-His or anti-Fc antibody. Purified proteins were aliquoted and stored at −80° C. until further use. FIG. 1 shows examples of successfully purified fusion proteins.

### Example 3: Cleavage of Fusion Protein by MMP Proteases

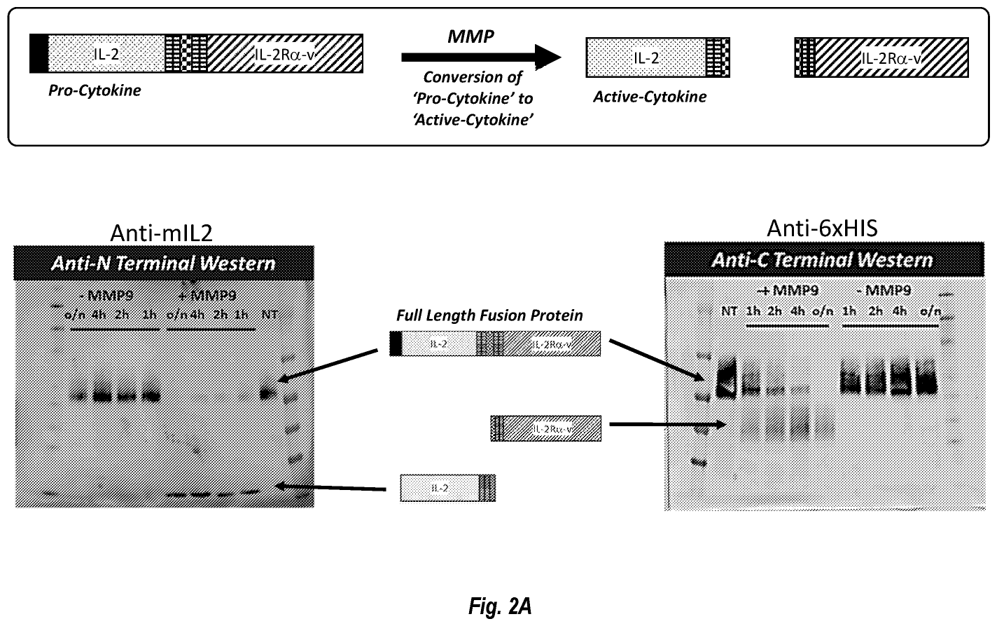

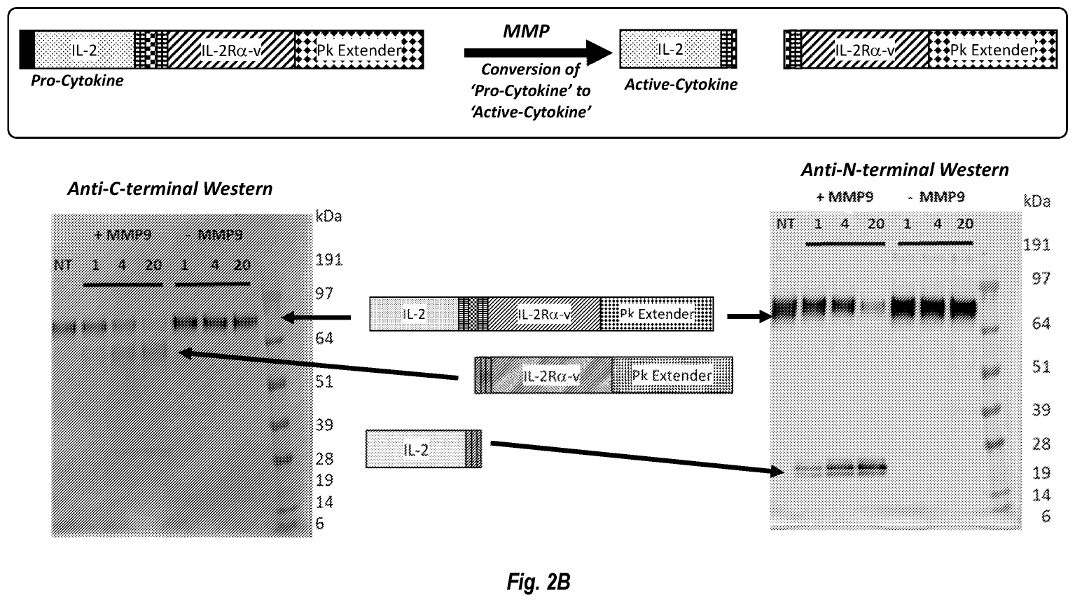

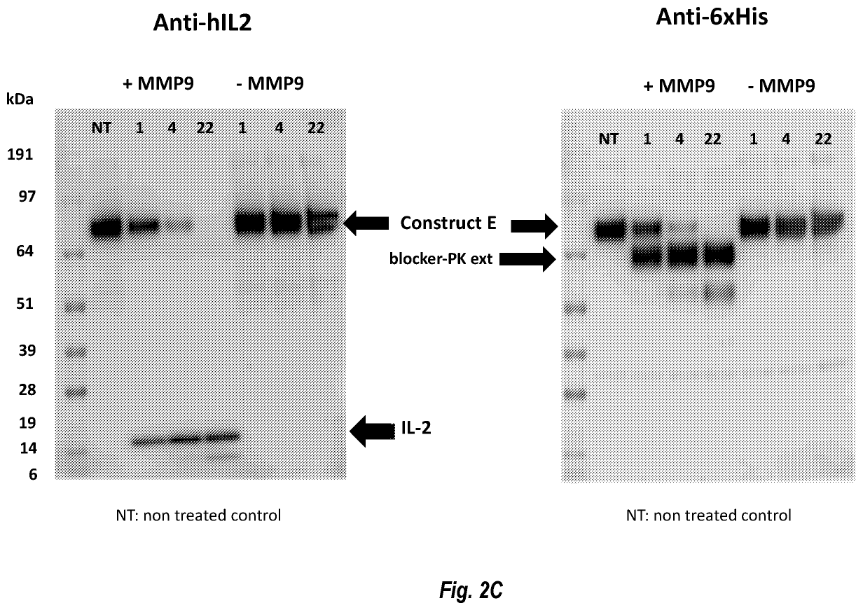

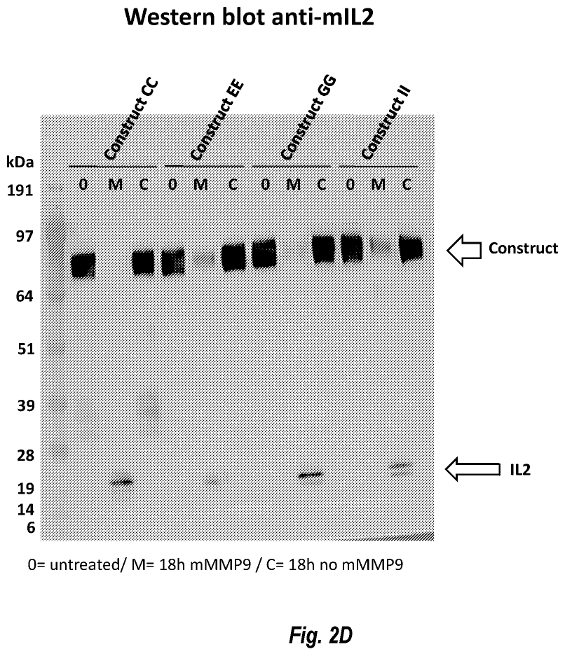

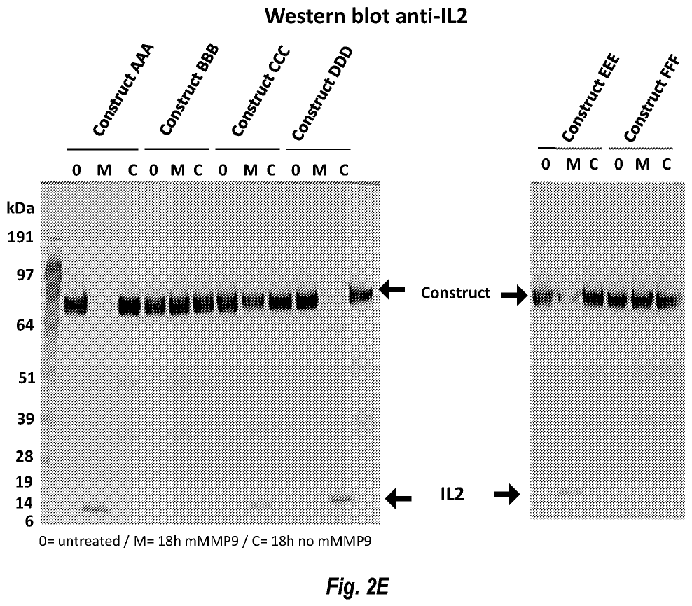

Recombinant MMP9 and/or MMP2 (R&D Systems) was first activated with p-aminophenylmercuric acetate and this activated protease or equivalent amount of activating solution without the protease was used to digest or mock digest the fusion protein for 1 hr, 2 hr, 4 hr and overnight (18-22 hr) at 37 C. Cleavage assays are set up in TCNB buffer: 50 mM Tris, 10 mM CaCl2), 150 mM NaCl, 0.05% Brij-35 (w/v), pH 7.5. Digested protein was aliquoted and stored at −80° C. prior to testing. Aliquots of digests were subsequently analyzed by SDS-PAGE followed by Western blotting to evaluate the extent of cleavage. Digests were also assessed in functional assays such as CTLL-2 proliferation and HEK-Blue Interleukin reporter assays. As shown in FIGS. 2A-E, essentially complete cleavage by MMP9 protease of the fusion proteins with functional site is seen after overnight incubation. In contrast, proteins containing a scrambled MMP cleavage site are not cut (FIG. 2E).

### Example 4: Detection of Mouse IL-2/IL-2Rα Fusion Proteins and Mouse IL-2 by ELISA

We developed an ELISA assay to detect and quantify fusion proteins comprising IL-2 and IL-2Rα moieties. Wells of a 96-well plate are coated overnight with 100 uL of a rat anti-mouse IL-2 monoclonal antibody (JES6-1A12; ThermoFisher) at 1 mg/ml in PBS. After washing, wells are blocked with TBS/0.05% Tween 20/1% BSA, then fusion proteins and/or unknown biological samples are added for 1 hr at room temperature. After washing, an anti-mouse IL-2Rα biotin-labelled detection antibody (BAF2438, R&D systems) is added and binding is detected using Ultra Strepavidin HRP (ThermoFisher). The ELISA plate was developed by adding the chromogenic tetramethylbenzidine substrate (Ultra TMB, ThermoFisher). The reaction is stopped by addition of 0.5M H2SO4 and the absorbance is read at 450-650 nm.

We developed a second ELISA assay to detect and quantify mouse IL-2 and/or fusion proteins comprising an IL-2 moiety. Wells of a 96-well plate are coated overnight with 100 uL of a rat anti-mouse IL-2 monoclonal antibody (JES6-1A12; ThermoFisher) at 1 mg/ml in PBS. After washing, wells are blocked with TBS/0.05% Tween 20/1% BSA, then fusion proteins and/or unknown biological samples are added for 1 hr at room temperature. After washing, an anti-mouse IL-2 biotin-labelled detection antibody (JES6-5H4, ThermoFisher) is added and binding is detected using Ultra Strepavidin HRP (ThermoFisher). The ELISA plate was developed by adding the chromogenic tetramethylbenzidine substrate (Ultra TMB, ThermoFisher). The reaction is stopped by addition of 0.5M H2SO4 and the absorbance is read at 450-650 nm. This assay is able to simultaneously detect both free mouse IL-2 as well as mouse IL-2 in the context of pro-drug fusion proteins.

### Example 5: IL-2, IL-2Ra,6xHistidine and Fc Immunoblot Analyses

Untreated and digested fusion proteins were evaluated for cleavage products by Western blot. The following monoclonal antibodies were used: rat anti-mouse IL-2 antibody (JES6-1A12; ThermoFisher), goat anti-mouse IL-2 polyclonal antibody (AF-402-NA; R&D systems), mouse anti-6xHis monoclonal antibody (MA1-21315, ThermoFisher), Anti-mIgG Fc HRP conjugated (ThermoFisher cat #A16084), and Anti-human IL2 antibody (Invitrogen, cat #MA5-17097, mouse IgG1). Detection was performed using either a goat anti-rat HRP-conjugated antibody, Donkey Anti-goat HRP-conjugated antibody or Goat Anti-mouse HIRP conjugated (Jackson Immuno Research, West Grove, PA) and developed using the SuperSignal West Femto Maximum sensitivity detection reagent (ThermoFisher) following the manufacturer's recommendations.

### Example 6: IL-2 Functional Cell-Based Assays

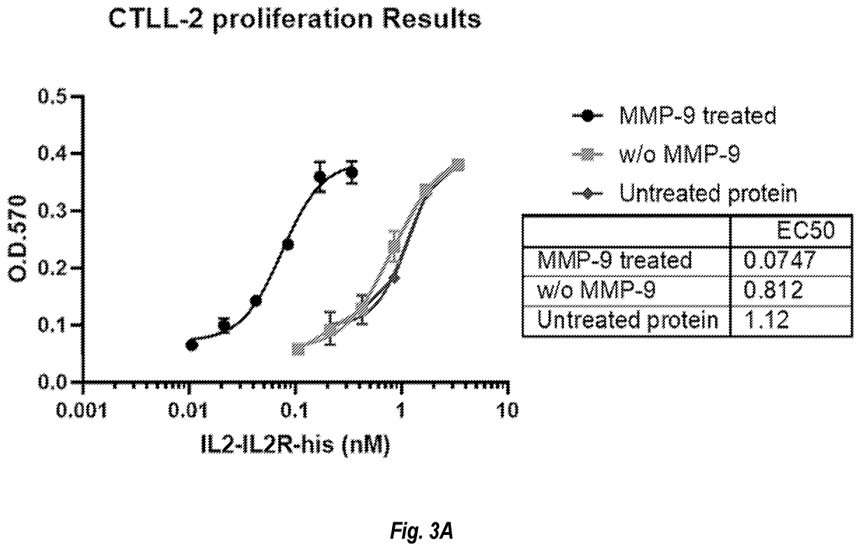

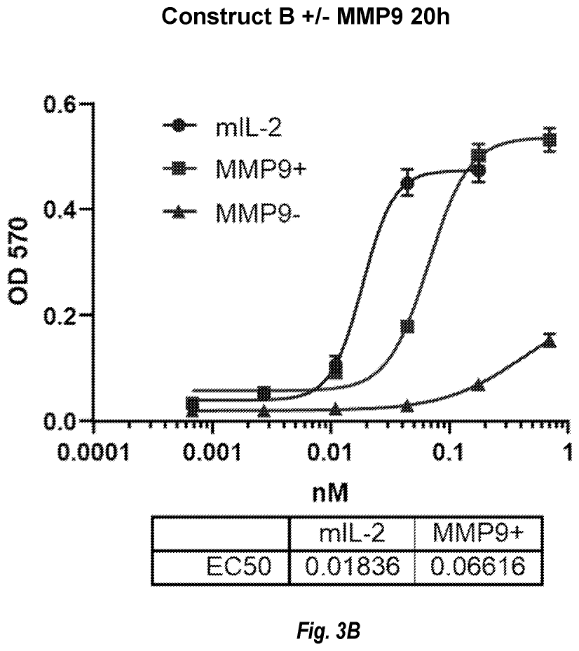

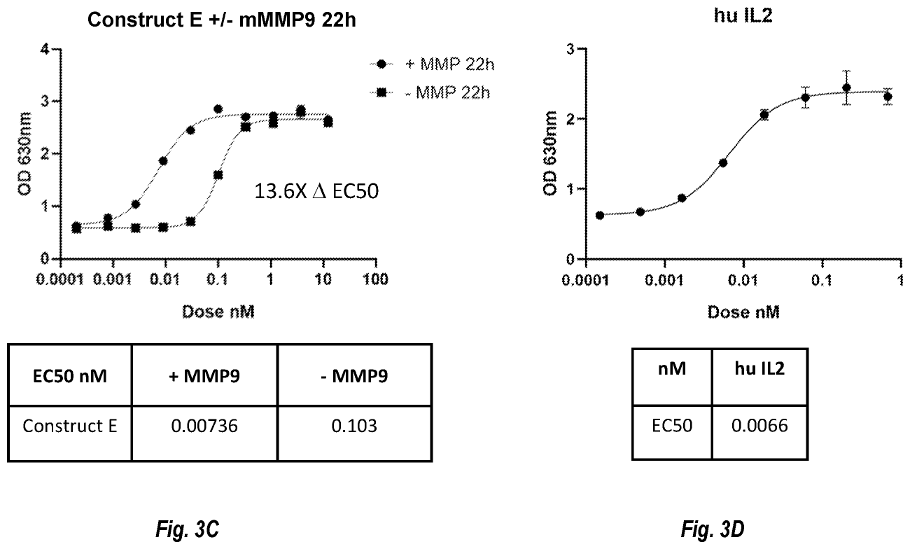

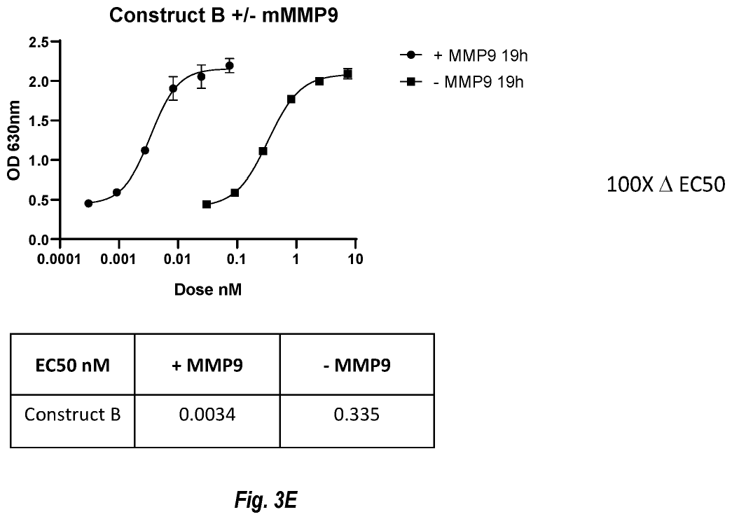

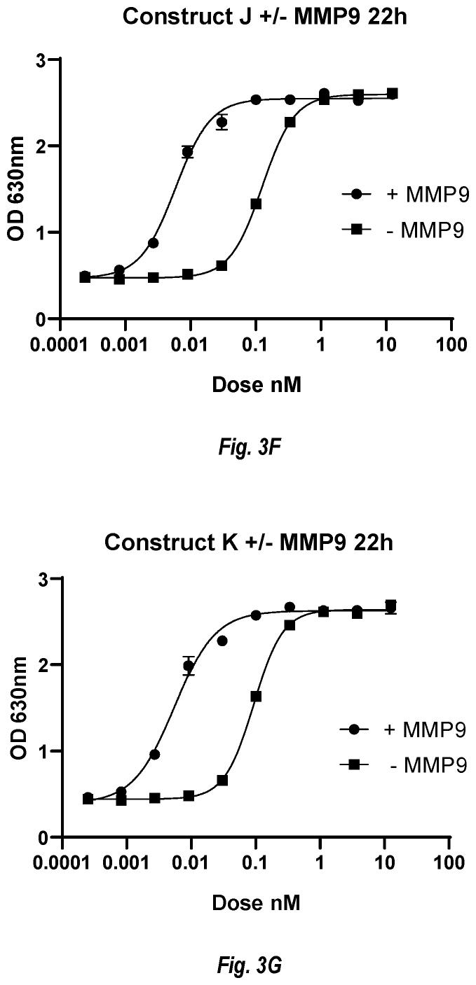

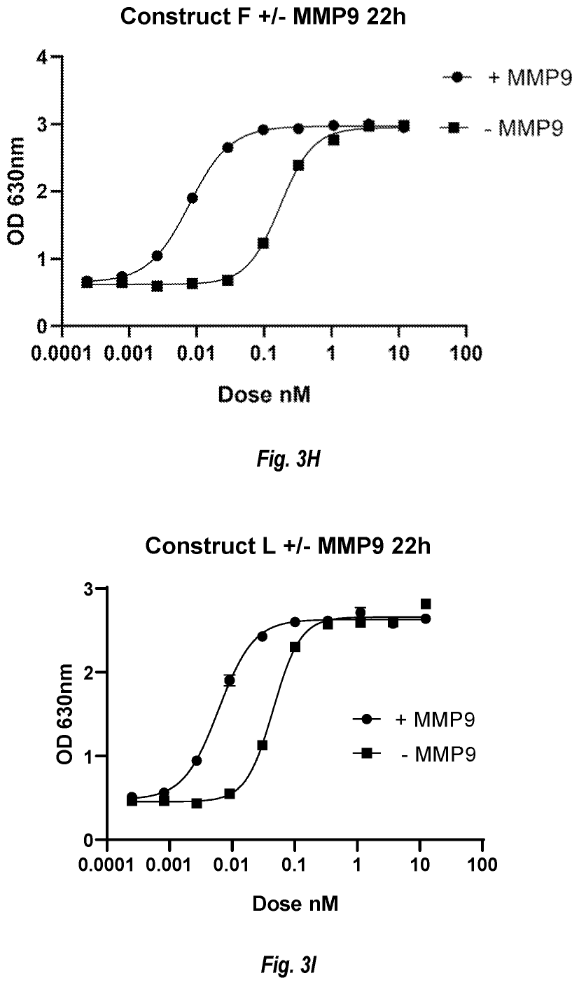

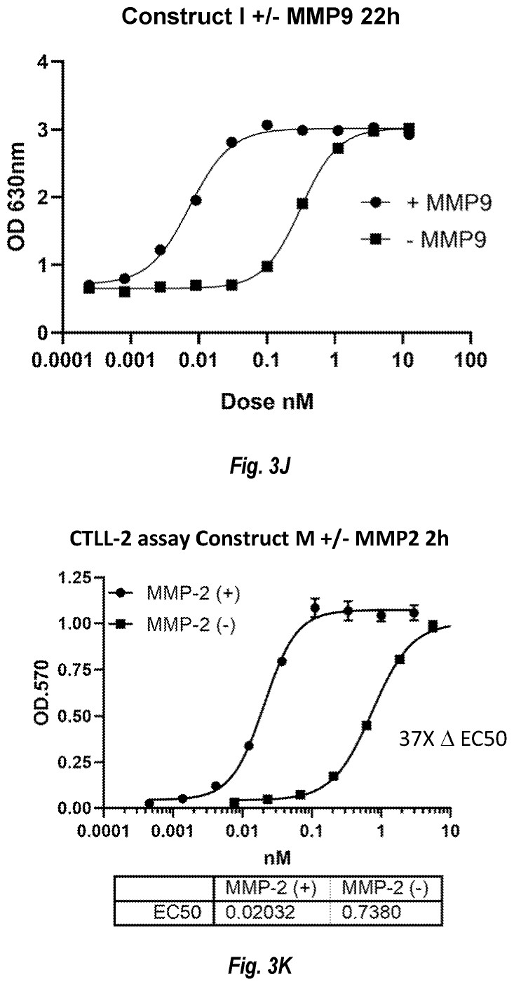

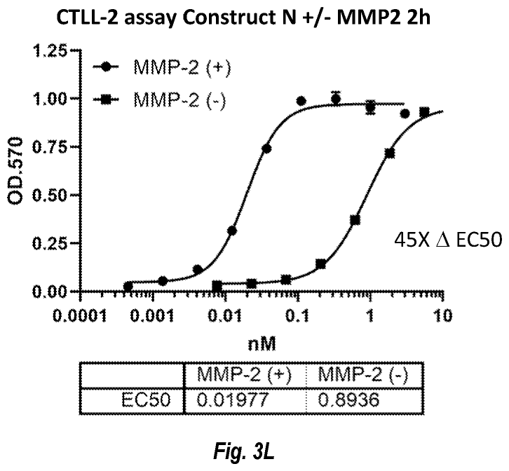

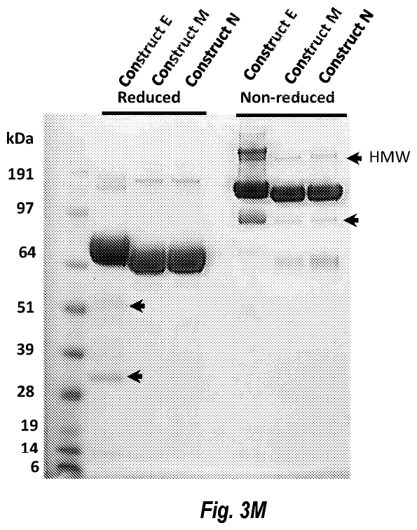

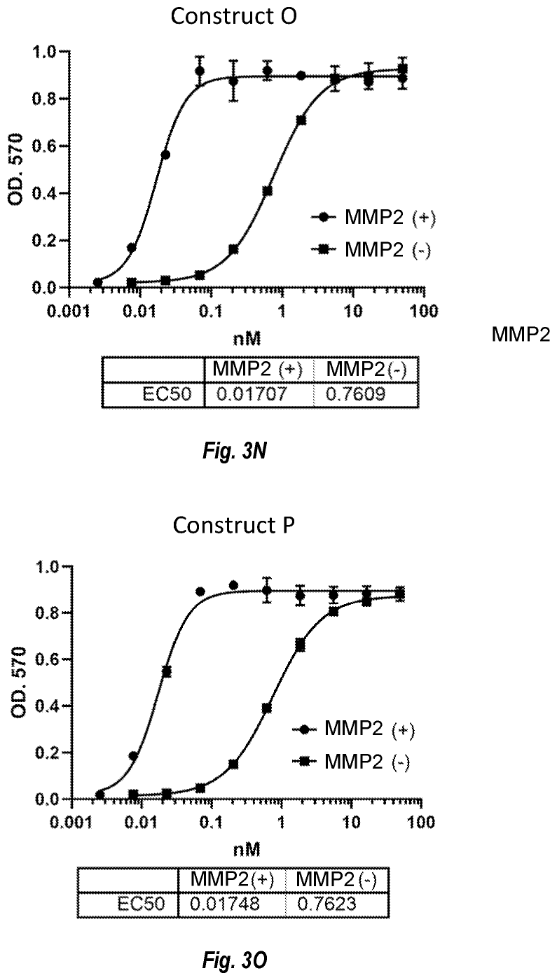

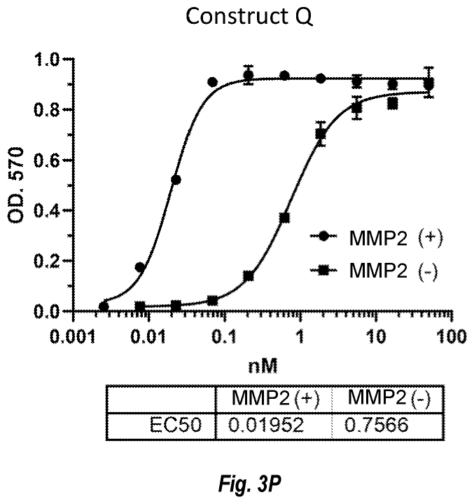

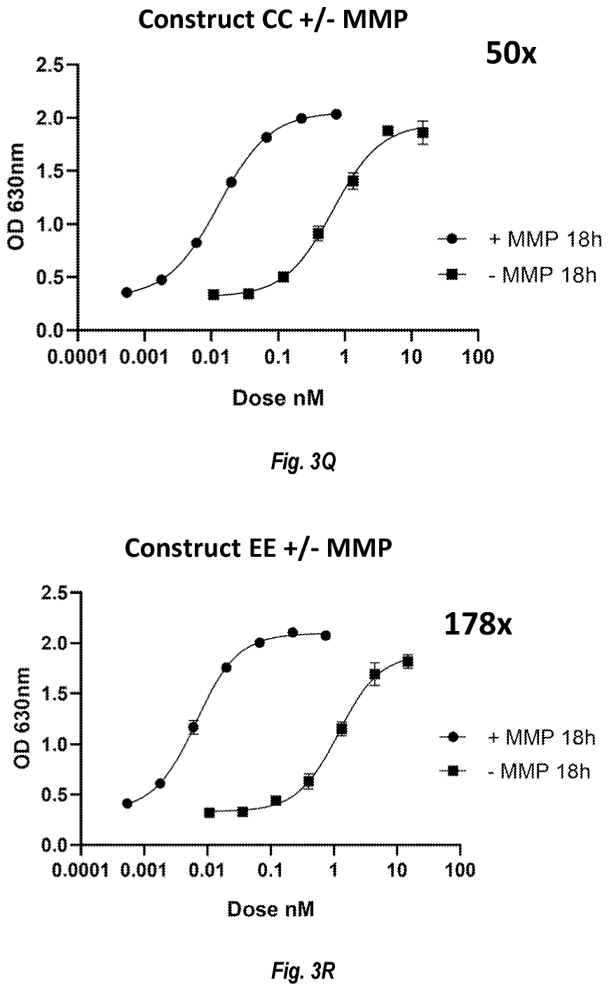

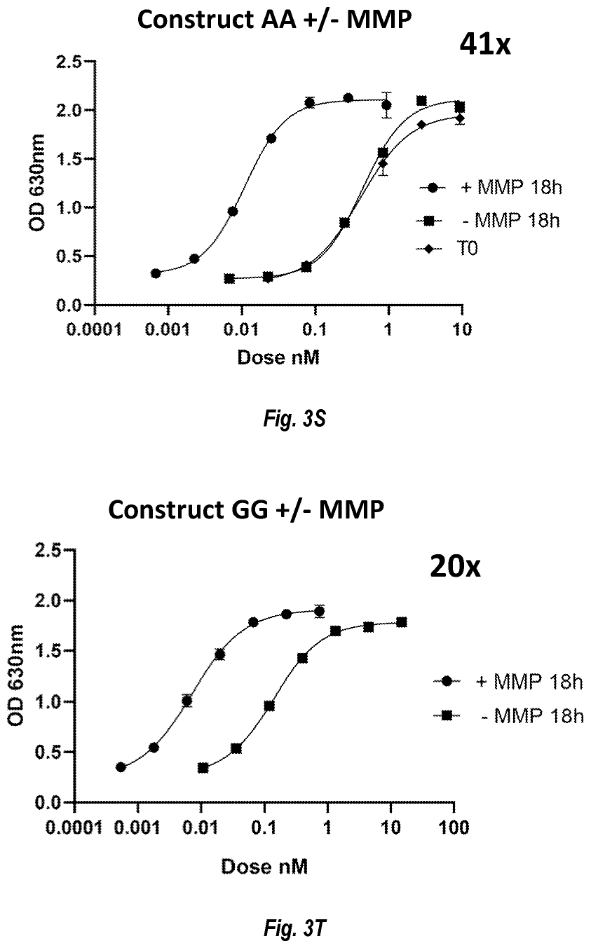

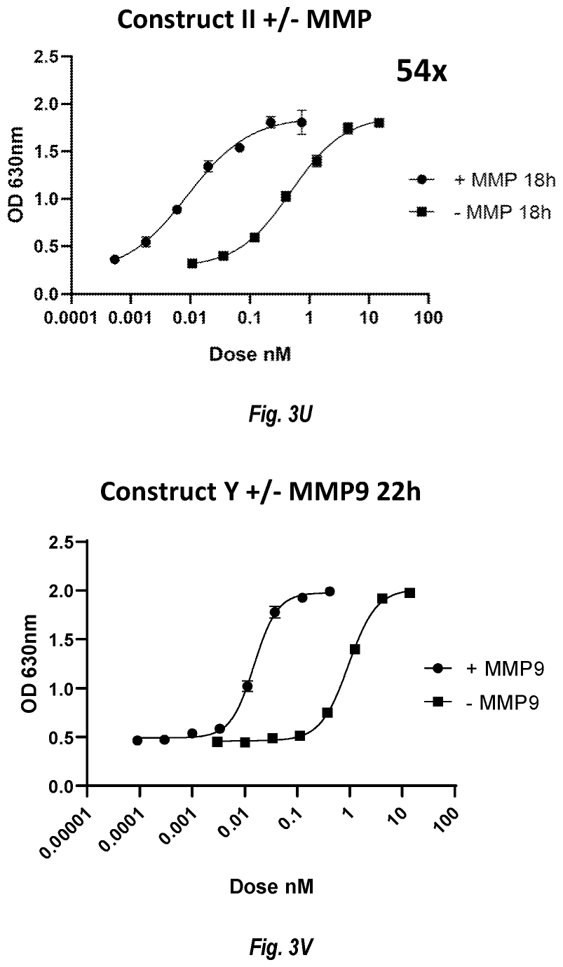

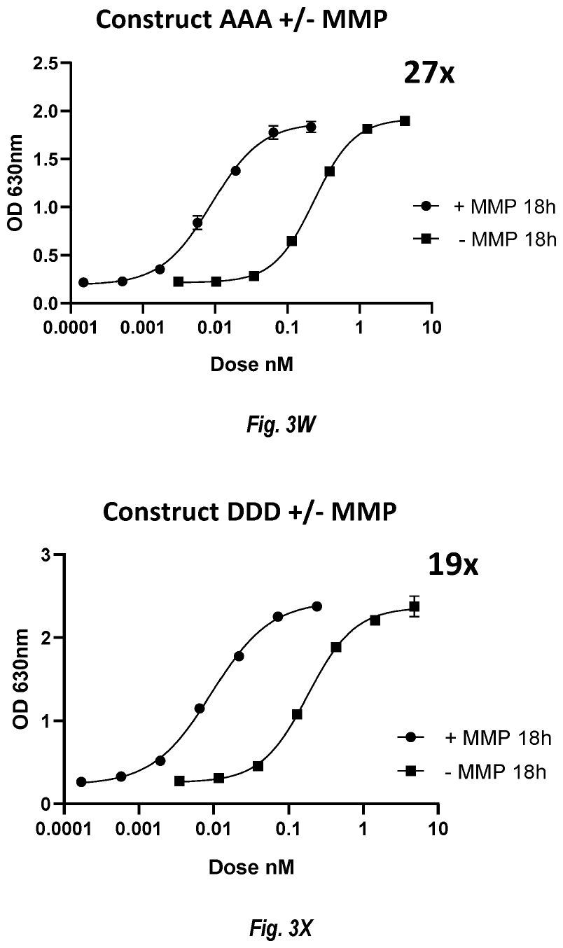

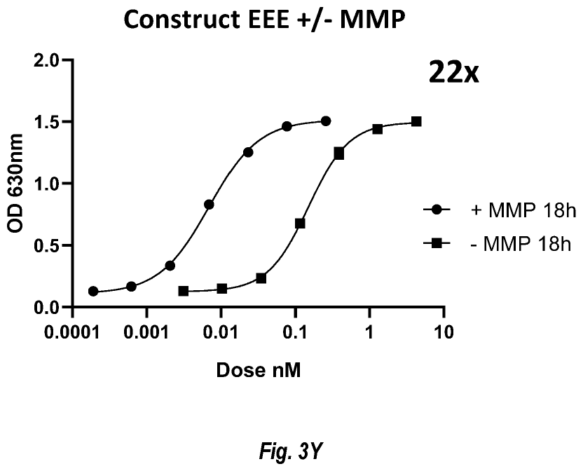

IL-2 activity was measured using either CTLL-2 cells (ATCC) or the reporter cell line HEK Blue IL2 (Invivogen, San Diego). In brief, for the CTLL-2 assay a titration of untreated and digested samples is added to 40 000 CTLL-2 cells per well in 100 ul medium in a 96-well plate and incubated at 37C in 5% CO2 for 18-22 hr. At the end of this period, 50ug/well Thiazolyl Blue Tetrazolium Bromide (MTT) (Sigma-Aldrich) was added and the plate was incubated for 5 hr at 37C in 5% CO2. Cells were lysed with 100 u1/well 10% SDS (Sigma) acidified with HCl, incubated at 37C for 4 hr, and absorbance was read at 570 nm. Recombinant human or mouse IL-2 (Peprotech and R&D systems respectively) was used as a positive control. FIGS. 3A-B, 3K-L and 3N-P show examples of untreated and digested fusion proteins evaluated in CTLL-2 proliferation assay.

HEK-Blue™ IL-2 cells are specifically designed to monitor the activation of the JAK-STAT pathway induced by IL-2. Indeed, stimulation with human or murine IL-2 triggers the JAK/STAT5 pathway and induces secreted embryonic alkaline phosphatase (SEAP) production. SEAP can be readily monitored when using QUANTI-Blue™, a SEAP detection medium. These cells respond to human and murine IL-2. For the HEK Blue assay, untreated and digested samples are titrated and added to 50 000 HEK Blue cells per well in 200 ul medium in a 96-well plate and incubated at 37C in 5% CO2 for 20-24 hr. The following day, levels of SEAP are measured by adding 20 uL of cell supernatant to QuantiBlue reagent, followed by 1-3 h incubation at 37C and reading absorbance at 630 nm. FIGS. 3C-J, 3Q-Y and Table 5B-5C show results obtained from IL2 fusion proteins tested in HEK Blue IL2 assay.

TABLE 5B

| | +MMP | −MMP | FOLD DIFFERENCE |
|---|---|---|---|
| | EC50 | EC50 | EC50 |
| CANDIDATE | (nM) | (nM) | (HEK BLUE IL2) |
| Construct E | 0.0073 | 0.103 | 14 |
| Construct L | 0.0061 | 0.0453 | 7.3 |
| Construct K | 0.0055 | 0.0933 | 17 |
| Construct J | 0.0059 | 0.1264 | 21 |
| Construct F | 0.0078 | 0.1736 | 22 |
| Construct I | 0.0074 | 0.3165 | 43 |

TABLE 5C

| | +MMP EC50 | −MMP EC50 |
|---|---|---|
| CANDIDATE | (nM) | (nM) |
| Construct AA | 0.01088 | 0.4423 |
| Construct Y | 0.0109 | 1.232 |
| Construct CC | 0.013 | 0.66 |
| Construct EE | 0.0066 | 1.18 |
| Construct GG | 0.0072 | 0.14 |
| Construct II | 0.009 | 0.489 |
| Construct AAA | 0.0089 | 0.23 |
| Construct DDD | 0.0094 | 0.181 |
| Construct EEE | 0.0069 | 0.149 |

Aggregation, stability, and homogeneity of Construct E, Construct M, and Construct N were compared using Coomassie-stained SDS-PAGE analysis (FIG. 3M). Construct M and Construct N showed decreased aggregation and greater stability and homogeneity, consistent with there being an improvement resulting from deletion of O-glycosylation sites.

### Example 7: In Vitro Serum Stability of Fusion Protein

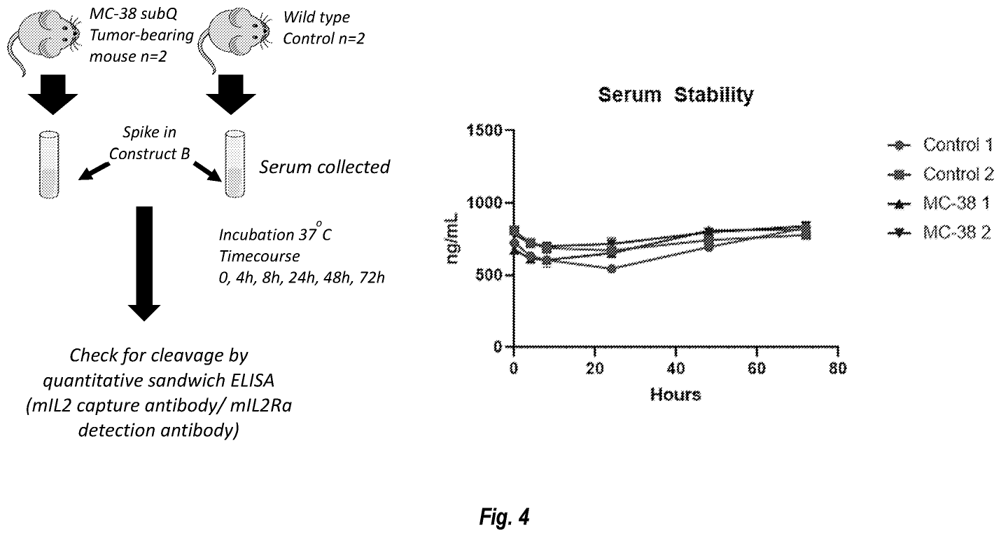

Construct B was incubated at 37C for up to 72 h with serum collected from 8 weeks old female C57BL/6 naive and MC38 tumor bearing mice respectively (n=2 per serum type, tumor volume >3000 mm3 at time of collection), in order to examine both non-specific cleavage as well as MMP-specific off-target cleavage. Samples were collected at 0 h, 4 h, 8 h, 24 h, 48 h and 72 h and the intact non-MMP cleaved fusion protein was quantified using an in-house developed sandwich ELISA. Results (see FIG. 4) show that the levels of fusion protein are stable in both serum types, indicating 1) a lack of off-target protein cleavage up to 72 hrs and 2) no active MMPs in circulation.

### Example 8: Pharmacokinetic Evaluation of Fusion Protein in Non-Tumor Bearing Mice

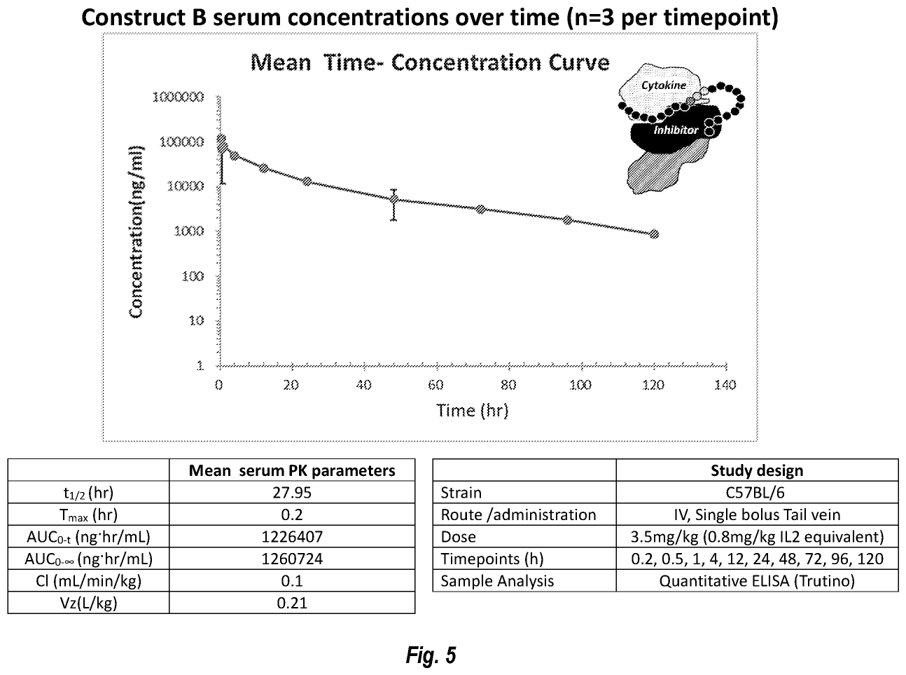

For this study, C57BL/6 8-10 weeks old female mice (Jackson Labs) were assigned to different groups (3 mice per treatment group). Mice received a single dose of fusion protein via IV injection (3.5 mg/kg). 3 mice/group/time point were bled at the following time points: pre-dose (0 h), 10 min, 30 min, 1 h, 4 h, 12 h, 24 h, 48 h, 72 h, 96 h and 120 h post dose. Blood samples were collected in Eppendorf tubes and processed to serum, then stored at −80C until testing. Samples were then evaluated by ELISA to quantify intact fusion protein levels. Mean serum concentrations of fusion protein were plotted over time and PK parameters were calculated using WinNonlin 7.0 (non-compartmental model) as shown in FIG. 5.

### Example 9: In Vivo Efficacy of Fusion Proteins in Syngeneic MC38 Colorectal Cancer Model

a. Intra-Tumoral Injection of Construct A

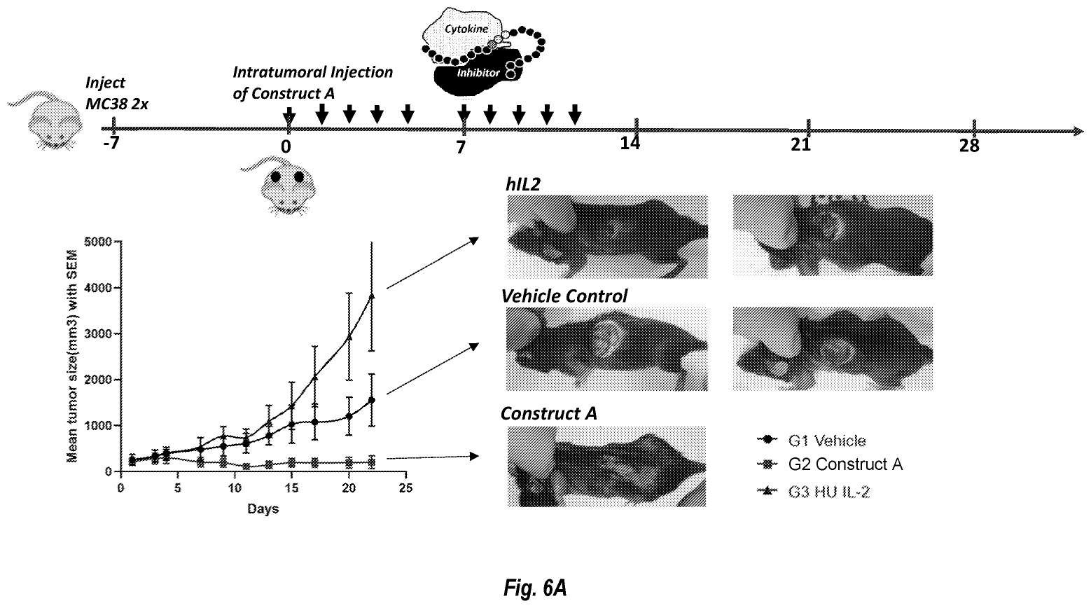

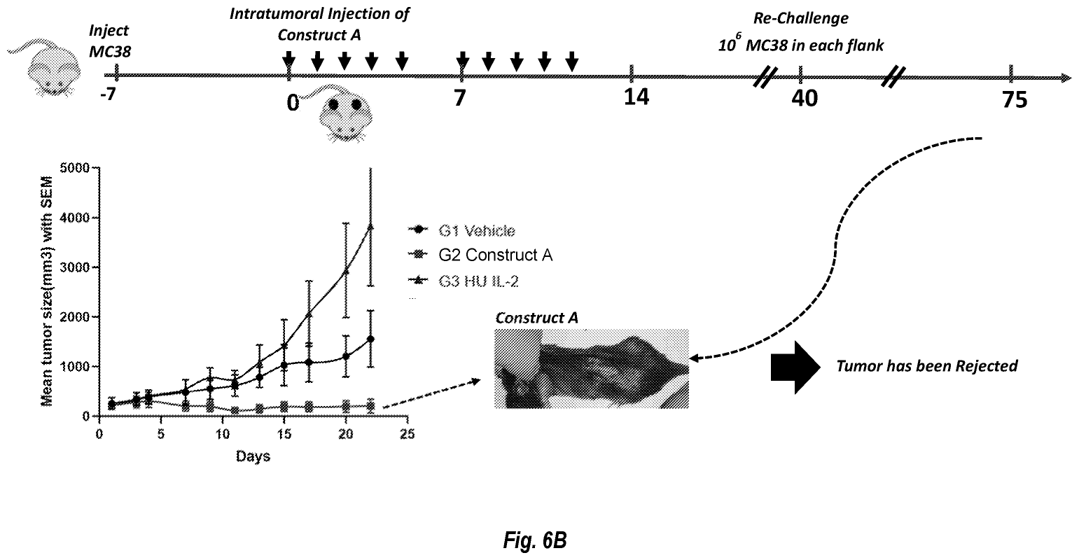

Pilot PK data indicates that Construct A is rapidly cleared from circulation (˜30-fold drop in serum levels within 30 min of IV injection). This is common for small therapeutic proteins whose molecular weight is below the renal glomerular filtration cut-off of ˜ 60-70 kDa. Hence, we reasoned this fusion protein was not amenable to systemic IV dosing for our POC in vivo efficacy study. Instead, we chose a direct intra-tumoral delivery design with 3 arms: vehicle, recombinant human IL-2 (r hIL2) and Construct A (n=3 mice/arm). IL-2 has previously demonstrated anti-tumor activity in a variety of syngeneic models by direct tumor injection, and based on this data, we selected to dose r hIL2 at 5 ug/day (equivalent to 50 000 U/day. Construct A was dosed at 70ug/day, which represents a 5 molar excess compared to recombinant IL-2 to compensate for the EC50 difference observed in the CTLL-2 assay. All agents and vehicle were injected daily into subcutaneous MC38 tumor mass (˜200 mm3 in size upon initiation of dosing) growing on the flank of C57BL/6 mice for 12 days with 2-day holiday after first 5 injections (total of 10 injections). Tumors and body weights were measured twice a week for the duration of the study. Tumor volumes were calculated using the following equation: (longest diameter*shortest diameter²)/2. As shown in FIG. 6A, remarkable anti-tumor activity was observed for Construct A. Indeed, a complete elimination of tumor was observed in Construct A treatment group while no tumor regression was observed in either vehicle or r hIL2 treatment groups. When ‘cured’ Construct A-treated mice were re-inoculated with MC38 tumor cells (10⁶ cells on opposite flank) on Day 40, no tumor mass was established a month after re-challenge suggesting the existence of a ‘memory’ immune response in these mice (FIG. 6B).

b. Systemic IV Injection of Construct B

The objective of this study is to evaluate efficacy of Construct B in the MC38-bearing female C57BL/6 mice. For this study, C57BL/6 6-8 weeks old female mice (Jackson Labs) were subcutaneously inoculated with MC38 cells (10⁶ cells/animal), and when the average tumor volume reached about 80 mm³, animals were randomized into 2 groups based on tumor volumes (8 mice per treatment group). Animals were dosed according to the following study design:

| | | | | Dosing | Dose | Dose |
|---|---|---|---|---|---|---|
| | | | Dose | Frequency & | Level | Volume |
| Group | Treatment | N | Route | Duration | (mpk) | (ul) |
| 1 | Vehicle Control | 8 | IV | Q3D for 21 D | N/A | 100 |
| 2 | Construct B | 8 | IV | Q3D for 21 D | 10 | 100 |

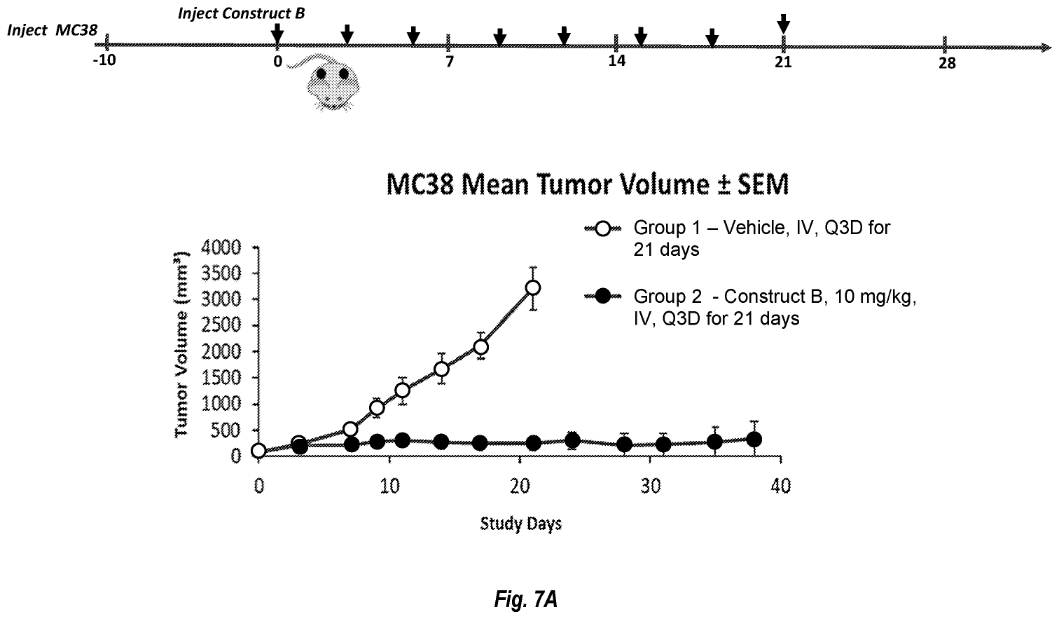

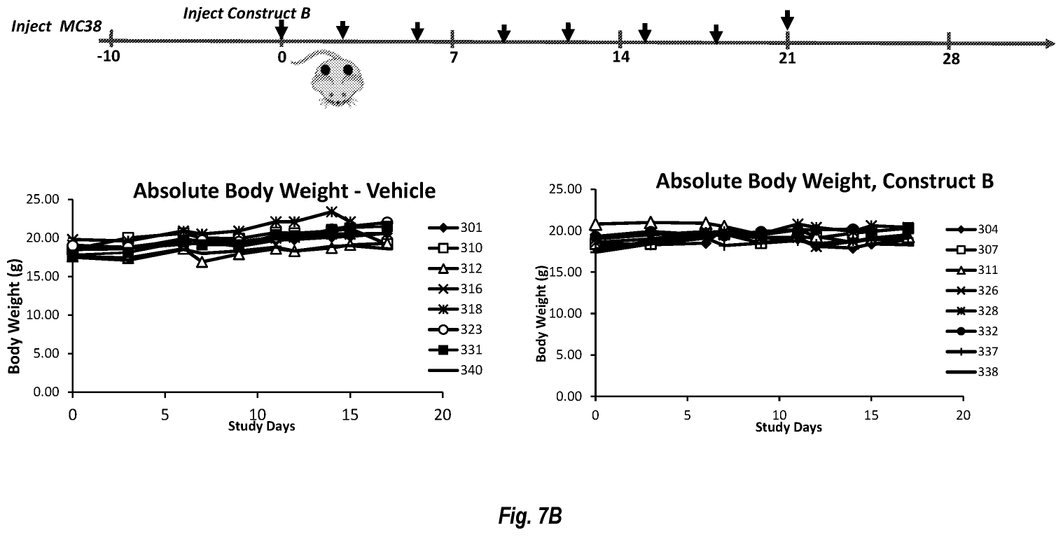

Mice were dosed over a 21 day period then further observed for an additional week. Tumors and body weights were measured twice a week for the duration of the study. Tumor volumes were calculated using the following equation: (longest diameter*shortest diameter²)/2. FIG. 7 shows the mean tumor volume over time for both groups (FIG. 7a) and individual body weights of vehicle and treated (FIG. 7B) animals.

The results showed excellent efficacy for the treatment group, with 92% inhibition of tumor growth at Day 21, while no adverse effect was observed. Remarkably, out of 8 cases, 3 complete tumor regressions (‘cures’) occurred in the colorectal cancer syngeneic setting

### Example 10: Evaluation of Immune Cell Populations by Immunohistochemistry (ruC) in MC38 Colorectal Cancer Samples

The objective of this study is to evaluate immune targets in tumor samples by IHC. See below for details:
CD4+Foxp3 double immunofluorescence staining
CD8, CD25, CD3, CD4 and CD335 single IHC staining

Note that prior to performing IHC, H&E staining was ran for all control and Construct B treated tumors to check the tissue quality.

7 tumor samples were selected from the systemic in vivo efficacy study and formalin-fixed paraffin embedded (FFPE) blocks were prepared following standard embedding process.

Model type: MC38

| | | Number of |
|---|---|---|
| Group | Treatment | FFPE blocks |
| 1 | Vehicle, IV, Q3D for 21 days | 4 |
| 2 | Construct B, 10 mg/kg, IV, Q3D for 21 days | 3 |

The following antibodies were used:

| Antibody | Company | Cat# | Type | Reactivity |
|---|---|---|---|---|
| CD4 | Cell | 25229 | Rabbit IgG mAb | Mouse |
| | Signaling | | | |
| FoxP3 | Cell | 12653 | Rabbit IgG mAb | Mouse |
| | Signaling | | | |
| CD8 | Cell | 98941 | Rabbit IgG mAb | Mouse |
| | Signaling | | | |
| CD25 | abcam | ab227834 | Rabbit IgG mAb | Mouse |
| CD3 | Cell | 99940 | Rabbit IgG mAb | Mouse |
| | Signaling | | | |
| CD335 | R&D | AF2225- | Goat IgG pAb | Mouse |
| | Systems | SP | | |
| Bond | Leica | DS9800 | Anti-rabbit Poly-HRP-IgG | |
| Polymer | | | (<25 μg/mL) containing 10% | |
| Refine | | | (v/v) animal serum in tris- | |
| Detection | | | buffered saline/0.09% ProClin ™ | |
| | | | 950 (ready-to-use) | |
| ImmPRESS | Vector | MP-7405 | Anti-goat Poly-HRP-IgG | |
| HRP Anti- | | | (<25 μg/mL) containing 10% | |
| Goat Ig | | | (v/v) animal serum in tris- | |
| | | | buffered saline (ready-to-use) | |
| | | | and House serum (2.5%) | |
| Rabbit | Cell | 3900 | Isotype control | |
| (DA1E) | Signaling | | | |
| mAb IgG | | | | |
| TRITC | PerkinElmer | | NEL742001KT | Fluorescent |
| TSA(Red) | | | | double |
| | | | | staining |
| FITC | PerkinElmer | | NEL741001KT | Fluorescent |
| TSA(Green) | | | | double |
| | | | | staining |

FFPE blocks were sectioned with a manual rotary microtome (4 μm thickness/section) and optimized IHC assay protocols for all the antibodies were used. All stained sections were scanned with NanoZoomer-S60 Image system with 40× magnification. High resolution picture for whole section was generated and further analyzed.

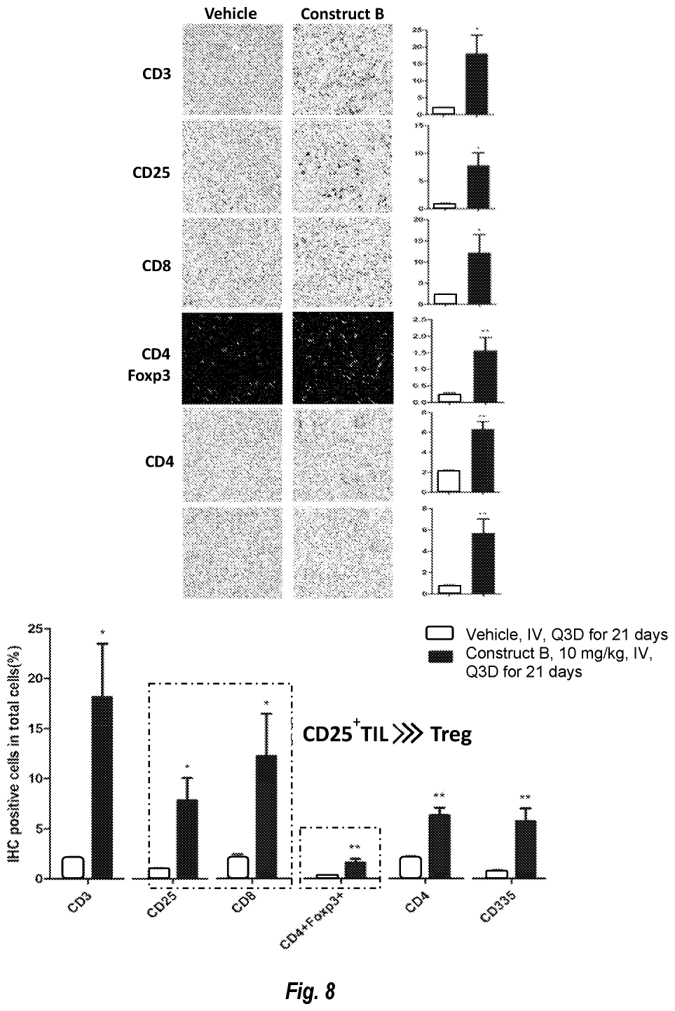

Scoring Method: All the images were analyzed with HALO™ Image Analysis platform. The whole slide image was analyzed and necrosis area was excluded. The total cells and IHC positive cells were counted. IHC score is presented as the ratio of the positive cell counts against the total cell numbers within whole section and shown in FIG. 8. Results show that there is a significant increase in tumor infiltrating immune cells post Construct B treatment.

### Example 11: In Vivo MMP Activity Evaluation in Diverse Syngeneic Tumor Models

We assessed the degree of MMP activity in the models in vivo utilizing an MMP-activatable fluorescent probe, MMPSense 680™. This probe is optically silent in its intact state and becomes highly fluorescent following MMP-mediated cleavage and is designed to be used as a real-time in vivo imaging tool (Perkin Elmer). Following a single dose IV injection of the probe to tumor-bearing mice, fluorescent images were captured over 6 days and the fluorescence intensity in tumor area, which is directly proportional to MMP activity present, was quantified (FIG. 9). All models showed intrinsically different levels of MMP activity.

### Example 12: In Vivo Efficacy of Construct B in Diverse Syngeneic Tumor Models

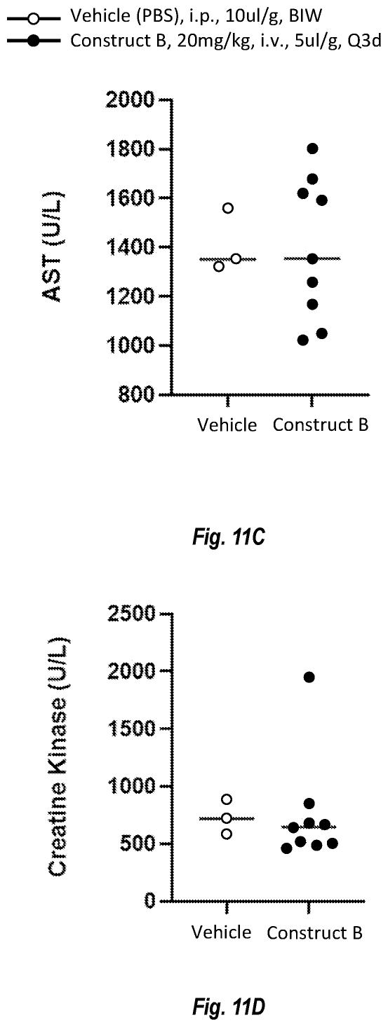

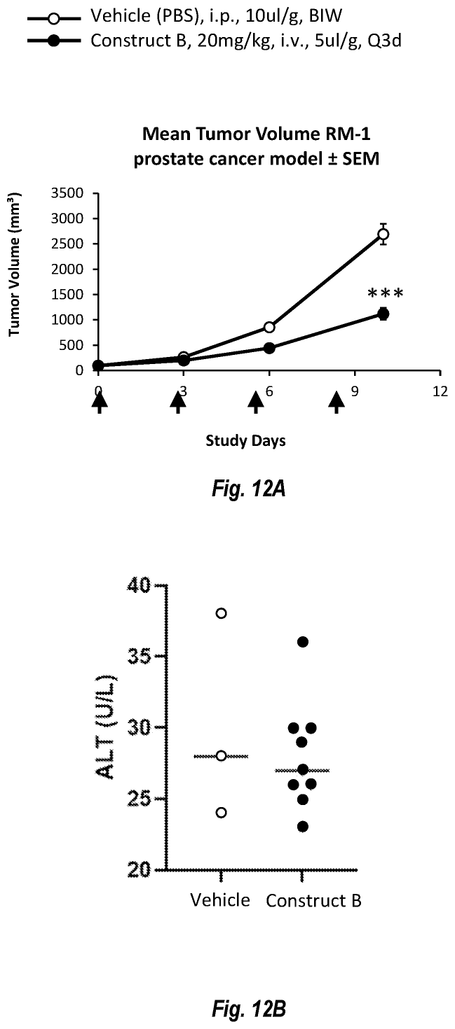

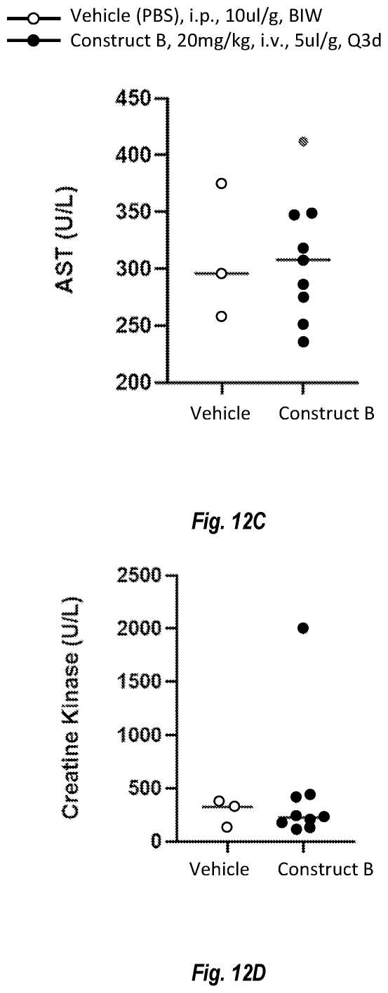

For the efficacy studies, C57BL/6 or BALB/c mice were subcutaneously inoculated with malignant cells and when the average tumor volume reached on average 90 mm³, animals were randomized into 2 groups based on tumor volumes (n=10 mice per treatment group). Mice were dosed intravenously every 3 days (Q3D) at 20 mg/kg. Tumors, body weights and clinical observations were measured/collected twice a week for the duration of the study. Tumor volume is shown in FIGS. 10A-D, 11A, 12A, and 13B-C. Robust anti-tumor activity was observed in several models, notably 49% tumor growth inhibition (TGI) was observed at D12 in the B16F10 melanoma model and 58% tumor TGI at Day 10 in the aggressive Ras/Myc transformed RM-1 prostate cancer model (FIG. 10C-D and Table 6). Notably, no signs of toxicity, including body weight loss and elevated levels of liver and/or kidney enzymes, were noted and clinical observations were normal in these models. Liver and kidney enzyme results corresponding to FIGS. 11A and 12A are shown in FIGS. 11B-D and 12B-D, respectively.

TABLE 6

| | | | | Max | | |
|---|---|---|---|---|---|---|
| Cancer | | | Dosing | TGI | | MMP |
| type | strain | Model | Regimen | % | T test | score |
| Breast | BALB/c | EMT06 | 20 mpk, | 43 | P = | HIGH |
| | | | IV Q3D | (D20) | 0.0006 | |
| Melanoma | C57BL/6 | B16F10 | 20 mpk, | 49 | P = | LOW |
| | | | IV Q3D | (D12) | 0.0004 | |
| colorectal | BALB/c | CT-26 | 20 mpk, | 46 | P = | MED/ |
| | | | IV Q3D | (D13) | 0.0114 | HIGH |
| colorectal | C57BL/6 | MC-38 | 10 mpk, | 92 | P < | MED |
| | | | IV Q3D | (D21) | 0.0001 | |
| prostate | C57BL/6 | RM_1 | 20 mpk, | 58 | P < | NOT |
| | | | IV Q3D | (D10) | 0.0001 | DETER- |
| | | | | | | MINED |

P values represent unpaired t test (graphpad prism) between vehicle and Construct B groups on Day of max TGI.

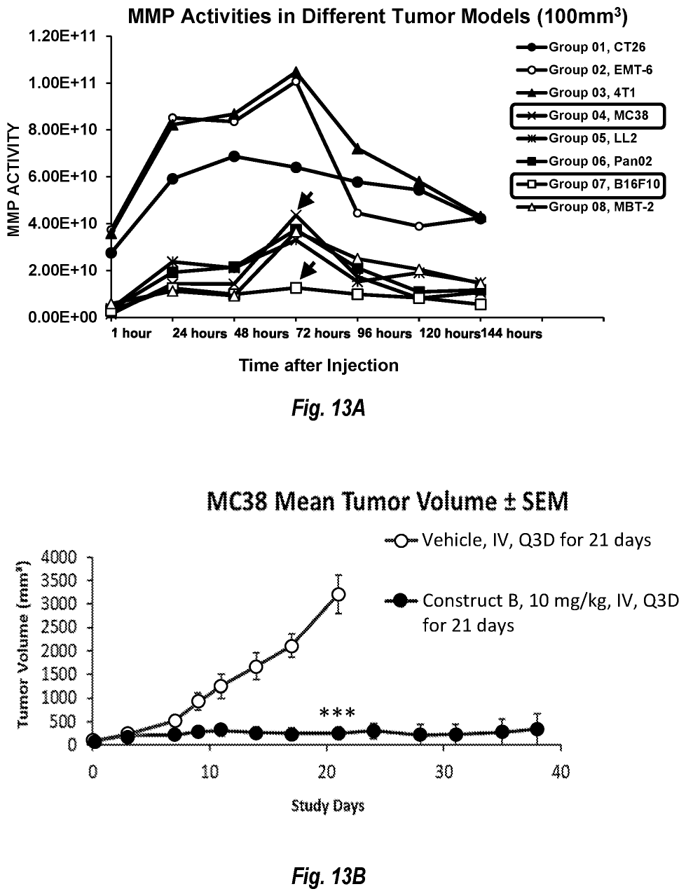

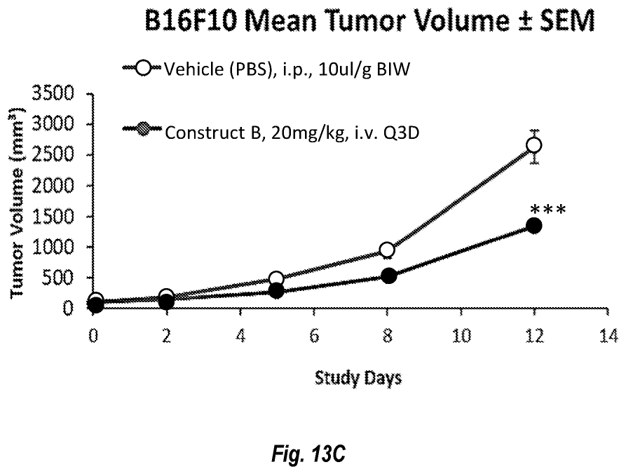

The difference in efficacy between MC38 and B16F10 models may in part be due to the lower MMP activity measured in B16F10 tumors, resulting in less functional IL-2 being released in the TME relative to the MC38 setting (FIG. 13A).

### Example 13: Next Generation Retention Linker Peptide Binding Assay

A series of peptides comprising an MMP cleavable site with or without the addition of a tumor retention sequence were synthesized and conjugated to the fluorophore EDANS (5-((2-Aminoethyl)amino)naphthalene-1-sulfonic acid) (custom synthesis, ThermoFisher). Table 7 shows the list of peptides. These peptides were then tested for their ability to bind ECM proteins such as heparin, fibronectin and collagen which are found in abundance in the tumor stroma.

TABLE 7

| SEQ | | |
|---|---|---|
| ID | | Target of |
| NO | Sequence | retention motif |
| 901 | GGGSGGGGPLGVRG-* | None (1^{st} gen) |
| 902 | GG [symbol] GPLGVRG-* | pH dependent heparin |
| 903 | G [symbol] -* | heparin |
| 904 | [symbol] GPLGVRG-* | heparin |
| 907 | GGGSGGGPAALIGG-* | None (1^{st} gen) |
| 913 | G [symbol] GPLGVRG-* | pH dependent fibronectin |
| 914 | [symbol] GPLGVRG-* | Collagen IV |
| 915 | GGGSG [symbol] | Collagen I |

Underlining indicates MMP cleavage site. Bold italics indicates retention motif when present.
-*represents Edans fluorophore conjugated to peptide.

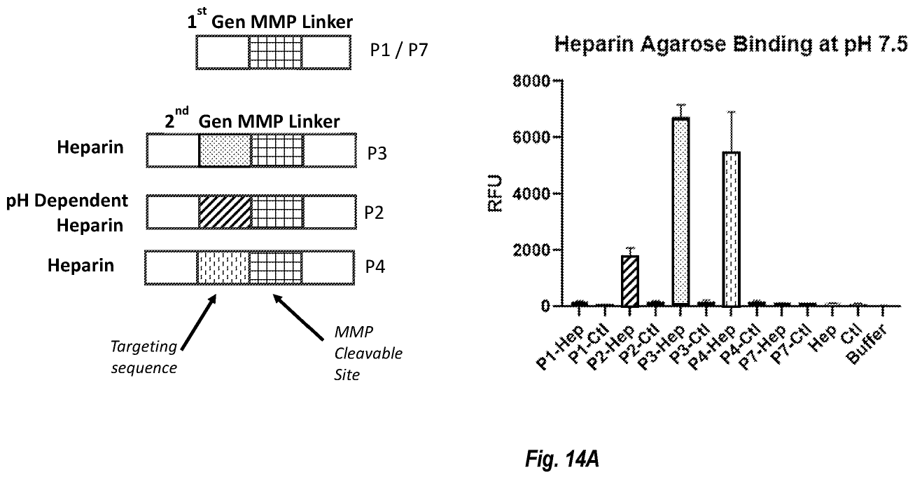

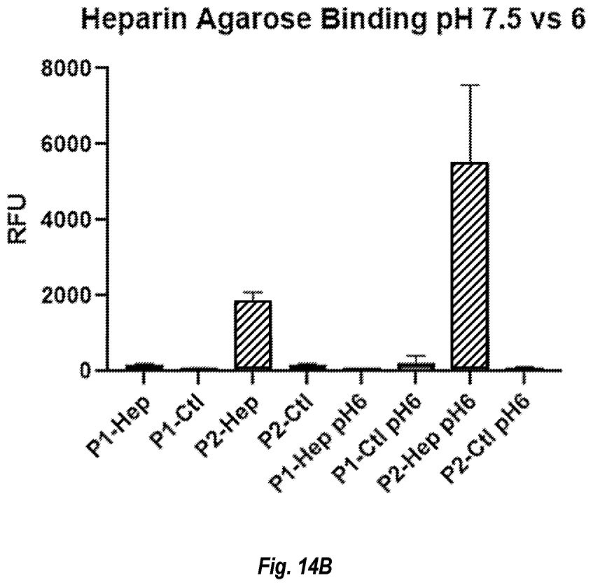

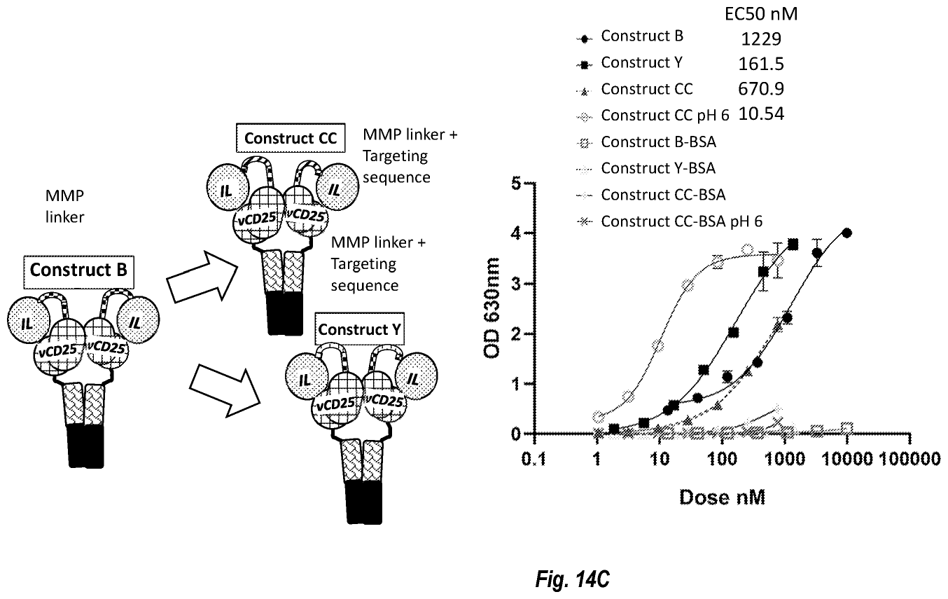

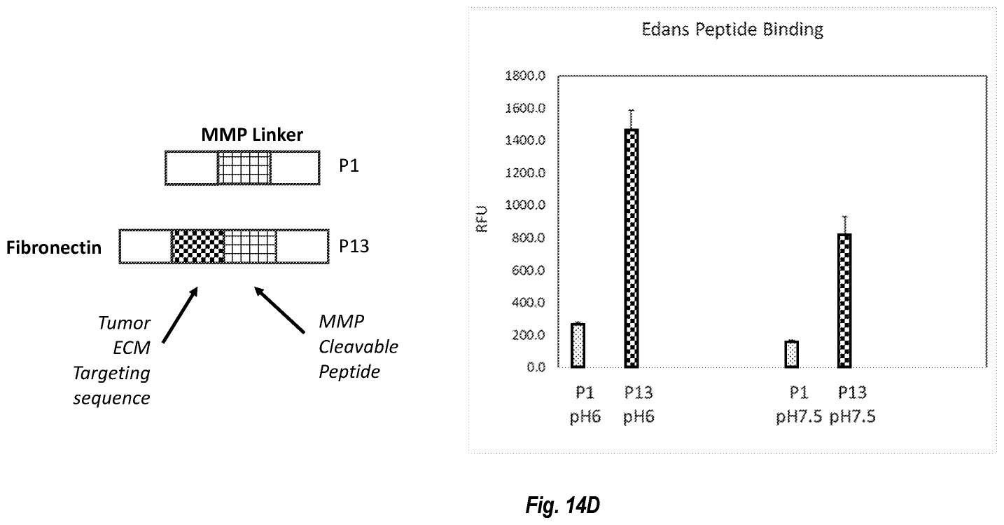

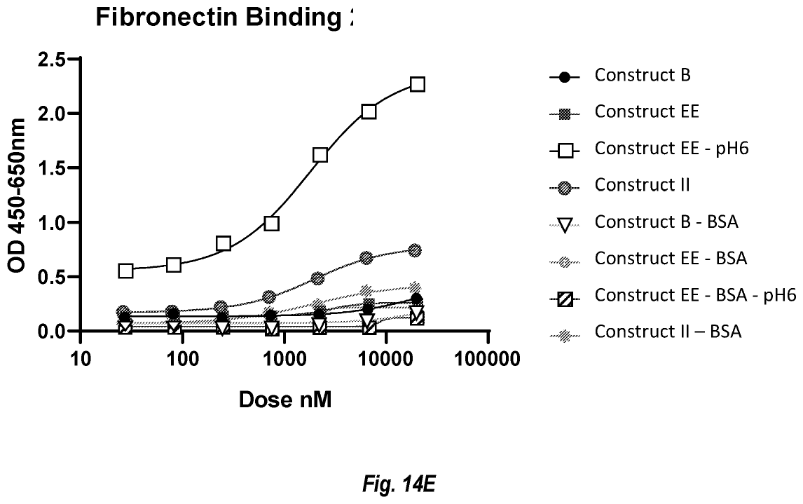

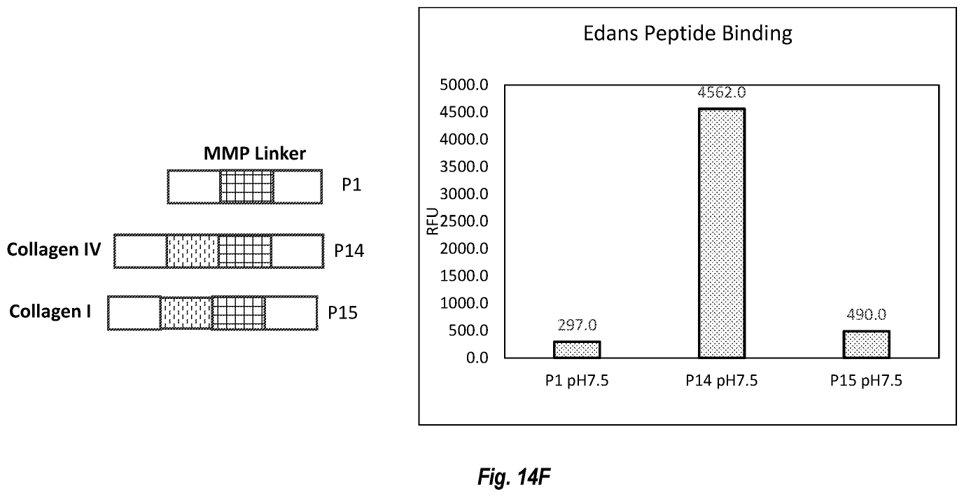

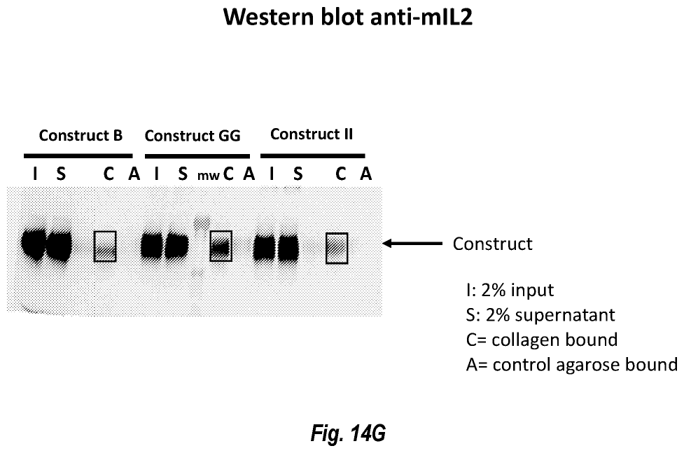

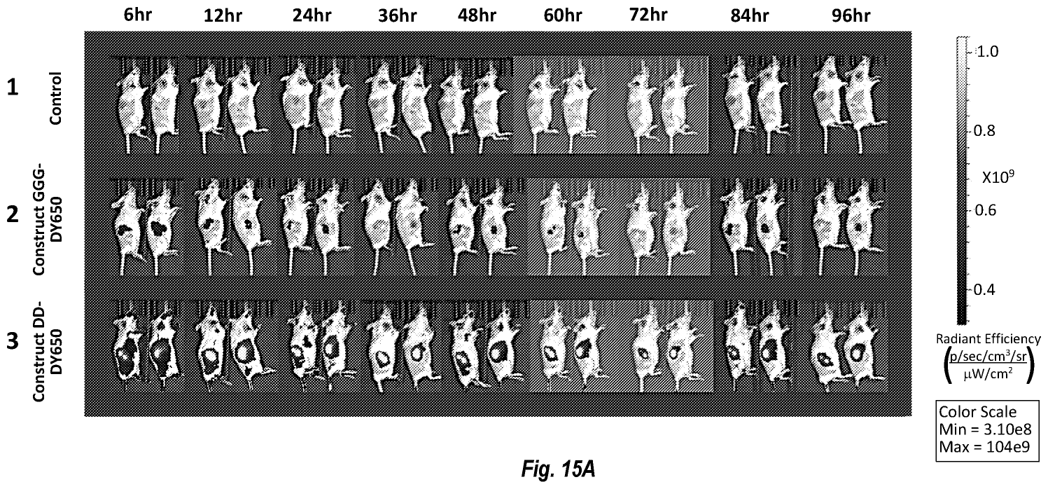

All binding assays were set up in 10 mM TrisHCl pH 7.5 and/or 10 mM TrisHCl pH 6. Peptides (20 uM) were incubated on a shaker for 2 hrs at room temperature with agarose cross-linked to heparin or control agarose beads (Sigma and Pierce respectively). The beads were then washed 4 times and resuspended in 100 uL of binding buffer in a black 96-well plate. Peptide binding was quantified by measuring the fluorescence of samples using excitation/emission spectra of EDANS (Ex 340/Em 490). FIG. 14A shows that several next generation MMP linker peptides containing heparin binding motifs bind to the heparin-agarose beads while 1^{st} generation MMP linkers lacking these retention sequences do not. One such peptide displays enhanced binding to heparin at pH6 (the pH of tumors) vs pH 7.5 (pH of normal tissues) (FIG. 14B).

For fibronectin and collagen binding assays, streptavidin coupled magnetic beads (Mag Sepharose, Cytiva and Dynabeads, ThermoFisher, respectively) were first incubated with biotin-labelled fibronectin (Cytoskeleton) or biotin-labelled collagen IV (Prospec) for 1 Hr with gentle shaking. Following multiple washes, the ECM-coated beads were then incubated with Edans Peptides (20 uM) for 2 hours at room temperature with shaking in neutral or acidic binding buffer. Beads were then washed and resuspended in 100 uL of binding buffer in a black 96-well plate. Peptide binding was quantified by measuring the fluorescence of samples using excitation/emission spectra of EDANS (Ex 340/Em 490). FIG. 14D shows that peptide 13 is able to bind fibronectin and displays enhanced binding at pH6 (the pH of tumors) vs pH 7.5 (pH of normal tissues). FIG. 14F shows that peptide 14 strongly binds collagen IV while peptide 15 binds to a lesser extent.

### Example 14: Next Generation Tumor Retention IL-2 Fusion Protein Binding Assays

A series of IL-2 fusion proteins comprising tumor retention sequences in the linker regions were designed and successfully manufactured (Table 3 and FIGS. 1C-D). These proteins were then tested for their ability to bind ECM proteins such as heparin, fibronectin and collagen which are found in abundance in the tumor stroma.

96-well plates were coated with 25 ug/mL of Heparin-BSA conjugate (provided by Dr. Mueller, Boerhinger Ingelheim) or control BSA for 18-22 h at room temperature on shaker (350 rpm). After washing, wells are blocked with PBS-0.05% Tween 20/1% BSA for 90 min, then fusion proteins are titrated in 1% BSA/PBS-0.05% Tween 20 pH 7.5 and/or pH 6 and added for 2 hr at room temperature with shaking. After washing, an anti-mouse IL-2 biotin-labelled detection antibody (JES6-5H4, ThermoFisher) is added and binding is detected using Ultra Strepavidin HIRP (ThermoFisher). The plate was developed by adding the chromogenic tetramethylbenzidine substrate (Ultra TMB, ThermoFisher). The reaction is stopped by addition of 0.5M H2SO4 and the absorbance is read at 450-650 nm. IL-2 fusion variants Construct Y and Construct CC at acidic pH bind heparin in dose-dependent manner and with higher affinity than Construct B (FIG. 14C). Strikingly, Construct CC preferentially binds heparin at acidic pH and shows the most robust binding with EC50 10 nM, while Construct B's binding is much weaker with >100-fold higher EC50 value.

A similar plate-based assay was developed to interrogate binding of IL-2 fusion variants to fibronectin. 96-well plates were coated with 4 ug/mL of fibronectin (Sigma) or control BSA for 18-22 h at room temperature on shaker (350 rpm). After washing, wells are blocked with protein-free blocking buffer (Pierce) for 90 min, then fusion proteins are titrated in blocking buffer-0.1% Tween 20 pH 7.5 and/or pH 6 and added for 1 hr at room temperature with shaking. After washing, an anti-mouse IL-2 biotin-labelled detection antibody (JES6-5H4, ThermoFisher) is added and binding is detected using Ultra Streptavidin HRP (ThermoFisher). The plate was developed by adding the chromogenic tetramethylbenzidine substrate (Ultra TMB, ThermoFisher). The reaction is stopped by addition of 0.5M H2SO4 and the absorbance is read at 450-650 nm. Construct EE preferentially binds fibronectin at acidic pH and shows dose-dependent binding, while no binding is observed at pH 7.5 (FIG. 14E). No significant binding of Construct B is seen in either neutral or acidic conditions.

To test binding to collagen, a pulldown assay using agarose cross-linked to collagen (Sigma) was performed. IL-2 fusion proteins were incubated with collagen-agarose or control agarose beads for 18-22 h at 4C with gentle rotation in 1% BSA/PBS-0.05% Tween 20. After washing, proteins bound to the beads were eluted by resuspending beads in SDS sample buffer (Life Technologies). Bound proteins were then separated by SDS-PAGE on 4-12% BisTris gradient gel followed by immunoblotting with goat anti-mouse IL-2 polyclonal antibody (AF-402-NA; R&D systems). Donkey Anti-goat RP-conjugated antibody was used for detection (Jackson Immuno Research, West Grove, PA) and the blot was developed using the SuperSignal West Femto Maximum sensitivity detection reagent (ThermoFisher) following the manufacturer's recommendations. The blot image is shown in FIG. 14G. Construct GG and Construct II were specifically bound by collagen-agarose beads, while no IL-2 fusion protein bound the control agarose beads. Quantitation of the blot using iBright imaging system (Invitrogen), shows that although the fraction of bound Construct GG and Construct II was low (<1% of input), it was 2.5 and 1.4-fold higher than the fraction of bound Construct B (Table 8).

TABLE 8

| | Input | | Bound | |
|---|---|---|---|---|
| | (2%) | Bound | (% input) | Normalized |
| Construct | 16707 | 2306 | 0.3% | 1 |
| B | | | | |
| Construct | 15267 | 5191 | 0.7% | 2.5 |
| GG | | | | |
| Construct | 12094 | 2277 | 0.4% | 1.4 |
| II | | | | |

### Example 15: Next Generation Retention Linker IL-2 Fusion Proteins Show Greater Retention in Tumor In Vivo

We assessed the levels of IL-2 fusion proteins present in tumors in vivo by utilizing fluorescently labelled proteins and real-time whole-body imaging. Non-cleavable Construct GGG and Construct DD were conjugated to Dylight 650 probe according to the manufacturer's protocol (Dylight 650 Antibody labeling kit, ThermoFisher). We confirmed the conjugation did not significantly alter the proteins' binding to heparin. BALB/c mice were subcutaneously inoculated with EMT6 breast cancer syngeneic model and when the average tumor volume reached 240 mm³, animals were randomized into 3 groups based on tumor volumes (n=2 mice per treatment group). Table below shows study design:

| | | | | Dosing | Dose | Dose |
|---|---|---|---|---|---|---|
| | | | Dose | Frequency | Level | Volume |
| Group | Treatment | N | Route | & Duration | (mg/kg) | (mL/kg) |
| 1 | Control-PBS | 2 | IV | Once | NA | 4 |
| 2 | Construct | 2 | IV | Once | 8 | 4 |
| | GGG-DY650 | | | | | |
| 3 | Construct | 2 | IV | Once | 8 | 4 |
| | DD-DY650 | | | | | |

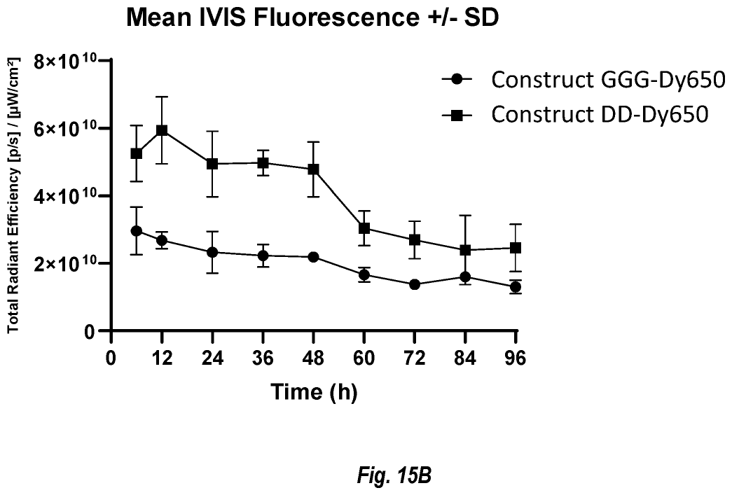

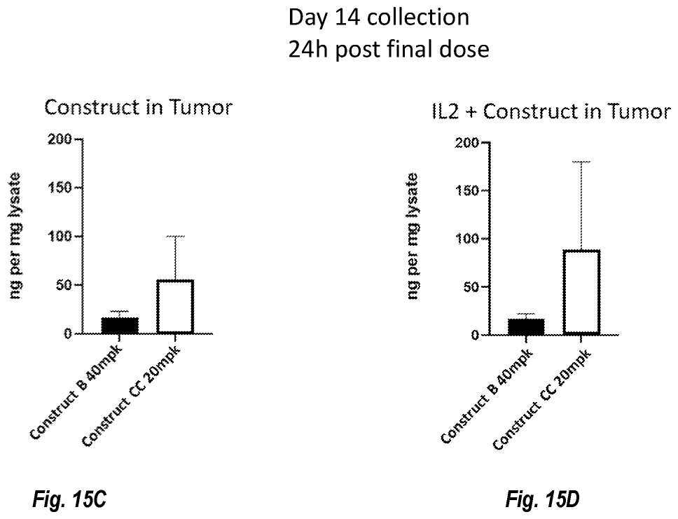

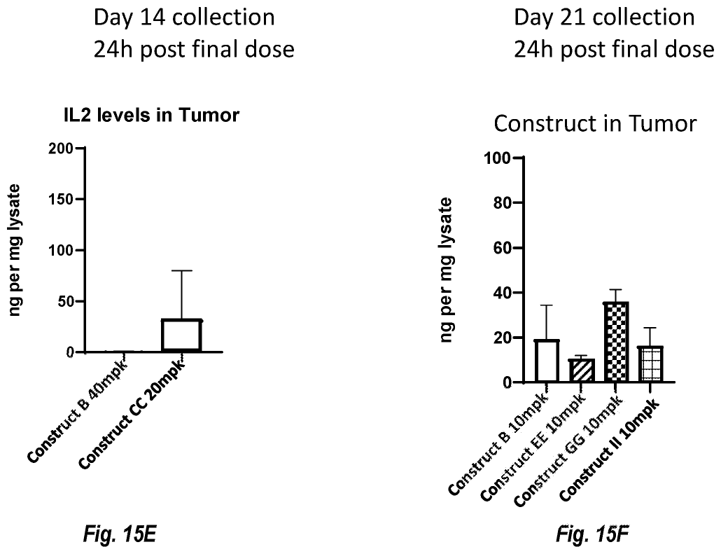

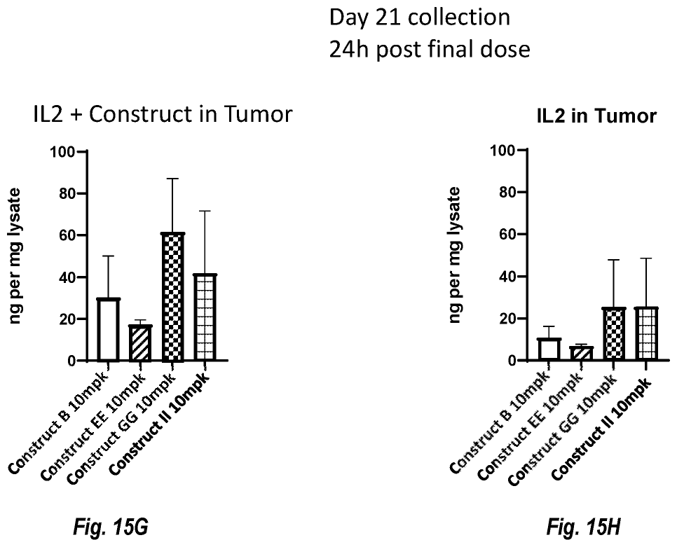

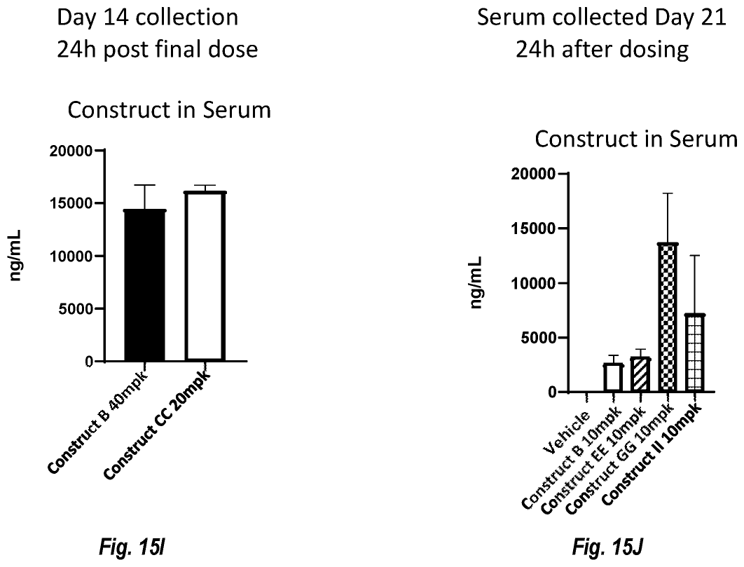

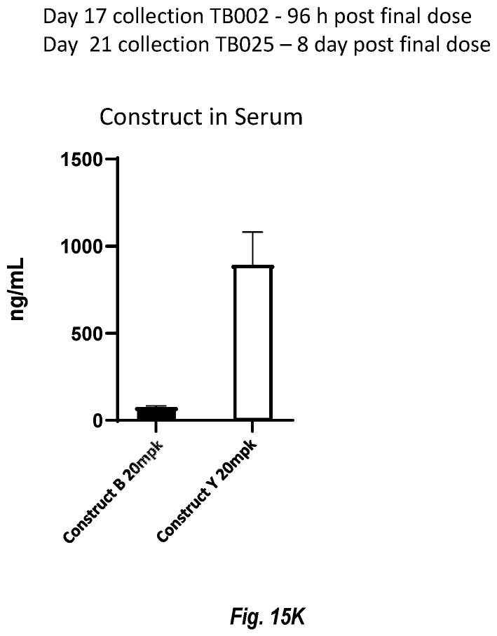

Following a single dose of the labeled IL-2 fusion proteins to tumor-bearing mice, fluorescent images (excitation 640/emission 680 consistent with Dylight 650 probe ex/em spectra) were captured over 96 hrs on an IVIS system (PerkinElmer, IVIS Lumina Series III) and are shown in FIG. 15A. The fluorescence intensity in tumor area was quantified across the groups, average background tumor fluorescence (group 1) was subtracted from group 2 and 3 values at each time-point, and data was normalized to the initial fluorescence intensity of same amount of each labeled protein. FIG. 15B shows that the tumor-associated fluorescence with group 3 is roughly 2-fold higher than that of group 2 at each of the time-points tested. This signifies next generation retention linker Construct DD accumulates and is retained in tumors at 2-fold higher levels compared to 1^{st} generation IL-2 fusion protein Construct GGG.

### Example 16: Next Generation Retention MMP-Linker Leads to Increased Levels of Drug and IL2 in Tumors and Serum In Vivo

We quantified levels of full-length IL2-IL2Ra fusion proteins and IL-2 in tumor samples collected during pre-clinical efficacy studies comparing Construct B and retention linker IL-2 fusion drugs (see example 17).

Tumors (n=3 per group) were collected 24 h after the last dose injection, flash frozen and stored at −80C until further processing. Tumor lysates were generated using tissue extraction reagent (ThermoFisher) supplemented with protease and phosphatase inhibitors and standard techniques and protein concentrations were determined using the BCA assay (Pierce).

Lysates were tested with in-house ELISAs to measure full-length IL-2 fusion proteins (IL-2 capture/IL-2Rα detection) and IL-2 fusion proteins+free IL-2 (IL-2 capture/IL-2 detection). Free IL-2 levels in tumor were calculated by subtracting drug levels from the drug+IL-2 data set. Results were normalized to 1 mg of tumor lysate and mean values are shown in FIG. 15C-H. Levels of Construct CC (20 mg/kg dose) in tumor are roughly 3-fold higher compared to Construct B levels, despite Construct B being dosed at 40 mg/kg (FIG. C). Drug level comparison in samples from the 10 mg/kg dosing cohort shows highest levels present in collagen binding Construct GG treated tumors (FIG. 15F). This indicates that retention linker technology may lead to a robust increase in drug amounts in tumor in vivo. Likewise, IL-2 levels in Construct CC, Construct GG and Construct II treated tumors are elevated compared to Construct B treated tumors (FIG. 15E/H). This implies that next generation retention linker technology is able to retain in TME both full-length drug and released IL-2 post-cleavage.

The equivalent serum samples (n=3 per group) were also tested with in-house ELISAs to quantify full-length IL-2 fusion drugs and results are shown in FIG. 15I-K. 24 hrs after dosing, circulating drug levels of Construct B (40 mg/kg) and Construct CC (20 mg/kg) are roughly similar despite the dosing difference, whilst in the 10 mg/kg cohort, Construct GG and Construct II drug levels in serum are roughly 5-fold and 3-fold higher than Construct B serum levels (FIG. 15J). Additional serum samples collected at Day 17 (Construct B 20 mg/kg) and Day 21 (Construct Y 20 mg/kg), 4 days and 8 days respectively after the last IV injection, were assayed for full-length IL-2 fusion drug. FIG. 15K shows that Construct Y drug levels in circulation are strikingly more than 10-fold higher than Construct B despite serum being collected 4 days later. Collectively, these data indicate retention linker technology leads to increased levels of drug in circulation.

### Example 17: In Vivo Efficacy of Retention Linker IL-2 Drugs in B16F10 Syngeneic Model

In a first efficacy study, C57BL/6 mice were subcutaneously inoculated with B16F10 melanoma cells and when the average tumor volume reached on average 70-90 mm³, animals were randomized into 5 groups based on tumor volumes (n=6 mice per treatment group). Mice were dosed intravenously every 3 days (Q3D) for a total of 5 doses according to following design:

| | | | | Dosing | Dose | Dose |
|---|---|---|---|---|---|---|
| | | | Dose | Frequency | Level | Volume |
| Group | Treatment | N | Route | & Duration | (mg/kg) | (mL/kg) |
| 1 | PBS- | 6 | IV | Q3D for 14 | NA | 4 |
| | Vehicle | | | days (5 | | |
| | | | | doses) | | |
| 2 | Construct B | 6 | IV | Q3D for 14 | 20 | 4 |
| | | | | days (5 | | |
| | | | | doses) | | |
| 3 | Construct B | 6 | IV | Q3D for 14 | 40 | 8 |
| | | | | days (5 | | |
| | | | | doses) | | |
| 4 | Construct Y | 6 | IV | Q3D for 14 | 20 | 4.45 |
| | | | | days (5 | | |
| | | | | doses) | | |
| 5 | Construct | 6 | IV | Q3D for 14 | 20 | 5 |
| | CC | | | days (5 | | |
| | | | | doses) | | |

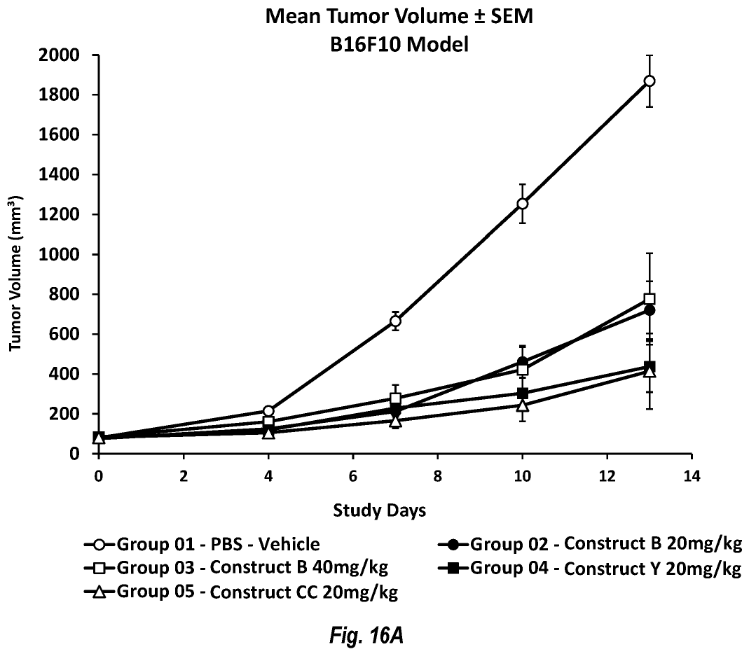

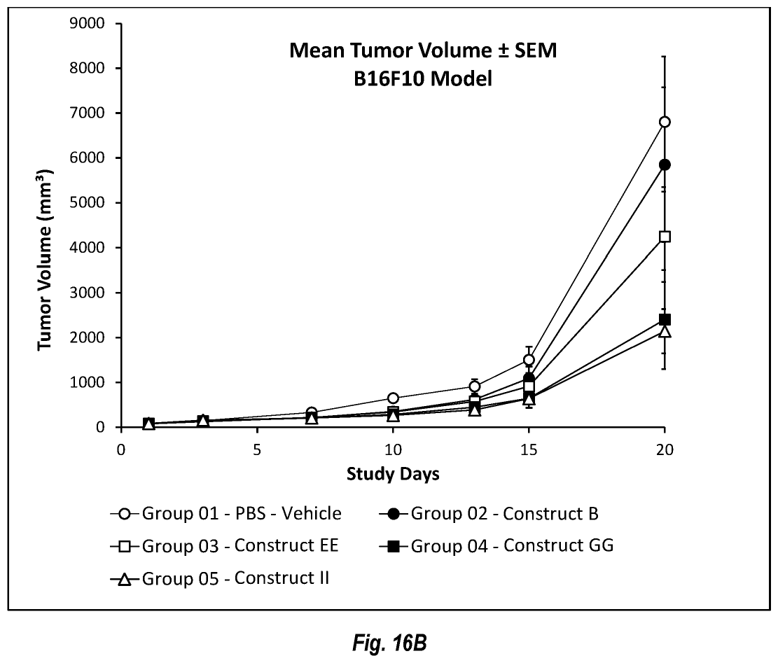

Tumor volumes were measured twice a week for the duration of the study. Mean tumor volume is shown in FIG. 16A. Anti-tumor activity was observed in all treatment groups, however the most robust tumor growth inhibition (TGI) was observed in the retention linker drugs Construct Y and Construct CC (77 and 7800 respectively) compared to ˜60% o TGI in Construct B treated groups (regardless of dose, Table 9).

TABLE 9

| Group | Drug | Dose | TGI (%) D 13 | P value |
|---|---|---|---|---|
| 2 | Construct B | 20 mg/kg | 61.43 | 0.0002 |
| 3 | Construct B | 40 mg/kg | 58.44 | 0.0019 |
| 4 | Construct Y | 20 mg/kg | 76.59 | 0.0001 |
| 5 | Construct CC | 20 mg/kg | 77.84 | 0.0002 |

P values represent unpaired t test (graphpad prism) between vehicle and Test article groups on Day 13.

In a second efficacy study in the same model, C57BL/6 mice were subcutaneously inoculated with B16F10 melanoma cells and when the average tumor volume reached on average 70-90 mm³, animals were randomized into 5 groups based on tumor volumes (n=6 mice per treatment group). Mice were dosed intravenously every 3 days (Q3D) for a total of 5 doses according to following design:

| | | | | Dosing | Dose | Dose |
|---|---|---|---|---|---|---|
| | | | Dose | Frequency | Level | Volume |
| Group | Treatment | N | Route | & Duration | (mg/kg) | (mL/kg) |
| 1 | PBS-Vehicle | 6 | IV | Q3D for 14 | NA | 5 |
| | | | | days (5 | | |
| | | | | doses) | | |
| 2 | Construct B | 6 | IV | Q3D for 14 | 10 | 5 |
| | | | | days (5 | | |
| | | | | doses) | | |
| 3 | Construct EE | 6 | IV | Q3D for 14 | 10 | 5 |
| | | | | days (5 | | |
| | | | | doses) | | |
| 4 | Construct GG | 6 | IV | Q3D for 14 | 10 | 5 |
| | | | | days (5 | | |
| | | | | doses) | | |
| 5 | Construct II | 6 | IV | Q3D for 14 | 10 | 5 |
| | | | | days (5 | | |
| | | | | doses) | | |

Tumor volumes were measured twice a week for the duration of the study up until Day 20, 7 days following the fifth dose. On Day 20, mice received an additional dose of drug, animals were sacrificed 24 hrs later and tissues and blood (processed to serum) were collected and stored at −80C for further testing. Mean tumor volume is shown in FIG. 16B. Only modest anti-tumor activity was observed with Construct B at 10 mg/kg in this aggressive model (27% TGI Day 15, Table 10). Strikingly, at equivalent dosage all retention linker IL-2 fusion drugs showed superior TGI (Table 10). In particular, collagen binding drugs Construct GG and Construct II (10 mg/kg dosing) showed robust tumor control similar to what was previously observed for Construct B at twice higher dose (57% TGI Day 15 Table 10 compared to 61% TGI Day 13 Table 9 respectively). Furthermore, after a dosing holiday of 7 days Construct B showed diminished efficacy whilst all retention linker drugs maintained similar levels of tumor control at Day 20. Collectively, FIGS. 16A-B demonstrate that retention linker IL-2 drugs have superior anti-tumor efficacy in a pre-clinical melanoma model. This is most likely due to the higher levels of both circulating drugs in serum and resident drug in TME, which can exert prolonged anti-tumor activity even after an extended dosing holiday.

TABLE 10

| Group | Drug | Dose | TGI (%) D 15 | TGI (%) D 20 |
|---|---|---|---|---|
| 2 | Construct B | 10 mg/kg | 27.43 | 14.02 |
| 3 | Construct EE | 10 mg/kg | 39.32 | 37.61 |
| 4 | Construct GG | 10 mg/kg | 57.18 | 64.70 |
| 5 | Construct II | 10 mg/kg | 57.68 | 68.52 |

### Example 18: IFN-γ Levels in Tumor Samples

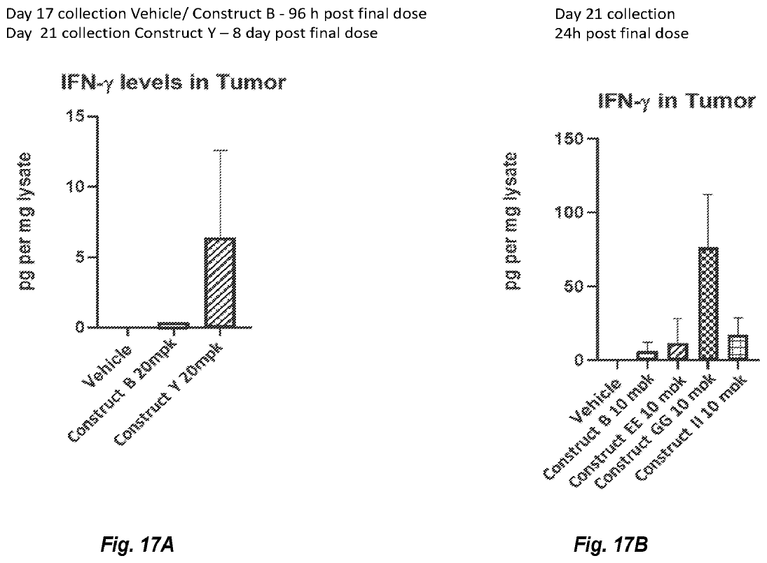

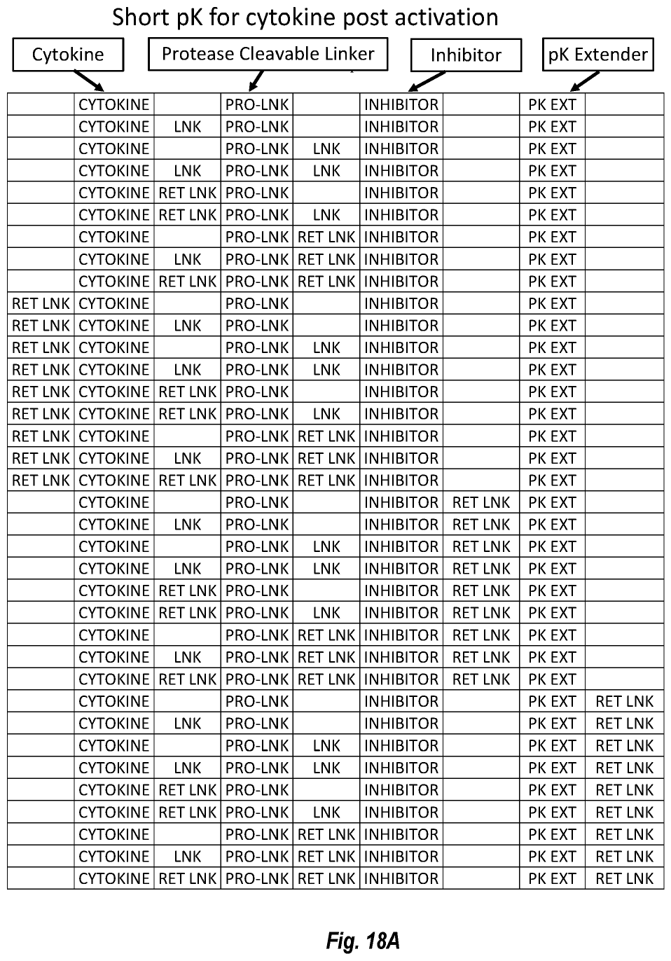

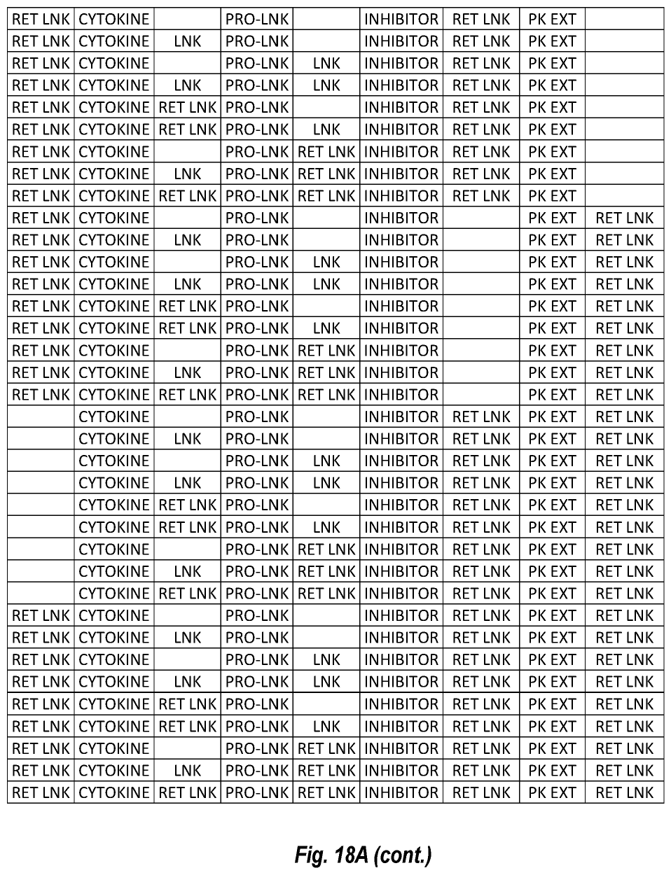

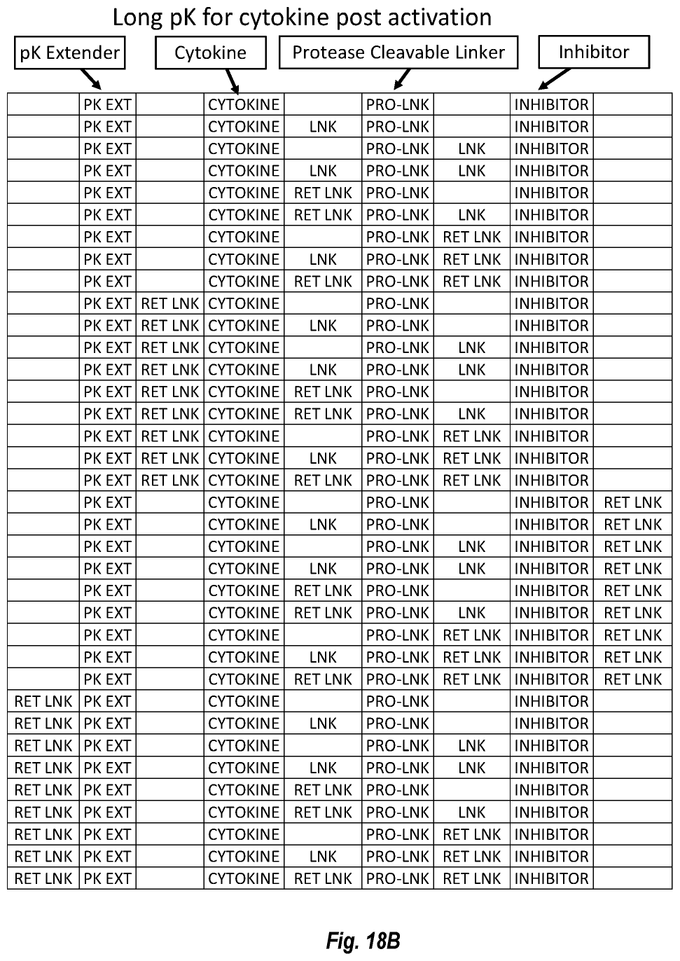

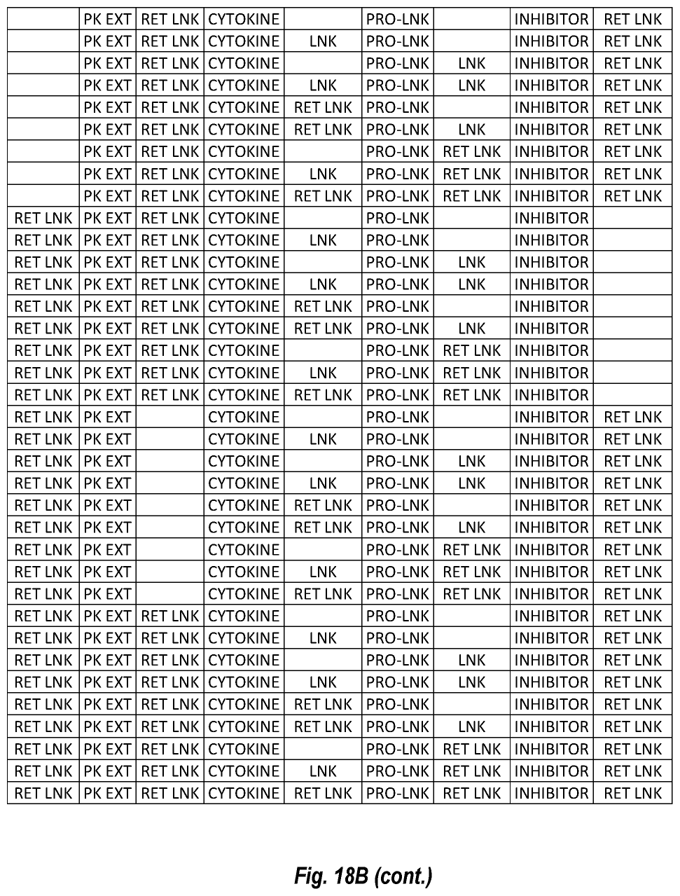

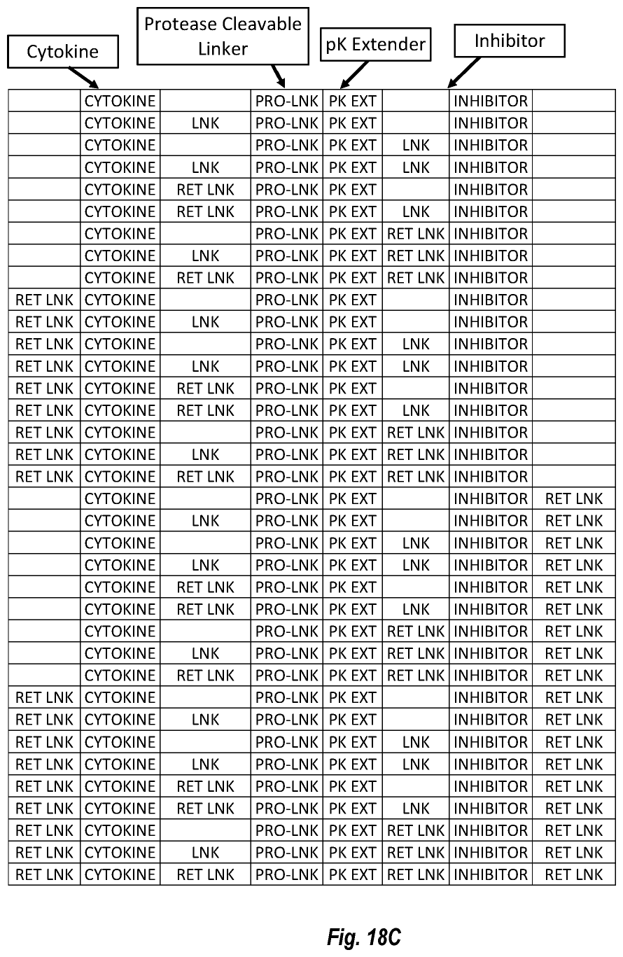

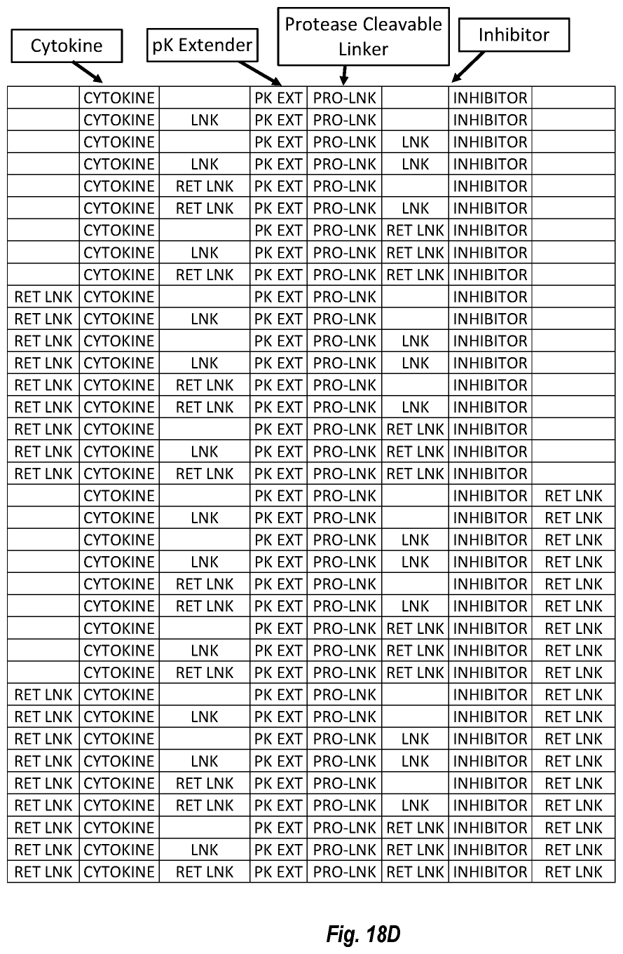

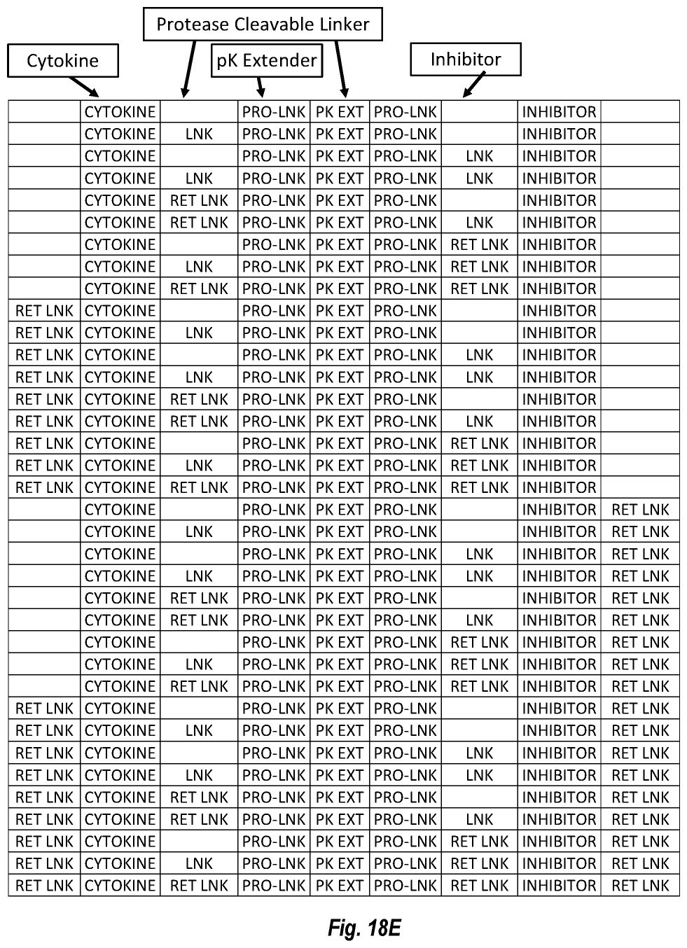

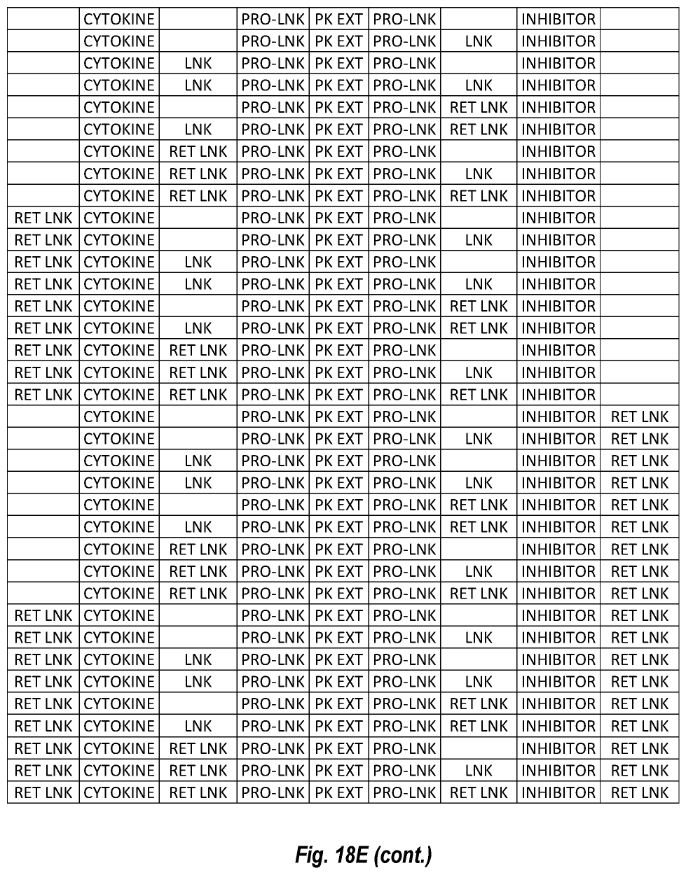

IFN-γ cytokine levels in tumor lysates (n=3 per group) were measured using a Luminex kit according to manufacturer's protocol (Invitrogen). Results were normalized to 1 mg of lysate and mean values are shown in FIG. 17A/B. Elevated levels of IFN-γ were measured in all retention linker TL-2 drug treated tumors compared to Construct B treated tumors. IFN-γ was undetectable in vehicle treated tumors.

We claim:

1. A protease-activated pro-cytokine comprising:
a cytokine polypeptide sequence;
a inhibitory polypeptide sequence capable of blocking an activity of the cytokine polypeptide sequence;
a linker between the cytokine polypeptide sequence and the inhibitory polypeptide sequence, the linker comprising a protease-cleavable polypeptide sequence; and
a targeting sequence, wherein the targeting sequence is configured to bind an extracellular matrix component, an integrin, or a syndecan; or is configured to bind, in a pH-sensitive manner, an extracellular matrix component, IgB (CD79b), an integrin, a cadherin, a heparan sulfate proteoglycan, a syndecan, or a fibronectin; or the targeting sequence comprises the sequence of any one of SEQ ID NOs: 180-662 or a variant having one or two mismatches relative to the sequence of any one of SEQ ID NOs: 180-662.

2. The protease-activated pro-cytokine of the immediately preceding claim, further comprising a pharmacokinetic modulator.

3. The protease-activated pro-cytokine of the immediately preceding claim, wherein the pharmacokinetic modulator comprises an immunoglobulin constant domain.

4. The protease-activated pro-cytokine of claim 2, wherein the pharmacokinetic modulator comprises an immunoglobulin Fc region.

5. The protease-activated pro-cytokine of the immediately preceding claim, wherein the immunoglobulin is a human immunoglobulin.

6. The protease-activated pro-cytokine of any one of claims 4-5, wherein the immunoglobulin is IgG.

7. The protease-activated pro-cytokine of the immediately preceding claim, wherein the IgG is IgG1, IgG2, IgG3, or IgG4.

8. The protease-activated pro-cytokine of claim 2, wherein the pharmacokinetic modulator comprises an albumin.

9. The protease-activated pro-cytokine of the immediately preceding claim, wherein the albumin is a serum albumin.

10. The protease-activated pro-cytokine of any one of claims 8-9, wherein the albumin is a human albumin.

11. The protease-activated pro-cytokine of claim 2, wherein the pharmacokinetic modulator comprises PEG.

12. The protease-activated pro-cytokine of claim 2, wherein the pharmacokinetic modulator comprises XTEN.

13. The protease-activated pro-cytokine of claim 2, wherein the pharmacokinetic modulator comprises CTP.

14. The protease-activated pro-cytokine of any one of claims 2-13, wherein the protease-cleavable polypeptide sequence is between the cytokine polypeptide sequence and the pharmacokinetic modulator.

15. The protease-activated pro-cytokine of any one of claims 2-13, wherein the pharmacokinetic modulator is between the cytokine polypeptide sequence and the protease-cleavable polypeptide sequence.

16. The protease-activated pro-cytokine of any one of the preceding claims, comprising a plurality of protease-cleavable polypeptide sequences.

17. The protease-activated pro-cytokine of the immediately preceding claim, wherein the cytokine polypeptide sequence is flanked by protease cleavable polypeptide sequences.

18. The protease-activated pro-cytokine of the immediately preceding claim, having the structure PM-CL-CY-CL-IN (from N- to C-terminus or from C- to N-terminus), where PM is the pharmacokinetic modulator, each CL independently is a protease-cleavable polypeptide sequence, CY is the cytokine polypeptide sequence, and IN is the inhibitory polypeptide sequence.

19. The protease-activated pro-cytokine of any one of the preceding claims, comprising the targeting sequence, wherein the targeting sequence is between the cytokine polypeptide sequence and the protease-cleavable polypeptide sequence or one of the protease-cleavable polypeptide sequences.

20. The protease-activated pro-cytokine of any one of the preceding claims, wherein the cytokine polypeptide sequence comprises a modification to prevent disulfide bond formation, and optionally otherwise comprises wild-type sequence.

21. The protease-activated pro-cytokine of any one of the preceding claims, wherein the cytokine polypeptide sequence has at least 80, 85, 90, 95, 97, 98, or 99 percent identity to the sequence of a wild-type cytokine polypeptide sequence or to a cytokine polypeptide sequence in Table 1.

22. The protease-activated pro-cytokine of the immediately preceding claim, wherein the cytokine polypeptide sequence is a wild-type cytokine polypeptide sequence.

23. The protease-activated pro-cytokine of any one of the preceding claims, wherein the cytokine polypeptide sequence is a monomeric cytokine, or wherein the cytokine polypeptide sequence is a dimeric cytokine polypeptide sequence comprising monomers that are associated covalently (optionally via a polypeptide linker) or noncovalently.

24. The protease-activated pro-cytokine of any one of the preceding claims, wherein the inhibitory polypeptide sequence comprises a cytokine-binding domain.

25. The protease-activated pro-cytokine of the immediately preceding claim, wherein the cytokine-binding domain is a cytokine-binding domain of a cytokine receptor or a cytokine-binding domain of a fibronectin.

26. The protease-activated pro-cytokine of claim 24, wherein the cytokine-binding domain is an immunoglobulin cytokine-binding domain.

27. The protease-activated pro-cytokine of the immediately preceding claim, wherein the immunoglobulin cytokine-binding domain comprises a light chain variable domain and a heavy chain variable domain that bind the cytokine.

28. The protease-activated pro-cytokine of any one of claims 26-27, wherein the immunoglobulin cytokine-binding domain is an scFv, Fab, or VHH.

29. The protease-activated pro-cytokine of any one of the preceding claims, wherein the protease-cleavable polypeptide sequence is recognized by a metalloprotease, a serine protease, a cysteine protease, an aspartate protease, a threonine protease, a glutamate protease, a gelatinase, an asparagine peptide lyase, a cathepsin, a kallikrein, a plasmin, a collagenase, a hK1, a hK10, a hK15, a stromelysin, a Factor Xa, a chymotrypsin-like protease, a trypsin-like protease, a elastase-like protease, a subtilisin-like protease, an actinidain, a bromelain, a calpain, a caspase, a Mir 1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, an ADAM 10, an ADAM17, an ADAM 12, an urokinase plasminogen activator (uPA), an enterokinase, a prostate-specific target (PSA, hK3), an interleukin-1b converting enzyme, a thrombin, a FAP (FAP-a), a dipeptidyl peptidase, or dipeptidyl peptidase IV (DPPIV/CD26), a type II transmembrane serine protease (TTSP), a neutrophil elastase, a proteinase 3, a mast cell chymase, a mast cell tryptase, or a dipeptidyl peptidase.

30. The protease-activated pro-cytokine of any one of the preceding claims, wherein the protease-cleavable polypeptide sequence comprises the sequence of any one of SEQ ID NOs: 700-741, or a variant having one or two mismatches relative to the sequence of any one of SEQ ID NOs: 700-741.

31. The protease-activated pro-cytokine of any one of the preceding claims, wherein the protease-cleavable polypeptide sequence is recognized by a matrix metalloprotease.

32. The protease-activated pro-cytokine of any one of the preceding claims, wherein the protease-cleavable polypeptide sequence is recognized by MMP-1.

33. The protease-activated pro-cytokine of any one of the preceding claims, wherein the protease-cleavable polypeptide sequence is recognized by MMP-2.

34. The protease-activated pro-cytokine of any one of the preceding claims, wherein the protease-cleavable polypeptide sequence is recognized by MMP-3.

35. The protease-activated pro-cytokine of any one of the preceding claims, wherein the protease-cleavable polypeptide sequence is recognized by MMP-7.

36. The protease-activated pro-cytokine of any one of the preceding claims, wherein the protease-cleavable polypeptide sequence is recognized by MMP-8.

37. The protease-activated pro-cytokine of any one of the preceding claims, wherein the protease-cleavable polypeptide sequence is recognized by MMP-9.

38. The protease-activated pro-cytokine of any one of the preceding claims, wherein the protease-cleavable polypeptide sequence is recognized by MMP-12.

39. The protease-activated pro-cytokine of any one of the preceding claims, wherein the protease-cleavable polypeptide sequence is recognized by MMP-13.

40. The protease-activated pro-cytokine of any one of the preceding claims, wherein the protease-cleavable polypeptide sequence is recognized by MMP-14.

41. The protease-activated pro-cytokine of any one of the preceding claims, wherein the protease-cleavable polypeptide sequence is recognized by more than one MMP.

42. The protease-activated pro-cytokine of any one of the preceding claims, wherein the protease-cleavable polypeptide sequence is recognized by two, three, four, five, six, or seven of MMP-2, MMP-7, MMP-8, MMP-9, MMP-12, MMP-13, and MMP-14.

43. The protease-activated pro-cytokine of any one of the preceding claims, wherein the protease-cleavable polypeptide sequence comprises the sequence of any one of SEQ ID NOs: 80-94 or a variant sequence having one or two mismatches relative to the sequence of any one of SEQ ID NOs: 80-90.

44. The protease-activated pro-cytokine of the immediately preceding claim, wherein the protease-cleavable polypeptide sequence comprises the sequence of SEQ ID NO: 80 or a variant sequence having one or two mismatches relative thereto.

45. The protease-activated pro-cytokine of any one of claims 1-43, wherein the protease-cleavable polypeptide sequence comprises the sequence of SEQ ID NO: 81 or a variant sequence having one or two mismatches relative thereto.

46. The protease-activated pro-cytokine of any one of claims 1-43, wherein the protease-cleavable polypeptide sequence comprises the sequence of SEQ ID NO: 82 or a variant sequence having one or two mismatches relative thereto.

47. The protease-activated pro-cytokine of any one of claims 1-43, wherein the protease-cleavable polypeptide sequence comprises the sequence of SEQ ID NO: 83 or a variant sequence having one or two mismatches relative thereto.

48. The protease-activated pro-cytokine of any one of claims 1-43, wherein the protease-cleavable polypeptide sequence comprises the sequence of SEQ ID NO: 84 or a variant sequence having one or two mismatches relative thereto.

49. The protease-activated pro-cytokine of any one of claims 1-43, wherein the protease-cleavable polypeptide sequence comprises the sequence of SEQ ID NO: 85 or a variant sequence having one or two mismatches relative thereto.

50. The protease-activated pro-cytokine of any one of claims 1-43, wherein the protease-cleavable polypeptide sequence comprises the sequence of SEQ ID NO: 86 or a variant sequence having one or two mismatches relative thereto.

51. The protease-activated pro-cytokine of any one of claims 1-43, wherein the protease-cleavable polypeptide sequence comprises the sequence of SEQ ID NO: 87 or a variant sequence having one or two mismatches relative thereto.

52. The protease-activated pro-cytokine of any one of claims 1-43, wherein the protease-cleavable polypeptide sequence comprises the sequence of SEQ ID NO: 88 or a variant sequence having one or two mismatches relative thereto.

53. The protease-activated pro-cytokine of any one of claims 1-43, wherein the protease-cleavable polypeptide sequence comprises the sequence of SEQ ID NO: 89 or a variant sequence having one or two mismatches relative thereto.

54. The protease-activated pro-cytokine of any one of claims 1-43, wherein the protease-cleavable polypeptide sequence comprises the sequence of SEQ ID NO: 90 or a variant sequence having one or two mismatches relative thereto.

55. The protease-activated pro-cytokine of any one of claims 1-43, wherein the protease-cleavable polypeptide sequence comprises the sequence of SEQ ID NO: 80-89 or 90.

56. The protease-activated pro-cytokine of any one of claims 1-43, wherein the protease-cleavable polypeptide sequence comprises the sequence of SEQ ID NO: 91.

57. The protease-activated pro-cytokine of any one of claims 1-43, wherein the protease-cleavable polypeptide sequence comprises the sequence of SEQ ID NO: 92.

58. The protease-activated pro-cytokine of any one of claims 1-43, wherein the protease-cleavable polypeptide sequence comprises the sequence of SEQ ID NO: 93.

59. The protease-activated pro-cytokine of any one of claims 1-43, wherein the protease-cleavable polypeptide sequence comprises the sequence of SEQ ID NO: 94.

60. The protease-activated pro-cytokine of any one of the preceding claims, wherein the targeting sequence comprises the sequence of any one of SEQ ID NOs: 180-662, or a variant having one or two mismatches relative to the sequence of any one of SEQ ID NOs: 180-662.

61. The protease-activated pro-cytokine of the immediately preceding claim, wherein the targeting sequence comprises the sequence of any one of SEQ ID NOs: 180-662.

62. The protease-activated pro-cytokine of any one of the preceding claims, wherein the targeting sequence binds to denatured collagen.

63. The protease-activated pro-cytokine of any one of claims 1-61, wherein the targeting sequence binds to collagen.

64. The protease-activated pro-cytokine of any one of claims 62-63, wherein the collagen is collagen I.

65. The protease-activated pro-cytokine of any one of claims 62-63, wherein the collagen is collagen II.

66. The protease-activated pro-cytokine of any one of claims 62-63, wherein the collagen is collagen III.

67. The protease-activated pro-cytokine of any one of claims 62-63, wherein the collagen is collagen IV.

68. The protease-activated pro-cytokine of any one of claims 1-61, wherein the targeting sequence binds to integrin.

69. The protease-activated pro-cytokine of the immediately preceding claim, wherein the integrin is one or more of α1β1 integrin, α2β1 integrin, α3β1 integrin, α4β1 integrin, α5β1 integrin, α6β1 integrin, α7β1 integrin, α9β1 integrin, α4β7 integrin, αvβ3 integrin, αvβ5 integrin, αIIbβ3 integrin, αIIIbβ3 integrin, αMβ2 integrin, or αIIbβ3 integrin.

70. The protease-activated pro-cytokine of any one of claims 1-61, wherein the targeting sequence binds to von Willebrand factor.

71. The protease-activated pro-cytokine of any one of claims 1-61, wherein the targeting sequence binds to IgB.

72. The protease-activated pro-cytokine of any one of claims 1-61, wherein the targeting sequence binds to heparin.

73. The protease-activated pro-cytokine of the immediately preceding claim, wherein the targeting sequence binds to heparin and a syndecan, a heparan sulfate proteoglycan, or an integrin, optionally wherein the integrin is one or more of α1β1 integrin, α2β1 integrin, α3β1 integrin, α4β1 integrin, α5β1 integrin, α6β1 integrin, α7β1 integrin, α9β1 integrin, α4β7 integrin, αvβ3 integrin, αvβ5 integrin, αIIbβ3 integrin, αIIIbβ3 integrin, αMβ2 integrin, or αIIbβ3 integrin.

74. The protease-activated pro-cytokine of any one of claims 72-73, wherein the syndecan is one of more of syndecan-1, syndecan-4, and syndecan-2(w).

75. The protease-activated pro-cytokine of any one of claims 1-61, wherein the targeting sequence binds to a heparan sulfate proteoglycan.

76. The protease-activated pro-cytokine of any one of claims 1-61, wherein the targeting sequence binds to a sulfated glycoprotein.

77. The protease-activated pro-cytokine of any one of claims 1-61, wherein the targeting sequence binds to hyaluronic acid.

78. The protease-activated pro-cytokine of any one of claims 1-61, wherein the targeting sequence binds to fibronectin.

79. The protease-activated pro-cytokine of any one of claims 1-61, wherein the targeting sequence binds to cadherin.

80. The protease-activated pro-cytokine of any one of the preceding claims, wherein the targeting sequence is configured to bind its target in a pH-sensitive manner.

81. The protease-activated pro-cytokine of the immediately preceding claim, wherein the targeting sequence has a higher affinity for its target at a pH below normal physiological pH than at normal physiological pH, optionally wherein the pH below normal physiological pH is below 7, or below 6.

82. The protease-activated pro-cytokine of the immediately preceding claim, wherein the targeting sequence has a higher affinity for its target at a pH in the range of 5-7, e.g., 5-5.5, 5.5-6, 6-6.5, or 6.5-7, than at normal physiological pH.

83. The protease-activated pro-cytokine of any one of the preceding claims, wherein the targeting sequence comprises one or more histidines, e.g., 1, 2, 3, 4, 5, 6, 7, 8, 9, or 10 histidines.

84. The protease-activated pro-cytokine of any one of the preceding claims, wherein the targeting sequence comprises the sequence of any one of SEQ ID NOs: 641-662, or a variant having one or two mismatches relative to the sequence of any one of SEQ ID NOs: 641-662.

85. The protease-activated pro-cytokine of the immediately preceding claim, wherein the targeting sequence comprises the sequence of any one of SEQ ID NOs: 641-662.

86. The protease-activated pro-cytokine of any one of claims 80-86, wherein the targeting sequence is configured to bind, in a pH-sensitive manner, an extracellular matrix component, IgB (CD79b), an integrin, a cadherin, a heparan sulfate proteoglycan, a syndecan, or a fibronectin.

87. The protease-activated pro-cytokine of the immediately preceding claim, wherein the extracellular matrix component is hyaluronic acid, heparin, heparan sulfate, or a sulfated glycoprotein.

88. The protease-activated pro-cytokine of claim 86, wherein the targeting sequence is configured to bind a fibronectin in a pH-sensitive manner.

89. The protease-activated pro-cytokine of any one of the preceding claims, wherein the cytokine polypeptide sequence is an interleukin polypeptide sequence.

90. The protease-activated pro-cytokine of any one of the preceding claims, wherein the cytokine polypeptide sequence is capable of binding a receptor comprising CD132.

91. The protease-activated pro-cytokine of any one of the preceding claims, wherein the cytokine polypeptide sequence is capable of binding a receptor comprising CD122.

92. The protease-activated pro-cytokine of any one of the preceding claims, wherein the cytokine polypeptide sequence is capable of binding a receptor comprising CD25.

93. The protease-activated pro-cytokine of any one of the preceding claims, wherein the cytokine polypeptide sequence is an IL-2 polypeptide sequence.

94. The protease-activated pro-cytokine of the immediately preceding claim, wherein the IL-2 polypeptide sequence has at least 80, 85, 90, 95, 97, 98, or 99 percent identity to the sequence of any one of SEQ ID NOs: 1-4.

95. The protease-activated pro-cytokine of the immediately preceding claim, wherein the IL-2 polypeptide sequence comprises the sequence of any one of SEQ ID NOs: 1-4.

96. The protease-activated pro-cytokine of any one of claims 93-95, wherein the IL-2 polypeptide sequence is a human TL-2 polypeptide sequence.

97. The protease-activated pro-cytokine of the immediately preceding claim, wherein the IL-2 polypeptide sequence comprises the sequence of SEQ ID NO: 1.

98. The protease-activated pro-cytokine of any one of claims 93-95, wherein the IL-2 polypeptide sequence comprises the sequence of SEQ ID NO: 2.

99. The protease-activated pro-cytokine of any one of claims 93-98, wherein the inhibitory polypeptide sequence comprises an IL-2 binding domain of an IL-2 receptor (IL-2R).

100. The protease-activated pro-cytokine of the immediately preceding claim, wherein the inhibitory polypeptide sequence comprises an amino acid sequence having at least 80, 85, 90, 95, 97, 98, or 99 percent identity to the sequence of any one of SEQ ID NOs: 10-19.

101. The protease-activated pro-cytokine of the immediately preceding claim, wherein the IL-2R is a human IL-2R.

102. The protease-activated pro-cytokine of any one of claims 93-98, wherein the inhibitory polypeptide sequence comprises an IL-2-binding immunoglobulin domain.

103. The protease-activated pro-cytokine of any one of claims 93-98, wherein the IL-2-binding immunoglobulin domain is a human IL-2-binding immunoglobulin domain.

104. The protease-activated pro-cytokine of the immediately preceding claim, wherein the IL-2-binding immunoglobulin domain comprises a VL region comprising hypervariable regions (HVRs) HVR-1, HVR-2, and HVR-3 having the sequences of SEQ ID NOs: 33, 34, and 35, respectively, and a VH region comprising HVR-1, HVR-2, and HVR-3 having the sequences of SEQ ID NOs: 36, 37, and 38, respectively.

105. The protease-activated pro-cytokine of any one of claims 102-104, wherein the IL-2-binding immunoglobulin domain comprises a VL region comprising an amino acid sequence having at least 80, 85, 90, 95, 97, 98, or 99 percent identity to the sequence of SEQ ID NO: 32 and a VH region comprising an amino acid sequence having at least 80, 85, 90, 95, 97, 98, or 99 percent identity to the sequence of SEQ ID NO: 33.

106. The protease-activated pro-cytokine of the immediately preceding claim, wherein the IL-2-binding immunoglobulin domain comprises a VL region comprising the sequence of SEQ ID NO: 32 and a VH region comprising the sequence of SEQ ID NO: 33.

107. The protease-activated pro-cytokine of any one of claims 102-104, wherein the IL-2-binding immunoglobulin domain is an scFv.

108. The protease-activated pro-cytokine of the immediately preceding claim, wherein the IL-2-binding immunoglobulin domain comprises an amino acid sequence having at least 80, 85, 90, 95, 97, 98, or 99 percent identity to the sequence of SEQ ID NO: 30 or 31.

109. The protease-activated pro-cytokine of the immediately preceding claim, wherein the IL-2-binding immunoglobulin domain comprises the sequence of SEQ ID NO: 30 or 31.

110. The protease-activated pro-cytokine of claim 1, comprising the sequence of any one of SEQ ID NOs: 803-852.

111. A pharmaceutical composition comprising the protease-activated pro-cytokine of any one of the preceding claims.

112. The protease-activated pro-cytokine or pharmaceutical composition of any one of the preceding claims, for use in therapy.

113. The protease-activated pro-cytokine or pharmaceutical composition of any one of the preceding claims, for use in treating a cancer.

114. A method of treating a cancer, comprising administering the protease-activated pro-cytokine or pharmaceutical composition of any one of the preceding claims to a subject in need thereof.

115. Use of the protease-activated pro-cytokine or pharmaceutical composition of any one of claims 1-110 for the manufacture of a medicament for treating cancer.

116. The method, use, or protease-activated pro-cytokine for use of any one of claims 113-115, wherein the cancer is a solid tumor.

117. The method, use, or protease-activated pro-cytokine for use of the immediately preceding claim, wherein the solid tumor is metastatic and/or unresectable.

118. The method, use, or protease-activated pro-cytokine for use of any one of claims 113-117, wherein the cancer is a PD-L1-expressing cancer.

119. The method, use, or protease-activated pro-cytokine for use of any one of claims 113-118, wherein the cancer is a melanoma, a colorectal cancer, a breast cancer, a pancreatic cancer, a lung cancer, a prostate cancer, an ovarian cancer, a cervical cancer, a gastric or gastrointestinal cancer, a lymphoma, a colon or colorectal cancer, an endometrial cancer, a thyroid cancer, or a bladder cancer.

120. The method, use, or protease-activated pro-cytokine for use of any one of claims 113-119, wherein the cancer is a microsatellite instability-high cancer.

121. The method, use, or protease-activated pro-cytokine for use of any one of claims 113-120, wherein the cancer is mismatch repair deficient.

122. A nucleic acid encoding the protease-activated pro-cytokine of any one of claims 1-110.

123. An expression vector comprising the nucleic acid of claim 121.

124. A host cell comprising the nucleic acid of claim 121 or the vector of claim 122.

125. A method of producing a protease-activated pro-cytokine, comprising culturing the host cell of claim 124 under conditions wherein the protease-activated pro-cytokine is produced.

126. The method of the immediately preceding claim, further comprising isolating the protease-activated pro-cytokine.

127. A method of boosting T regulatory cells and/or reducing inflammation or autoimmune activity, comprising administering the protease-activated pro-cytokine of any one of claims 1-110 to an area of interest in a subject, e.g., an area of inflammation in the subject.

128. A method of treating an inflammatory or autoimmune disease or disorder in a subject, comprising administering the protease-activated pro-cytokine of any one of claims 1-110 to an area of interest in a subject, e.g., an area of inflammation or autoimmune activity in the subject.
